(19) United States
(12) Patent Application Publication
(10) Pub. No.: US 2025/0207166 A1
(43) Pub. Date: Jun. 26, 2025

(54) BIOSYNTHESIS

(71) Applicant: PLANT BIOSCIENCE LIMITED, Norwich Norfolk (GB)

(72) Inventors: Anne OSBOURN, Norwich Norfolk (GB); James REED, Norwich Norfolk (GB); Anastasia ORME, Norwich Norfolk (GB); Laetitia MARTIN, Norwich Norfolk (GB); Charlotte OWEN, Norwich Norfolk (GB)

(21) Appl. No.: 18/849,851

(22) PCT Filed: Dec. 23, 2022
(86) PCT No.: PCT/GB2022/053385

§ 371 Date: Sep. 23, 2024

(30) Foreign Application Priority Data
Mar. 25, 2022 (GB) 2204252.7
Jun. 29, 2022 (GB) 2209590.5

(51) Int. Cl. C12P 19/56; A61K 39/00; A61K 39/39; C12N 5/04; C12N 9/00; C12N 9/04; C12N 9/10

(52) U.S. Cl. CPC C12P 19/56; A61K 39/39; C12N 5/04; C12N 9/0006; C12N 9/1037; C12N 9/1051; C12N 9/93; A61K 2039/55577; C12Y 203/01074; C12Y 204/01017; C12Y 602/01003

(57) ABSTRACT

The present invention relates to a biosynthetic route to the QS-21 and QS-18 molecules including the C-18 acyl chain and precursors thereof, as well as enzymes involved, the products produced and uses of the products.

The present invention relates to a biosynthetic route to the QS-21 and QS-18 molecules including the C-18 acyl chain and precursors thereof, as well as enzymes involved, the products produced and uses of the products.

### BACKGROUND

QS-21 is a natural saponin extract from the bark of the Chilean ‘soapbark’ tree, Quillaja saponaria. QS-21 extract was originally identified as a purified fraction of a crude bark extract of Quillaja Saponaria Molina obtained by RP-HPLC purification (peak 21) (Kensil et al. 1991). QS-21 extract, or fraction, comprises several distinct saponin molecules. Two principal isomeric molecular constituents of the fraction were reported (Ragupathi et al. 2011). Both incorporate a central triterpene core, to which a branched trisaccharide is attached at the triterpene C-3 oxygen functionality, and a linear tetrasaccharide is linked to the triterpene C-28 carboxylate group. The isomeric components differ in the constitution of the terminal sugar residue of the tetrasaccharide, in which the major and minor compounds incorporate either an apiose (65%) or a xylose (35%) carbohydrate, respectively (see FIG. 1, where R₁ is a β-D-xylose). A fourth component within the saponin structure is a glycosylated pseudo-dimeric acyl chain attached to the fucose residue via a hydrolytically labile ester linkage. This 18-carbon acyl chain terminates in an arabinofuranose sugar and is important to the immune enhancement activity of the molecule (Marciani, 2018).

Likewise, QS-18 extract, or fraction, was originally identified as a purified fraction of a crude bark extract of Quillaja Saponaria Molina obtained by RP-HPLC purification (peak 18) (Kensil et al. 1991). As compared with the structure of QS-21, QS-18 differs from the structure of QS-21 in that a glucose residue is incorporated at the C-3 position of the rhamnose residue of the linear tetrasaccharide at the C-28 position of quillaic acid (FIG. 2).

Saponins from Q. saponaria, including the QS-21 and QS-18 fractions, have been known for many years to have potent immunostimulatory properties, although varying in haemolytic activity and toxicity (Kensil, et al. 1991), capable of enhancing antibody production and specific T-cell responses. These properties have resulted in the development of Quillaja saponin-based adjuvants for vaccines. Of particular note, the ASO1 adjuvant features a liposomal formulation of QS-21 and 3-O-desacyl-4′-monophosphoryl lipid A (the production of which is described in WO2013/041572) and is currently licenced in vaccine formulations for diseases including shingles (Shingrix) and malaria (Mosquirix).

The present invention describes methods to synthesise the two principal isomeric constituents of the QS-21 and QS-18 fractions, and precursors and variants thereof (e.g. rhamnose residue at the triterpene C-3 position instead of a xylose residue), other than by purification from the native Q. saponaria plant, as well as the resulting product, which is useful as an adjuvant in vaccine formulations. The present invention also relates to enzymes involved in the methods, vectors, host cells and biological systems to produce the product.

### BRIEF DESCRIPTION OF THE INVENTION

The present invention relates, in particular, to the addition of the C-18 acyl chain to the C-4 position of the fucose residue of the C-28 linear tetrasaccharide of a molecule comprising a quillaic acid backbone (QA), and the resulting QA derivatives (collectively referred to as QA*-F*-C18-A). The invention includes the biosynthetic preparation of QA*-F*-C18-A as well as precursors thereof, such as, for example, QA*-F*-C9, or QA*-F*-C18. The invention also relates to the uses of QA*-F*-C18-A, the two principal isomeric constituents of the QS-21 fraction (QA-TriX-FRXX-C18-A and QA-TriX-FRXA-C18-A), and precursors thereof.

The present invention also relates to the addition of a D-glucose residue to the C-3 position of the rhamnose residue of the C-28 linear tetrasaccharide of a molecule comprising a QA*-F*-C18-A backbone or a molecule comprising a QA*-F* backbone, and the resulting QA derivatives (collectively referred to as “QA*-F*-C18-A-G”). When the D-glucose residue is added to a molecule comprising QA*-F* the C-18 acyl chain is then subsequently added.

### QA Synthesis

QA derives from the simple triterpene β-amyrin, which is synthesised through cyclisation of the universal linear precursor 2,3-oxidosqualene (OS) by an oxidosqualene cyclase (OSC). This biosynthesis is known in the art, such as in WO2019/122259, the content of which is incorporated by reference. This β-amyrin scaffold is further oxidised with a carboxylic acid, alcohol and aldehyde at the C-28, C-16a and C-23 positions, respectively, by a series of three cytochrome P450 monooxygenases, forming quillaic acid (QA). The OSC and C-28, C-16a and C-23 oxidases are referred to herein as QsbAS (β-amyrin synthase), QsCYP716-C-28, QsCYP716-C-16a and QsCYP714-C-23 oxidases, respectively.

### C-3 Branched Trisaccharide Synthesis

The branched trisaccharide chain is initiated with a D-glucopyranuronic acid (D-GlcpA) residue attached with a β-linkage at the C-3 position of the QA backbone. The D-GlcpA residue has two sugars linked to it: a D-galactopyranose (D-Galp) residue attached with a β-1,2-linkage and a D-xylopyranose (D-Xylp) residue or an L-rhamnopyranose (L-Rhap) residue (R₁ in FIG. 1), attached with a β-1,3-linkage or an α-1,3-linkage, respectively.

Seven enzymes have been identified that have activity relevant to the production of this QA 3-0 trisaccharide (QA-TriX or QA-TriR), such as reported in WO 2020/260475. These include two functionally-redundant glucuronosyltransferases, CSL1 and CsIG2, that can add the initial β-D-glucopyranuronic acid residue at the C-3 position of QA; a galactosyltransferase, Qs-3-O-GaIT, that adds the β-D-galactopyranose residue to the C-2 position of the β-D-glucopyranuronic acid residue; a xylosyltransferase, Qs_0283870, that adds the β-D-xylopyranose residue at the C-3 position of the β-D-glucopyranuronic acid residue; two rhamnosyltransferases, DN20529_c0_g2_i8 and Qs_0283850, that add an α-L-rhamnopyranose residue at the C-3 position of the β-D-glucopyranuronic acid residue; and a bifunctional enzyme, Qs-3-O-RhaT/XylT that can add either a β-D-xylopyranose residue or a α-L-rhamnopyranose residue to the C-3 position of the β-D-glucopyranuronic acid residue.

For simplicity, throughout the application, a QA derivative including the branched trisaccharide at position C-3 may be designated as “QA-TriX”, “QA-TriR” or “QA-Tri(X/R)” (see the Abbreviation list herein).

### C-28 Linear Tetrasaccharide Synthesis

The linear tetrasaccharide chain (F*) is initiated by attaching D-fucose residue with a β-linkage at the C-28 position of the QA backbone (F). This step is followed by attaching A L-rhamnose residue with an α-linkage to the fucose residue (FR), then attaching a D-xylose residue with a β-linkage to the rhamnose residue (FRX). Finally, a D-xylose residue or D-apiose residue is attached with a β-linkage to the xylose residue (FRXX and FRXA, respectively.

Ten enzymes have been identified that have activity relevant to the production of the C-28 linear tetrasaccharide, such as reported in PCT/EP2021/087323 (GB2020623.1). These include Qs-28-O-FucT (SEQ ID NO 2), which transfers a D-fucose residue with a β-linkage to the C-28 position of the QA backbone; Qs-28-O-RhaT (SEQ ID NO 4) which transfers a L-rhamnose residue to a D-fucose residue; Qs-28-O-XyIT3 (SEQ ID NO 6) which transfers a D-xylose residue to a L-rhamnose residue; Qs-28-O-XyIT4 (SEQ ID NO 8) which attaches a β-D-xylose residue to a β-D-xylose residue; Qs-28-O-ApiT4 (SEQ ID NO 10) which attaches a β-D-apiose residue to a β-D-xylose residue. An oxidoreductase enzyme QsFucSyn (SEQ ID No. 12), and QsFucSyn-Like enzymes, such as QsFSL-1 (SEQ ID No. 48), QsFSL-2 (SEQ ID No 50) or SoFSL-1 (SEQ ID No 52) which may increase the production of UDP-D-fucose and/or reduce the 4-keto group of 4-keto-6-deoxy-glucose after it has been added to the QA backbone have also been identified that have activity relevant to the production of the C-28 linear tetrasaccharide. A UDP-apiose/UDP-xylose synthase enzyme QsAXS1 (SEQ ID NO 14) which enhances the activity of an apiosyltransferase by increasing the availability of the UDP-α-D-apiose has also been identified.

### Synthesis of the Acyl Chain Attached to the C-28 Linear Tetrasaccharide

The present invention describes, for the first time, the biosynthetic route for the addition of the acyl chain to the D-fucose residue of the linear tetrasaccharide at the C-28 position of the QA backbone and the resulting QA derivatives, such as, for example, QA*-F*-C9 and QA*-F*-C18.

### Addition of a D-Glucose Residue to the Rhamnose Residue of the C-28 Linear Tetrasaccharide

The present invention also describes for the first time, the biosynthetic route for the addition of a D-glucose residue at the C-3 position of the rhamnose residue of the linear tetrasaccharide at the C-28 position of the QA backbone and the resulting QA derivatives, such as, for example, QA*-F*-C18-A-G.

Accordingly, the present invention provides methods for making the two principal isomeric constituents of the QS-21 and QS-18 fractions and precursors thereof (e.g. QA*-F*-G, QA*-F*-C9-G, QA*-F*-C18-G, QA*-F*-C9, QA*-F*-C18, QA*-F*-C18-A and QA*-F*-C18-A-G), enzymes used in the methods, polynucleotides encoding the enzymes, vectors comprising the polynucleotides, host cells transformed with the vectors and uses of the two principal isomeric constituents of the QS-21 and QS-18 fractions and precursors thereof (e.g. QA*-F*-G, QA*-F*-C9-G, QA*-F*-C18-G, QA*-F*-C9, QA*-F*-C18, QA*-F*-C18-A and QA*-F*-C18-A-G) as adjuvant.

### DESCRIPTION OF THE FIGURES

FIG. 1 shows the structure of the two principal isomeric constituents of the QS-21 fraction. The core backbone is formed from the triterpene quillaic acid (QA). The C-3 position features a branched trisaccharide consisting of β-D-glucopyranuronic acid (D-GlcpA) residue, β-D-galactopyranose (D-Galp) residue and β-D-xylopyranose (D-xylp) residue at (R₁). A α-L-rhamnopyranose (L-rhap) residue at (R₁) was also used in this work. The C-28 position features a linear tetrasaccharide consisting of β-D-fucopyranose (D-fucp) residue, α-L-rhamnopyranose residue, β-D-xylopyranose residue and either a terminal β-D-apiofuranose (D-apif) residue or β-D-xylopyranose residue (R₂). The D-fucose residue also features an 18-carbon acyl chain which terminates with α-L-arabinofuranose (L-Araf) residue.

FIG. 2 shows the structure of QS-18 and QS-21. As compared with the structure of QS-21, QS-18 incorporates a D-glucose residue at the C-3 position of the rhamnose residue of the C-28 linear tetrasaccharide.

FIG. 3 shows the proposed biosynthetic pathway of the acyl chain, predicted based on the structure of the acyl chain. The hypothesized enzymes catalysing the listed reactions are written next to the solid arrows. To note that the order of the keto reductions is randomly represented. BCAT, branched-chain amino acid aminotransferase; BCKDH, branched-chain α-ketoacid dehydrogenase complex; TE, thioesterase; CCL, carboxyl coenzyme A (CoA) ligase; PKSIII/ChS, type III polyketide synthase/chalcone synthase-like; KR, keto reductase; BAHD, acyltransferase; ArafT, arabinofuranosyl transferase.

FIG. 4 shows the efficacy of the Q. saponaria CCL (QsCCLs) enzyme candidates to generate isobutyric acid to form PIBP (phlorisobutyrophenone) and 2-methylbutyryl-CoA and/or isovaleryl-CoA to form PMBP (phlormethylbutanophenone) and/or PIVP (phlorisovalerophenone). A. PIBP levels in yeast strains expressing the hop valerophenone synthase (HIVPS) and the QsCCL enzyme candidates. The hop CCL genes (HICCL2 and HICCL4) either by themselves or co-expressed with HIVPS were added as controls. The data were normalized on absorbance and naringenin, n=3. B, PMBP/PIVP production of yeast strains expressing HIVPS and the QsCCL enzyme candidates. The hop CCL genes (HICCL2 and HICCL4) either by themselves or co-expressed with HIVPS were added as controls. The QsCCL enzyme candidates were also tested individually without any PKSIII enzyme (data not shown): no PMBP/PIVP was detected in these samples. The data were normalized on absorbance and naringenin, n=3. C, The QsCCL enzyme candidates were tested in Nicotiania benthamiana leaves, n=4. The data were normalized on dry weight and naringenin. Dashed bars, hop CCL controls; solid bars, QsCCL enzyme candidates; RA, relative amounts.

FIG. 5 shows the efficacy of the QsCCLs enzyme candidates to generate 2-methylbutyryl-CoA by measuring the QA-TriR-FRX-C9 downstream product. The gene sets necessary to produce QA-TriR-FRX were co-expressed in N. benthamiana along with AstHMGR, the six ChS candidates, KR11, KR23′ and DMOT9. The QsCCLs were co-expressed with them as relevant. The data were normalized on dry weight and the digitoxin internal standard, n=4.

FIG. 6 shows the amount of 2-methylbutyryl-CoA and/or isovaleryl-CoA produced by the QsCCL enzyme candidates in yeast (A) and N. benthamiana (B). The data (n=3) were normalized on absorbance in yeast and on fresh weight in N. benthamiana (n=4). RA, relative amounts.

FIG. 7 shows the amount of isobutyryl-CoA produced by the QsCCL enzyme candidates in yeast (A) and N. benthamiana (B). The data (n=3) were normalized on absorbance in yeast and on fresh weight in N. benthamiana (n=4). RA, relative amounts.

FIG. 8 shows the proposed chemical reactions catalysed by putative CCL and ChS enzymes to form (S)-6-methyl-3,5-dioxooctanoyl-CoA. It was hypothesized that a CCL enzyme ligates a CoA to (S)-2-methylbutyric acid to form 2-methylbutyryl-CoA. A ChS enzyme would use that as a substrate and extend its chain by successively adding two molecules of malonyl-CoA.

FIG. 9 shows 2-methylbutyryl-CoA and malonyl-CoA substrate depletion by the Q. saponaria ChS enzyme candidates. A, 2-methylbutyryl-CoA substrate depletion in yeast, n=3. B, 2-methylbutyryl-CoA substrate depletion in N. benthamiana, n=4. The hop CCL4 enzyme was expressed to provide enough 2-methylbutyryl-CoA to test the ChS enzyme candidates. Although the LC/MS method used here does not distinguish between 2-methylbutyryl-CoA and isovaleryl-CoA, HICCL4 does not use isovaleric acid as a substrate (Xu et al. 2013), implying that these results mainly represent the levels of 2-methylbutyryl-CoA. C, Malonyl-CoA substrate depletion in N. benthamiana, n=4. IV-CoA/2 MB-CoA (for isovaleric-CoA/2-methylbutyryl-CoA) and malonyl-CoA peak areas were normalized on fresh weights.

FIG. 10 shows the analysis of the ChSs downstream product QA-TriR-FRX-C9. The ChS enzyme candidates were transiently co-expressed in N. benthamiana leaves with the genes required to produce QA-TriR-FRX and 3CCL, KR11, KR23′ and DMOT9. “No ChS” is the negative control that does not contain any of the ChS candidates. “ChSA-F” contains all 6 ChS candidates. The samples were normalized on dry weight and the internal standard digitoxin, n=4.

FIG. 11 shows the effect of increased Agrobacterium concentration on downstream product formation. The ChS enzyme candidates were transiently co-expressed in N. benthamiana leaves with the genes required to produce QA-TriR-FRX and 3CCL, KR11, KR23′ and DMOT9. “No ChS” is the negative control that does not contain any of the ChS candidates. “ChSA-F” contains all six ChS candidates. The total concentration of the Agrobacterium strains varies between samples: it is of one-fold for ChSC and six-fold for ChSA-F and ChSC×6. The samples were normalized on dry weight and the internal standard digitoxin, n=4.

FIG. 12 shows that ChSE provides effective production of QA-TriR-FRX-C9. The ChS enzyme candidates were transiently co-expressed in N. benthamiana leaves with the genes required to produce QA-TriR-FRX and 3CCL, KR11, KR23′ and DMOT9. “No ChS” is the negative control that does not contain any of the ChS candidates. ChSA-F contains all 6 ChS candidates. The other conditions contain five ChS candidates with the missing one being indicated. The samples were normalized on dry weight and the internal standard digitoxin, n=4.

FIG. 13 shows the ChSs efficiency by pairs. The ChS enzyme candidates were transiently co-expressed in N. benthamiana leaves with the genes required to produce QA-TriR-FRX and 3CCL, KR11, KR23′ and DMOT9. Pairs of ChSs are tested as indicated and compared to the efficiency given by the six ChS together (ChSA-F), by all but ChSE (ChSA,B,C,D,F) and by ChSE by itself. The samples were normalized on dry weight and the internal standard digitoxin, n=4.

FIG. 14 shows the testing when adding a third ChS to the ChSD/ChSE pair. The ChS enzyme candidates were transiently co-expressed in N. benthamiana leaves with the genes required to produce QA-TriR-FRX and 3CCL, KR11, KR23′ and DMOT9. The samples were normalized on dry weight and the internal standard digitoxin, n=4.

FIG. 15 shows the analysis of KR23′ and KR11. A, KR23′ and KR11 functions were tested with the gene sets required to produce QA-TriX-FRX along with 3CCL, ChSA-F and DMOT9. The presence of KR11 allowed for the formation of QA-TriX-FRX-C9, but the product formation was boosted when both KRs were expressed. B, KR23′ and KR11 functions were tested with the gene sets required to produce QA-TriX-FRX along with 3CCL, ChSA-F, DMOT9 and DMOT4. The presence of KR11 led to the formation of QA-TriX-FRX-C18, but the product formation was greater when both KRs were present. C, Hypothesized enzymatic reactions. The screened masses of the hydrogen adducts in negative mode are as follows: QA-TriX-FRX-C9 m/z=1551.7146-1551.7302. QA-TriX-FRX-C18 m/z=1723.8238-1723.8410. The controls contained all the gene sets previously listed but not the KRs.

FIG. 16 DMOT9 and DMOT4 functions were tested with the gene sets required to produce QA-TriX-FRXX along with 3CCL, the six ChS enzyme candidates, KR11 and KR23′. A, QA-TriX-FRXX-C9 was synthesised in DMOT9 presence. B, DMOT9 and DMOT4 needed to be co-infiltrated to produce QA-TriX-FRXX-C18. C, Shows a summary of the activities of DMOT4 and DMOT9. The screened masses of the hydrogen adducts in negative mode are as follows: QA-TriX-FRXX-C9 m/z=1683.7563-1683.7731. QA-TriX-FRXX-C18 m/z=1855.8653-1855.8839. The controls included the gene sets previously stated but not the DMOTs.

FIG. 17 shows that the UGT-Ls were able to transfer an arabinofuranose to the acyl chain. A UGT-L-long and UGT-L-short were tested on the QA-TriR-FRX-C18 substrate. The mixes contained the gene sets required to make QA-TriR-FRX, 3CCL, ChSB, ChSC, ChSE, ChSF, KR11, KR23′, DMOT4 and DOMT9. UGT-L-long and UGT-L-short were added where specified. B, UGT-L-long was able to add an arabinofuranose to QA-TriX-FRXX-C18. The six ChSs were used here. C, UGT-L-long added an arabinofuranose to the C9 acyl chain. The six ChSs were also used in this mix. The screened masses of the hydrogen adducts in negative mode are as follows: QA-TriR-FRX-C18-A m/z=1869.8810-1869.8996, QA-TriX-FRXX-C18-A m/z=1987.9070-1987.9268, QA-TriX-FRXX-C9-A m/z=1815.7979-1815.8161.

FIG. 18 shows the in vitro transfer of Araf onto QA-TriX-FRXA-C18. The in vitro reactions were composed of purified QA-TriX-FRXA-C18, UDP-β-L-arabinofuranose and purified UGT-L-long-His. CAD chromatograms of the reaction products are shown. Upper, no Enzyme control; Middle, His-tagged UGT-L long added; Lower, semi-pure QS-21 for the reference (fraction 8 described in Materials and Methods). (1) QA-TriX-FRXA-C18, (2) QA-TriX-FRXA-C18-A. (3) semi-pure QS-21 standard.

FIG. 19 shows the production of QS-18 (QA-TriX-FRXA-C18-A-G). Transient co-expression of the QS-18 gene set (AstHMGR, QsbAS, QsCYP716-C-28, QsCYP716-C-16a, QsCYP714-C-23, QsCSL2, Qs-3-O-GaIT, Qs-3-O-XylT, Qs-28-O-FucT, QsFucSyn, Qs-28-0-RhaT, Qs-28-O-XyIT3, Qs-28-O-ApiT4, QsAXS, 3CCL, ChSA, ChSB, ChSC, ChSD, ChSE, ChSF, KR11, KR23′, DMOT4, DMOT9. UGT-L-long, and QS-7-GIcT) in N. benthamiana results in production of QS-18. LC-MS extract ion chromatograms are shown (negative mode, m/z=2149.9590-2149.9804 (C₉₈H₁₅₇O₅₁)) for N. benthamiana leaf extracts following transient expression of the QS-18 gene set. An extract from leaves expressing the QA-TriX-FRXA scaffold genes is shown as a negative control (top) and a QS-18 standard purified from a Q. saponaria bark extract is shown at the bottom.

FIG. 20 shows that the set of enzymes identified in this work (3CCL, ChSA-F, KR11, KR23′, DMOT9, DMOT4 and UGT-L) were able to add the acyl chain and associated arabinofuranose to QA-TriR-FRXX, QA-TriR-FRXA, QA-TriX-FRXX and QA-TriX-FRXA. A, The 10 M QS-21 standard, procured from Desert King (extracted and purified from Q. saponaria bark), had the same retention time and mass spectrum as QA-TriX-FRXX-C18-A and QA-TriX-FRXA-C18-A produced in N. benthamiana. B, The MS2 spectra show that a similar fragmentation occurred for the QS-21 obtained in N. benthamiana and the 0.1 M QS-21 standard. The screened masses of the hydrogen adducts in negative mode are as follow: QA-TriR-FRX(X/A)-C18-A m/z=2001.9225-2001-9425, QA-TriX-FRX(X/A)-C18-A m/z=1987.9070-1987.9268.

FIG. 21 shows the results of boosting the amounts of QA-TriX-FRXA-C18-A by optimizing the formation of the C9 acyl unit (or monomer). 2-methylbutyric acid was added to the infiltration solution containing the Agrobacterium strains necessary to build QS-21. n=4.

FIG. 22 shows ¹H NMR spectrum_(4.00-5.40 ppm) for QA-TriR-FRXA-C18 in MeOH-d₄ (600 MHz), purified from a Q. saponaria saponin crude extract.

FIG. 23 shows ¹H and ³C NMR spectroscopic data for QA-TriX-FRXA-C18 in MeOH-d₄/D₂O, 9:1 (400, 100 MHz), purified from a Q. saponaria saponin crude extract.

FIG. 24 shows semi-quantification of QS-21 in N. benthamiana. The QS-21 gene set was transiently expressed in N. benthamiana (left bar; QS-1 genes only). In addition, the wild-type Quillaja threonine deaminase (QsTD) or a feedback-insensitive QsTD P540L mutant were expressed (middle two bars). For comparison, 1 mM of the QS-21 yield-boosting 2-methylbutyric acid was included (right bar; 2 MB). Data represent average peak area of QS-21 normalised to an internal standard (digitoxin, 1 mg/g dry leaf weight), with relative abundance versus internal standard plotted on the Y-axis. Two replicates were included for each.

FIG. 25 shows UDP sugar donor specificity of the arabinofuranose transferase UGT-L-long (UGT73CZ2). A, Conversion of des-Araf QS-21 (QA-TriX-FRXA-C18; (11)) to QS-21 (1). The position where sugar is added by the transferase is indicated with a circle shown in dotted lines. B, Evaluation of the UDP sugar donor specificity of UGT-L-long. UGT-L-long was expressed with a carboxy-terminal hexahistidine tag in N. benthamiana and purified for use in in vitro assays (see Example 13). A starter molecule, 0.1 mM des-arabinosyl-QS-21 (Des-Araf-QS-21, QA-TriX-FRXA-C18; 11) was mixed with 0.5 mM of each UDP sugar (UDP-Araf; UDP-Arap; UDP-Glc; UDP-Gal; UDP-Xyl; UDP-Rha; UDP-D-Fuc) in a final volume of 50 μL. Reactions were initiated by addition of 0.8 μg of purified UGT-L-long to the reaction mixtures and incubated at 25° C. for 14 h. After quenching with methanol, the mixtures were analysed with a QExactive Hybrid Quadrupole-Orbitrap mass spectrometer equipped with CAD and RP-C18 column. The negative control is at the top (No Enzyme), and a QS-21 standard is shown at the bottom (Semi pure QS-21). UGT-L-long is able to transfer L-arabinofuranose and also D-xylose to 11 (des-Araf-QS-21; QA-TriX-FRXA-C18) with almost 100% efficiency, as shown by the absence of the substrate peaks. Charged Aerosol Detector (CAD) chromatograms are shown. QS-21-Xyl (QA-TriX-FRXA-C18-Xyl), an example of QA*-F*-C18-Xyl, eluted earlier than QS-21 (1).

FIG. 26 shows characterisation of QS-21 C-28 apiose chemotype purified from N. benthamiana. A, ¹H NMR spectral data for key resonances for the QS-21 standard and the product purified from N. benthamiana, recorded in MeOH-d₄, 600 MHz. B and C: Expanded ¹H NMR spectral comparisons with X axis: f1 (ppm) with QS-21 standard in the top panel and product produced in N. benthamiana below (B: 4.00-4.50 ppm; C: 4.96-5.40 ppm). D, From the left hand side: Retention time (X-axis: Time (min), HR-MS data and HR-MS2 data (X-axiz: m/z). Top panel: QS-21 standard. Bottom panel: Compound purified from N. Benthamiana.

FIG. 27 shows C-23 aldehyde (A) and full ¹H NMR spectral comparison between the QS-21 standard (B) and the product generated by large-scale agro-infiltration of N. benthamiana (C) (recorded in MeOH-d4, 600 MHz, X-axis: f1(ppm)).

### DETAILED DESCRIPTION OF THE INVENTION

A first aspect of the invention is a method of making QA*-F*-C18-A, wherein the F* chain is at the C-28 position of QA*, and the C-18-A chain is attached to the D-fucose of the F* chain. The method comprises combining QA*-F*-C18 with an enzyme capable of transferring UDP-3-L-arabinofuranose to QA*-F*-C18 to form QA*-F*-C18-A. The method of making QA*-F*-C18-A can also make QA*-F*-C18-Xyl. In this aspect of the invention QA* is 3-O-{β-D-glucopyranosiduronic acid}-quillaic acid (QA-Mono), 3-O-{3-D-galactopyranosyl-(1->2)-β-D-glucopyranosiduronic acid}-quillaic acid (QA-Di); 3-O-{α-L-rhamnopyranosyl-(1->3)-[β-D-galactopyranosyl-(1->2)]-β-D-glucopyranosiduronic acid}-quillaic acid (QA-TriR); 3-O-{β-D-xylopyranosyl-(1->3)-[β-D-galactopyranosyl-(1->2)]-β-D-glucopyranosiduronic acid}-quillaic acid (QA-TriX) or quillaic acid glycosylated at the C-3 position with a branched trisaccharide which is either QA-TriX or QA-TriR; F* is a disaccharide made of a β-D-fucose residue (F) and a rhamnose residue (R), also referred to as FR; a trisaccharide made of F, R and a xylose residue (X), also referred to as FRX; a tetrasaccharide made of F, R, X and X, also referred to as FRXX; a tetrasaccharide made of F, R, X and a β-D-apiose residue (A), also referred to as FRXA; C18 is (3S,5S,6S)-5-((3S,5S,6S)-3,5-dihydroxy-6-methyloctanoyloxy)-3-hydroxy-6-methyloctanoic acid; and

A is an arabinofuranose residue.

In this aspect of the invention QA*-F*-C18 may be formed by combining QA*-F*-C9 with an enzyme capable of transferring an acyl unit to QA*-F*-C9.

In this aspect of the invention QA*-F*-C9 may be formed by combining (3S,5S,6S)-3,5-dihydroxy-6-methyloctanoyl-CoA and QA*-F* with an enzyme capable of transferring an acyl unit to QA*-F*.

In this aspect of the invention (3S,5S,6S)-3,5-dihydroxy-6-methyloctanoyl-CoA may be formed by combining 2-methylbutyryl-CoA and malonyl-CoA with one or more enzymes capable of adding malonyl-CoA, and one or more enzymes capable of reducing a ketone.

In this aspect of the invention 2-methylbutyryl-CoA may be formed by combining 2-methylbutyric acid with one or more enzymes capable of transferring a CoA to 2-methylbutyric acid.

In this aspect of the invention F* may be FRX, FRXX, FRXA or mixtures thereof. Preferably F* is FRXA.

The method of the first aspect of the invention may comprise the steps of:
i. combining 2-methylbutyric acid with one or more enzymes capable of transferring a CoA to 2-methylbutyric acid to form 2-methylbutyryl-CoA;
ii. combining 2-methylbutyryl-CoA and malonyl-CoA with one or more enzymes capable of adding malonyl-CoA, and one or more enzymes capable of reducing a ketone, to form (3S,5S,6S)-3,5-dihydroxy-6-methyloctanoyl-CoA;
iii. combining (3S,5S,6S)-3,5-dihydroxy-6-methyloctanoyl-CoA and QA*-F* with an enzyme capable of transferring an acyl unit to QA*-F* to form QA*-F*-C9;
iv. combining QA*-F*-C9 with an enzyme capable of transferring an acyl unit to QA*-F*-C9 to form QA*-F*-C18;
v. combining QA*-F*-C18 with an enzyme capable of transferring UDP-β-L-arabinofuranose to QA*-F*-C18 to form QA*-F*-C18-A.
Step (v) can also make QA*-F*-C18-Xyl, by combining QA*-F*-C18 with an enzyme capable of transferring UDP-Xylose to QA*-F*-C18 to form QA*-F*-C18-Xyl. The enzyme capable of transferring UDP-Xylose to QA*-F*-C18 to form QA*-F*-C18-Xyl can be the same enzyme as the enzyme capable of transferring UDP-β-L-arabinofuranose to QA*-F*-C18 to form QA*-F*-C18-A.

A second aspect of the invention is a method of making QA*-F*-C18-A-G, wherein the F* chain is at the C-28 position of QA*, the C-18-A chain is attached to the D-fucose of the F* chain and the G residue is attached at the C-3 position of the rhamnose residue of the F* chain, comprising making QA*-F*-C18-A by the method of the first aspect of the invention, and combining QA*-F*-C18-A with an enzyme capable of transferring a D-glucose residue to QA*-F*-C18-A to form QA*-F*-C18-A-G. In this aspect of the invention F* is FRX, FRXX, FRXA or mixtures thereof, preferably wherein F* is FRXA.

A third aspect of the invention is a method of making QA*-F*-C18-A-G wherein the F* chain is at the C-28 position of QA*, the C-18-A chain is attached to the D-fucose of the F* chain and the G residue is attached at the C-3 position of the rhamnose residue of the F* chain, wherein F* is FRX, FRXX, FRXA or mixtures thereof, preferably wherein F* is FRXA, and wherein the method comprises the step of combining QA*-F* with an enzyme capable of transferring a D-glucose residue to QA*-F* to form QA*-F*-G. In this aspect of the invention QA* is 3-O-{β-D-glucopyranosiduronic acid}-quillaic acid (QA-Mono), 3-O-{3-D-galactopyranosyl-(1->2)-β-D-glucopyranosiduronic acid}-quillaic acid (QA-Di); 3-O-{α-L-rhamnopyranosyl-(1->3)-[β-D-galactopyranosyl-(1->2)]-β-D-glucopyranosiduronic acid}-quillaic acid (QA-TriR); 3-O-{β-D-xylopyranosyl-(1->3)-[β-D-galactopyranosyl-(1->2)]-β-D-glucopyranosiduronic acid}-quillaic acid (QA-TriX) or quillaic acid glycosylated at the C-3 position with a branched trisaccharide which is either QA-TriX or QA-TriR; F* is a disaccharide of a β-D-fucose residue (F) and a rhamnose residue (R), also referred to as FR; a trisaccharide of F, R and a xylose residue (X), also referred to as FRX; a tetrasaccharide of F, R, X and X, also referred to as FRXX; a tetrasaccharide of F, R, X and a β-D-apiose residue (A), also referred to as FRXA; and G is a glucose residue.

In this aspect of the invention QA*-F*-C9-G may be formed by combining QA*-F*-G with (3S,5S,6S)-3,5-dihydroxy-6-methyloctanoyl-CoA and an enzyme capable of transferring an acyl unit to QA*-F*-G to form QA*-F*-C9-G. In this aspect of the invention C9 is (3S,5S,6S)-3,5-dihydroxy-6-methyloctanoyl-CoA or (3S,5S,6S)-3,5-dihydroxy-6-methyloctanoic acid.

In this aspect of the invention QA*-F*-C18-G may be formed by combining QA*-F*-C9-G with an enzyme capable of transferring an acyl unit to QA*-F*-C9-G to form QA*-F*-C18-G.

In this aspect of the invention, QA*-F*-C18-A-G may be formed by combining QA*-F*-C18-G with an enzyme capable of transferring UDP-β-L-arabinofuranose to QA*-F*-C18-G to form QA*-F*-C18-A-G. In this aspect of the invention A is an arabinofuranose residue.

The method of the third aspect of the invention may comprise the steps of:
i. combining 2-methylbutyric acid with one or more enzymes capable of transferring a CoA to 2-methylbutyric acid to form 2-methylbutyryl-CoA;
ii. combining 2-methylbutyryl-CoA andmalonyl-CoA with one or more enzymes capable of adding malonyl-CoA, and one or more enzymes capable of reducing a ketone, to form (3S,5S,6S)-3,5-dihydroxy-6-methyloctanoyl-CoA;
iii. combining QA*-F* with an enzyme capable of transferring a D-glucose residue to QA*-F* to form QA*-F*-G
iv. combining (3S,5S,6S)-3,5-dihydroxy-6-methyloctanoyl-CoA and QA*-F*-G with an enzyme capable of transferring an acyl unit to QA*-F*-G to form QA*-F*-C9-G;
v. combining QA*-F*-C9-G with an enzyme capable of transferring an acyl unit to QA*-F*-C9-G to form QA*-F*-C18-G;
vi. combining QA*-F*-C18-G with an enzyme capable of transferring UDP-β-L-arabinofuranose to QA*-F*-C18-G to form QA*-F*-C18-A-G.

A fourth aspect of the invention is a method of making a biosynthetic QA*-F*-C18-A in a host, wherein the F* chain is at the C-28 position of QA*, and the C-18-A chain is attached to the D-fucose of the F* chain, the method comprising the steps of:
a) expressing genes required for the biosynthesis of QA*-F* into the host, and
b) introducing a polynucleotide encoding:
   i. at least one or more enzymes capable of transferring a CoA to 2-methylbutyric acid to form 2-methylbutyryl-CoA;
   ii. at least one or more enzymes capable of adding malonyl-CoA, and one or more enzymes capable of reducing a ketone, to form (3S,5S,6S)-3,5-dihydroxy-6-methyloctanoyl-CoA;
   iii. at least one or more enzymes capable of transferring an acyl unit to QA*-F* to form QA*-F*-C9;
   iv. at least one or more enzymes capable of transferring an acyl unit to QA*-F*-C9 to form QA*-F*-C18; and
   v. at least one or more enzymes capable of transferring UDP-β-L-arabinofuranose to QA*-F*-C18 to form QA*-F*-C18-A,
      into the host.

In this aspect of the invention QA* is 3-O-{β-D-glucopyranosiduronic acid}-quillaic acid (QA-Mono), 3-O-{β-D-galactopyranosyl-(1->2)-β-D-glucopyranosiduronic acid}-quillaic acid (QA-Di); 3-O-{α-L-rhamnopyranosyl-(1->3)-[β-D-galactopyranoyl-(1->2)]-β-D-glucopyranosiduronic acid}-quillaic acid (QA-TriR); 3-O-{β-D-xylopyranosyl-(1->3)-[β-D-galactopyranosyl-(1->2)]-β-D-glucopyranosiduronic acid}-quillaic acid (QA-TriX) or quillaic acid glycosylated at the C-3 position with a branched trisaccharide which is either QA-TriX or QA-TriR;
F* is a disaccharide made of a β-D-fucose residue (F) and a rhamnose residue (R), also referred to as FR; a trisaccharide made of F, R and a xylose residue (X), also referred to as FRX; a tetrasaccharide made of F, R, X and X, also referred to as FRXX; a tetrasaccharide made of F, R, X and a β-D-apiose residue (A), also referred to as FRXA;
C9 is (3S,5S,6S)-3,5-dihydroxy-6-methyloctanoyl-CoA or (3S,5S,6S)-3,5-dihydroxy-6-methyloctanoic acid C18 is (3S,5S,6S)-5-((3S,5S,6S)-3,5-dihydroxy-6-methyloctanoyloxy)-3-hydroxy-6-methyloctanoic acid; and
A is an arabinofuranose residue.

In this aspect of the invention F* may be FRX, FRXX, FRXA or mixtures thereof. Preferably F* is FRXA.

A fifth aspect of the invention is a method of making a biosynthetic QA*-F*-C18-A-G, wherein the F* chain is at the C-28 position of QA*, the C-18-A chain is attached to the D-fucose of the F* chain and the G residue is attached at the C-3 position of the rhamnose residue of the F* chain, comprising making QA*-F*-C18-A by the method of the [insert] aspect of the invention, wherein step b) further comprises introducing a polynucleotide encoding at least one or more enzymes capable of transferring a glucose residue to QA*-F*-C18-A to form QA*-F*-C18-A-G, into the host. In this aspect of the invention F* is FRX, FRXX, FRXA or mixtures thereof. Preferably F* is FRXA.

In the fifth aspect of the invention QA* is 3-O-{β-D-glucopyranosiduronic acid}-quillaic acid (QA-Mono), 3-O-{β-D-galactopyranosyl-(1->2)-β-D-glucopyranosiduronic acid}-quillaic acid (QA-Di); 3-O-{α-L-rhamnopyranosyl-(1->3)-[β-D-galactopyranosyl-(1->2)]-β-D-glucopyranosiduronic acid}-quillaic acid (QA-TriR); 3-O-{β-D-xylopyranosyl-(1->3)-[β-D-galactopyranosyl-(1->2)]-β-D-glucopyranosiduronic acid}-quillaic acid (QA-TriX) or quillaic acid glycosylated at the C-3 position with a branched trisaccharide which is either QA-TriX or QA-TriR;
F* is a disaccharide of a R-D-fucose residue (F) and a rhamnose residue (R), also referred to as FR; a trisaccharide of F, R and a xylose residue (X), also referred to as FRX; a tetrasaccharide of F, R, X and X, also referred to as FRXX; a tetrasaccharide of F, R, X and a β-D-apiose residue (A), also referred to as FRXA;
C9 is (3S,5S,6S)-3,5-dihydroxy-6-methyloctanoyl-CoA or (3S,5S,6S)-3,5-dihydroxy-6-methyloctanoic acid C18 is (3S,5S,6S)-5-((3S,5S,6S)-3,5-dihydroxy-6-methyloctanoyloxy)-3-hydroxy-6-methyloctanoic acid;
A is an arabinofuranose residue; and
G is a glucose residue.

In these aspects of the invention, QA* may be QA, QA-Mono, QA-Di, QA-TriX, QA-TriR, or a mixture thereof. The Mono, Di or Tri(X/R) chain is added at the C-3 position of the QA backbone.

When QA* is a mixture comprising QA-TriX and QA-TriR, the ratio of QA-TriX to QA-TriR may vary. The ratio of QA-TriX to QA-TriR within the mixture may vary in percentage. Suitably, the mixture comprises from 10 to 90% of QA-TriX, such as 10%, 20%, 30%, 40%, 50%, 60%, 70%, 80%, or 90% and from 90 to 10% of QA-TriR, such as 90%, 80%, 70%, 60%, 50%, 40%, 30%, 20%, or 10%.

In these aspects of the invention, F* is F, FR, FRX, FRXA, FRXX, or a mixture thereof. The sugars are added at the C-28 position of QA*. When F* is a mixture comprising FRXX and FRXA, the ratio of FRXX to FRXA may vary. The ratio of FRXX to FRXA within the mixture may vary in percentage. Suitably, the mixture comprises from 10 to 90% of FRXX, such as 10%, 20%, 30%, 40%, 50%, 60%, 70%, 80%, or 90% and from 90 to 10% of FRXA, such as 90%, 80%, 70%, 60%, 50%, 40%, 30%, 20%, or 10%. Preferably, the mixture comprises 60% of FRXX and 40% of FRXA, or 50% of each. When the glucose residue is attached to the F* chain, F* is FRX, FRXA, FRXX or mixtures thereof.

In these aspects of the invention, QA*-F* may be QA-F, QA-Mono-F, QA-Di-F, QA-TriX-F, QA-TriR-F, QA-FR, QA-Mono-FR, QA-Di-FR, QA-TriX-FR, QA-TriR-FR, QA-FRX, QA-Mono-FRX, QA-Di-FRX, QA-TriX-FRX, QA-TriR-FRX, QA-FRXX, QA-Mono-FRXX, QA-Di-FRXX, QA-TriX-FRXX, QA-TriR-FRXX, QA-FRXA, QA-Mono-FRXA, QA-Di-FRXA, QA-TriX-FRXA, QA-TriR-FRXAor a mixture thereof. QA*-F* has at least a D-fucose residue attached at the C-28 position of QA* and when the glucose residue is added QA*-F* has at least a fucose, rhamnose and xylose residue attached at the C-28 position of QA*.

### Synthesis of the Acyl Chain and Glucose Residue Attached to the C-28 Linear Tetrasaccharide

The QS-21 acyl chain is made of two identical acyl units (or monomers) with an arabinofuranose attached to the C5 hydroxy of the second unit (see FIG. 3 and FIG. 8 for an illustration of the carbon numbering). The inventors identified enzymes which allowed the acyl chain to be built starting from 2-methylbutyric acid, and then added to QA* derivatives to form QA*-F*-C18-A, including e.g. QA-TriX-FRXX-C18-A and QA-TriX-FRXA-C18-A (the two main isomeric constituents of the QS-21 fraction), in vitro and in vivo: CoA is attached to 2-methylbutyric acid to form 2-methylbutyryl-CoA. Two molecules of malonyl-CoA are then used to extend 2-methylbutyryl-CoA into (S)-6-methyl-3,5-dioxooctanoyl-CoA. The two keto groups are then reduced stereoselectively to form the acyl unit as seen in QS-21. This acyl unit is transferred onto the C-4 position of the fucose residue on the QA*-F* derivative. An additional acyl unit is then added to the C-5 hydroxy group of the first acyl unit. Lastly, an arabinofuranose is attached to the C-5 hydroxy group of the acyl chain to produce QA*-F*-C18-A. The inventors also identified enzymes which allowed a D-glucose residue to be attached to the C-3 position of the rhamnose residue of the C-28 linear tetrasaccharide chain of a molecule comprising QA*-F*-C18-A to form QA*-F*-C18-A-G or a molecule comprising QA*-F* to form QA*-F*-G. When the D-glucose residue is added to QS-21, QS-18 is formed.

In the following description, the methods of the invention are described for the situation when the acyl unit, (3S,5S,6S)-3,5-dihydroxy-6-methyloctanoyl-CoA, is produced. Then two acyl units are sequentially added to QA*-F* before an arabinofuranose is attached to the C-5 hydroxy of the acyl chain and optionally a glucose residue is attached at the C-3 position of the rhamnose residue of the F* chain. However, the steps can be performed in a specific order or in any order or simultaneously. For example, the glucose residue may be added to QA*-F* before two acyl units are sequentially added then an arabinofuranose is attached to the C-5 hydroxy of the acyl chain.

In the methods of these aspects of the invention, the transfer of a CoA to 2-methylbutyric acid to form 2-methylbutyryl-CoA may be carried out by one or more enzymes selected from carboxyl CoA ligase 6 (6CCL) (SEQ ID NO 64), carboxyl CoA ligase 5 (5CCL) (SEQ ID NO 62), carboxyl CoA ligase 4 (4CCL) (SEQ ID NO 60), carboxyl CoA ligase 3 (3CCL) (SEQ ID NO 58), carboxyl CoA ligase 2 (2CCL) (SEQ ID NO 56), carboxyl CoA ligase 1 (1CCL) (SEQ ID NO 54) and an enzyme having an amino acid sequence with at least 60% sequence identity to SEQ ID NO 64, 62, 60, 58, 56 or 54. Preferably the enzyme is 3CCL. The enzymes are capable of transferring CoA to 2-methylbutyric acid. The function of the enzyme can be determined for example as described in Example 3.

The function of CCL enzymes may be determined by in vitro analysis wherein the substrates 2-methylbutyric acid and CoA are mixed with the candidate enzyme and the product formation is determined by LC-MS analysis. Alternatively, in vivo analysis using a heterologous host such as N. benthamiana or yeast expressing the CCL enzyme candidate may be used wherein 2-methylbutyric acid may be supplemented and 2-methylbutyryl-CoA measured by LC-MS.

Throughout this description when referring to an “enzyme (SEQ ID NO)”, this is referring to an enzyme according to that SEQ ID NO, i.e. an enzyme having the amino acid sequence of that SEQ ID NO. For example, “6CCL (SEQ ID NO 64)” means the enzyme 6CCL according to SEQ ID NO 64, i.e. the enzyme 6CCL having the amino acid sequence of SEQ ID NO 64.

Enzymes for use in the present invention may include one or more conservative amino acid substitutions, such that the resulting enzyme has a similar amino acid sequence and/or retains the same function. The skilled person is aware that various amino acids have similar biochemical properties and thus are “conservative”. One or more such amino acids of a protein (e.g. enzymes), polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, the methyl group on an alanine may be replaced with an ethyl group, and/or minor changes may be made to the peptide backbone. Whether or not natural or synthetic amino acids are used, it is preferred that only L-amino acids are present. Substitutions of this nature are often referred to as “conservative” amino acid substitutions.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exists a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score.

The percentage of identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percentage of identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules. BLAST protein searches can be performed with the XBLAST program, score=50, wordlength=3 to obtain amino acid sequences homologous to protein molecules for use in the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

Mutations, including conservative substitutions, insertions and deletions, may be introduced into the sequences using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6.

In the methods of these aspects of the invention, the transfer of a CoA to 2-methylbutyric acid to form 2-methylbutyryl-CoA may be carried out by one or more carboxyl CoA enzymes selected from enzymes having at least 60% sequence identity to the sequences for 1CCL, 2CCL, 3CCL, 4CCL, 5CCL and 6CCL (SEQ ID NO 54, 56, 58, 60, 62 or 64, respectively). The amino acid sequence of the 1CCL enzyme may have at least 60%, 65%, 70%, 75%, 80%, 85%, 90%, 95%, 96%, 97%, 98% or 99% identity to SEQ ID NO 54. The amino acid sequence of the 2CCL enzyme may have at least 60%, 65%, 70%, 75%, 80%, 85%, 90%, 95%, 96%, 97%, 98% or 99% identity to SEQ ID NO 56. The amino acid sequence of the 3CCL enzyme may have at least 60%, 65%, 70%, 75%, 80%, 85%, 90%, 95%, 96%, 97%, 98% or 99% identity to SEQ ID NO 58. The amino acid sequence of the 4CCL enzyme may have at least 60%,65%, 70%, 75%, 80%, 85%, 90%, 95%, 96%, 97%, 98% or 99% identity to SEQ ID NO 60. The amino acid sequence of the 5CCL enzyme may have at least 60%, 65%, 70%, 75%, 80%, 85%, 90%, 95%, 96%, 97%, 98% or 99% identity to SEQ ID NO 62. The amino acid sequence of the 6CCL enzyme may have at least 60%, 65%, 70%, 75%, 80%, 85%, 90%, 95%, 96%, 97%, 98% or 99% identity to SEQ ID NO 64. Accordingly, in some embodiments, the 1CCL, 2CCL, 3CCL, 4CCL, 5CCL and/or 6CCL enzyme has at least 60%, 65%, 70%, 75%, 80%, 85%, 90%, 95%, 96%, 97%, 98% or 99% identity to SEQ ID NO 54, 56, 58, 60, 62 or 64, suitably at least 90%, more suitably at least 95%. In respect of the enzymes defined here in terms of sequence identity, they typically retain the function of transferring a CoA to 2-methylbutyric acid to form 2-methylbutyryl-CoA.

The percentage sequence identities discussed in this application are the percentage sequence identities across the full length of the sequences identified by the SEQ. ID NOs. This may include shortened sequences which have the same sequence identity measured across the length of the shortened sequence. The shortened sequences may have the same homology of the percentage sequence identity of the SEQ ID NO regardless of the length of the shortened sequence. The shortened sequence may be at least half the length of the full-length sequence, preferably at least three quarters of the length of the full sequence.

In the methods of these aspects of the invention, the extension of 2-methylbutyryl-CoA using at least two molecules of malonyl-CoA may be carried out by one or more enzymes selected from chalcone synthase-like A (ChSA) (SEQ ID NO 66), chalcone synthase-like B (ChSB) (SEQ ID NO 68), chalcone synthase-like C (ChSC) (SEQ ID NO 70), chalcone synthase-like D (ChSD) (SEQ ID NO 72), chalcone synthase-like E (ChSE) (SEQ ID NO 74), chalcone synthase-like F (ChSF) (SEQ ID NO 76) and an enzyme having an amino acid sequence with at least 50% sequence identity to SEQ ID NO 66, 68, 70, 72, 74 or 76. This extension may be carried out by two or more enzymes. The enzymes are capable of converting 2-methylbutyryl-CoA to (S)-6-methyl-3,5-dioxooctanoyl-CoA (diketone). The function of the enzymes can be determined for example as described in Example 4. The enzymes may be ChSE and/or ChSD.

The function of the ChS enzymes may be determined by in vitro analysis wherein the substrates 2-methylbutyryl-CoA and malonyl-CoA are mixed with the candidate enzyme and the product formation is observed by LC-MS analysis and identified by NMR. Alternatively, in vivo analysis using a heterologous host such as N. benthamiana or yeast expressing enzymes necessary to form the substrate 2-methylbutyryl-CoA (e.g. 3CCL) and the ChS enzyme candidate may be used wherein 2-methylbutyric acid may be supplemented and the product formation measured by LC-MS or indirectly measured by co-expressing the necessary enzymes to produce a downstream product more easily detectable (e.g. QA*-F*-C9).

In the methods of these aspects of the invention, extension of 2-methylbutyryl-CoA using at least two molecules of malonyl-CoA may be carried out by one or more carboxyl CoA enzymes selected from enzymes having at least 50% sequence identity to the sequences for ChSA, ChSB, ChSC, ChSD, ChSE and ChSF (SEQ ID NO 66, 68, 70, 72, 74, 76 respectively). The amino acid sequence of the ChSA enzyme may have at least 50%, 55%, 60%, 65%, 70%, 75%, 80%, 85%, 90%, 95%, 96%, 97%, 98% or 99% identity to SEQ ID NO 66. The amino acid sequence of the ChSB enzyme may have at least 50%, 55%, 60%, 65%, 70%, 75%, 80%, 85%, 90%, 95%, 96%, 97%, 98% or 99% identity to SEQ ID NO 68. The amino acid sequence of the ChSC enzyme may have at least 50%, 55%, 60%, 65%, 70%, 75%, 80%, 85%, 90%, 95%, 96%, 97%, 98% or 99% identity to SEQ ID NO 70. The amino acid sequence of the ChSD enzyme may have at least 50%, 55%, 60%, 65%, 70%, 75%, 80%, 85%, 90%, 95%, 96%, 97%, 98% or 99% identity to SEQ ID NO 72. The amino acid sequence of the ChSE enzyme may have at least 50%, 55%, 60%, 65%, 70%, 75%, 80%, 85%, 90%, 95%, 96%, 97%, 98% or 99% identity to SEQ ID NO 74. The amino acid sequence of the ChSF enzyme may have at least 50%, 55%, 60%, 65%, 70%, 75%, 80%, 85%, 90%, 95%, 96%, 97%, 98% or 99% identity to SEQ ID NO 76. Accordingly, in some embodiments, the ChSA, ChSB, ChSC, ChSD, ChSE and/or ChSF enzyme has at least 50%, 55%, 60%, 65%, 70%, 75%, 80%, 85%, 90%, 95%, 96%, 97%, 98% or 99% identity to SEQ ID NO 66, 68, 70, 72 or 74, suitably at least 90%, more suitably at least 95%. In respect of the enzymes defined here in terms of sequence identity, they typically retain the function of converting 2-methylbutyryl-CoA to (S)-6-methyl-3,5-dioxooctanoyl-CoA (diketone).

In the methods of these aspects of the invention, the reduction of the ketones in (S)-6-methyl-3,5-dioxooctanoyl-CoA to form (3S,5S,6S)-3,5-dihydroxy-6-methyloctanoyl-CoA may be carried out by an enzyme selected from keto reductase 11 (KR11) (SEQ ID NO 78) and an enzyme having an amino acid sequence with at least 20% sequence identity to SEQ ID NO 78, optionally in combination with an enzyme selected from keto reductase 23′ (KR23′) (SEQ ID NO 80) and an enzyme having an amino acid sequence with at least 15% sequence identity to SEQ ID NO 80. The enzymes are capable of converting (S)-6-methyl-3,5-dioxooctanoyl-CoA to (3S,5S,6S)-3,5-dihydroxy-6-methyloctanoyl-CoA. The function of the enzyme can be determined for example as described in Example 5.

The function of the KR enzymes may be determined by in vitro analysis wherein the substrates (S)-6-methyl-3,5-dioxooctanoyl-CoA is mixed with the candidate enzyme and the product formation is observed by LC-MS analysis and identified by NMR. Alternatively, in vivo analysis using a heterologous host such as N. benthamiana or yeast expressing enzymes necessary to form the substrate (S)-6-methyl-3,5-dioxooctanoyl-CoA (e.g. 3CCL and ChSE and ChSD) and the KR candidate may be used wherein 2-methylbutyric acid may be supplemented and the product formation measured by LC-MS or indirectly measured by co-expressing the necessary enzymes to produce a downstream product more easily detectable (e.g. QA*-F*-C9).

In the methods of these aspects of the invention, the reduction of the ketones in (S)-6-methyl-3,5-dioxooctanoyl-CoA to form (3S,5S,6S)-3,5-dihydroxy-6-methyloctanoyl-CoA may be carried out by a keto reductase enzyme having at least 20% sequence identity to the sequence for KR11 (SEQ ID NO 78), optionally in combination with an enzyme having at least 15% sequence identity to the sequence of KR23′ (SEQ ID NO 80). The amino acid sequence of the KR11 enzyme may have at least 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 78. The amino acid sequence of the KR23′ enzyme may have at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 80. Accordingly, in some embodiments, the KR11 or KR23′ enzyme has at least 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 78 or 80 respectively, suitably at least 90%, more suitably at least 95%. In respect of the enzymes defined here in terms of sequence identity, they typically retain the function of converting (S)-6-methyl-3,5-dioxooctanoyl-CoA to (3S,5S,6S)-3,5-dihydroxy-6-methyloctanoyl-CoA.

The addition of two molecules of malonyl-CoA and reduction of the ketones in the methods of these aspects of the invention may occur in any order or simultaneously.

In the methods of these aspects of the invention, the addition of an acyl unit (from (3S,5S,6S)-3,5-dihydroxy-6-methyloctanoyl-CoA) to QA*-F* or QA*-F*-G to form QA*-F*-C9 or QA*-F*-C9-G respectively may be carried out by (3S,5S,6S)-3,5-dihydroxy-6-methyloctanoyl-CoA transferase 9 (DMOT9) (SEQ ID NO 82) or an enzyme having an amino acid sequence with at least 25% sequence identity to SEQ ID NO 82. The enzyme is capable of adding (3S,5S,6S)-3,5-dihydroxy-6-methyloctanoyl-CoA to QA*-F* or QA*-F*-G. The function of the enzyme can be determined for example as described in Example 6.

The function of the DMOT9 enzyme may be determined by in vitro analysis wherein the acyl unit and QA*-F* or QA*-F*-G are mixed with the candidate enzyme and the product formation is identified by LC-MS analysis. Alternatively, in vivo analysis using a heterologous host such as N. benthamiana or yeast expressing enzymes necessary to form the acyl unit (e.g. 3CCL, ChSE, ChSD, KR11, KR23′) and the DMOT9 candidate may be used wherein 2-methylbutyric acid may be supplemented and the product formation measured by LC-MS.

In the methods of these aspects of the invention, addition of (3S,5S,6S)-3,5-dihydroxy-6-methyloctanoyl-CoA to QA*-F* or QA*-F*-G may be carried out by an acyl transferase enzyme having at least 40% sequence identity to the sequence for DMOT9 (SEQ ID No 82). The amino acid sequence of the DMOT9 enzyme may have at least 25%, 30%, 35%, 40%, 45%, 50%, 55%, 60%, 65%, 70%, 75%, 80%, 85%, 90%, 95%, 96%, 97%, 98% or 99% identity to SEQ ID NO 82. Accordingly, in some embodiments, the DMOT9 enzyme has at least 25%, 30%, 35%, 40%, 45%, 50%, 55%, 60%, 65%, 70%, 75%, 80%, 85%, 90%, 95%, 96%, 97%, 98% or 99% identity to SEQ ID NO 82, suitably at least 90%, more suitably at least 95%. In respect of the enzymes defined here in terms of sequence identity, they typically retain the function of transferring (3S,5S,6S)-3,5-dihydroxy-6-methyloctanoyl-CoA to QA*-F* or QA*-F*-G.

In the methods of these aspects of the invention, the transfer of an acyl unit to QA*-F*-C9 or QA*-F*-C9-G to form QA*-F*-C18 or QA*-F*-C18-G respectively may be carried out by the enzyme (3S,5S,6S)-3,5-dihydroxy-6-methyloctanoyl-CoA transferase 4 (DMOT4) (SEQ ID NO 84) or an enzyme having an amino acid sequence with at least 15% sequence identity to SEQ ID NO 84. The enzyme is capable of transferring an acyl unit to QA*-F*-C9 or QA*-F*-C9-G. The function of the enzyme can be determined for example as described in Example 6.

The function of the DMOT4 enzyme may be determined by in vitro analysis wherein the acyl unit and QA*-F*-C9 or QA*-F*-C9-G are mixed with the candidate enzyme and the product formation is identified by LC-MS analysis. Alternatively, in vivo analysis using a heterologous host such as N. benthamiana or yeast expressing enzymes necessary to form QA*-F*-C9 or QA*-F*-C9-G and the DMOT4 candidate may be used wherein 2-methylbutyric acid may be supplemented and the product formation measured by LC-MS.

In the methods of these aspects of the invention, transfer of an acyl unit to QA*-F*-C9 or QA*-F*-C9-G to form QA*-F*-C18 or QA*-F*-C18-G may be carried out by an acyl transferase enzyme having at least 15% sequence identity to the sequence for DMOT4 (SEQ ID No 84). The amino acid sequence of the DMOT4 enzyme may have at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 84. Accordingly, in some embodiments, the DMOT4 enzyme has at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 84, suitably at least 90%, more suitably at least 95%. In respect of the enzymes defined here in terms of sequence identity, they typically retain the function of transferring an acyl unit to QA*-F*-C9 or QA*-F*-C9-G.

In the methods of these aspects of the invention, the attachment of UDP-β-L-arabinofuranose to an acyl unit on QA*-F*-C18 to form QA*-F*-C18-A or to an acyl unit on QA*-F*-C18-G to form QA*-F*-C18-A-G, may be carried out by an enzyme selected from uridine diphosphate glycosyltransferase-L-short (UGT-L-short) (SEQ ID NO 86), uridine diphosphate glycosyltransferase-L-long (UGT-L-long) (SEQ ID NO 88) and an enzyme having an amino acid sequence with at least 45% sequence identity to SEQ ID NO 86 or 88. The enzyme is capable of transferring UDP-β-L-arabinofuranose (arafT enzyme). The function of the enzyme can be determined for example as described in Example 7.

The function of the arafT enzyme may be determined by in vitro analysis wherein arabinofuranose and QA*-F*-C18 or QA*-F*-C18-G are mixed with the candidate enzyme and the product formation is identified by LC-MS analysis. Alternatively, in vivo analysis using a heterologous host such as N. benthamiana or yeast expressing enzymes necessary to form QA*-F*-C18 or QA*-F*-C18-G and the arafT enzyme candidate may be used wherein 2-methylbutyric acid may be supplemented and the product formation measured by LC-MS.

In the methods of these aspects of the invention, the attachment of UDP-β-L-arabinofuranose to an acyl unit on QA*-F*-C18 or QA*-F*-C18-G may be carried out by an arabinofuranosyl transferase enzyme selected from enzymes having at least 50% sequence identity to the sequences for UGT-L-short and UGT-L-long (SEQ ID NO 86 and 88 respectively). The amino acid sequence of the UGT-L-short enzyme may have at least 45%, 50%, 55%, 60%, 65%, 70%, 75%, 80%, 85%, 90%, 95%, 96%, 97%, 98% or 99% identity to SEQ ID NO 86. The amino acid sequence of the UGT-L-long enzyme may have at least 45%, 50%, 55%, 60%, 65%, 70%, 75%, 80%, 85%, 90%, 95%, 96%, 97%, 98% or 99% identity to SEQ ID NO 88. Accordingly, in some embodiments, the UGT-L-short enzyme and/or UGT-L-long enzyme have at least 45%, 50%, 55%, 60%, 65%, 70%, 75%, 80%, 85%, 90%, 95%, 96%, 97%, 98% or 99% identity to SEQ ID NO 86 or 88, suitably at least 90%, more suitably at least 95%. In respect of the enzymes defined here in terms of sequence identity, they typically retain the function of transferring UDP-β-L-arabinofuranose (arafT enzyme) to QA*-F*-C18 or QA*-F*-C18-G.

In the methods of these aspects of the invention, the transfer of a D-glucose residue to QA*-F*-C18-A to form QA*-F*-C18-A-G or the transfer of a D-glucose residue to QA*-F* to form QA*-F*-G may be carried out by the enzyme quillaic acid 28-O-fucoside [1,2]-rhamnoside [1,3]glucosyltransferase (QS-7-GIcT) (SEQ ID NO 90) or an enzyme having an amino acid sequence with at least 70% sequence identity to SEQ ID NO 90. The enzyme is capable of transferring D-glucose residue to QA*-F*-C18-A or QA*-F* to form QA*-F*-C18-A-G or QA*-F*-G respectively. When the D-glucose residue is transferred the F* chain must have at least the fucose, rhamnose and xylose sugars in the linear chain, i.e. F* is FRX, FRXA, FRXX or mixtures thereof. The function of the enzyme can be determined for example as described in Example 8.

The function of QS-7-GIcT may be determined by expressing in a heterologous host such as N. benthamiana or yeast the enzymes necessary to generate QA*-F*-C18-A or QA*-F* and the QS-7-GIcT candidate. The presence of the expected product may be assessed by LC-MS analysis, eventually complemented by NMR analysis. Alternatively, in vitro testing may be preferred in which QA*-F*-C18-A or QA*-F* is either purified from a plant extract or generated in vitro in an assay containing quillaic acid and the glycosyl transferases necessary to generate QA*-F*-C18-A or QA*-F*, or β-amyrin and the enzymes necessary to produce QA*-F*-C18-A or QA*-F*. The activity of the candidate QS-7-GIcT is then tested in vitro on the QA*-F*-C18-A or QA*-F* substrate and the product formation is determined by LC-MS analysis.

In the methods of the invention, the transfer of a glucose residue to a molecule comprising QA*-F*-C18-A or QA*-F* to form QA*-F*-C18-A-G or QA*-F*-G respectively may be carried out by the enzyme QS-7-GIcT (SEQ ID NO 90), or an enzyme having an amino acid sequence with at least 70% sequence identity to the sequence for QS-7-GIcT (SEQ ID No 90). The amino acid sequence of the QS-7-GIcT enzyme may have at least 70%, 75%, 80%, 85%, 90%, 95%, 96%, 97%, 98% or 99% identity to SEQ ID NO 90. Accordingly, in some embodiments, the QS-7-GIcT has at least 70%, 75%, 80%, 85%, 90%, 95%, 96%, 97%, 98% or 99% identity to SEQ ID NO 90, suitably at least 90%, more suitably at least 95%. In respect of the enzymes defined here in terms of sequence identity, they typically retain the function of transferring a glucose residue to a molecule comprising QA*-F*-C18-A or QA*-F* to form QA*-F*-C18-A-G or QA*-F*-G respectively.

The percentage sequence identity of the sequences to 6CCL, 5CCL, 4CCL, 3CCL, 2CCL, 1CCL, ChSA, ChSB, ChSC, ChSD, ChSE, ChSF, KR11, KR23′, DMOT9, DMOT4, UGT-L-short, UGT-L-long and QS-7-GIcT may all be the same or different.

As mentioned above, the methods of these aspects of the invention comprise adding acyl units ((3S,5S,6S)-3,5-dihydroxy-6-methyloctanoyl-CoA) and/or a glucose residue to QA*-F*. QA*-F* is described above. An additional feature of the methods of these aspects of the invention is the steps for forming the QA backbone, the branched trisaccharide at the C-3 position of the molecule comprising a QA backbone and the linear tetrasaccharide at the C-28 position of the molecule comprising a QA backbone.

### QA Backbone Synthesis

One step of the method of forming the QA backbone of a molecule comprising QA*-F* is the cyclisation of 2,3-oxidosqualene to form a molecule comprising triterpene β-amyrin. This step is carried out by an oxidosqualene cyclase. In particular the oxidosqualene cyclase may be an enzyme according to QsbAS (SEQ ID NO 18) or an enzyme having an amino acid sequence with at least 50% sequence identity to SEQ ID NO 18. The oxidosqualene cyclase may be encoded by the polynucleotide sequence of SEQ ID NO 17.

This step encompasses the use of oxidosqualene cyclase enzymes having at least 50% sequence identity to the sequence for QsbAS (SEQ ID NO 18). The amino acid sequence of the QsbAS enzyme may have at least 50%, 55%, 65%, 70%, 75%, 80%, 85%, 90%, 95%, 96%, 97%, 98% or 99% identity to SEQ ID NO 18. Accordingly, in some embodiments, the QsbAS enzyme has at least 50%, 55%, 65%, 70%, 75%, 80%, 85%, 90%, 95%, 96%, 97%, 98% or 99% identity to SEQ ID NO 18, suitably at least 90%, more suitably at least 95%. In respect of the enzymes defined here in terms of sequence identity, they typically retain the function of the cyclisation of 2,3-oxidosqualene to form a molecule comprising triterpene β-amyrin.

The molecule comprising the β-amyrin scaffold is further oxidised to a carboxylic acid, alcohol and aldehyde at the C-28, C-16a and C-23 positions, respectively.

Another step of this feature of the invention is the oxidation of the molecule comprising the β-amyrin scaffold to form a carboxylic acid at the C-28 position. This step is carried out by a cytochrome P450 monooxygenase. The cytochrome P450 monooxygenase may be a Q. saponaria C-28 oxidase QsCYP716-C-28. For example, the C-28 oxidase QsCYP716-C-28 may be according to SEQ ID NO 20 or a sequence with at least 50% sequence identity to SEQ ID NO 20. QsCYP716-C-28 may be encoded by the polynucleotide sequence of SEQ ID NO 19 or a sequence with at least 50% sequence identity to SEQ ID NO 19. Alternative cytochrome P450 monooxygenase enzymes which catalyse oxidation of the molecule comprising the β-amyrin scaffold to form a carboxylic acid at the C-28 position are disclosed in Table 8 of WO2019/122259, the content of which is incorporated by reference.

This step encompasses the use of cytochrome P450 monooxygenases having at least 50% sequence identity to the sequence for QsCYP716-C-28 (SEQ ID NO 20). The amino acid sequence of the QsCYP716-C-28 enzyme may have at least 50%, 55%, 65%, 70%, 75%, 80%, 85%, 90%, 95%, 96%, 97%, 98% or 99% identity to SEQ ID NO 20. Accordingly, in some embodiments, the QsCYP716-C-28 enzyme has at least 50%, 55%, 65%, 70%, 75%, 80%, 85%, 90%, 95%, 96%, 97%, 98% or 99% identity to SEQ ID NO 20, suitably at least 90%, more suitably at least 95%. In respect of the enzymes defined here in terms of sequence identity, they typically retain the function of oxidising a molecule comprising the β-amyrin scaffold to form a carboxylic acid at the C-28 position.

Another step of this feature of the invention is the oxidation of the molecule comprising the β-amyrin scaffold to form an alcohol at the C-16 position. This step is performed by a cytochrome P450 monooxygenase. The cytochrome P450 monooxygenase may be a Q. saponaria C-16a oxidase QsCYP716-C-16a. For example, the C-16a oxidase QsCYP716-C-16a may be according to SEQ ID NO 22 or a sequence with at least 50% sequence identity to SEQ ID NO 22. QsCYP716-C-16a may be encoded by the polynucleotide sequence of SEQ ID NO 21 or a sequence with at least 50% sequence identity to SEQ ID NO 21. Alternative cytochrome P450 monooxygenase enzymes which catalyse oxidation of the molecule comprising the β-amyrin scaffold to form a carboxylic acid at the C-16a position are disclosed in Table 8 of WO2019/122259, the content of which is incorporated by reference.

This step encompasses the use of cytochrome P450 monooxygenases having at least 50% sequence identity to the sequence for QsCYP716-C-16a (SEQ ID NO 22). The amino acid sequence of the QsCYP716-C-16a enzyme may have at least 50%, 55%, 65%, 70%, 75%, 80%, 85%, 90%, 95%, 96%, 97%, 98% or 99% identity to SEQ ID NO 22. Accordingly, in some embodiments, the QsCYP716-C-16a enzyme has at least 50%, 55%, 65%, 70%, 75%, 80%, 85%, 90%, 95%, 96%, 97%, 98% or 99% identity to SEQ ID NO 22, suitably at least 90%, more suitably at least 95%. In respect of the enzymes defined here in terms of sequence identity, they typically retain the function of oxidising a molecule comprising the β-amyrin scaffold to form an alcohol at the C-16 position.

A further step of this feature of the invention is the oxidation of the molecule comprising the β-amyrin scaffold to form an aldehyde at the C-23 position. This step is performed by a cytochrome P450 monooxygenase. The cytochrome P450 monooxygenase may be a Q. saponaria C-23 oxidase QsCYP714-C-23. For example, the C-23 oxidase QsCYP714-C-23 may be according to SEQ ID NO 24 or a sequence with at least 50% sequence identity to SEQ ID NO 24. QsCYP714-C-23 may be encoded by the polynucleotide sequence of SEQ ID NO 23 or a sequence with at least 50% sequence identity to SEQ ID NO 23. Alternative cytochrome P450 monooxygenase enzymes which catalyse oxidation of the molecule comprising the β-amyrin scaffold to form a carboxylic acid at the C-23 position are disclosed in Table 8 of WO2019/122259, the content of which is incorporated by reference.

This step encompasses the use of cytochrome P450 monooxygenases having at least 50% sequence identity to the sequence for QsCYP714-C-23 (SEQ ID NO 24). The amino acid sequence of the QsCYP714-C-23 enzyme may have at least 50%, 55%, 65%, 70%, 75%, 80%, 85%, 90%, 95%, 96%, 97%, 98% or 99% identity to SEQ ID NO 24. Accordingly, in some embodiments, the QsCYP714-C-23 enzyme has at least 50%, 55%, 65%, 70%, 75%, 80%, 85%, 90%, 95%, 96%, 97%, 98% or 99% identity to SEQ ID NO 24, suitably at least 90%, more suitably at least 95%. In respect of the enzymes defined here in terms of sequence identity, they typically retain the function of oxidising a molecule comprising the β-amyrin scaffold to form an aldehyde at the C-23 position.

These steps form the QA backbone.

This feature of the invention relates to a method of forming a molecule comprising the QA backbone involving a number of steps. The steps can be performed in a specific order or in any order or simultaneously. Preferably, this molecule is formed by the production of the β-amyrin scaffold followed by the sequential oxidation at the C-28, C-16a and C-23 positions, respectively. The steps of this feature of the invention are described for the preferable situation mentioned above. However, the steps may occur in any order.

The sugar units forming the C-3 branched trisaccharide and C-28 linear tetrasaccharide chains are then added. Preferably the molecule comprising the QA backbone is formed, then the steps for adding the C-3 chain are carried out, followed by the steps for adding the C-28 tetrasaccharide chain. However, these steps can be performed in a specific order or in any order or simultaneously.

### C-3 Branched Trisaccharide Synthesis

The steps of the formation of QA*-F* are described for the situation when the branched trisaccharide at the C-3 position of the molecule comprising the QA backbone is initiated by attaching a β-D-glucopyranuronic acid residue to a molecule comprising QA to form a molecule comprising QA-Mono. However, the steps may occur in any order.

The first step of forming the C-3 chain is attaching a β-D-glucopyranuronic acid residue to a molecule comprising QA to form a molecule comprising QA-Mono. The step may be carried out by an enzyme QsCSL1 according to SEQ ID NO 26 or an enzyme QsCsIG2 according to SEQ ID NO 28, or a sequence with at least 70% sequence identity to SEQ ID NO 26 or 28. QsCSL1 may be encoded by the polynucleotide sequence of SEQ ID NO 25 or a sequence with at least 70% sequence identity to SEQ ID NO 25. QsCsIG2 may be encoded by the polynucleotide sequence of SEQ ID NO 27 or a sequence with at least 70% sequence identity to SEQ ID NO 27.

This step encompasses the use of enzymes having at least 70% sequence identity to the sequences for QsCSL1 and QsCsIG2 (SEQ ID NO 26 or 28 respectively). The amino acid sequence of the QsCSL1 enzyme may have at least 70%, 75%, 80%, 85%, 90%, 95%, 96%, 97%, 98% or 99% identity to SEQ ID NO 26. The amino acid sequence of the QsCsIG2 enzyme may have at least 70%, 75%, 80%, 85%, 90%, 95%, 96%, 97%, 98% or 99% identity to SEQ ID NO 28. Accordingly, in some embodiments, the QsCSL1 and/or QsCsIG2 enzyme has at least 70%, 75%, 80%, 85%, 90%, 95%, 96%, 97%, 98% or 99% identity to SEQ ID NO 26 or 28, suitably at least 90%, more suitably at least 95%. In respect of the enzymes defined here in terms of sequence identity, they typically retain the function of attaching a β-D-glucopyranuronic acid residue to a molecule comprising QA to form a molecule comprising QA-Mono.

Another step of the method of forming the C-3 chain is attaching a D-galactopyranose residue to a β-D-glucopyranuronic acid residue on a molecule comprising QA-Mono to form a molecule comprising QA-Di. The step may be carried out by an enzyme Qs-3-O-GaIT according to SEQ ID NO 30 or a sequence with at least 70% sequence identity to SEQ ID NO 30. Qs-3-O-GaIT may be encoded by the polynucleotide sequence of SEQ ID NO 29 or a sequence with at least 70% sequence identity to SEQ ID NO 29.

This step encompasses the use of enzymes having at least 70% sequence identity to the sequence for Qs-3-O-GaIT (SEQ ID NO 30). The amino acid sequence of the Qs-3-O-GaIT enzyme may have at least 70%, 75%, 80%, 85%, 90%, 95%, 96%, 97%, 98% or 99% identity to SEQ ID NO 30. Accordingly, in some embodiments, the Qs-3-O-GaIT enzyme has at least 70%, 75%, 80%, 85%, 90%, 95%, 96%, 97%, 98% or 99% identity to SEQ ID NO 30, suitably at least 90%, more suitably at least 95%. In respect of the enzymes defined here in terms of sequence identity, they typically retain the function of attaching a D-galactopyranose residue to a β-D-glucopyranuronic acid residue on a molecule comprising QA-Mono to form a molecule comprising QA-Di.

A further step of the method of forming the C-3 chain is attaching a L-rhamnopyranose residue to a β-D-glucopyranuronic acid residue on a molecule comprising QA-Di, to form a molecule comprising QA-TriR. The step may be carried out by an enzyme DN20529_c0_g2_i8 according to SEQ ID NO 36, Qs_0283850 according to SEQ ID NO 34, or an enzyme Qs-3-O-RhaT/XyIT according to SEQ ID NO 32, or a sequence with at least 70% sequence identity to SEQ ID NO 36, 34 or 32. DN20529_c0_g2_i8 may be encoded by the polynucleotide sequence of SEQ ID NO 35 or a sequence with at least 70% sequence identity to SEQ ID NO 35. Qs_0283850 may be encoded by the polynucleotide sequence of SEQ ID NO 33 or a sequence with at least 70% sequence identity to SEQ ID NO 33. Qs-3-O-RhaT/XyIT may be encoded by the polynucleotide sequence of SEQ ID NO 31 or a sequence with at least 70% sequence identity to SEQ ID NO 31.

This step encompasses the use of enzymes having at least 70% sequence identity to the sequences for DN20529_c0_g2_i8,Qs_0283850, or Qs-3-O-RhaT/XyIT (SEQ ID NO 36, 34 or 32 respectively). The amino acid sequence of the DN20529_c0_g2_i8 enzyme may have at least 70%, 75%, 80%, 85%, 90%, 95%, 96%, 97%, 98% or 99% identity to SEQ ID NO 36. The amino acid sequence of the Qs_0283850 enzyme may have at least 70%, 75%, 80%, 85%, 90%, 95%, 96%, 97%, 98% or 99% identity to SEQ ID NO 34. The amino acid sequence of the Qs-3-O-RhaT/XyIT enzyme may have at least 70%, 75%, 80%, 85%, 90%, 95%, 96%, 97%, 98% or 99% identity to SEQ ID NO 32. Accordingly, in some embodiments, the DN20529_c0_g2_i8, Qs_0283850 enzyme, and/or Qs-3-O-RhaT/XyIT enzyme has at least 70%, 75%, 80%, 85%, 90%, 95%, 96%, 97%, 98% or 99% identity to SEQ ID NO 36, 34 or 32, suitably at least 90%, more suitably at least 95%. In respect of the enzymes defined here in terms of sequence identity, they typically retain the function of attaching a L-rhamnopyranose residue to a β-D-glucopyranuronic acid residue on a molecule comprising QA-Di, to form a molecule comprising QA-TriR.

Yet a further step of the method of forming the C-3 chain is attaching a β-D-xylopyranose residue to a β-D-glucopyranuronic acid residue on a molecule comprising QA-Di, to form a molecule comprising QA-TriX. This step may be carried out by an enzyme Qs_0283870 according to SEQ ID NO 38, or an enzyme Qs-3-O-RhaT/XyIT according to SEQ ID NO 32, or a sequence with at least 70% sequence identity to SEQ ID NO 38 or 32. Qs_0283870 may be encoded by the polynucleotide sequence of SEQ ID NO 37 or a sequence with at least 70% sequence identity to SEQ ID NO 37. Qs-3-O-RhaT/XyIT may be encoded by the polynucleotide sequence of SEQ ID NO 31 or a sequence with at least 70% sequence identity to SEQ ID NO 31.

This step encompasses the use of enzymes having at least 70% sequence identity to the sequences for Qs_0283870 or Qs-3-O-RhaT/XyIT (SEQ ID NO 38 or 32 respectively). The amino acid sequence of the Qs_0283870 enzyme may have at least 70%, 75%, 80%, 85%, 90%, 95%, 96%, 97%, 98% or 99% identity to SEQ ID NO 38. The amino acid sequence of the Qs-3-O-RhaT/XyIT enzyme may have at least 70%, 75%, 80%, 85%, 90%, 95%, 96%, 97%, 98% or 99% identity to SEQ ID NO 32. Accordingly, in some embodiments, the Qs_0283870 and/or Qs-3-O-RhaT/XyIT enzyme has at least 70%, 75%, 80%, 85%, 90%, 95%, 96%, 97%, 98% or 99% identity to SEQ ID NO 38 or 32, suitably at least 90%, more suitably at least 95%. In respect of the enzymes defined here in terms of sequence identity, they typically retain the function of attaching a β-D-xylopyranose residue to a β-D-glucopyranuronic acid residue on a molecule comprising QA-Di, to form a molecule comprising QA-TriX.

These steps form the C-3 branched trisaccharide on the QA backbone.

### C-28 Linear Tetrasaccharide Synthesis

The steps of the formation of the C-28 linear tetrasaccharide (sugar) chain of a molecule comprising QA*-F* are described for the situation when the linear tetrasaccharide at the C-28 position of the molecule comprising the QA backbone is initiated by attaching a UDP-α-D-fucose residue to a molecule comprising QA* to form a molecule comprising QA*-F. However, the steps may occur in any order. For example, the C-28 linear tetrasaccharide chain, FRX(X/A), may be produced and then attached to the QA* backbone.

The first step of forming the C-28 sugar chain may be attaching a UDP-α-D-fucose residue to the C-28 position of a molecule comprising QA, QA-Mono, QA-Di and/or QA-Tri(R/X), to form a molecule comprising QA-F, QA-Mono-F, QA-Di-F and/or QA-Tri(R/X)-F. This step may be carried out by an enzyme Qs-28-O-FucT according to SEQ ID NO 2 or a sequence with at least 70% sequence identity to SEQ ID NO 2. Qs-28-O-FucT may be encoded by the polynucleotide sequence of SEQ ID NO 1 or a sequence with at least 70% sequence identity to SEQ ID NO 1. The first step of forming the C-28 linear tetrasaccharide chain may also be attaching UDP-4-keto, 6-deoxy-D-glucose to a molecule comprising QA, QA-Mono, QA-Di and/or QA-Tri(R/X), to form a molecule comprising QA-F, QA-Mono-F, QA-Di-F and/or QA-Tri(R/X)-F. This step may be carried out by the enzymes Qs-28-O-FucT according to SEQ ID NO 2 or a sequence with at least 70% sequence identity to SEQ ID NO 2 and QsFucSyn according to SEQ ID NO 12 or a sequence with at least 45% sequence identity to SEQ ID NO 12. Qs-28-O-FucT may be encoded by the polynucleotide sequence of SEQ ID NO 1 or a sequence with at least 70% sequence identity to SEQ ID NO 1. QsFucSyn may be encoded by the polynucleotide sequence of SEQ ID NO 11 or a sequence with at least 45% sequence identity to SEQ ID NO 11.

This step encompasses the use of enzymes having at least 70% sequence identity to the sequence for Qs-28-O-FucT (SEQ ID NO 2). The amino acid sequence of the Qs-28-O-FucT enzyme may have at least 70%, 75%, 80%, 85%, 90%, 95%, 96%, 97%, 98% or 99% identity to SEQ ID NO 2. Accordingly, in some embodiments, the Qs-28-O-FucT has at least 70%, 75%, 80%, 85%, 90%, 95%, 96%, 97%, 98% or 99% identity to SEQ ID NO 2, suitably at least 90%, more suitably at least 95%. In respect of the enzymes defined here in terms of sequence identity, they typically retain the function of attaching a UDP-α-D-fucose residue to the C-28 position of a molecule comprising QA, QA-Mono, QA-Di and/or QA-Tri(R/X), to form a molecule comprising QA-F, QA-Mono-F, QA-Di-F and/or QA-Tri(R/X)-F; or attaching UDP-4-keto, 6-deoxy-D-glucose to a molecule comprising QA, QA-Mono, QA-Di and/or QA-Tri(R/X), to form a molecule comprising QA-F, QA-Mono-F, QA-Di-F and/or QA-Tri(R/X)-F.

This step also encompasses the use of enzymes having at least 45% sequence identity to the sequence for QsFucSyn (SEQ ID NO 12). The amino acid sequence of the QsFucSyn enzyme may have at least 45%, 50%, 55%, 60%, 65%, 70%, 75%, 80%, 85%, 90%, 95%, 96%, 97%, 98% or 99% identity to SEQ ID NO 12. Accordingly, in some embodiments, the QsFucSyn has at least 45%, 50%, 55%, 60%, 65%, 70%, 75%, 80%, 85%, 90%, 95%, 96%, 97%, 98% or 99% identity to SEQ ID NO 12, suitably at least 90%, more suitably at least 95%. In respect of the enzymes defined here in terms of sequence identity, they typically retain the function of attaching a UDP-α-D-fucose residue to the C-28 position of a molecule comprising QA, QA-Mono, QA-Di and/or QA-Tri(R/X), to form a molecule comprising QA-F, QA-Mono-F, QA-Di-F and/or QA-Tri(R/X)-F; or attaching UDP-4-keto, 6-deoxy-D-glucose to a molecule comprising QA, QA-Mono, QA-Di and/or QA-Tri(R/X), to form a molecule comprising QA-F, QA-Mono-F, QA-Di-F and/or QA-Tri(R/X)-F.

Another step of forming the C-28 linear tetrasaccharide chain is attaching a UDP-β-L-rhamnose residue to a UDP-α-D-fucose residue on a molecule comprising QA-F, QA-Mono-F, QA-Di-F and/or QA-Tri(R/X)-F, to form a molecule comprising QA-FR, QA-Mono-FR, QA-Di-FR and/or QA-Tri(R/X)-FR. This step may be carried out by an enzyme Qs-28-O-RhaT according to SEQ ID NO 4 or a sequence with at least 70% sequence identity to SEQ ID NO 4. Qs-28-O-RhaT may be encoded by the polynucleotide sequence of SEQ ID NO 3 or a sequence with at least 70% sequence identity to SEQ ID NO 3.

This step also encompasses the use of enzymes having at least 70% sequence identity to the sequence for Qs-28-O-RhaT (SEQ ID NO 4). The amino acid sequence of the Qs-28-O-RhaT enzyme may have at least 70%, 75%, 80%, 85%, 90%, 95%, 96%, 97%, 98% or 99% identity to SEQ ID NO 4. Accordingly, in some embodiments, the Qs-28-O-RhaT has at least 70%, 75%, 80%, 85%, 90%, 95%, 96%, 97%, 98% or 99% identity to SEQ ID NO 4, suitably at least 90%, more suitably at least 95%. In respect of the enzymes defined here in terms of sequence identity, they typically retain the function of attaching a UDP-β-L-rhamnose residue to a UDP-α-D-fucose residue on a molecule comprising QA-F, QA-Mono-F, QA-Di-F and/or QA-Tri(R/X)-F, to form a molecule comprising QA-FR, QA-Mono-FR, QA-Di-FR and/or QA-Tri(R/X)-FR.

A further step for forming the C-28 linear tetrasaccharide chain is attaching a UDP-α-D-xylose residue to a UDP-β-L-rhamnose residue on a molecule comprising QA-FR, QA-Mono-FR, QA-Di-FR and/or QA-Tri(R/X)-FR, to form a molecule comprising QA-FRX, QA-Mono-FRX, QA-Di-FRX and/or QA-Tri(R/X)-FRX. This step may be carried out by an enzyme Qs-28-O-XyIT3 according to SEQ ID NO 6 or a sequence with at least 70% sequence identity to SEQ ID NO 6. Qs-28-O-XyIT3 may be encoded by the polynucleotide sequence of SEQ ID NO 5 or a sequence with at least 70% sequence identity to SEQ ID NO 5.

This step also encompasses the use of enzymes having at least 70% sequence identity to the sequence for Qs-28-O-XyIT3 (SEQ ID NO 6). The amino acid sequence of the Qs-28-O-XyIT3 enzyme may have at least 70%, 75%, 80%, 85%, 90%, 95%, 96%, 97%, 98% or 99% identity to SEQ ID NO 6. Accordingly, in some embodiments, the Qs-28-O-XyIT3 has at least 70%, 75%, 80%, 85%, 90%, 95%, 96%, 97%, 98% or 99% identity to SEQ ID NO 6, suitably at least 90%, more suitably at least 95%. In respect of the enzymes defined here in terms of sequence identity, they typically retain the function of attaching a UDP-α-D-xylose residue to a UDP-β-L-rhamnose residue on a molecule comprising QA-FR, QA-Mono-FR, QA-Di-FR and/or QA-Tri(R/X)-FR, to form a molecule comprising QA-FRX, QA-Mono-FRX, QA-Di-FRX and/or QA-Tri(R/X)-FRX.

Yet another step for forming the C-28 linear tetrasaccharide chain may be attaching a UDP-α-D-xylose residue to a UDP-α-D-xylose residue on a molecule comprising QA-Mono-FRX, QA-Di-FRX and/or QA-Tri(R/X)-FRX to form a molecule comprising QA-Mono-FRXX, QA-Di-FRXX and/or QA-Tri(R/X)-FRXX. This step may be carried out by an enzyme Qs-28-O-XyIT4 according to SEQ ID NO 8 or a sequence with at least 70% sequence identity to SEQ ID NO 8. Qs-28-O-XyIT4 may be encoded by the polynucleotide sequence of SEQ ID NO 7 or a sequence with at least 70% sequence identity to SEQ ID NO 7.

This step also encompasses the use of enzymes having at least 70% sequence identity to the sequence for Qs-28-O-XyIT4 (SEQ ID NO 8). The amino acid sequence of the Qs-28-O-XyIT4 enzyme may have at least 70%, 75%, 80%, 85%, 90%, 95%, 96%, 97%, 98% or 99% identity to SEQ ID NO 8. Accordingly, in some embodiments, the Qs-28-O-XyIT4 has at least 70%, 75%, 80%, 85%, 90%, 95%, 96%, 97%, 98% or 99% identity to SEQ ID NO 8, suitably at least 90%, more suitably at least 95%. In respect of the enzymes defined here in terms of sequence identity, they typically retain the function of attaching a UDP-α-D-xylose residue to a UDP-α-D-xylose residue on a molecule comprising QA-Mono-FRX, QA-Di-FRX and/or QA-Tri(R/X)-FRX to form a molecule comprising QA-Mono-FRXX, QA-Di-FRXX and/or QA-Tri(R/X)-FRXX.

Another step for forming the C-28 linear tetrasaccharide chain may be attaching a UDP-α-D-apiose residue to a UDP-α-D-xylose residue on a molecule comprising QA-Mono-FRX, QA-Di-FRX and/or QA-Tri(R/X)-FRX to form a molecule comprising QA-Mono-FRXA, QA-Di-FRXA and/or QA-Tri(R/X)-FRXA. This step may be carried out by an enzyme Qs-28-O-ApiT4 according to SEQ ID NO 10 or a sequence with at least 70% sequence identity to SEQ ID NO 10. Qs-28-O-ApiT4 may be encoded by the polynucleotide sequence of SEQ ID NO 9 or a sequence with at least 70% sequence identity to SEQ ID NO 9.

This step also encompasses the use of enzymes having at least 70% sequence identity to the sequence for Qs-28-O-ApiT4 (SEQ ID NO 10). The amino acid sequence of the Qs-28-O-ApiT4 enzyme may have at least 70%, 75%, 80%, 85%, 90%, 95%, 96%, 97%, 98% or 99% identity to SEQ ID NO 10. Accordingly, in some embodiments, the Qs-28-O-ApiT4 has at least 70%, 75%, 80%, 85%, 90%, 95%, 96%, 97%, 98% or 99% identity to SEQ ID NO 10, suitably at least 90%, more suitably at least 95%. In respect of the enzymes defined here in terms of sequence identity, they typically retain the function of attaching a UDP-α-D-apiose residue to a UDP-α-D-xylose residue on a molecule comprising QA-Mono-FRX, QA-Di-FRX and/or QA-Tri(R/X)-FRX to form a molecule comprising QA-Mono-FRXA, QA-Di-FRXA and/or QA-Tri(R/X)-FRXA.

The method of the fourth aspect of the invention is carried out in a biological system, or host. The polynucleotides encoding for one or more of the above enzymes are introduced and expressed in the biological system. In most cases, the biological system will not naturally express any of the enzymes of the first aspect of the invention and thus the biological system will be engineered to express all the enzymes.

The biological system may be a plant or a microorganism. When the biological system is a plant, the plant may be row crops for example sunflower, potato, canola, dry bean, field pea, flax, safflower, buckwheat, cotton, maize, soybeans and sugar beets. The plant may also be corn, wheat, oilseed rape and rice. Preferably the plant may be Nicotiana benthamiana. In certain embodiments of the methods of the fourth aspect of the invention, the biological system is not Quillaja saponaria.

When the biological system is a microorganism, the microorganism may be bacteria or yeast.

Yeast (Saccharomyces cerevisiae) is a heterologous host used for the production of high value small molecules, including terpenes. Like plants, yeast endogenously produces the triterpenoid precursor 2,3-oxidosqualene, and so is a promising host for industrial-scale production of triterpenoids. It is also a highly effective host for the functional expression of plant CYPs at endoplasmic reticulum membranes. There is minimal modification of triterpenoid scaffolds by endogenous yeast enzymes, facilitating product purification. Yeast can be a production host producing triterpenes with diverse glycoside conjugates comprising multiple types of sugars in linear and branched configuration. Glycosylation reactions in yeast are restricted by the limited palette of endogenous sugar donors. By expressing genes from higher plants, however, the nucleotide sugar metabolism of yeast can be expanded beyond UDP-glucose and UDP-galactose, to include UDP-rhamnose, -glucuronic acid, -xylose, -arabinose and others.

The method of the first aspect of the invention may be performed in vitro. By “in vitro”, it is meant in the sense of the present invention to have appropriate QA*-F* derivatives enzymatically treated with appropriate enzymes of the invention. QA*-F* derivatives may be either biosynthetically produced or chemically synthesized. Enzymes may be either cloned or purified from their native environment. It is within the skilled person's ambit to determine the optimal conditions (e.g. duration, temperature, buffer etc.) of the enzymatic treatment.

In one embodiment, the in vitro method of the first aspect of the invention to make QA*-F*-C18-A comprises treating a molecule comprising QA*-F* with a mixture of enzymes comprising:
(i) at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64, 62, 60, 58, 56 or 54.
(ii) at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74) ChSF (SEQ ID NO 76) and an enzyme having an amino acid sequence with at least 50% sequence identity to SEQ ID NO 66, 68, 70, 72, 74 or 76;
(iii) at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80), and an enzyme having an amino acid sequence with at least 15% sequence identity to SEQ ID NO 80,
(iv) at least one or more enzymes selected from DMOT9 (SEQ ID NO 82) and an enzyme having an amino acid sequence with at least 25% sequence identity to SEQ ID NO 82,
(v) at least one or more enzymes selected from DMOT4 (SEQ ID NO 84) and an enzyme having an amino acid sequence with at least 15% sequence identity to SEQ ID NO 84, and
(vi) at least one or more enzymes selected from UGT-L-short (SEQ ID NO 86), UGT-L-long (SEQ ID NO 88), and an enzyme having an amino acid sequence with at least 45% sequence identity to SEQ ID NO 86 or 88, in the presence of 2-methylbutyric acid, malonyl-CoA and UDP-β-L-arabinofuranose.

In the in vitro method of the first aspect of the invention, F* may be FRX, FRXX, FRXA or mixtures thereof. Preferably F* is FRXA.

In one embodiment, the in vitro method of the [insert] aspect of the invention to make QA*-F*-C18-A-G comprises treating a molecule comprising QA*-F* with a mixture of enzymes comprising:
(i) at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64, 62, 60, 58, 56 or 54.
(ii) at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74) ChSF (SEQ ID NO 76) and an enzyme having an amino acid sequence with at least 50% sequence identity to SEQ ID NO 66, 68, 70, 72, 74 or 76;
(iii) at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80), and an enzyme having an amino acid sequence with at least 15% sequence identity to SEQ ID NO 80,
(iv) at least one or more enzymes selected from DMOT9 (SEQ ID NO 82) and an enzyme having an amino acid sequence with at least 25% sequence identity to SEQ ID NO 82,
(v) at least one or more enzymes selected from DMOT4 (SEQ ID NO 84) and an enzyme having an amino acid sequence with at least 15% sequence identity to SEQ ID NO 84,
(vi) at least one or more enzymes selected from UGT-L-short (SEQ ID NO 86), UGT-L-long (SEQ ID NO 88), and an enzyme having an amino acid sequence with at least 45% sequence identity to SEQ ID NO 86 or 88, and
(vii) at least one or more enzymes selected from QS-7-GIcT (SEQ ID NO 90) and an enzyme having an amino acid sequence with at least 90% sequence identity to SEQ ID NO 90;
in the presence of 2-methylbutyric acid, malonyl-CoA and UDP-β-L-arabinofuranose.

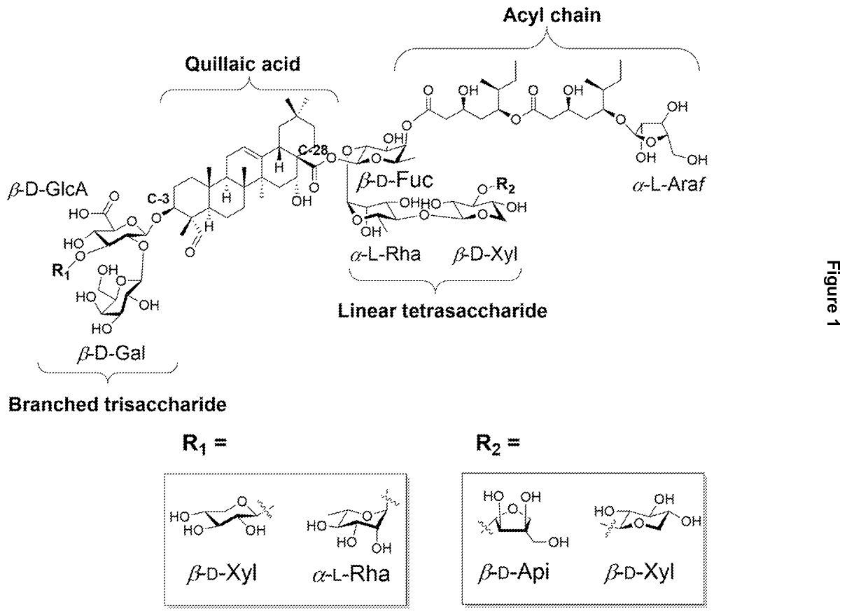

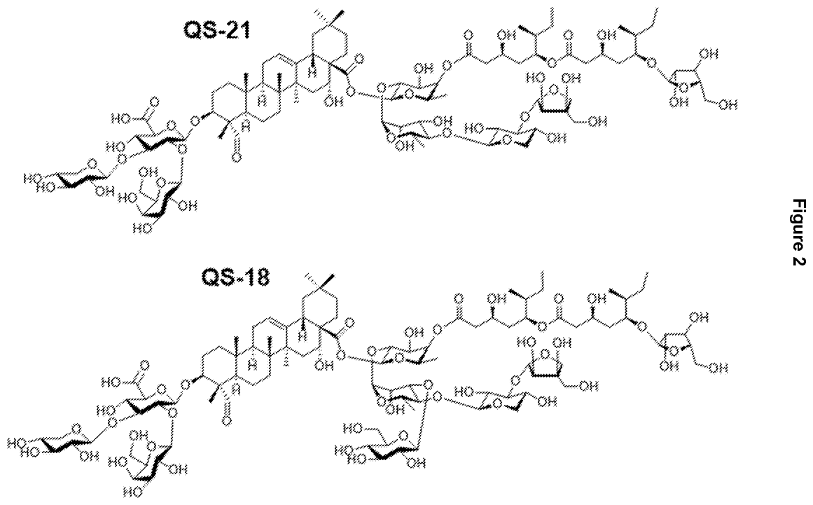

The methods of these aspects of the invention involve a number of steps which may be in any order. In summary, an acyl unit is produced, then attached to a molecule comprising QA*-F* (see FIG. 1) according to the first and fourth aspects of the invention. The molecule comprising QA*-F* may be QA-TriX-FRX, QA-TriR-FRX, QA-TriX-FRXX, QA-TriR-FRXX, QA-TriX-FRXA, QA-TriR-FRXA or mixtures thereof. A second acyl unit and arabinofuranose are then added. A D-glucose residue may then be added to the molecule comprising QA*-F* and the two acyl units (QA*-F*-C18-A) to make QA*-F*-C18-A-G, wherein F* is FRX, FRXX, FRXA or mixtures thereof, preferably wherein F* is FRXA. The glucose residue may also be added to a molecule comprising QA*-F* then two acyl units and arabinofuranose may be added.

In one embodiment of the fourth aspect of the invention, a method of making a biosynthetic QA*-F*-C18-A in a host, comprises the steps of a) expressing genes required for the biosynthesis of QA*-F* into the host, and b) introducing a polynucleotide encoding:
(i) at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56),1CCL (SEQ ID NO 54) and an enzyme having an amino acid sequence with at least 60% sequence identity to SEQ ID NO 64, 62, 60, 58, 56 or 54;
(ii) at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74), ChSF (SEQ ID NO 76) and an enzyme having an amino acid sequence with at least 50% sequence identity to SEQ ID NO 66, 68, 70, 72, 74 or 76;
(iii) at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80) and an enzyme having an amino acid sequence with at least 15% sequence identity to SEQ ID NO 80;
(iv) at least one or more enzymes selected from DMOT9 (SEQ ID NO 82), an enzyme having an amino acid sequence with at least 25% sequence identity to SEQ ID NO 82, DMOT4 (SEQ ID NO 84) and an enzyme having an amino acid sequence with at least 15% sequence identity to SEQ ID NO 84; and
(v) at least one or more enzymes selected from UGT-L-short (SEQ ID NO 86), UGT-L-long (SEQ ID NO 88) and an enzyme having an amino acid sequence with at least 45% sequence identity to SEQ ID NO 86 or 88,
into the host.

In one embodiment of the [insert] aspect of the invention, a method of making a biosynthetic QA*-F*-C18-A-G in a host, comprises the steps of a) expressing genes required for the biosynthesis of QA*-F* into the host, and b) introducing a polynucleotide encoding:
(i) at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64, 62, 60, 58, 56 or 54;
(ii) at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74), ChSF (SEQ ID NO 76) and an enzyme having an amino acid sequence with at least 50% sequence identity to SEQ ID NO 66, 68, 70, 72, 74 or 76;
(iii) at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80) and an enzyme having an amino acid sequence with at least 15% sequence identity to SEQ ID NO 80;
(iv) at least one or more enzymes selected from DMOT9 (SEQ ID NO 82), an enzyme having an amino acid sequence with at least 25% sequence identity to SEQ ID NO 82, DMOT4 (SEQ ID NO 84) and an enzyme having an amino acid sequence with at least 15% sequence identity to SEQ ID NO 84;
(v) at least one or more enzymes selected from UGT-L-short (SEQ ID NO 86), UGT-L-long (SEQ ID NO 88) and an enzyme having an amino acid sequence with at least 45% sequence identity to SEQ ID NO 86 or 88, and
(vi) at least one or more enzymes selected from QS-7-GIcT (SEQ ID NO 90) or an enzyme having an amino acid sequence with at least 70% sequence identity to SEQ ID NO 90. into the host.

The biosynthesis of QA*-F* may be obtained by introducing polynucleotide molecules into the host encoding:
(a) (i) QsbAS (SEQ ID NO 18); (ii) QsCYP716-C-28 (SEQ ID NO 20); (iii) QsCYP716-C-16a (SEQ ID NO 22); and (iv) QsCYP714-C-23 (SEQ ID NO 24);
(b) (i) QsCSL1 (SEQ ID NO 26) or QsCsIG2 (SEQ ID NO 28); optionally (ii) Qs-3-O-GaIT (SEQ ID NO 30); and/or optionally (iii) DN20529_c0_g2_i8 (SEQ ID NO 36), Qs_0283850 (SEQ ID NO 34), or Qs-3-O-RhaT/XyIT (SEQ ID NO 32), and/or Qs_0283870 (SEQ ID NO 38) or Qs-3-O-RhaT/XyIT (SEQ ID NO 32);
(c) (i) Qs-28-O-FucT (SEQ ID NO 2) and optionally QsFucSyn (SEQ ID NO 12); optionally (ii) Qs-28-O-RhaT (SEQ ID NO 4); optionally (iii) Qs-28-O-XyIT3 (SEQ ID NO 6); optionally (iv) Qs-28-O-XyIT4 (SEQ ID NO 8) and/or (v) Qs-28-O-ApiT4 (SEQ ID NO 10).

In the fourth aspect of the invention, amino acid SEQ ID NO 54 is encoded by polynucleotide SEQ ID NO 53; amino acid SEQ ID NO 56 is encoded by polynucleotide SEQ ID NO 55; amino acid SEQ ID NO 58 is encoded by polynucleotide SEQ ID NO 57; amino acid SEQ ID NO 60 is encoded by polynucleotide SEQ ID NO 59; amino acid SEQ ID NO 62 is encoded by polynucleotide SEQ ID NO 61; amino acid SEQ ID NO 64 is encoded by polynucleotide SEQ ID NO 63; amino acid SEQ ID NO 66 is encoded by polynucleotide SEQ ID NO 65; amino acid SEQ ID NO 68 is encoded by polynucleotide SEQ ID NO 67; amino acid SEQ ID NO 70 is encoded by polynucleotide SEQ ID NO 69; amino acid SEQ ID NO 72 is encoded by polynucleotide SEQ ID NO 71; amino acid SEQ ID NO 74 is encoded by polynucleotide SEQ ID NO 73; amino acid SEQ ID NO 76 is encoded by polynucleotide SEQ ID NO 75; amino acid SEQ ID NO 78 is encoded by polynucleotide SEQ ID NO 77; amino acid SEQ ID NO 80 is encoded by polynucleotide SEQ ID NO 79; amino acid SEQ ID NO 82 is encoded by polynucleotide SEQ ID NO 81; amino acid SEQ ID NO 84 is encoded by polynucleotide SEQ ID NO 83; amino acid SEQ ID NO 86 is encoded by polynucleotide SEQ ID NO 85; amino acid SEQ ID NO 88 is encoded by polynucleotide SEQ ID NO 87; amino acid SEQ ID NO 90 is encoded by polynucleotide SEQ ID NO 89.

The method of the fourth aspect of the invention includes transforming the host with polynucleotides by introducing the polynucleotides required for the biosynthesis of a molecule comprising QA*-F*-C18-A and/or QA*-F*-C18-A-G into the host cells via a vector(s). Recombination may occur between the vector(s) and the host cell genome to introduce the polynucleotides into the host cell genome.

The method of this aspect of the invention comprises the step of adding 2-methylbutyric acid. One option to increase the amounts of the C9 acyl unit available is to add 2-methylbutyric acid. The addition of 2-methylbutyric acid should increase the intra-cellular concentration of 2-methylbutyryl-CoA, and therefore increase the availability of the C9 acyl unit. To boost the QS-21 yields, 2-methylbutyric acid may be added to an infiltration solution. The infiltration solution may be an Agrobacterium buffer. 2-Methylbutyric acid may be provided to the infiltrated plant.

A sixth aspect of the invention is a carboxyl CoA enzyme having the amino acid sequence of SEQ ID NO 54 (1CCL), SEQ ID NO 56 (2CCL), SEQ ID NO 58 (3CCL), SEQ ID NO 60 (4CCL), SEQ ID NO 62 (5CCL), SEQ ID NO 64 (6CCL) or an enzyme having an amino acid sequence with at least 65% sequence identity to SEQ ID NO 54, 56, 58, 60, 62 or 64. The enzyme is capable of transferring a CoA to 2-methylbutyric acid. This enzyme is as described in the method of the first aspect of the invention and has the same properties and function as described in relation to the method of the first aspect of the invention.

The carboxyl CoA enzyme 1CCL may be encoded by a polynucleotide sequence of SEQ ID NO 53. The carboxyl CoA enzyme 2CCL is encoded by a polynucleotide sequence of SEQ ID NO 55. The carboxyl CoA enzyme 3CCL is encoded by a polynucleotide sequence of SEQ ID NO 57. The carboxyl CoA enzyme 4CCL is encoded by a polynucleotide sequence of SEQ ID NO 59. The carboxyl CoA enzyme 5CCL is encoded by a polynucleotide sequence of SEQ ID NO 61. The carboxyl CoA enzyme 6CCL is encoded by a polynucleotide sequence of SEQ ID NO 63.

The sixth aspect of the invention encompasses carboxyl CoA enzymes having an amino acid sequence with at least 60% sequence identity to the sequences for 1CCL, 2CCL, 3CCL, 4CCL, 5CCL and 6CCL (SEQ ID NO 54, 56, 58, 60, 62 or 64 respectively). The amino acid sequence of the 1CCL enzyme may have at least 60%, 65%, 70%, 75%, 80%, 85%, 90%, 95%, 96%, 97%, 98% or 99% identity to SEQ ID NO 54. The amino acid sequence of the 2CCL enzyme may have at least 60%, 65%, 70%, 75%, 80%, 85%, 90%, 95%, 96%, 97%, 98% or 99% identity to SEQ ID NO 56. The amino acid sequence of the 3CCL enzyme may have at least 60%, 65%, 70%, 75%, 80%, 85%, 90%, 95%, 96%, 97%, 98% or 99% identity to SEQ ID NO 58. The amino acid sequence of the 4CCL enzyme may have at least 60%, 65%, 70%, 75%, 80%, 85%, 90%, 95%, 96%, 97%, 98% or 99% identity to SEQ ID NO 60. The amino acid sequence of the 5CCL enzyme may have at least 60%, 65%, 70%, 75%, 80%, 85%, 90%, 95%, 96%, 97%, 98% or 99% identity to SEQ ID NO 62. The amino acid sequence of the 6CCL enzyme may have at least 60%, 65%, 70%, 75%, 80%, 85%, 90%, 95%, 96%, 97%, 98% or 99% identity to SEQ ID NO 64. Accordingly, in some embodiments, the 1CCL, 2CCL, 3CCL, 4CCL, 5CCL and/or 6CCL enzyme has at least 60%, 65%, 70%, 75%, 80%, 85%, 90%, 95%, 96%, 97%, 98% or 99% identity to SEQ ID NO 54, 56, 58, 60, 62 or 64, suitably at least 90%, more suitably at least 95%. In respect of the enzymes defined here in terms of sequence identity, they typically retain the function of transferring a CoA to 2-methylbutyric acid to form 2-methylbutyryl-CoA.

A seventh aspect of the invention is a chalcone synthase-like enzyme having the amino acid sequence of SEQ ID NO 66 (ChSA), SEQ ID NO 68 (ChSB), SEQ ID NO 70 (ChSC), SEQ ID NO 72 (ChSD), SEQ ID NO 74 (ChSE), SEQ ID NO 76 (ChSF) and/or an enzyme having an amino acid sequence with at least 50% sequence identity to SEQ ID NO 66, 68, 70, 72, 74 or 76. The enzyme is capable of converting 2-methylbutyryl-CoA to (S)-6-methyl-3,5-dioxooctanoyl-CoA. This enzyme is as described in the method of the first aspect of the invention and has the same properties and function as described in relation to the method of the first aspect of the invention. Preferably the chalcone synthase-like enzyme is ChSE (SEQ ID NO 74) or an enzyme having an amino acid sequence with at least 50% sequence identity to SEQ ID NO 74.

The chalcone synthase-like enzyme ChSA may be encoded by a polynucleotide sequence of SEQ ID NO 65. The chalcone synthase-like enzyme ChSB is encoded by a polynucleotide sequence of SEQ ID NO 67. The chalcone synthase-like enzyme ChSC is encoded by a polynucleotide sequence of SEQ ID NO 69. The chalcone synthase-like enzyme ChSD is encoded by a polynucleotide sequence of SEQ ID NO 71. The chalcone synthase-like enzyme ChSE is encoded by a polynucleotide sequence of SEQ ID NO 73. The chalcone synthase-like enzyme ChSF is encoded by a polynucleotide sequence of SEQ ID NO 75.

The seventh aspect of the invention encompasses chalcone synthase-like enzymes having an amino acid sequence with at least 50% sequence identity to the sequences for ChSA, ChSB, ChSC, ChSD, ChSE and ChSF (SEQ ID NO 66, 68, 70, 72, 74, 76 respectively). The amino acid sequence of the ChSA enzyme may have at least 50%, 55%, 60%, 65%, 70%, 75%, 80%, 85%, 90%, 95%, 96%, 97%, 98% or 99% identity to SEQ ID NO 66. The amino acid sequence of the ChSB enzyme may have at least 50%, 55%, 60%, 65%, 70%, 75%, 80%, 85%, 90%, 95%, 96%, 97%, 98% or 99% identity to SEQ ID NO 68. The amino acid sequence of the ChSC enzyme may have at least 50%, 55%, 60%, 65%, 70%, 75%, 80%, 85%, 90%, 95%, 96%, 97%, 98% or 99% identity to SEQ ID NO 70. The amino acid sequence of the ChSD enzyme may have at least 50%, 55%, 60%, 65%, 70%, 75%, 80%, 85%, 90%, 95%, 96%, 97%, 98% or 99% identity to SEQ ID NO 72. The amino acid sequence of the ChSE enzyme may have at least 50%, 55%, 60%, 65%, 70%, 75%, 80%, 85%, 90%, 95%, 96%, 97%, 98% or 99% identity to SEQ ID NO 74. The amino acid sequence of the ChSF enzyme may have at least 50%, 55%, 60%, 65%, 70%, 75%, 80%, 85%, 90%, 95%, 96%, 97%, 98% or 99% identity to SEQ ID NO 76. Accordingly, in some embodiments, the ChSA, ChSB, ChSC, ChSD, ChSE and/or ChSF has at least 50%, 55%, 60%, 65%, 70%, 75%, 80%, 85%, 90%, 95%, 96%, 97%, 98% or 99% identity to SEQ ID NO 66, 68, 70, 72 or 74, suitably at least 90%, more suitably at least 95%. In respect of the enzymes defined here in terms of sequence identity, they typically retain the function of converting 2-methylbutyryl-CoA to (S)-6-methyl-3,5-dioxooctanoyl-CoA (diketone).

A eighth aspect of the invention is a keto reductase enzyme having the amino acid sequence of SEQ ID NO 78 (KR11) or an enzyme having an amino acid sequence with at least 20% sequence identity to SEQ ID NO 78. This enzyme is capable of reducing a ketone and/or converting (S)-6-methyl-3,5-dioxooctanoyl-CoA to (3S,5S,6S)-3,5-dihydroxy-6-methyloctanoyl-CoA. This enzyme is as described in the method of the first aspect of the invention and has the same properties and function as described in relation to the method of the first aspect of the invention. The keto reductase enzyme KR11 may be encoded by a polynucleotide sequence of SEQ ID NO 77.

The eighth aspect of the invention encompasses keto reductase enzymes having an amino acid sequence with at least 20% sequence identity to the sequence for KR11 (SEQ ID NO 78). The amino acid sequence of the KR11 enzyme may have at least 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 78. Accordingly, in some embodiments, the KR11 has at least 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 78, suitably at least 90%, more suitably at least 95%. In respect of the enzymes defined here in terms of sequence identity, they typically retain the function of reducing a ketone and/or converting (S)-6-methyl-3,5-dioxooctanoyl-CoA to (3S,5S,6S)-3,5-dihydroxy-6-methyloctanoyl-CoA.

A ninth aspect of the invention is a keto reductase enzyme having the amino acid sequence of SEQ ID NO 80 (KR23′) or an enzyme having an amino acid sequence with at least 15% sequence identity to SEQ ID NO 80. This enzyme is capable of reducing a ketone and/or converting (S)-6-methyl-3,5-dioxooctanoyl-CoA to (3S,5S,6S)-3,5-dihydroxy-6-methyloctanoyl-CoA. This enzyme is as described in the method of the first aspect of the invention and has the same properties and function as described in relation to the method of the first aspect of the invention. The keto reductase enzyme KR23′ may be encoded by a polynucleotide sequence of SEQ ID NO 79.

The ninth aspect of the invention encompasses keto reductase enzymes having an amino acid sequence with at least 15% sequence identity of the sequence for KR23′ (SEQ ID NO 80). The amino acid sequence of the KR23′ enzyme may have at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 80. Accordingly, in some embodiments, the KR23′ has at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 80, suitably at least 90%, more suitably at least 95%. In respect of the enzymes defined here in terms of sequence identity, they typically retain the function of reducing a ketone and/or converting (S)-6-methyl-3,5-dioxooctanoyl-CoA to (3S,5S,6S)-3,5-dihydroxy-6-methyloctanoyl-CoA.

A tenth aspect of the invention is an acyl transferase enzyme having the amino acid sequence of SEQ ID NO 82 (DMOT9) or an enzyme having an amino acid sequence with at least 25% sequence identity to SEQ ID NO 82. This enzyme is capable of transferring an acyl unit to QA*-F* to form QA*-F*-C9 or transferring an acyl unit to QA*-F*-G to form QA*-F*-C9-G. This enzyme is as described in the method of the first aspect of the invention and has the same properties and function as described in relation to the method of the first aspect of the invention. The acyl transferase enzyme DMOT9 may be encoded by a polynucleotide sequence of SEQ ID NO 81.

The tenth aspect of the invention encompasses acyl transferase enzymes having an amino acid sequence with at least 25% sequence identity to the sequence for DMOT9 (SEQ ID NO 82). The amino acid sequence of the DMOT9 enzyme may have at least 25%, 30%, 35%, 40%, 45%, 50%, 55%, 60%, 65%, 70%, 75%, 80%, 85%, 90%, 95%, 96%, 97%, 98% or 99% identity to SEQ ID NO 82. Accordingly, in some embodiments, the DMOT9 has at least 25%, 30%, 35%, 40%, 45%, 50%, 55%, 60%, 65%, 70%, 75%, 80%, 85%, 90%, 95%, 96%, 97%, 98% or 99% identity to SEQ ID NO 82, suitably at least 90%, more suitably at least 95%. In respect of the enzymes defined here in terms of sequence identity, they typically retain the function of transferring (3S,5S,6S)-3,5-dihydroxy-6-methyloctanoyl-CoA to QA*-F* or QA*-F*-G.

An eleventh aspect of the invention is an acyl transferase enzyme having the amino acid sequence of SEQ ID NO 84 (DMOT4) or an enzyme having an amino acid sequence with at least 15% sequence identity to SEQ ID NO 84. This enzyme is capable of transferring an acyl unit to QA*-F*-C9 to form QA*-F*-C18 or transferring an acyl unit to QA*-F*-C9-G to form QA*-F*-C18-G. This enzyme is as described in the method of the first aspect of the invention and has the same properties and function as described in relation to the method of the first aspect of the invention. The acyl transferase enzyme DMOT4 may be encoded by a polynucleotide sequence of SEQ ID NO 83.

The eleventh aspect of the invention encompasses acyl transferase enzymes having an amino acid sequence with at least 15% sequence identity to the sequence of DMOT4 (SEQ ID NO 84). The amino acid sequence of the DMOT4 enzyme may have at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 84. Accordingly, in some embodiments, the DMOT4 has at least 15%, 20%, 25%, 30%, 35%, 40%, 45%, 50%, 55%, 60%, 65%, 70%, 75%, 80%, 85%, 90%, 95%, 96%, 97%, 98% or 99% identity to SEQ ID NO 84, suitably at least 90%, more suitably at least 95%. In respect of the enzymes defined here in terms of sequence identity, they typically retain the function of transferring an acyl unit to QA*-F*-C9 or QA*-F*-C9-G

A twelfth aspect of the invention is an arabinofuranosyl transferase enzyme having the amino acid sequence of SEQ ID NO 86 (UGT-L-short), SEQ ID NO 88 (UGT-L-long), or an enzyme having an amino acid sequence with at least 45% sequence identity to SEQ ID NO 86 or 88. The enzyme is capable of transferring a UDP-β-L-arabinofuranose residue to QA*-F*-C18 or QA*-F*-C18-G. This enzyme is as described in the method of the first aspect of the invention and has the same properties and, function as described in relation to the method of the first aspect of the invention.

The arabinofuranosyl transferase enzyme UGT-L-short may be encoded by a polynucleotide sequence of SEQ ID NO 85. The arabinofuranosyl transferase enzyme UGT-L-long may be encoded by a polynucleotide sequence of SEQ ID NO 87.

The twelfth aspect of the invention encompasses arabinofuranosyl transferase enzymes having an amino acid sequence with at least 45% sequence identity to the sequences for UGT-L-short and UGT-L-long (SEQ ID NO 86 and 88 respectively). The amino acid sequence of the UGT-L-short enzyme may have at least 45%, 50%, 55%, 60%, 65%, 70%, 75%, 80%, 85%, 90%, 95%, 96%, 97%, 98% or 99% identity to SEQ ID NO 86. The amino acid sequence of the UGT-L-long enzyme may have at least 45%, 50%, 55%, 60%, 65%, 70%, 75%, 80%, 85%, 90%, 95%, 96%, 97%, 98% or 99% identity to SEQ ID NO 88. Accordingly, in some embodiments, the UGT-L-short and/or UGT-L-long have at least 45%, 50%, 55%, 60%, 65%, 70%, 75%, 80%, 85%, 90%, 95%, 96%, 97%, 98% or 99% identity to SEQ ID NO 86 or 88, suitably at least 90%, more suitably at least 95%. In respect of the enzymes defined here in terms of sequence identity, they typically retain the function of transferring UDP-β-L-arabinofuranose (arafT enzyme) to QA*-F*-C18 or QA*-F*-C18-G.

The invention also includes a glucosyltransferase enzyme having the amino acid sequence of SEQ ID NO 90 (QS-7-GIcT) or an enzyme having an amino acid sequence with at least 70% sequence identity to SEQ ID NO 90. This enzyme is capable of transferring a glucose residue to the C-3 position of the rhamnose residue of the F* of a molecule comprising QA*-F*-C18-A or QA*-F*, wherein F* is FRX, FRXX, FRXA or mixtures thereof. This enzyme is as described in relation to the methods of the first aspect of the invention and has the same properties and function as described in relation to the method of the first aspect of the invention.

The glucosyltransferase enzyme may be encoded by a polynucleotide of SEQ ID NO 89 or a polynucleotide molecule which also encodes for the amino acid sequence with at least 70% sequence identity to SEQ ID NO 90. The QS-7-GIcT enzyme may, for example, be encoded by the polynucleotide sequence according to SEQ ID NO 89 or by a sequence which, by virtue of the degenerative code, also encodes a glucosyltransferase enzyme as described.

The invention also includes glucosyltransferase enzymes having an amino acid sequence with at least 70% sequence identity to the sequence for QS-7-GIcT (SEQ ID NO 90). The amino acid sequence of the QS-7-GIcT enzyme may have at least 70%, 75%, 80%, 85%, 90%, 95%, 96%, 97%, 98% or 99% identity to SEQ ID NO 90. Accordingly, in some embodiments, the QS-7-GIcT has at least 70%, 75%, 80%, 85%, 90%, 95%, 96%, 97%, 98% or 99% identity to SEQ ID NO 90, suitably at least 90%, more suitably at least 95%. In respect of the enzymes defined here in terms of sequence identity, they typically retain the function of transferring a glucose residue to a molecule comprising QA*-F*-C18-A to form QA*-F*-C18-A-G or transferring a glucose residue to a molecule comprising QA*-F* to form QA*-F*-G.

A nucleic acid sequence which encodes the
enzyme according to SEQ ID NO 90.
SEQ ID NO 89
ATGGCGGATCGAGTCATAAACAGCTACAAAAAGCTTCACGTAGTGCTGTT
TCCATGGCTGGCCTTTGGTCACATGATACCATTTTTAGAGCTTGCAAAGC
TGATAGCCCAAAAAGGTCACAAAATTTCCTATATCTCAACACCCAGAAAC
ATCCGACGCCTACCTAAAATTCCATCCCATTTATCCAACAACCTTAATTT
CATAGAATTCCCACTGCCTCATATACCCAATCTTCATGAAAATGTGGAAT
CAACAAACGACGTCACACATGACAATCCTATTGCCTATTATTTACTTATT
AAAGCCCTTGAGGGTTTGCAACAACCTATCACCACCTTCCTTGAAACCTC
AGATCCAGATTGGATAATTCATGACGTTTTTCCTCAATGGATAACCGCAA
CAGCCAGTAGGCTCCGCATTTCACATGCTTTTTACACAACTTCATCTGCT
TTGAGAACGGCTTCCAATTATTCTCCGTTAATGTCCTCTGAATTACCACA
GGATATTGCTACCGATTTCACTACTAAATCCACAAATCTTCTTGCTGCCA
AGGTTTTGGTTATCCGTAGCTGTCTAGAGCTTGAACCCAAGGAATTTGAA
CAGTGCAAAAATCTATGCGTGTCCAAAACGGTAATTCCATTGGGCGTTGT
CCCACCGTCAATTCAGGTGAACGATAACATTTCTAATATTAATGATGACG
ACAACGACTGGGTTAAGATTGTTGAGTGGTTAAACCAAGGGAAAGAGAAA
GGTTCTGTCATTTATGTAGCACTTGGTTCTGAGGTTTCACTGAGTGAACA
AGATTTGAAGGAGTTTGCACTTGGTTTGGAACTATCAGGGTTGTCATTCT
TTTGGGTGTTCAGGAATACTGGATCGTATGGGTTACCGGCTGGGTTTGAA
GACCGAGTTAAGGGTCGGGGAATTGTCTGGACTAGCTGGGCTCCGCAGGT
GAGTATATTAGGACACGAGTCAATTGGTGGATTCTTGAGTCATGGGGGTT
GGAGTTCTGTAATAGAAAGTCTAAGTTTTGGGATTCCACTTGTTGTTTTT
CCATTTGGAGCTGATCAAGGGATTAATGCAAAGCAATTGGAGGGTAAAAA
TGCAGGGGTGGAAATACCCAGATCAGAAGGTACTGGGTCATTTACAAGGA
AGTCTGTGGCTGATTTGTTGAGGCTGGTGGTGGTGGAGGAGGAGGGAAAG
GTTTACAGGGATGGTGCCAAGGAATTGAGGAAACTATTTGGGGACAAGGA
TTTGAACCACAAATACATTGACAACTTTGTTAAGTACATGGAAGAACATA
TCACGAATGCAGCTAATTGA*
A glucosyltransferase enzyme capable of
transferring a glucose residue to the C-3
position of the C-28 rhamnose residue of
a QA-Tri(X/A)-F* derivative (Qs-7-GlcT)
SEQ ID NO 90
MADRVINSYKKLHVVLFPWLAFGHMIPFLELAKLIAQKGHKISYISTPRN
IRRLPKIPSHLSNNLNFIEFPLPHIPNLHENVESTNDVTHDNPIAYYLLI
KALEGLQQPITTFLETSDPDWIIHDVFPQWITATASRLRISHAFYTTSSA
LRTASNYSPLMSSELPQDIATDFTTKSTNLLAAKVLVIRSCLELEPKEFE
QCKNLCVSKTVIPLGVVPPSIQVNDNISNINDDDNDWVKIVEWLNQGKEK
GSVIYVALGSEVSLSEQDLKEFALGLELSGLSFFWVFRNTGSYGLPAGFE
DRVKGRGIVWTSWAPQVSILGHESIGGFLSHGGWSSVIESLSFGIPLVVF
PFGADQGINAKQLEGKNAGVEIPRSEGTGSFTRKSVADLLRLVVVEEEGK
VYRDGAKELRKLFGDKDLNHKYIDNFVKYMEEHITNAAN

Any sequence identity percentage of the sixth, seventh, eighth, ninth, tenth, eleventh and twelfth aspects of the invention can be combined with any other sequence identity percentage of the sixth, seventh, eighth, ninth, tenth, eleventh and twelfth aspects of the invention.

A thirteenth aspect of the invention is a polynucleotide which encodes one or more of the enzymes of the sixth to twelfth aspects of the invention.

A fourteenth aspect of the invention is a vector comprising one of more of the polynucleotides according to the thirteenth aspect of the invention.

The vector may comprise one or more of the polynucleotides encoding the enzymes of the sixth to twelfth aspects of the invention. Preferably, the vector will comprise seven or eight of the polynucleotides encoding the enzymes of the sixth to twelfth aspects of the invention or a number of vectors which, together, comprise the seven or eight polynucleotides.

A fifteenth aspect of the invention is a host cell comprising the polynucleotides according to the thirteenth aspect of the invention.

The host cell may be a plant cell or microbial cell. When the host cell is a microbial cell it is preferably a yeast cell. When the host cell is a plant cell, the plant is preferably Nicotiana benthamiana.

An additional feature of the fifteenth aspect of the invention is the method of introducing the polynucleotides of the thirteenth aspect of the invention, into the host cell. The polynucleotides may be introduced into the host cells via a vector. Recombination may occur between the vector and host cell genome to introduce the polynucleotides into the host cell genome. Alternatively, the polynucleotides may be introduced into the host cells by co-infiltration with a plurality of recombinant vectors. The recombinant vectors may be Agrobacterium tumefaciens strains, discussed below.

A sixteenth aspect of the invention is a host cell transformed with the vector according to the fourteenth aspect of the invention.

A seventeenth aspect of the invention is a biological system of a plant or a microorganism comprising host cells as set out according to the fifteenth and sixteenth aspects of the invention. The biological system may be a plant or a microorganism. When the biological system is a plant, it may be Nicotiana benthamiana or any of the plants described above. The method of producing the plant comprises the steps of introducing the polynucleotides of the invention into the host plant cell and regenerating a plant from the transformed host plant cell. When the biological system is a microorganism, it may be yeast.

The invention also includes the method of making each enzyme and each polynucleotide of the above aspects of the invention, as well as a method of making a vector comprising one or more of the polynucleotides of the invention, as well as the host cells of the fifteenth and sixteenth aspects of the invention and a method of making the biological system of the seventeenth aspect of the invention. These methods use techniques and products well known in the art, such as in WO2019/122259 and WO2020/260475, and are described in more detail as follows:

The polynucleotides of the invention can be included in a vector, in particular an expression vector, as described in the Example section. The vector may be any plasmid, cosmid, phage or Agrobacterium vector in double or single stranded linear or circular form which can transform a prokaryotic or eukaryotic host either by integration into the cellular genome or other. The vector may be an expression vector, including an inducible promoter, operably linked to the polynucleotide sequence. Typically, the vector may include, between the inducible promoter and the polynucleotide sequence, an enhancer sequence. The vector may also include a terminator sequence and optionally a 3′-UTR located upstream of said terminator sequence. The vector may include one or more polynucleotides encoding enzymes of the first aspect of the invention, preferably all sequences needed to produce one version of the molecule as set out according to the first aspect of the invention. The vector may be a plant vector or a microbial vector.

The polynucleotide in the vector may be under the control of, and operably linked to, an appropriate promoter or other regulatory elements for transcription in a host cell. The host cell may be a yeast cell, bacterial cell or plant cell. The vector may be a bi-functional expression vector which functions in multiple hosts. In the case of genomic DNA, this may contain its own promoter or other regulatory elements. The advantage of using a native promoter is that this may avoid pleiotropic responses. In the case of cDNA this may be under the control of an appropriate promoter or other regulatory elements for expression in the host cell.

Preferred vectors for use in plants comprise border sequences which permit the transfer and integration of the expression vector into the plant genome. The vector may be a plant binary vector.

The vector may be transfected into a host cell in any biological system. The host may be a microbe, such as E. coli, or yeast. The vector may be part of an Agrobacterium tumefaciens strain and used to infect a biological plant host system. The Agrobacterium tumefaciens may each contain one of the required polynucleotides encoding for the invention and can be combined to co-infect a host cell, such that the host cell contains all the necessary polynucleotides to encode for the enzyme of the first aspect of the invention.

The present invention also includes the steps of culturing the host or growing the host for the production, harvest and isolation of the desired QA*-F*-C18-A and/or QA*-F*-C18-A-G.

An additional feature of the first, second, third and fourth aspects of the invention is the step of isolating the QA*-F*-C18-A and/or QA*-F*-C18-A-G.

The eighteenth aspect of the invention is a QA*-F*-C18-A derivative obtainable by the method of the first or fourth aspect of the invention. A QA*-F*-C18-A derivative obtainable by the methods of the invention may be isolated from the biological system. The isolated QA*-F*-C18-A is QA-Mono-F-C18-A, QA-Di-F-C18-A, QA-TriX-F-C18-A, QA-TriR-F-C18-A, QA-FR-C18-A, QA-Mono-FR-C18-A, QA-Di-FR-C18-A, QA-TriX-FR-C18-A, QA-TriR-FR-C18-A, QA-FRX-C18-A, QA-Mono-FRX-C18-A, QA-Di-FRX-C18-A, QA-TriX-FRX-C18-A, QA-TriR-FRX-C18-A, QA-FRXA-C18-A, QA-Mono-FRXA-C18-A, QA-Di-FRXA-C18-A, QA-TriX-FRXA-C18-A, QA-TriR-FRXA-C18-A, QA-FRXX-C18-A, QA-Mono-FRXX-C18-A, QA-Di-FRXX-C18-A, QA-TriX-FRXX-C18-A and/or QA-Tri(X/R)-FRXX-C18-A, or mixtures thereof. Preferably the isolated QA*-F*-C18-A is QA-TriX-FRXA-C18-A. The QA*-F*-C18-A of this aspect of the invention may be obtainable by the methods of the invention, or any other method. The QA*-F*-C18-A of this aspect of the invention may be obtained by the methods of the invention.

The nineteenth aspect of the invention is a QA*-F*-C18-A-G derivative obtainable by the method of the second aspect of the invention. A QA*-F*-C18-A-G derivative obtainable by the methods of the invention may be isolated from the biological system. The isolated QA*-F*-C18-A-G is QA-FRX-C18-A-G, QA-Mono-FRX-C18-A-G, QA-Di-FRX-C18-A-G, QA-TriX-FRX-C18-A-G, QA-TriR-FRX-C18-A-G, QA-FRXA-C18-A-G, QA-Mono-FRXA-C18-A-G, QA-Di-FRXA-C18-A-G, QA-TriX-FRXA-C18-A-G, QA-TriR-FRXA-C18-A-G, QA-FRXX-C18-A-G, QA-Mono-FRXX-C18-A-G, QA-Di-FRXX-C18-A-G, QA-TriX-FRXX-C18-A-G and/or QA-Tri(X/R)-FRXX-C18-A-G, or mixtures thereof. Preferably the isolated QA*-F*-C18-A-G is QA-TriX-FRXA-C18-A-G. The QA*-F*-C18-A-G of this aspect of the invention may be obtainable by the methods of the invention, or any other method. The QA*-F*-C18-A-G of this aspect of the invention may be obtained by the methods of the invention.

The twentieth aspect of the invention is the use of the QA*-F*-C18-A, preferably QA-TriX-FRXA-C18-A, or QA*-F*-C18-A-G, preferably QA-TriX-FRXA-C18-A-G, as an adjuvant to be included in a vaccine composition, once isolated from the biological system. The adjuvant may be a liposomal formulation.

An additional feature of the twentieth aspect of the invention is that the adjuvant further comprises a TLR4 agonist. The TLR4 agonist may be 3D-MPL. QA*-F*-C18-A of the present invention may be combined with further immuno-stimulants, such as a TLR4 agonist, in particular lipopolysaccharide TLR4 agonists, such as lipid A derivatives, especially a monophosphoryl lipid A, e.g. 3-de-O-acylated monophosphoryl lipid A (3D-MPL). 3D-MPL is sold under the name ‘MPL’ by GlaxoSmithKline Biologicals N.A. See, for example, U.S. Pat. Nos. 4,436,727; 4,877,611; 4,866,034 and 4,912,094. 3D-MPL can be produced according to the methods described in GB 2 220 211 A. Chemically, it is a mixture of 3-deacylated monophosphoryl lipid A with 4, 5 or 6 acylated chains.

Other TLR4 agonists which may be combined with QA*-F*-C18-A or QA*-F*-C18-A-G of the invention include Glucopyranosyl Lipid Adjuvant (GLA) such as described in WO2008/153541 or WO2009/143457 or literature articles (Coler et al. 2011 and Arias et al. 2012).

Adjuvants of the invention may also be formulated into a suitable carrier, such as an emulsion (e.g. an oil-in-water emulsion) or liposomes, as described below.

### Liposomes

The term liposome is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds. A method for making such liposomes is described in WO2013/041572.

Liposome size may vary from 30 nm to several m depending on the phospholipid composition and the method used for their preparation.

The liposome size will be in the range of 50 nm to 200 nm, especially 60 nm to 180 nm, such as 70-165 nm. Optimally, the liposomes should be stable and have a diameter of 100 nm to allow convenient sterilization by filtration.

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. The average particle size may be between 95 and 120 nm, and/or, the polydispersity (Pdl) index may not be more than 0.3 (such as not more than 0.2).

A twentieth aspect of the invention is an adjuvant composition comprising the QA*-F*-C18-A derivative according to the eighteenth aspect of the invention, or an adjuvant composition comprising the QA*-F*-C18-A-G derivative according to the nineteenth aspect of the invention.

### EXAMPLES

The present invention is described with reference to the following, non-limiting examples:

### Example 1—Predicting the Biosynthetic Route of QS-21 Acyl Chain

The QS-21 acyl chain is made of two identical acyl units (or monomers) with an arabinofuranose attached to the C-5 hydroxy of the second unit (see FIG. 3 for an illustration of the carbon numbering). Carbons 5-9 of the acyl unit were hypothesized to be derived from isoleucine which shares the same structure. Branched-chain amino acid catabolism occurs in the mitochondrion where branched-chain aminotransferases (BCATs) deaminate leucine, valine and isoleucine, forming branched-chain α-keto acids. In the case of isoleucine, the product 2-keto-3-methyl-valeric acid (FIG. 3) enters the branched-chain alpha-keto acid dehydrogenase (BCKDH) complex to undergo a decarboxylation forming 2-keto-3-methyl-valeric acid. Based on a similar process described for hop (Humulus lupulus) bitter acids (Xu et al, 2013), it is likely that the CoA is removed by a thioesterase (TE) to allow export to the cytosol, where a CoA is reattached. This ligation is likely to be the first step necessary to engineer the acyl chain in heterologous organisms such as yeast as it lacks cytosolic 2-methylbutyryl-CoA (Xu et al, 2013). It was hypothesised that the next step is catalysed by a member of the type III polyketide synthase (PKSIII) family, sometimes referred to as Chalcone Synthase-like (ChS in this work). In the case described here, a PKSIII enzyme would use two molecules of malonyl-CoA to extend 2-methylbutyryl-CoA into (S)-6-methyl-3,5-dioxooctanoyl-CoA. The two keto groups are thereafter reduced stereoselectively by two keto reductases (KRs) to form the acyl unit as seen in QS-21. This acyl unit is transferred onto the C-4 position of the fucose residue on the QS-21 scaffold by an acyl transferase (AT). Amongst the various plant ATs, the BAHD family was the strongest candidate as acyl-CoA thioesters are common substrates for them. A second BAHD family member would then add an additional acyl unit to the C-5 hydroxyl of the first acyl unit. Lastly, an arabinofuranosyltransferase (ArafT) would attach the sugar to the acyl chain.

### Example 2—Identifying QS-21 Acyl Chain Candidate Genes

Relevant enzyme candidates were selected, cloned and tested. In order to clone candidate genes, a series of oligonucleotide primers were designed which incorporated 5′-attB sites upstream of the target sequence to allow for Gateway® cloning. Using these primers, genes were amplified by PCR from Q. saponaria leaf cDNA and cloned into pDONR 207. The clones were sequenced before transfer into the plant expression vector pEAQ-HT-DEST1 (Sainsbury et al., 2009). The expression constructs were then transformed individually into Agrobacterium tumefaciens (LBA4404 and/or GV3101) for transient expression in N. benthamiana. To identify the keto reductases, acyl transferases and arabinosyl transferase, the candidates were co-expressed in N. benthamiana with the set of genes necessary to produce QA-TriR-FRX, that is the glycosylated quillaic acid with glucuronic acid, galactose and rhamnose residues at the C-3 position and fucose, rhamnose and xylose residues at the C-28 position. In addition, previously tested CCL and ChS enzyme candidates were also included in that screening to allow for the biosynthesis of (S)-6-methyl-3,5-dioxooctanoyl-CoA. Finally, a truncated, feedback-insensitive form of the Avena strigosa HMG-CoA reductase (AstHMGR) was included in any infiltration leading to the formation of QA, as this has previously been shown to increase the production of triterpenes produced in N. benthamiana (Reed et al., 2017).

### Example 3—Identification of Carboxyl CoA Ligases (CCLs)

Once exported from the mitochondrion, 2-methylbutyric acid needs to be chemically activated by the attachment of a CoA to allow further extension using two malonyl-CoAs. Six enzyme candidates were selected based on a phylogenetic tree of the Q. saponaria CCLs including the hop HICCL2 and HICCL4. The six Q. saponaria homologs of HICCL4 were selected for cloning. 3CCL was the strongest candidate based on its high co-expression score with QS-21 genes and its high expression level in primordium.

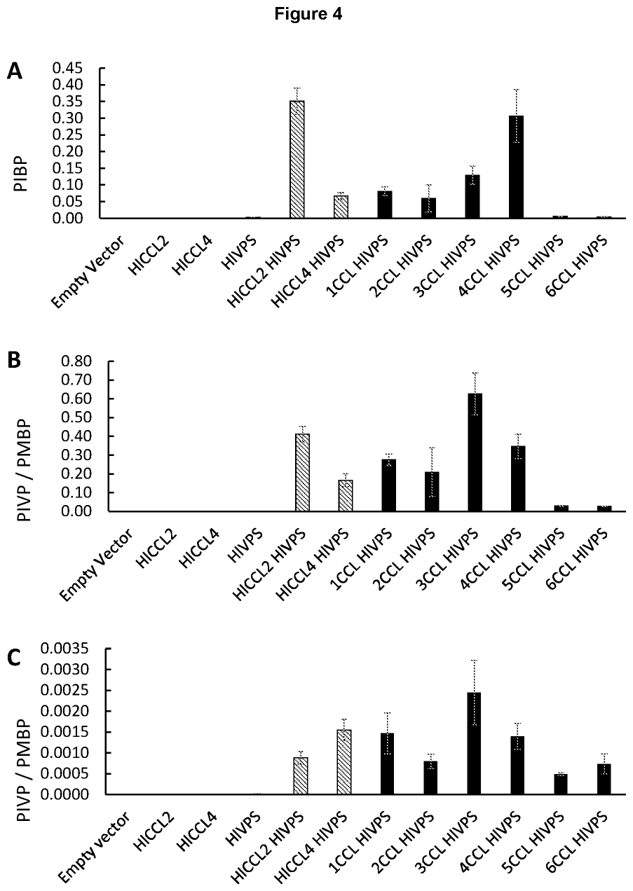

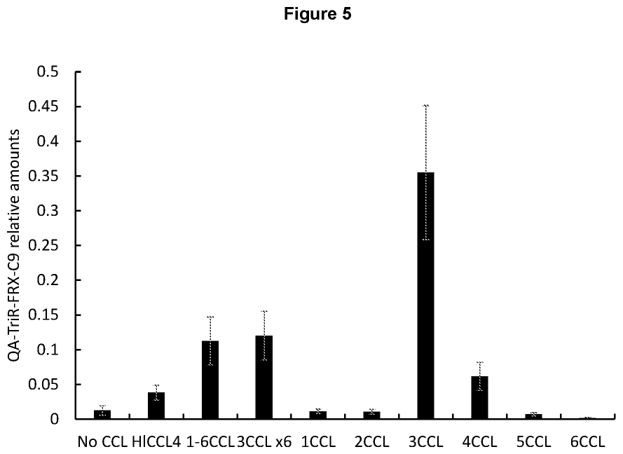

Detecting and measuring the products of the CCL enzyme candidates is technically challenging as acyl-CoAs are not easily detected using routine LC-MS. Consequently, their activities were screened indirectly by measuring downstream products. Firstly, they were heterologously expressed in yeast and N. benthamiana with the hop valerophenone synthase (HIVPS) which was shown to produce phlormethylbutanophenone (PMBP) and its isomer, phlorisovalerophenone (PIVP), from the products of HICCL2 and HICCL4 (Xu et al., 2013; FIG. 4). Secondly, they were screened in N. benthamiana leaves by transiently co-expressing the gene sets required for production of the QA-TriR-FRX (including AstHMGR) and the genes needed to make the C9 acyl unit and transferring it to QA-TriR-FRX. The QA-TriR-FRX-C9 levels were believed to be indicative of the six CCLs efficiency at converting 2-methylbutyric acid into 2-methylbuturyl-CoA (FIG. 5).

The six Q. saponaria CCL enzyme candidates were active in yeast and in N. benthamiana as downstream products were detected for all of them (FIG. 4). 3CCL efficiency was significantly higher than those of the other candidates when screening for PIVP/PMBP in the two heterologous hosts, and 1CCL, 2CCL and 4CCL producing intermediate amounts in both hosts (FIGS. 4B and C). Measurements of PIBP in yeast showed that 1CCL, 2CCL, 3CCL and above all, 4CCL were also able to ligate a CoA to isobutyric acid, the short-chain fatty acid derived from valine (FIG. 4A). 5CCL and 6CCL were overall the least active enzymes as they failed to lead to the synthesis of PIBP and led to a low amount of PIVP/PMBP in yeast (FIGS. 4A and B). 3CCL was further identified as the best enzyme candidate when measuring the amounts of QA-TriR-FRX-C9. Interestingly, co-expressing the six CCLs (1CCL, 2CCL, 3CCL, 4CCL, 5CCL and 6CCL) together led to an averaging effect instead of increasing the product level as could be expected by the increased amounts of CCLs (FIG. 5). There may be some competition between the enzymes or some regulatory effects from the N. benthamiana plant. However, increasing by six-fold the concentration of 3CCL (3CCL×6) also led to a decrease of the downstream product levels suggesting that the intracellular concentration of the enzyme affects the catalysis efficiency (FIG. 5). The measurements of QA-TriR-FRX-C9 confirmed that 3CCL is the most active enzyme candidate and 4CCL also allowed an increase of the downstream product compared to the empty vector control. In this specific experiment, the levels of QA-TriR-FRX-C9 were no different between the negative control and 1CCL, 2CCL, 5CCL and 6CCL. These results suggest that 3CCL likely provides most of the 2-methylbutyryl-CoA necessary for the synthesis of the acyl unit in Q. saponaria.

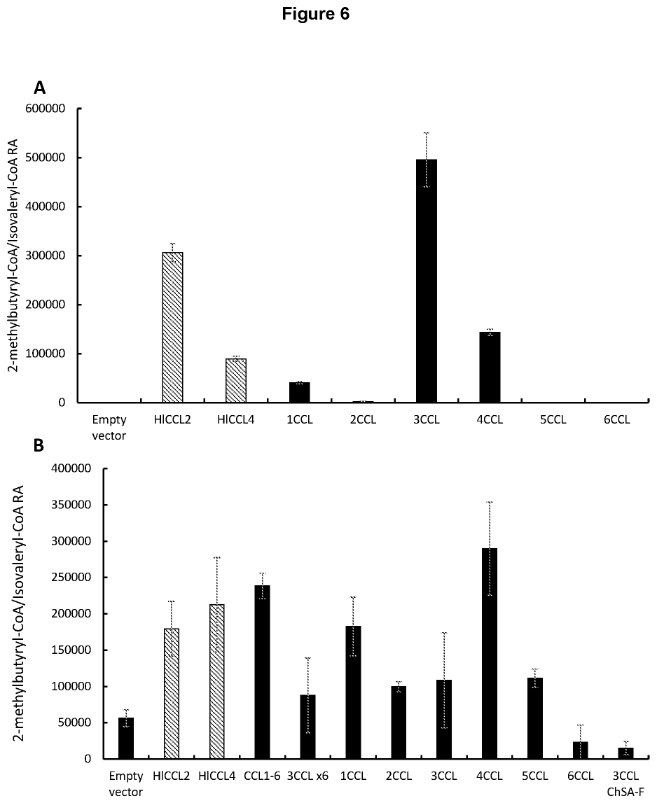

In order to measure the levels of the expected direct products of the CCL enzyme candidates, a method to detect and measure acyl-CoAs as described in the ChS section (Example 4) was developed. This method does not allow to distinguish between 2-methylbutyryl-CoA and isovaleryl-CoA given their isomeric nature. FIG. 6 shows that 3CCL and 4CCL produced the most 2-methylbutyryl-CoA and/or isovaleryl-CoA in yeast and N. benthamiana, respectively. 2CCL, 5CCL and 6CCL were not active in yeast and 6CCL was not in N. benthamiana. The sample combining 3CCL and the six ChS candidates showed that the ChSs are using the product of 3CCL (FIG. 6B). Similar to what was observed when measuring QA-TriR-FRX-C9, co-infiltrating the six CCLs or increasing by six folds the Agrobacterium strain containing the 3CCL gene did not lead to an increased efficiency. The fact that the levels of 2-methylbutyryl-CoA and isovaleryl-CoA were highest for 4CCL in N. benthamiana, but that the levels of QA-TriR-FRX-C9 were highest for 3CCL, suggests that 4CCL favours isovaleric acid as a substrate, whereas 3CCL, although producing overall less 2-methylbutyryl-CoA/isovaleryl-CoA, likely favours 2-methylbutyric acid as a substrate.

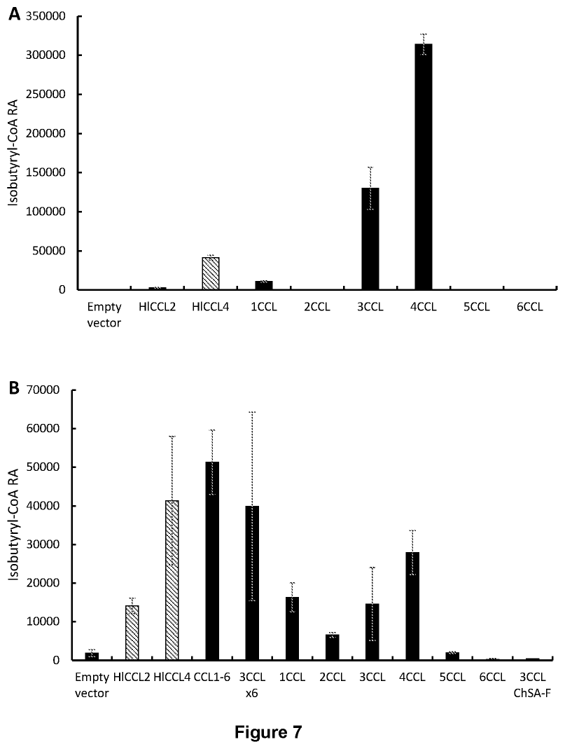

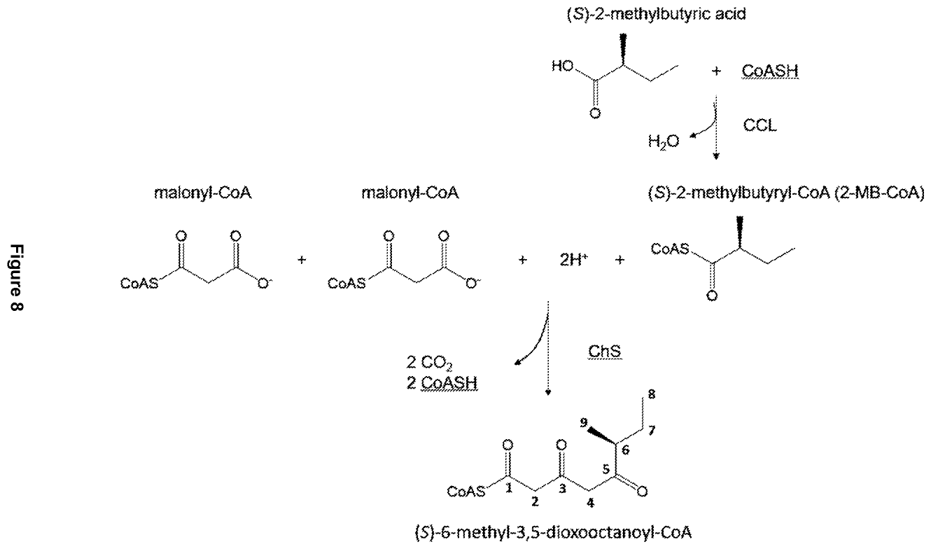

In addition, the CCL enzyme candidates were also able to catalyse the formation of isobutyryl-CoA, a metabolite derived from the catabolism of the branched-chain amino acid valine, with various efficiencies (FIG. 7), 4CCL and to a lesser extent, 3CCL, being the most productive. As for the 2-methylbutyryl-CoA/isovaleryl-CoA experiment, 1CCL was overall the third most active enzyme. The hop HICCL4 also catalysed this reaction, which is consistent with published in vitro data (Xu et al., 2013). The activity of HICCL2 on isovaleric acid was shown to be poor in yeast (Xu et al., 2013), which was confirmed in the yeast experiment. Its activity in N. benthamiana was much higher than that of the control. Co-infiltrating the six CCLs or increasing the concentration of 3CCL led to an increase of product formation contrary to what was seen for 2-methylbutyryl-CoA/isovaleryl-CoA. The combined ChS candidates were also able to utilize the isovaleryl-CoA formed by 3CCL indicating that they were capable of using at least two different substrates. Similarly, the CCL enzyme candidates were not restricted to a unique substrate.

### Example 4—Identification of Chalcone Synthase-Like Type III PKS Enzymes

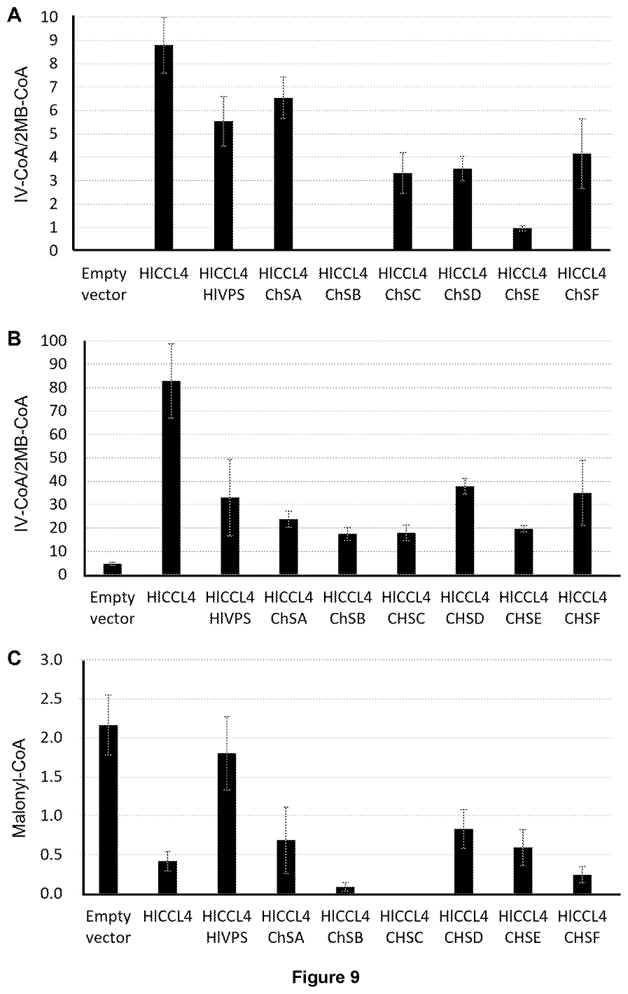

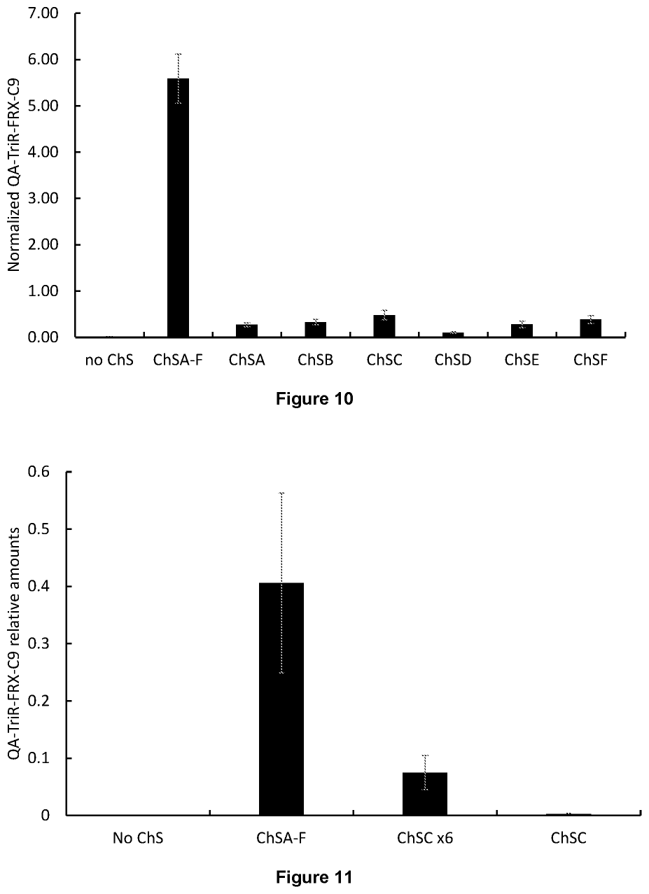

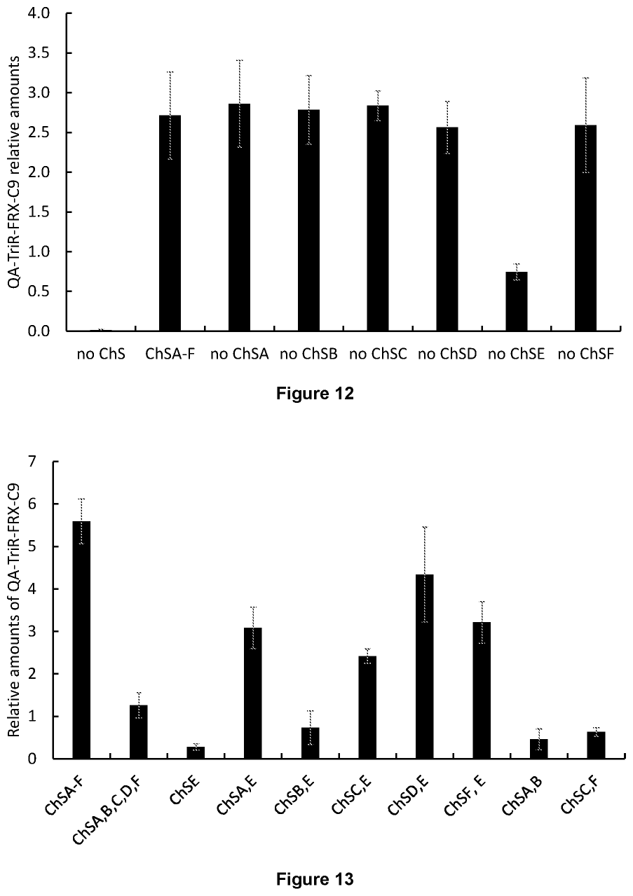

Q. saponaria transcriptome was screened to identify expressed ChS enzyme candidates. It was hypothesized that at least one of these candidates would extend 2-methylbutyryl-CoA using two molecules of malonyl-CoA (FIG. 8) to form (S)-6-methyl-3,5-dioxooctanoyl-CoA. Several protocols were tested. Standards of CoA, malonyl-CoA, 3-hydroxy-3-methylglutaryl-CoA (HMG-CoA), acetyl-CoA, isovaleryl-CoA and p-hydroxybutyryl-CoA were detected and separated following a protocol published by Glaser et al. (2020). All of these were detectable in N. benthamiana leaves indicating that the method was sensitive enough to detect (S)-6-methyl-3,5-dioxooctanoyl-CoA when the ChS candidates were expressed in N. benthamiana leaves. However, it could not be detected based on predicted ion transitions. To circumvent this issue, the depletion of the predicted substrate of the ChS enzyme candidate was measured. However, the LC chromatography does not allow to distinguish 2-methylbutyryl-CoA, the substrate, from its isomer isovaleryl-CoA. This point was addressed by co-expressing the ChS candidates with HICCL4 which uses 2-methylbutyric acid as a substrate, but not isovaleric acid (Xu et al, 2013). Empty vector controls showed that yeast did not have endogenous detectable amounts of 2-methylbutyl-CoA or isovaleryl-CoA, while N. benthamiana had negligible amounts (FIGS. 9A and B, respectively). Expression of HICCL4 in yeast and N. benthamiana leaves boosted the levels of 2-methylbutyryl-CoA (FIGS. 9A and B, respectively). The amounts of 2-methylbutyryl-CoA were lower when HIVPS and the ChS candidates were co-expressed, indicating that all of these enzymes utilised it as a substrate. The substrate depletion pattern was different in yeast and N. benthamiana, with little differences between the PKSIII enzymes in N. benthamiana and more extreme differences in yeast. Of note, ChSB entirely depleted the pool of 2-methylbutyryl-CoA generated by HICCL4 and ChSE significantly used more of it than the remaining ChSs in yeast (FIG. 9). The levels of malonyl-CoA, the second substrate used by the ChSs to form (S)-6-methyl-3,5-dioxooctanoyl-CoA were also affected by the presence of HICCL4 and the ChSs suggesting that malonyl-CoA may be limiting at physiological levels for this reaction to occur (FIG. 9C).

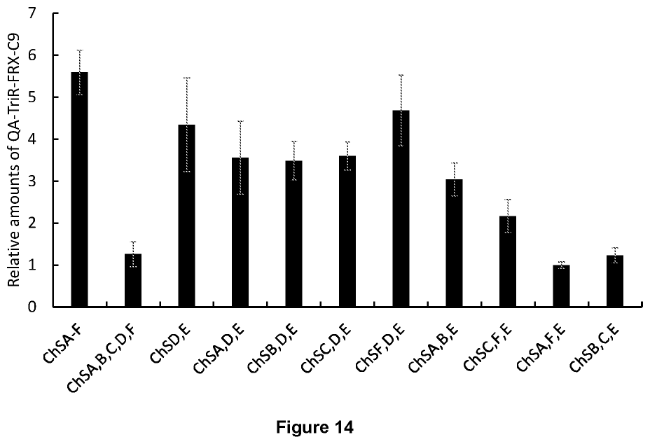

Furthermore, the activities of the six ChS enzyme candidates were tested by measuring the levels of the downstream product QA-TriR-FRX-C9. To this end, all the genes necessary for the production of QA-TriR-FRX were co-expressed along with AstHMGR and with the two reductases KR11 and KR23′ and the acyl transferase DMOT9 necessary to form the acyl unit (3S,5S,6S)-3,5-dihydroxy-6-methyloctanoyl-CoA, and ligate it to QA-TriR-FRX, respectively. The six candidates were able to produce QA-TriR-FRX-C9 (FIG. 10), ChSC leading to slightly higher amounts of it than the other candidates. Contrary to what was seen for the CCL candidates on 2-methylbutyryl-CoA/isovaleryl-CoA, adding the six ChS candidates in one infiltration boosted the level of QA-TriR-FRX-C9. This additive effect could result from a synergy between the candidates. In fact, PKSIII enzymes are known to often homodimerize, so maybe some of the candidates heterodimerize for a more efficient reaction. An alternative explanation is that there is a linear correlation between enzyme concentration and product output. Taken together, these results indicate that the six ChS candidates are able to use 2-methylbutyryl-CoA as substrate to make (S)-6-methyl-3,5-dioxooctanoyl-CoA.

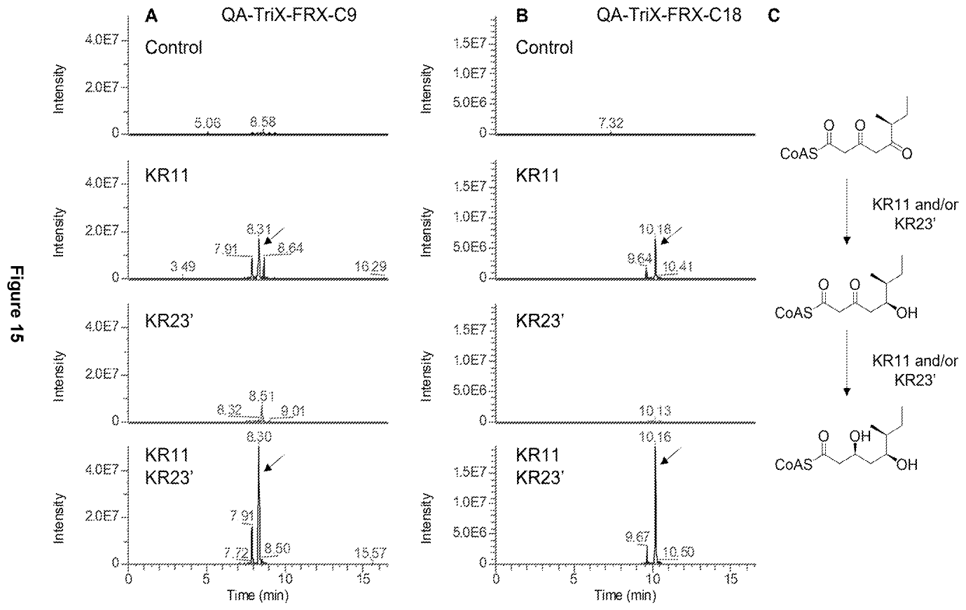

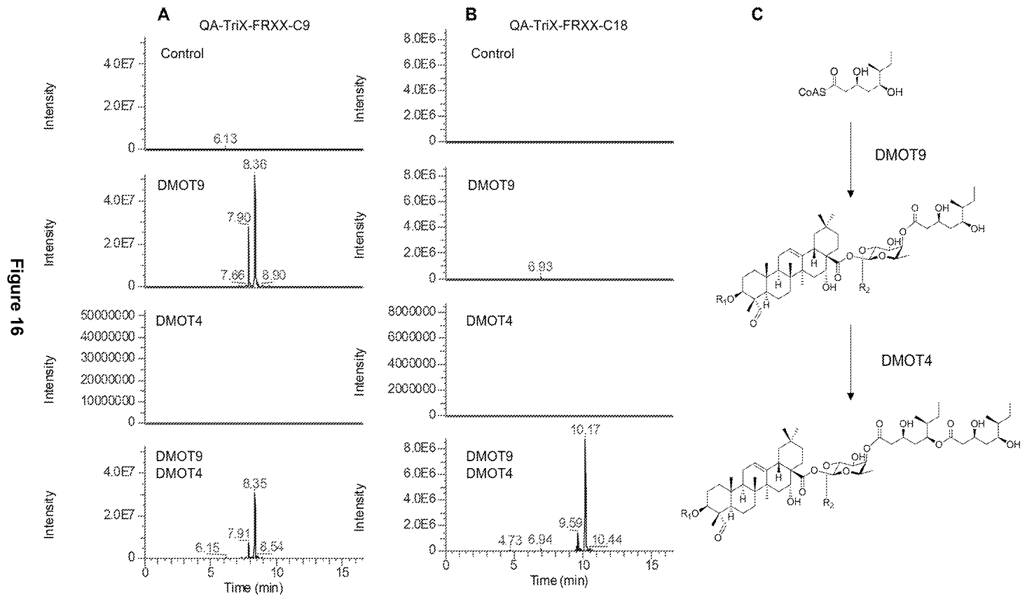

To assess whether the increased efficiency detected in the sample containing the six ChS enzyme candidates is due to an increase of the concentration of the Agrobacterium strains in this sample (the ChSA-F sample of FIG. 10 contains six times the amount of Agrobacterium strains than the samples testing an individual ChS), the concentration of the ChSC Agrobacterium strain was increased as such (FIG. 11). Despite a clear efficiency improvement compared to the sample with normal amount of the ChSC Agrobacterium strain, the ChSC×6 sample failed to reach the efficiency of the sample containing the six ChS genes. This result indicates that the higher Agrobacterium concentration of the ChA-F sample does not fully account for the major increase of downstream product levels.

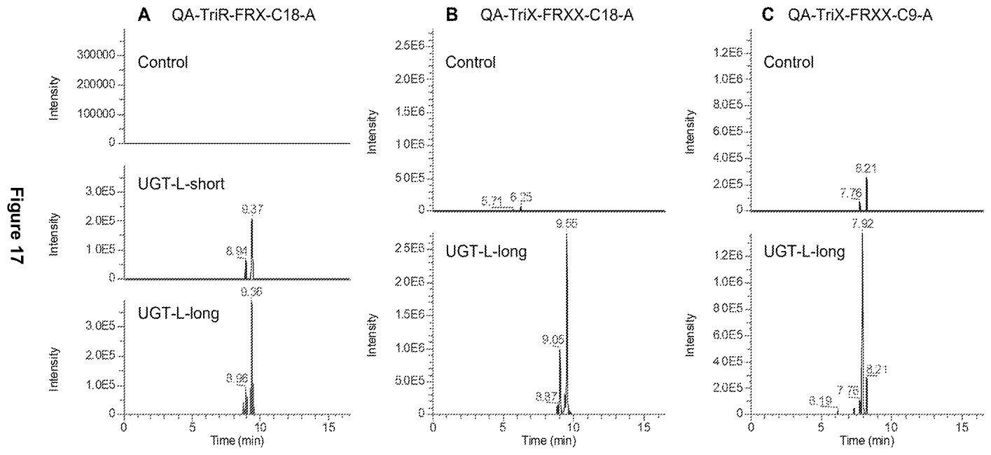

To explore the possibility that the ChS heterodimerise, a drop out experiment was conducted in which five of the six ChS candidates were co-infiltrated and compared with the levels obtained for the six co-infiltrated candidates (FIG. 12). The levels of QA-TriR-FRX-C9 were not affected except when ChSE was absent, showing that ChSE is required to obtain high amount of product and suggesting that it interacts with more than one of the other ChS candidates.

ChSE was then co-infiltrated with another ChS enzyme candidate (FIG. 13). Pairing ChSE with ChSA, ChSC, CHSD or CHSF increased the levels of product made compared to either ChSE by itself or to the combination of all ChSs but ChSE. This shows that ChSE interacts with these four ChSs. In a repeat experiment, ChSB paired with ChSE also led to a noticeable increase of product. The amounts of product formed by ChSA and ChSB and by ChSC and ChSF were low, confirming that ChSE is key to obtain high yields of products.

Adding a third ChS to the ChSD/ChSE mix did not lead to better yield (FIG. 14). Furthermore, the triads containing ChSD consistently had higher QA-TriR-FRX-C9 amounts than those without indicating that ChSE and ChSD are likely the two ChSs that interact to most efficiently produce (S)-6-methyl-3,5-dioxooctanoyl-CoA.

In parallel to the in vivo experiments mentioned above, some complementary in vitro experiments have been undertaken to assess the activity of the ChS enzyme candidates. The ChSs were heterologously expressed in Nicotiana benthamiana with His-tag and purified by metal affinity purification. They were then individually tested with 2-methylbutyryl-CoA and malonyl-CoA as substrates. A product peak was obtained for ChSA, ChSD, ChSE and ChSF. These peaks are likely to be (S)-6-methyl-3,5-dioxooctanoyl-CoA or a degradation product thereof.

### Example 5—Identification of the Keto Reductases

A total of twenty genes were selected. Two of them were especially strong candidates based on their high expression levels in primordium, their very high co-expression scores and their relevant acyl-CoA reductase annotations: Cinnamoyl-CoA reductase for KR11 and very-long-chain 3-oxoacyl-CoA reductase 1 for KR23′.

The two KRs were tested in N. benthamiana by co-infiltration of the gene sets necessary to produce QA-TriX-FRX, and the other genes necessary to produce and attach the acyl unit to the terpene backbone (CCL, ChSs and two acyl transferases). Co-infiltration of KR23′ and KR11 led to the detection of peaks of the right masses for the QS-21 scaffold with an acyl unit (QA-TriX-FRX-C9) and with two acyl units (QA-TriX-FRX-C18) (FIG. 15 A and B, respectively). KR11 by itself, produced small amounts of the acylated scaffold indicating that KR11 was able to reduce the two ketones of (S)-6-methyl-3,5-dioxooctanoyl-CoA (FIG. 15). However, both KRs were needed to produce relatively high levels of QA-TriX-FRX-C9 and QA-TriX-FRX-C18.

### Example 6—Identification of the Acyl Transferases (DMOT4 and DMOT9)

Thirty-six acyltransferase candidates were selected for cloning and testing. Two of these candidates, DMOT4 and DMOT9 ((3S,5S,6S)-3,5-Dihydroxy-6-MethylOctanoyl-CoA Transferases), showed relevant activities when co-expressed with the QA-TriR-FRX gene sets and with the acyl chain genes 3CCL, ChSs, KR11 and KR23′. The DMOT enzyme candidates were further tested on QA-TriX-FRXX (FIG. 16). QA-TriX-FRXX-C9 was detected in the sample mix containing DMOT9 without DMOT4, but not the other way around (FIG. 16A), indicating that DMOT9 transfers the first acyl unit to QA-TriX-FRXX. A peak with the predicted mass for QA-TriX-FRXX-C18 was detected at 10.17 min when DMOT9 and DMOT4 were co-expressed in this experiment (FIG. 16B). This indicates that DMOT4 ligates a second acyl unit to QA-TriX-FRXX-C9. DMOT9 is part of the biosynthetic gene cluster 45 that contains five genes required to produce the glycosylated scaffold of QS-21, including the genes necessary to attach the fucose and the rhamnose residues at the C-28 position. Noteworthily, DMOT9 was able to attach the C9 acyl unit to QA-TriR-F, QA-TriR-FR, QA-TriR-FRX and QA-TriR-FRXX with the highest amount of product obtained for QA-TriR-FR. In addition, it was also able to transfer an acetyl to QA-TriX-FRXA, QA-TriX-FRXX and QA-TriR-FRX.

### Example 7—Identification of the Arabinofuranosyl Transferase (UGT-L)

As described in WO2020/260475 UGT enzyme candidates were successfully selected based on their expression levels and co-expression scores to identify the enzymes that glycosylate quillaic acid to produce the QS-21 scaffold. Among these candidates, UGT-L was cloned twice: initially based on the 1 kp datasets, then on our genomic/transcriptomic datasets. Two clones were obtained with a discrepancy at the N-terminal end, with the first clone lacking the initial 14 amino acids. They were named UGT-L-short and UGT-L-long, respectively. UGT-L was a strong candidate for attaching an arabinofuranose at the end of the QS-21 acyl chain based on its expression pattern. Interestingly, whereas the UGTs identified as glycosylating the QS-21 scaffold belong to the group A, which is known to contain enzymes that add sugars to the sugar chain of various scaffolds (Louveau et al., 2019), UGT-L was the only Q. saponaria UGT that was both highly expressed and belonging to a different clade, namely group D. Some glucosylation activity towards the alcohol of the acyl chains of zeatin and dihydrozeatin have been shown for some group D UGTs (Hou et al., 2004).

To test the activities of the two versions of UGT-L (-short and -long), they were independently co-expressed with the gene sets providing the QA-TriR-FRX-C18 substrate. Both versions of UGT-L were able to add an arabinofuranose to QA-TriR-FRX-C18 (FIG. 17A). UGT-L-long was further tested on QA-TriX-FRXX-C18 and on QA-TriX-FRXX-C9. It added an arabinofuranose to both molecules (FIGS. 17B and C).

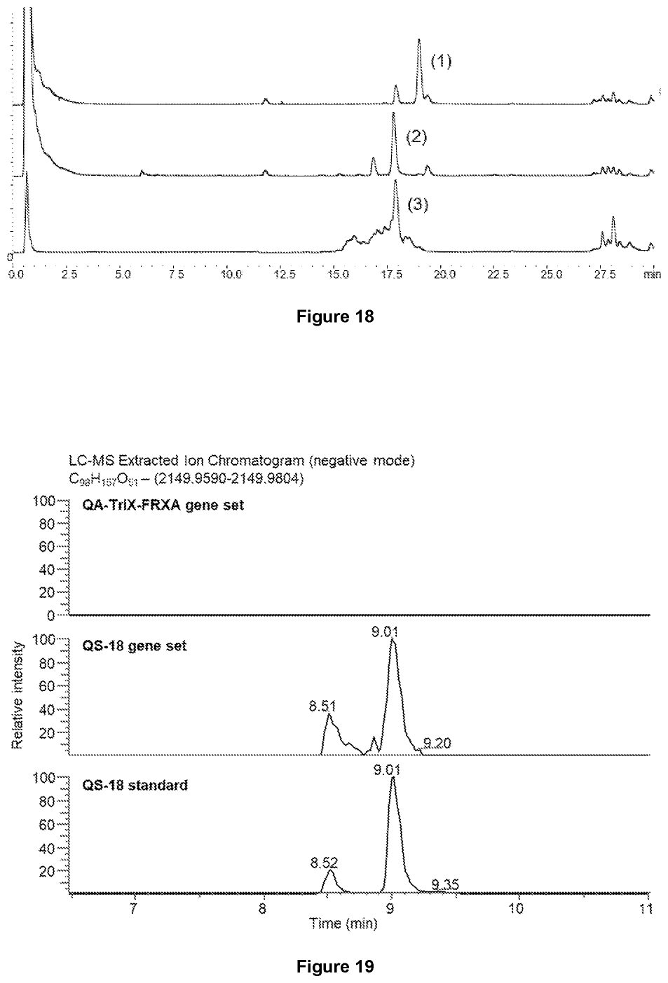

Furthermore, arabinofuranosyl transfer activity of UGT-L was assessed by in vitro experiments. Reactions were composed of purified sugar acceptor, Des-arabinosyl-QS-21 (QA-TriX-FRXA-C18), sugar donor, UDP-β-L-arabinofuranose and purified UGT-L enzyme. In the presence of UGT-L, most of Des-arabinosyl-QS-21 was converted to QS-21, confirming the identity of UGT-L as the arabinofuranosyl transferase (FIG. 18).

### Example 8—Identification of QS-7-GIcT

Previously, a series of genomic and transcriptomic sequence resources were generated for Q. saponaria and used to identify the genes required for the production of the saponin QA-Tri(X/R)-FRX(A/X). Through these sequence resources, it has been established that genes required for biosynthesis of this scaffold show co-expression between different Q. saponaria tissues and are highly expressed in the leaf primordiuma.

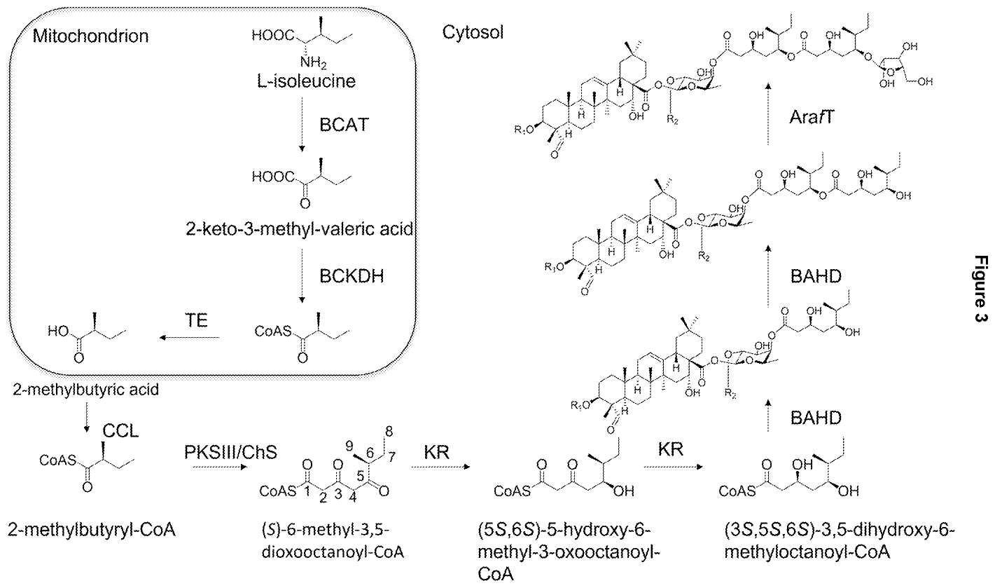

UDP-dependent glycosyltransferases (UGT) are commonly associated with glycosylation of plant natural products (Louveau & Osbourn, 2019) and several such enzymes are known to be required for the production of QA-Tri(X/R)-FRX(A/X). Using the sequence resources described above, one UGT was identified (QsUGT-BI) which showed a similar expression pattern to the previously characterised enzymes. Transient expression of QsUGT-BI with the genes from Q. saponaria required for biosynthesis of QA-TriX-FRXA scaffold resulted in identification of a new product by LC-MS with a mass that suggested the addition of a hexose residue. Several characterised saponins from Q. saponaria are known to feature glucose residues attached to the C-28 saccharide chain (Fleck et al., 2019), suggesting that the hexose added by QsUGT-BI was likely to be a glucose. One such saponin is QS-18, which features a D-glucose attached to the C-3 position of the rhamnose residue at C-28 (FIG. 2). The putative glucosyltransferase QsUGT-BI is also referred to herein as QS-7-GIcT.

### Example 9—Making QS-21 in N. benthamiana

The enzymes identified in this work (3CCL, ChSA-F, KR11, KR23′, DMOT9, DMOT4 and UGT-L-long) were used to produce the two isomers of the QS-21 fraction (QA-TriX-FRXA-C18-A and QA-TriX-FRXX-C18-A) in N. benthamiana (FIG. 20). This set of enzymes was also able to add the C18-A chain to QA-TriR-FRXA and QA-TriR-FRXX (FIG. 20A). The MS2 spectra of the QS-21 produced in N. benthamiana matched the one of the QS-21 standard (FIG. 20B).

### Example 10—Increasing the Yields of QS-21

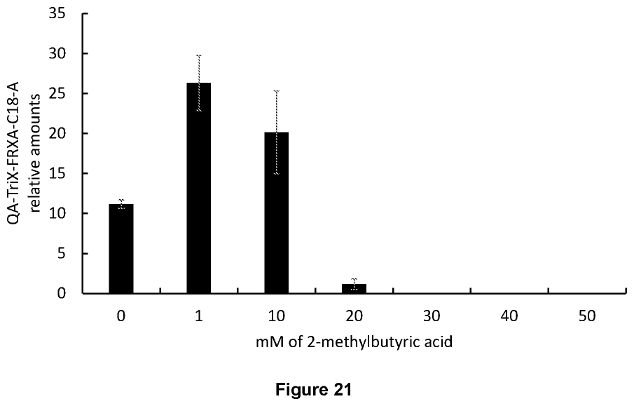

It was hypothesized that the production of QS-21 could be improved by increasing the amount of the available C9 acyl unit. 2-methylbutyric acid, as the putative substrate of 3CCL, should increase the intra-cellular concentration of 2-methylbutyryl-CoA, increasing the level of the substrate of the ChS enzyme candidates. FIG. 21 shows that the addition of 1 mM 2-methylbutyric acid in the A. tumefaciens infiltration buffer led to more than twice the amount of QA-TriX-FRXA-C18-A seen in the control. These data show that providing the infiltrated plant with 2-methylbutyric acid is a viable approach to boost QS-21 yields.

### Example 11—Making QS-18 in N. benthamiana

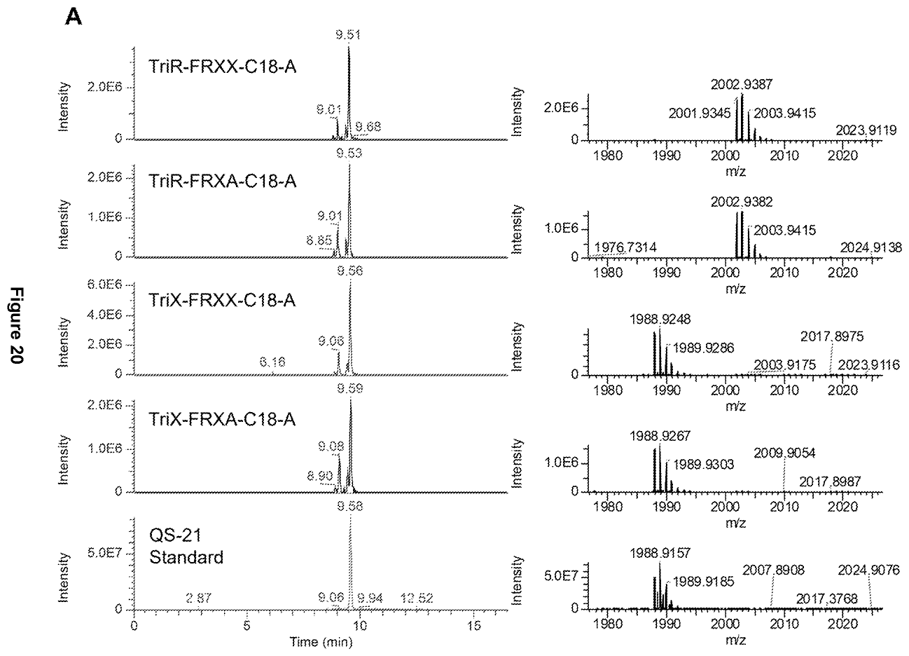

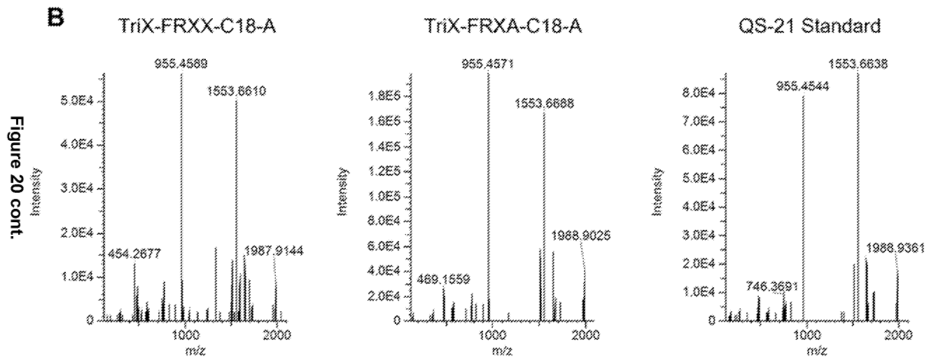

QS-18 is derived from QS-21 through addition of a single D-glucose residue at the C-3 position of a L-rhamnose residue within the C-28 linear tetrasacharide chain. Previously, the production of QS-21 has been demonstrated in N. benthamiana by transient expression of the relevant biosynthetic enzymes from Quillaja saponaria (See Example 9). Therefore, the QS-21 genes and glucosyltransferase (QS-7-GIcT) were transiently co-expressed in N. benthamiana. Subsequent LC-MS analysis of leaf extracts revealed the presence of a peak which matched the retention time and mass of a QS-18 standard, thereby confirming successful production of QS-18 in N. benthamiana (FIG. 19).

### Example 12—Employinq a Mutant Threonine Deaminase Boosts QS-21 Content in N. benthamiana

As demonstrated in Example 10, the addition of 2-methylbutyric acid can boost the production of QS-21. This is expected to be due to the ability of 3CCL to convert 2-methylbutyric acid to 2-methylbutyryl-CoA, which along with Malonyl-CoA, serves as a substrate of the ChS enzymes need to form the C9 acyl unit.

Physiologically, 2-methylbutryl-CoA (aka 2-methylbutanoyl-CoA) is derived from the degradation of isoleucine (Hildebrandt et al., 2015). However, based on profiling of Arabidopsis thaliana leaves, isoleucine appears to represent one of the least abundant free amino acids (Joshi et al., 2010; Watanabe et al., 2013). Assuming that N. benthamiana has a similar profile of free amino acids, it is possible that low levels of free isoleucine limits the availability of 2-methylbutyryl-CoA available.

Isoleucine can be synthesised by degradation of threonine by threonine deaminase (TD), an enzyme which is subject to feedback regulation by isoleucine. Recently, feedback insensitive mutants for the A. thaliana TD (AtTD) (At3g10050) were identified, including a one line featuring a proline→leucine mutation at position 519. This mutant accumulates 143-fold more isoleucine than the wild type (Xing & Last, 2017).

Using the Q. saponaria transcriptomic resources, a likely TD from Q. saponaria (Qs0222940) (referred to as QsTD) was identified using the A. thaliana TD as a BLAST query. A multiple sequence alignment between the AtTD and QsTD enzymes was performed. This revealed that AtTD P519 aligns to QsTD P540. Primers were therefore designed to clone and mutagenize the QsTD enzyme (P540L).

This QsTD was amplified by PCR from leaf cDNA using the QsTD_0222940_attB(1F/2R) primer pairs and cloned into pDONR 207. After sequencing to identify a suitable clone, mutagenesis was performed using the QsTD_P540L_Q5(F/R) primers with a Q5 site-directed mutagenesis kit (New England Biolabs) according to the manufacturer's instructions.

QsTD_0222940_attB1F
SEQ ID NO: 131: [symbol] [symbol]
ATGGAGACACTGTGTTAC
TCGCCATC);
QsTD_0222940_attB2R
SEQ ID NO: 132:
( [symbol] [symbol] TTAATGCATCAAAAGCTGG
AAATCTTCATC).
QsTD_P540L_Q5F
SEQ ID NO: 135:
(TTCATCTTTC [symbol] GGAGAGGCCTG)
QsTD_P540L_Q5R
SEQ ID NO: 136:
(ACGGCAAAGAACCTCATTC)

The wild-type (SEQ ID NO: 133) and P540L mutant (SEQ ID NO: 134) forms of QsTD were cloned into pEAQ-HT-DEST1 and transformed into A. tumefaciens. The resulting transformed bacteria were cultured and infiltrated into N. benthamiana along with the equivalent A. tumefaciens cultures harbouring the QS-21 genes. As a positive control, the QS-21 genes were also co-infiltrated with 2-methylbutryic acid.

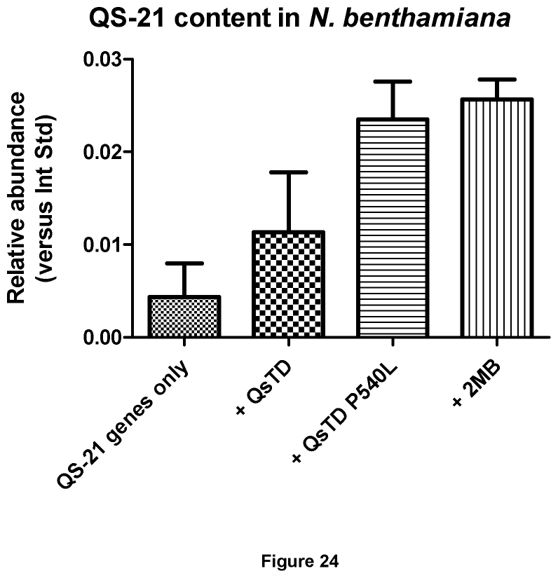

Five days after infiltrations, leaves were harvested and analysed by LC-MS. Quantification of the QS-21 content in leaves revealed that a minor (˜2.6-fold) boost in QS-21 content could be seen with use of the wild-type QsTD. In contrast, expression of the QsTDP540L mutant resulted in ˜5-fold increases in QS-21 content. These levels are comparable to the levels achieved with inclusion of 2-methylbutyric acid (FIG. 24).

This demonstrates that further metabolic engineering targeting the biosynthesis of the QS-21 acyl chain can be employed to further enhance content of QS-21 in heterologous hosts such as N. benthamiana.

### Example 13—Investigation of the Activity of the UGT73CZ2 (UGT-L-Long) Sugar Transferase In Vitro

### Generation of Purified UGT73CZ2

UGT73CZ2, a candidate arafT enzyme, (also known as UGT-L-long herein) was expressed with a carboxy-terminal hexahistidine tag in N. benthamiana by agro-infiltration. The purity of UGT-L-long was monitored by SDS-PAGE and CBB staining.

### Purification of the Des-Arabinosyl-QS-21 Acceptor (11)

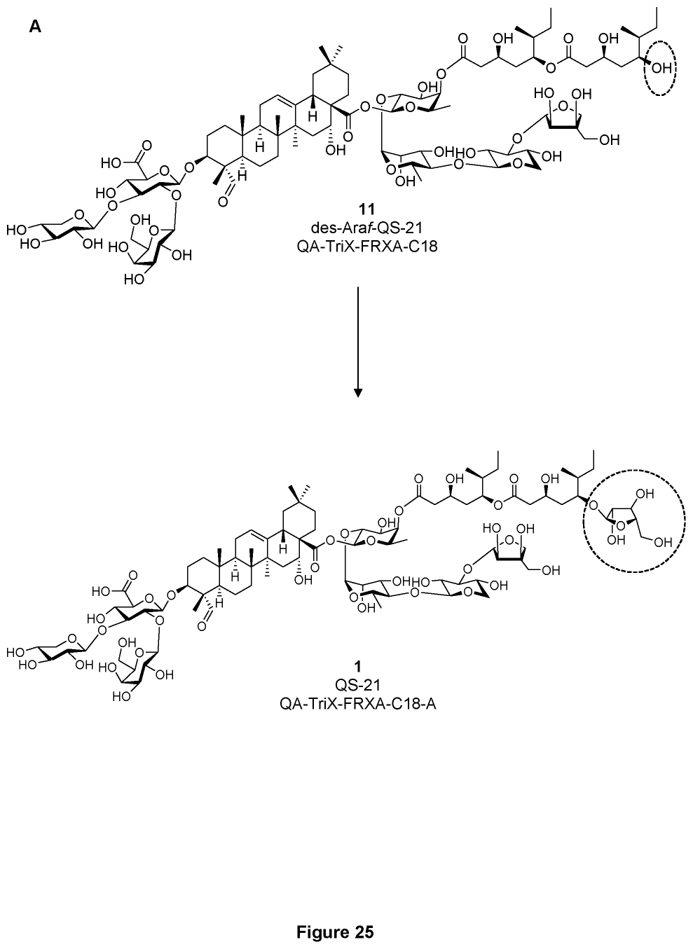

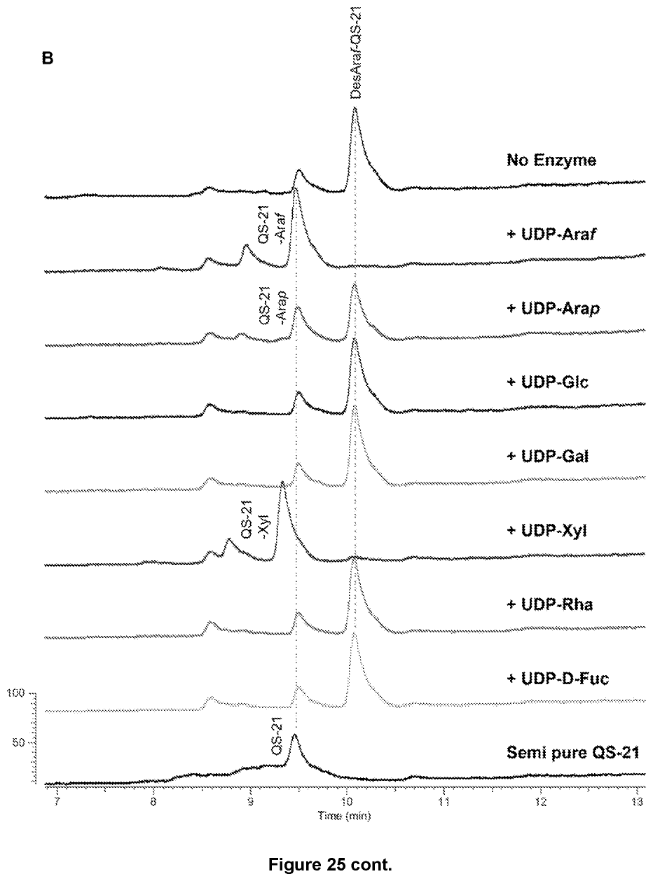

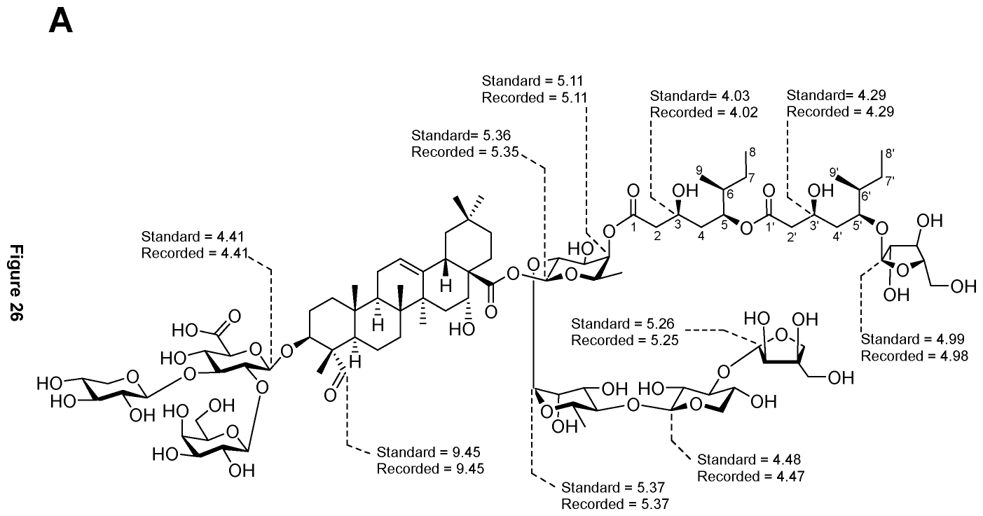

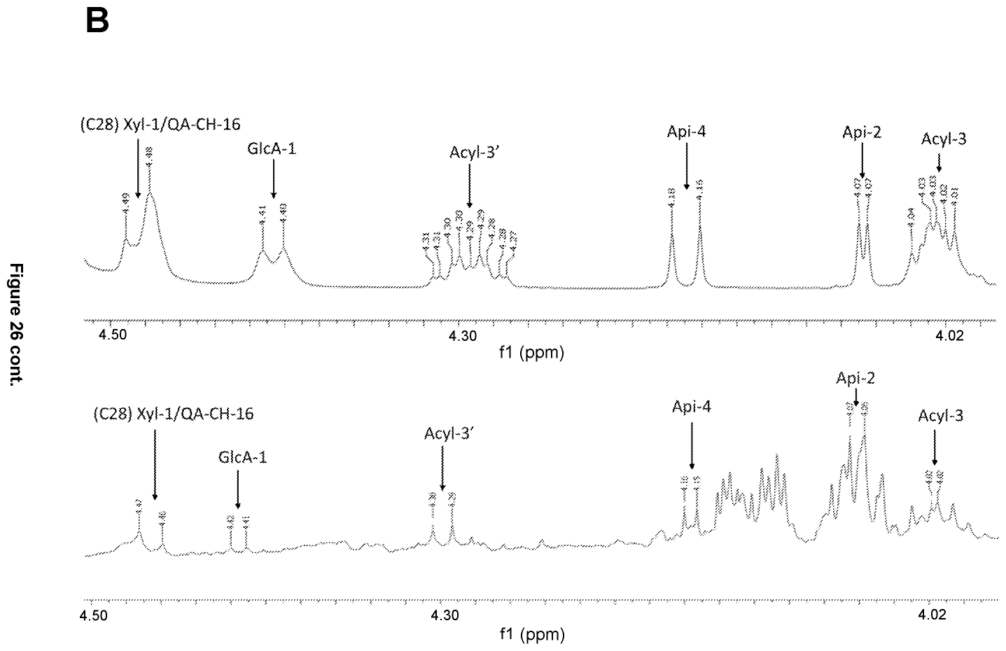

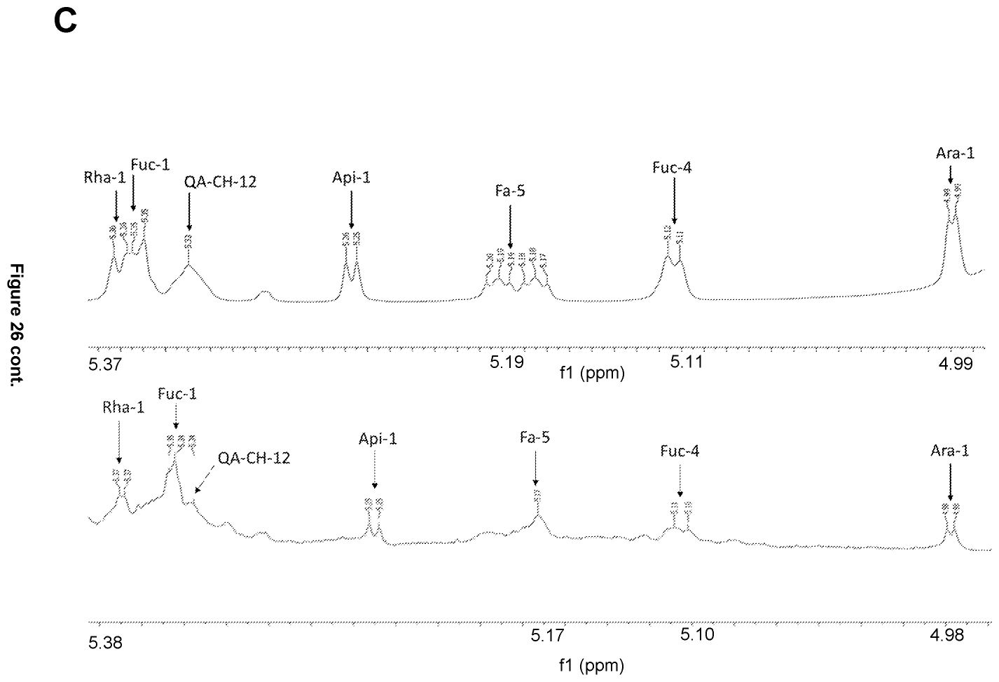

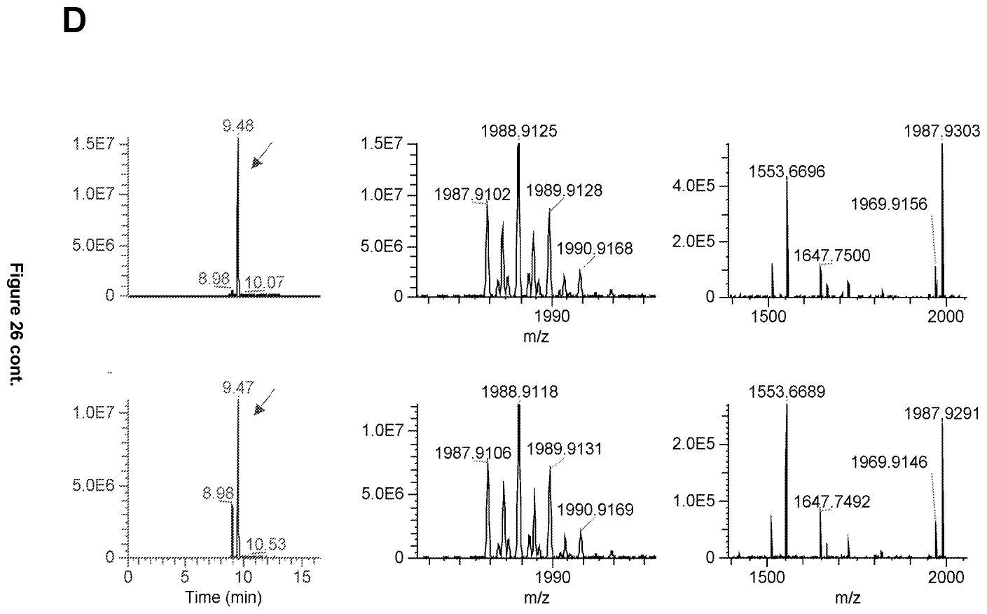

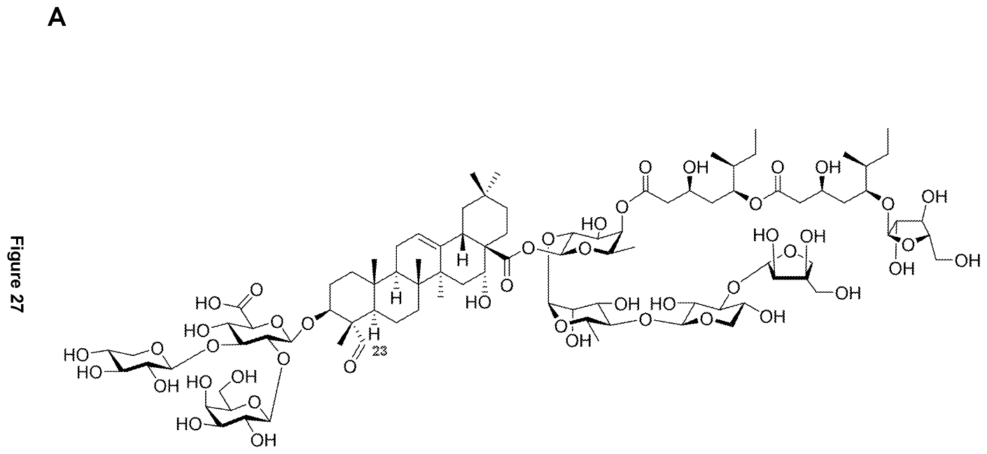

1 g of commercially available Q. saponaria (Sigma-Aldrich) bark was solubilized in methanol/water [80/20, V/V] and directly subjected to a Biotage C18-60 g reversed phase flash column chromatography using a long gradient of [H₂O/ACN+0.1% formic acid, (90/10 -30/70) for 60 minutes, 50 mL/min]. Fractions were monitored by LC-MS. A fraction containing QS-17, QS-18, and QS-21 along with des-arabinosyl QS-21 was subjected to further repetitive fractionation using an Agilent semi-preparative HPLC [in isocratic mode, H₂O/ACN+0.1% formic acid, (55/45) for 30 min, 4 mL/min, (Luna 5 m C18 (2), 250×10 mm)]. A peak corresponding to the des-arabinosyl form of QS-21 was collected and dried to give 3.5 mg of purified product. This was confirmed to be the des-arabinosylated form of QS-21 (D-apiose form) (1; see FIG. 25) by HR-MS and extensive 1 and 2D-NMR analysis. This compound (11; see FIG. 25) was used as the acceptor in assays of UGT-L-long activity (see below).

UGT-L-Long Enzyme Assays The reaction mixture was composed of 50 mM HEPES-KOH, pH 7.5, 2 mM MgCl₂, 0.3% 2-mercaptoethanol, 0.1 mM des-arabinosyl-QS-21 (QA-TriX-FRXA-C18) and 0.5 mM of each UDP sugar in a final volume of 50 μL (see FIG. 25). Reactions were initiated by addition of 0.8 μg of purified UGT-L-long to the reaction mixture and incubated at 25° C. for 14 h. After quenching with methanol (final 50%), the filtered reaction mixture (10 μL) was analysed with a QExactive Hybrid Quadrupole-Orbitrap mass spectrometer (Thermo Scientific) equipped with a Charged Aerosol Detector (CAD, Thermo Scientific) and a RP-C18 column (Kinetex XB-C18, 100 Å, particle size 2.6 μm, 50×2.1 mm, Phenomenex). UDP-β-L-arabinofuranose was obtained from Peptide Institute (Japan), UDP-α-D-glucose and UDP-α-D-galactose were from Sigma-Aldrich, UDP-α-D-xylose and UDP-β-L-rhamnose were from Carbosynth (Switzerland). UDP-β-L-arabinopyranose and UDP-α-D-fucose were prepared following published procedures (see J. C. Errey, B. Mukhopadhyay, K. P. R. Kartha, R. A. Field, Flexible enzymatic and chemo-enzymatic approaches to a broad range of uridine-diphospho-sugars. Chem. Commun. 2706-2707 (2004)). UGT-L-long is able to transfer L-arabinofuranose and also D-xylose to 11 (des-Araf-QS-21; QA-TriX-FRXA-C18) with almost 100% efficiency, as shown by the absence of the substrate peaks. Charged Aerosol Detector (CAD) chromatograms are shown (FIG. 25). QS-21-D-Xyl (QA-TriX-FRXA-C18-Xyl), an example of QA*-F*-C18-Xyl, eluted earlier than QS-21 (1).

### Example 14—Purification and Structural Determination of QS-21 Produced in N. benthamiana

Four hundred N. benthamiana plants were vacuum infiltrated as described in (37) with equal amounts of the A. tumefaciens strains containing the genes required to make QA-TriX-FRXA (the C-28 d-apiose variant of the QS-21 pathway intermediate) and 3CCL, ChSB, ChSC, ChSE, ChSF, KR11, KR23′, DMOT4 and DOMT9 and UTG-L-long. The infiltration mix was supplemented with 1 mM of 2-methylbutyric acid as we have shown that it increases the yields of QS-21. After five days, the leaves showing a phenotype were harvested, freeze-dried and prepped as described in Stephenson et al. (2018) for pressurized solvent extraction. The leaves were first defatted with hexane in the pressurized solvent extraction instrument (0 min hold time), then the extracts resulting from two cycles of 100% methanol (0 min hold time, then 5 min hold time, 100° C.) were pooled. The extract was dried on celite and flash chromatography (5 to 100% acetonitrile, flowrate of 50 mL/min, 1312 mL) was used as a first fractionation step. The fraction containing QS-21 was further purified using an Agilent 1260 prep LC-MS with water+0.1% formic acid (solvent A) and acetonitrile (solvent B): from 0 to 2 min, 15 to 40% B; from 2 to 34 min, 40 to 60% B (QS-21 elutes around 50% B); from 34 to 34.5 min, 60 to 100% B, held for 3.5 min, then returning to 15% B in 30 s, flow at 25 mL/min, on a Luna® 5 mm C18(2) 100 Å LC column 250×21.2 mm. The fractions containing QS-21 were further purified using an Agilent 1290 UHPLC with the same chromatography method used for the Agilent 1260 prep LC-MS but with a Luna 5 mm C18(2) 100 Å column 250×10 mm at 4 mL/min.

### Example 15—NMR Analysis of QS-21

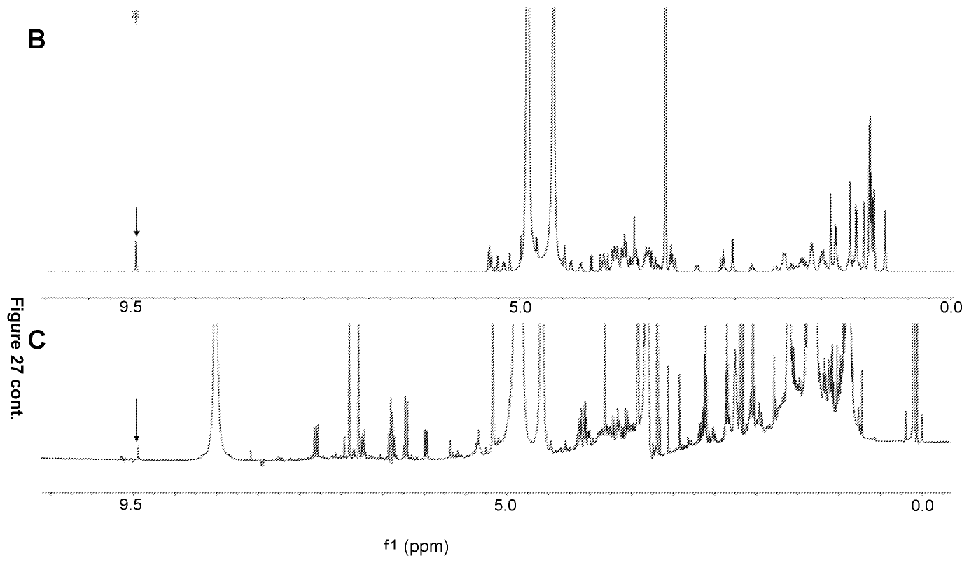

1H-NMR (600 MHz, MeOH-d4) spectra for the product produced following scale-up in N. benthamiana and a QS-21 standard (Desert King) were generated (see Table 1 and FIG. 27) and compared with the data reported for QS-21 in the literature (see N. E. Jacobsen et al., Structure of the saponin adjuvant QS-21 and its base-catalyzed isomerization product by 1H and natural abundance 13C NMR spectroscopy. Carbohydr. Res. 280, 1-14 (1996); N. T. Nyberg, L. Kenne, B. Rönnberg, B. G. Sundquist, Separation and structural analysis of some saponins from Quillaja saponaria Molina. Carbohydr. Res. 323, 87-97 (1999); and L. I. Nord, L. Kenne, Novel acetylated triterpenoid saponins in a chromatographic fraction from Quillaja saponaria Molina. Carbohydr. Res. 329, 817-829 (2000)).

TABLE 1
¹HNMR spectroscopic data comparison (key resonances) for the
QS-21 standard and the product produced in N. benthamiana with
the data reported for QS-21 in the literature (see Example 15). All
spectra were recorded in MeOH-d₄ (600 MHz).

| | | | Literature |
|---|---|---|---|
| | | Product produced in | (Jacobson et |
| Position | QS-21 standard | N. benthamiana. | al., 1996) |
| C-23 | 9.45, s | 9.45, s | 9.46 |
| C₃-GlcA | 4.41, d (7.1) | 4.41, d (5.2) | 4.45 |
| C₂₈-Fuc-1 | 5.36. app d | 5.35, app d | 5.36 |
| C₂₈-Fuc-4 | 5.11, d (3.2) | 5.11, d (3.9) | 5.11 |
| C₂₈-Rha-1 | 5.37, app d | 5.37, d (1.4) | 5.37 |
| C₂₈-Xyl-1 | 4.48, d (8.1) | 4.47, d (8) | 4.47 |
| C₂₈-Api-1 | 5.26, d (2.9) | 5.25, d (2.7) | 5.25 |
| C₂₈-Api-2 | 4.07, d (2.9) | 4.06, d (5) | 4.06 |
| C₂₈-Api-4-a | 4.17, d (9.7) | 4.16, d (4.3) | 4.17 |
| Ara-1 | 4.99, d (1.8) | 4.98, d (2) | 4.98 |
| Acyl-3 | 4.03, m | 4.02, m | 4.02 |
| Acyl-5 | 5.18, m | 5.17 br s | 5.19 |
| Acyl-3′ | 4.29 | 4.29 | 4.30 |

### Materials and Methods

### Primers and Cloning

The genes encoding the enzymes described herein (1CCL to 6CCL, ChSA to ChSF, KR11, KR23′, DMOT4, DMOT9 and QS-7-GIcT) were amplified by PCR from cDNA derived from leaf tissue of Q. saponaria. PCR was performed using the primers detailed in Table 2 and iProof polymerase with thermal cycling according to the manufacturer's recommendations. The resultant PCR products were purified (Qiagen PCR cleanup kit) and cloned into the pDONR207 vector using BP clonase according to the manufacturer's instructions. The BP reaction was transformed into E. coli, the resulting transformants were cultured and the plasmids isolated by miniprep (Qiagen). The isolated plasmids were sequenced (Genewiz) to verify the presence of the correct genes. Next, each of the genes were further subcloned into the pEAQ-HT-DEST1 and into the pAG423GAL-ccdB or pAG425GAL-ccdB expression vectors using LR clonase. The resulting pEAQ-HT-DEST1 vectors were used to transform A. tumefaciens LBA4404 or GV3101 by flash freezing in liquid N2 and the pAG423/425GAL-ccdB vectors were transformed into chemically competent yeast cells.

TABLE 2
Primers used to clone the sequences. Gene specific
sequences are shown in black, while the attB sites
required for Gateway ® cloning are shown in grey.

| Name | Sequence |
|---|---|
| Qs1CCL_attB1F | [symbol] [symbol] ATGGATCAATTAAATCCAAAGCCTGTAAAC |
| Qs1CCL_attB2R | [symbol] [symbol] TTACAACCGACTAGCTGTCAACG |
| Qs2CCL_attB1F | [symbol] [symbol] ATGGAAGATCTTAAGCCAAGCCCAG |
| Qs2CCL_attB2R | [symbol] [symbol] TCACATCCGACTCGATCTTAGTGGG |
| Qs3CCL_attB1F | [symbol] [symbol] ATGGAAGATCTTAAACCAAGCTCTGC |
| Qs3CCL_attB2R | [symbol] [symbol] CTATGTGTGGTTGGAGAGAGGC |
| Qs4CCL_attB1F | [symbol] [symbol] ATGGAAAACCTTAAGCCAACCCC |
| Qs4CCL_attB2R | [symbol] [symbol] TCACATCCGACTCGACCTTGG |
| Qs5CCL_attB1F | [symbol] [symbol] ATGGAGCAACTCAACCCAAAACC |
| Qs5CCL_attB2R | [symbol] [symbol] CTACAACCGACTATCTGTCAACGACC |
| Qs6CCL_attB1F | [symbol] [symbol] ATGGAGCAACTCAACCCAAAACC |
| Qs6CCL_attB2R | [symbol] [symbol] CTACAACCGACTATCTGTCAACGACC |
| QsChSA_attB1F | [symbol] [symbol] ATGGTGACTGTGGAGGAAGTTCG |
| QsChSA_attB2R | [symbol] [symbol] TTAAGCAGACACGCTATGAAGCAC |
| QsChSB_attB1F | [symbol] [symbol] ATGGCATCCATCGAAGCAATCC |
| QsChSB_attB2R | [symbol] [symbol] TTAATTGGTTTCAACCGGAACACTG |
| QsChSC_attB1F | [symbol] [symbol] ATGGTGACTGTGGAGGAAGTTCG |
| QsChSC_attB2R | [symbol] [symbol] TTAGGCAGCCAGACTATGAAGCAC |
| QsChSD_attB1F | [symbol] [symbol] ATGGCTCCGGTGACCAACAC |
| QsChSD_attB2R | [symbol] [symbol] TTACACAACTGGGACACTGCGC |
| QsChSE_attB1F | [symbol] [symbol] ATGGGATCCTTGGATCAAGATTTTC |
| QsChSE_attB2R | [symbol] [symbol] TTAATGGGCCTCATTTGTAGAAATGC |
| QsChSF_attB1F | [symbol] [symbol] ATGGCATCCGTCGAAGCAATC |
| QsChSF_attB2R | [symbol] [symbol] TCAATAACTTATCGGAACACTGTGCAAC |
| QsKR11_attB1F | [symbol] [symbol] ATGGCGGCGGCCGAAAGTGAAAG |
| QsKR11_attB2R | [symbol] [symbol] CTAGTTAGTTTCGAGAACTCCTGCCTTTCGATAGC |
| QsKR23′_attB1F | [symbol] [symbol] ATGCTGTATAGTAGCTAGGCTAAAAACC |
| QsKR23′_attB2R | [symbol] [symbol] TTATTCTTTCTTCCTTGCATCTTTAAGCTGCC |
| QsDMOT4_attB1F | [symbol] [symbol] ATGGCTGAAGCTTTGAAATTGAAAGTAGTTG |
| QsDMOT4_attB2R | [symbol] [symbol] TTAGAGTATCTTCAATGTTTGATCAAATGTCTGAATG |
| QsDMOT9_attB1F | [symbol] [symbol] ATGATGGAGGTACATACCACATCGG |
| QsDMOT9_attB2R | [symbol] [symbol] TCAAATGATAGTGGGATTAACCATGGCAG |
| QsUGT-L- | [symbol] [symbol] ATGGGTTCCAGTGCAAAGCCTC |
| short_attB1F | |
| QSUGT-L- | [symbol] [symbol] TCATTCCTGCGTGCTAGTTGATTGTTG |
| short_attB2R | |
| QSUGT-L- | [symbol] [symbol] ATGCCATTCATTCGTAATACTCC |
| long_attB1F | |
| QSUGT-L- | [symbol] [symbol] TCATTCCTGCGTGCTAGTTG |
| long_attB2R | |
| UGT-BI_attB1F | [symbol] [symbol] [symbol] [symbol] |
| UGT-BI_attB2R | [symbol] [symbol] [symbol] [symbol] |

Agroinfiltration of N. benthamiana Leaves

Agroinfiltration was performed using a needleless syringe as previously described (Reed et al., 2017). All genes were expressed from pEAQ-HT-DEST1 binary expression vectors (Sainsbury et al., 2009) in A. tumefaciens LBA4404 or GV3101 as described above. Cultivation of bacteria and plants is as described in (Reed et al., 2017).

Preparation of Q. saponaria and N. benthamiana Leaf Extracts for LC-MS Analysis

N. benthamiana leaves were harvested 5 days after agroinfiltration. Plant material (10-15 mg per sample) was disrupted with tungsten beads at 1000 rpm for 1 min (Geno/Grinder 2010, Spex SamplePrep), this step being repeated until fully ground. Metabolites were extracted in 700 μL 80% methanol containing 20 μg/mL of internal standard (digitoxin, Sigma-Aldrich) and incubated for 1 hour at 70° C., with shaking at 1000 rpm (Thermomixer Comfort, Eppendorf). Each sample was defatted by partitioning once with 400 μL hexane. The upper phase was discarded and the lower aqueous phase was dried under vacuum at 45° C. for 1.5 hour (EZ-2 Series Evaporator, Genevac). Dried material was resuspended in 130 μL of 80% methanol, filtered at 12,500×g for 30 s (0.2 μm, Spin-X, Costar), and used for LC-MS analysis.

### Yeast Competent Cell Preparation and Transformation

Yeasts were grown at 30° C.-220 rpm to OD 600 nm 0.6-0.8 in 50 mL YPD. The cells were pelleted by centrifugation at 2000 g for 2 min and resuspended in 25 mL sterile water. The centrifugation step was repeated and the pellet resuspended in 1 mL 100 mM Lithium Acetate (LiAc) and transferred into an Eppendorf tube, in which it was pelleted again and resuspended in 400 μL 100 mM LiAc. 50 μL of yeast suspension were used per transformation.

To transform the cells, the 50 μL suspensions were centrifuged at 250 g for 30 s. Over the pellets were added 240 μL PEG (MW 3350, 50% w/v), 35 μL freshly made 1 M lithium acetate, 8 μL denatured 2.0 mg/ml salmon sperm DNA (average size 5-7 kb) and 1 μg plasmid DNA. This was vigorously vortexed until the cells were in suspension. The cells were then gently shaken at 30° C. for 30 min then at 42° C. for 20 min. They were centrifuged at 500 g for 1 min and resuspended in 100 μL sterile H₂O. The cells were plated in SD medium lacking leucine or histidine for the pAG425GAL-ccdB and pAG423GAL-ccdB plasmids, respectively. After three days, glycerol stocks were made from single colonies.

### PIVP/PMBP Measurements

Yeast strains were grown for 3 days at 30° C. in SD+glucose drop out media (-Histidine for the CCL genes in pAG423GAL-ccdB, -Leucine for the HIVPS in pAG423GAL-ccdB and -Histidine-Leucine for the yeast strains containing both vectors, which included the empty vector control) on petri dishes from the respective glycerol stocks. Two 10 mL cultures per sample were started from grown colonies directly in SD+galactose drop out media for one day at 30° C. The cultures' absorbances were taken for later normalization of the data and centrifuged at 2000 g for 5 min. To the pellets were added 650 moles of naringenin as internal standard (so that the concentration of naringenin in the pooled extracts were of 10 μM for injection), ˜300 μL of acid-washed glass beads (G8772 sigma) and 800 μL 70% methanol. The cells were ground full speed in a Geno/Grinder for 15 min and the complete cell lysis was ascertained by light microscopy. The supernatants were transferred into 2 mL Eppendorf tubes, and pooled as appropriate, dried in an evaporator at 40° C., resuspended in 130 μL 70% methanol, filtered and analysed in negative mode on a Thermo Scientific Q Exactive Hybrid Quadrupole-Orbitrap Mass spectrometer HPLC system, calibrated using Pierce+ve/−ve calibration standards according to the manufacturer's instructions with a Kinetex column 2.6 μm XB-C18 100 Å, 50×2.1 mm (Phenomenex). The gradient (solvent A, 0.05% formic acid and 5 mM ammonium formate in 10% acetonitrile; solvent B, 0.05% formic acid and 5 mM ammonium formate in 90% acetonitrile) program was set as follows at a flow of 0.35 ml.min⁻¹: 0-0.5 min, a linear gradient from 0 to 40% of B; 0.5-2.5 min, 40% of B; 2.5-3.5 min, a linear gradient to 100% of B; 3.5-4.5 min, 100% of B; 4.5-4.51 min, a linear gradient to 100% of A; 4.51-6 min, 100% of A (Xu et al. 2013).

Around 10-15 mg of freeze-dried infiltrated N. benthamiana leaves were ground in the Geno/Grinder with two 3 mm tungsten carbide beads (Qiagen) for 1 min at 1000 rpm. Naringenin (10 M at injection) was used as an internal standard and 600 μL of 100% methanol were added to the samples. The samples were vortexed, centrifuged full speed for 5 min and the supernatants were transferred into a fresh Eppendorf tube. The extraction was repeated with 300 μL of 100% methanol and the supernatants of both extractions were pooled. 300 μL of hexane and 200 μL of water were added and, after vortexing, the samples were centrifuged at 4° C. for 5 min. The lower aqueous phases were transferred into a new Eppendorf tube and dried in a GeneVac at 40° C. for 2 h. The pellets were resuspended in 130 μL of 100% methanol, filtered (Whatman, VectaSpin micro Anopore, Diam 0.2 μm) and transferred into glass vials with conical glass inserts. A Waters Xevo TQ-S Tandem LC-MS system was used to measure PIVP/PMBP for the N. benthamiana samples. The method and column were as describe above. Semi quantitative analysis was done using the ion transitions 209.057>165.061, 24 V cone, 18 V collision, ES- for PIVP/PMBP and 271.036>150.942, 40V cone, 16 V collision, ES- for naringenin.

### 2-Methylbutyryl-CoA Measurements

Yeasts were grown and extracted as described in the PIVP/PMBP measurements section with some modifications. The cultures absorbances were taken for subsequent normalization (as for the PIVP/PMBP experiment, two 10 mL cultures were used per replicate) and the pellets were resuspended in 300 μL quenching/extraction buffer (95% acetonitrile, 25 mM formic acid, −20° C.) and 150 μL cold water. The cell lysis was as described above. After full speed centrifugation for 2 min in a microcentrifuge, the supernatants were transferred into a fresh 2 mL tube (pooling of relevant extracts was done as appropriate) and freeze-dried overnight. The pellets were resuspended in 130 μL resuspension buffer (25 mM ammonium formate, pH 3.0, 2% MeOH, 4° C.), filtered (0.2 μm, Spin-X, Costar) and transferred into glass vials with conical inserts for LC/MS analysis.

The N. benthamiana leaves were harvested five days after infiltration and ground in liquid nitrogen. Around 1.6 g of the material were weighted in 50 mL falcon tubes. The powders were resuspended in −15 mL quenching/extraction buffer (95% acetonitrile, 25 mM formic acid, −20° C.). −15 mL of cold water were then added and the samples were shaken for 20 min at 4° C. before being centrifuged at 4000 g for 10 min. The supernatants were freeze-dried. The pellets were resuspended in 1 mL water, centrifuged and the supernatants were transferred into 2 mL Eppendorf tubes with pierced lids for an additional freeze-drying step. The samples were resuspended in 130 μL resuspension buffer (25 mM ammonium formate, pH 3.0, 2% MeOH, 4° C.), filtered (0.2 μm, Spin-X, Costar), and transferred into glass vials with conical inserts.

The samples were then analysed on a Waters Xevo TQ-S Tandem LC-MS system. The buffers and methods were as described in Glässer et al. (2020), using a Kinetex column 2.6 μm XB-C18 100 Å, 50×2.1 mm (Phenomenex) and negative mode. The massLynx software was used to extract the peak areas using the ion transition of 850.2684>407.9766, 80V cone, 60 eV collision for 2-methylbutyryl-CoA and the ion transitions of 852>407.98 and 852>158.89 with 44V cone and 34 eV and 59 eV collision for malonyl-CoA (total ion chromatogram). These conditions were improved to measure the products of the CCL enzyme candidates: the more sensitive positive mode was used and the following ion transitions targeted for quantification: 852.205>345.183, 68 V cone and 32 eV collision energy for 2-methylbutyryl-CoA and 838.209>331.147, 70 V cone and 32 eV collision energy for isobutyryl-CoA.

HPLC-ESI-MS Analysis of N. benthamiana Leaf Extracts for QA*-F* and Derivatives Detection

Analysis was carried out using a Thermo Scientific Q Exactive Hybrid Quadrupole-Orbitrap Mass spectrometer HPLC system, calibrated using Pierce+ve/−ve calibration standards according to the manufacturer's instructions. Detection: MS (ESI ionization), scan range of 400-2500 m/z in negative mode, 70,000 resolution. Data dependent MS², isolation window of 4.0 m/z, collision energy of 30, resolution of 17,500, dynamic exclusion of 5.0 s. Method: Solvent A: [H2O+0.1% formic acid] Solvent B: [acetonitrile (CH3CN)]. Injection volume: 10 μL. Gradient: 15% [B] from 0 to 0.75 min, 15% to 60% [B] from 0.75 to 13 min, 60% to 100% [B] from 13 to 13.25 min, 100% to 15% [B] from 13.25 to 14.5 min, 15% [B] from 14.5 to 16.5 min. Method was performed using a flow rate of 0.6 mL·min-1 and a Kinetex column 2.6 μm XB-C18 100 Å, 50×2.1 mm (Phenomenex). Analysis was performed using Xcalibur and FreeStyle softwares (Thermo Scientific).

Purification of QA-TriX-FRXA-C18 and of QA-TriR-FRXA-C18 from Q. saponaria Extracts

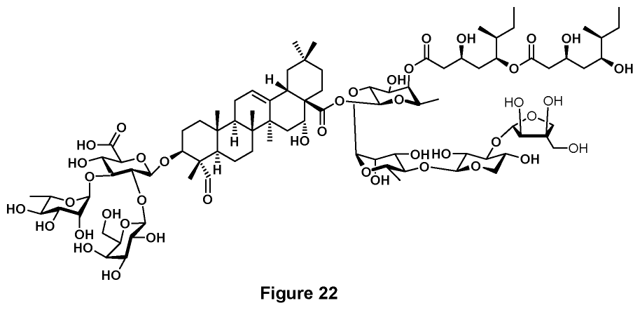

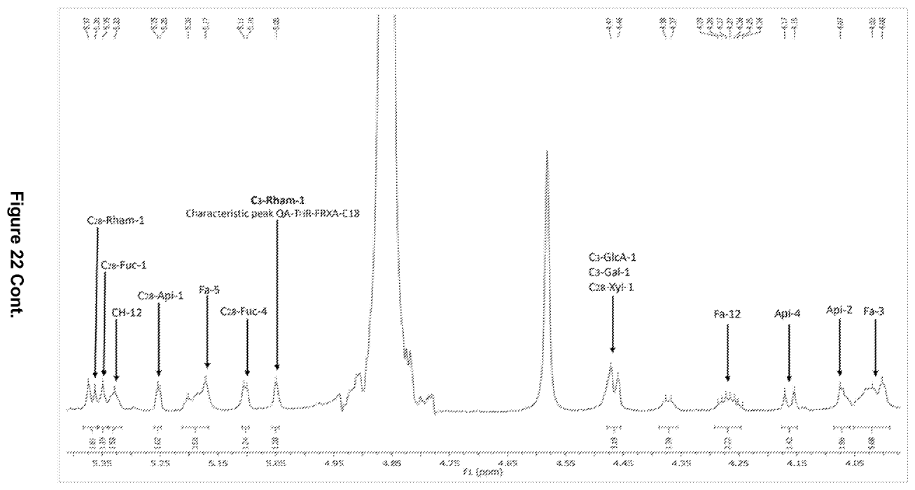

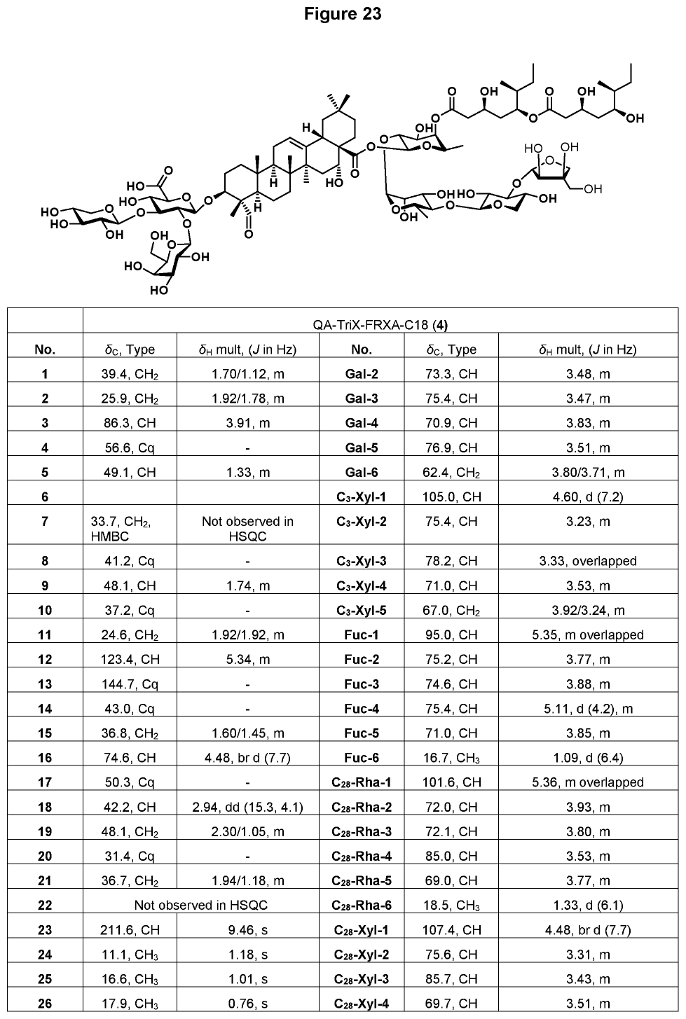

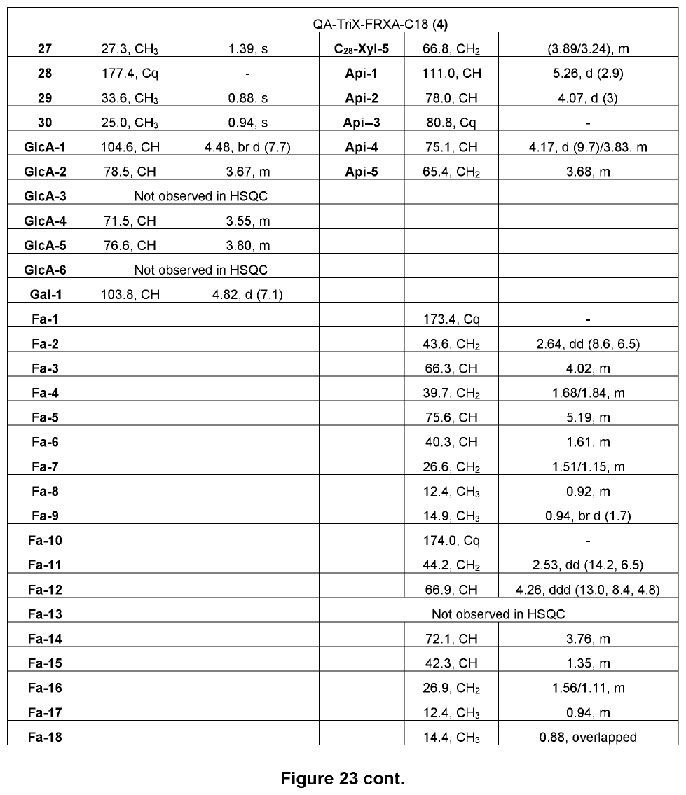

The purpose of this purification was to prepare appropriate des-arabinosyl substrate for the testing of UGT candidates. A 25 mL Quillaja saponaria crude extract was subjected to a C18-Biotage-60 g reversed phase flash chromatography purification using a long gradient of [H2O/ACN+0.1% formic acid, (90/10→30/70) for 60 minutes, 50 mL/min]. Twelve major sub-fractions were collected and the last six fractions were monitored by LC-MS, which revealed two main promising fractions, a semi-pure QS-21 fraction (fraction 8) and a semi-pure des-arabinosyl-QS-21 fraction (fraction 9—F9). Subsequentially, F9 was selected for further repetitive purifications using an Agilent semi-preparative HPLC machine [isocratic mode, H2O/ACN+0.1% formic acid, (55/45), for 30 minutes, 4 mL/min, (Luna 5 m C18 (2), 250×10 mm)], which led to 4 major peaks (named compounds 1 to 4) that were collected, dried and analysed. Based on extensive 1 and 2D-NMR along with HRESI-MS data interpretation, compounds 3 (1.9 mg) and 4, (3.5 mg) were identified as QA-TriR-FRXA-C18 and QA-TriX-FRXA-C18, respectively (see FIGS. 22 and 23).

### Expression and Purification of Arabinofuranosyl Transferase

The long version of arabinofuranosyl transferase (UGT-L-long) was overexpressed with a carboxy-terminal hexahistidine tag in N. benthamiana using the agroinfiltration transient expression system. The His-tag was added to UGT-L-long by PCR amplification using the oligonucleotides 5′-ATTCTGCCCAAATTCGATGCCATTCATTCGT-3′ and 5′-TGATGCATACCGGTCGTCAGTGGTGGTGGTGGTGGTGTTCCTGCGTGCTAGT-3′. The amplified fragment was thereafter inserted into a unique Nrul site of linearised pEAQ-HT vector by In-fusion cloning (TaKaRa Bio/Clontech). The resultant expression construct was transformed into the A. tumefaciens strain GV3101 and infiltrated into 3-week-old N. benthamiana leaves (Reed et al., 2017). After 6 days of incubation to allow sufficient accumulation of the enzyme, 10 g of infiltrated leaves were ground in 50 mL of HSorb buffer (50 mM HEPES-KOH, pH 7.8, 330 mM sorbitol) containing 1% polyvinylpolypyrrolidone, 7 mM 2-mercaptoethanol and 1 tablet of Complete EDTA-free protease inhibitor cocktail (Roche, 11 873 580 001) using mortar and pestle on ice. The homogenate was filtered through two layers of Miracloth (Calbiochem), centrifuged at 3220 g for 10 min to remove debris and centrifuged at 30000 g for 20 min to remove the microsomes. The cleared supernatant was split into several aliquots and flash-frozen in liquid nitrogen and stored at −70° C. in a freezer. The His-tagged UGT-L-long in 10 mL of the thawed supernatant was captured by 200 μL slurry of Ni Sepharose 6 Fast Flow (GE healthcare, 17-5318-01) for 1 h in a cold room with end-over-end mixing. The resin was washed four times with 1 mL of TBS-TX-Imi buffer (50 mM Tris-HCl, pH 7.5, 500 mM NaCl, 0.1% Triton X-100 and 20 mM imidazole), once with 1 mL of buffer A4 (20 mM HEPES, pH 7.5, and 150 mM NaCl) and eluted twice with 300 μL of Elution buffer (20 mM HEPES, pH 7.5, 150 mM NaCl, and 500 mM imidazole). The eluant was subjected to two cycles of dilution in buffer A4 and concentrated with Vivaspin 20, 50,000 MWCO PES (Sartorius, VS2031) to minimise imidazole content.

In Vitro Transfer of Arabinofuranose onto Des-Arabinosyl-QS-21

Reactions were composed of 50 mM HEPES-KOH, pH 7.5, 2 mM MgCl₂, 0.3% 2-mercaptoethanol, 0.1 mM QA-TriX-FRXA-C18 —compound 4 purified from Q. saponaria crude extract (as described in the above section) and 0.5 mM UDP-β-L-arabinofuranose (Peptide Institute, Japan) in a final volume of 50 μL. Reactions were initiated by addition of 0.75 μg of purified UGT-L-long-His to the reaction mix and incubated for 10 h at 25° C. with gentle shaking at 300 rpm. After the reactions were stopped by the addition of 50 μL methanol, the filtered reaction products were subjected to single quadrupole mass spectrometer LCMS-2020 (Shimadzu) or Q Exactive Hybrid Quadrupole-Orbitrap mass spectrometer (Thermo Scientific), which led to the results shown in FIG. 18.

Purification of QS-18 from Quillaia saponaria Bark Extract

A crude saponin extract from Quillaja saponaria bark (Sigma-Aldrich) was dissolved in 100 ml of methanol:water (8:2). The extract was subjected to C-18 reverse phase flash chromatography using a gradient of water [A] and acetonitrile [B] from 0-70% [B], collecting a total of 12×200 mL fractions. Following LC-MS analysis, fraction 7 was found to contain QS-18. This fraction was further purified by reverse phase semi-preparative HPLC using an isocratic mobile phase of 20 mM ammonium bicarbonate, pH 8.6: acetonitrile (65:35) (4 mL/min, 20 min total (Luna C18 column, 250×10 mm (particle size 5 μm) (Phenomenex)). QS-18 was found to elute in fraction 8. This fraction was dried and subjected to extensive 1D-and 2D-NMR analysis, which confirmed the structure of QS-18Api (QA-TriX-FRXA-C18-A-G).

### CLAUSES

Embodiments of the invention are set out in the claims and in the clauses below.
1a. A method of making a biosynthetic QA*-F*-C18-A in a host, which method comprises the steps of
   a) expressing genes required for the biosynthesis of QA*-F* into the host, and
   b) introducing a polynucleotide encoding:
      i. at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64. 62, 60, 58, 56 or 54;
      ii. at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74), ChSF (SEQ ID NO 76) and an enzyme having an amino acid sequence with at least 50% sequence identity to SEQ ID NO 66, 68, 70, 72, 74 or 76;
      iii. at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80) and an enzyme having an amino acid sequence with at least 15% sequence identity to SEQ ID NO 80;
      iv. at least one or more enzymes selected from DMOT9 (SEQ ID NO 82), an enzyme having an amino acid sequence with at least 25% sequence identity to SEQ ID NO 82, DMPT4 (SEQ ID NO 84) and an enzyme having an amino acid sequence with at least 15% sequence identity to SEQ ID NO 84; and
      v. at least one or more enzymes selected from UGT-L-short (SEQ ID NO 86), UGT-L-long (SEQ ID NO 88) and an enzyme having an amino acid sequence with at least 45% sequence identity to SEQ ID NO 86 or 88,
         into the host.
1b. A method of making a biosynthetic QA*-F*-C18-Xyl in a host, which method comprises the steps of
   a) expressing genes required for the biosynthesis of QA*-F* into the host, and
   b) introducing a polynucleotide encoding:
      i. at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64. 62, 60, 58, 56 or 54;
      ii. at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74), ChSF (SEQ ID NO 76) and an enzyme having an amino acid sequence with at least 50% sequence identity to SEQ ID NO 66, 68, 70, 72, 74 or 76;
      iii. at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80) and an enzyme having an amino acid sequence with at least 15% sequence identity to SEQ ID NO 80;
      iv. at least one or more enzymes selected from DMOT9 (SEQ ID NO 82), an enzyme having an amino acid sequence with at least 25% sequence identity to SEQ ID NO 82, DMPT4 (SEQ ID NO 84) and an enzyme having an amino acid sequence with at least 15% sequence identity to SEQ ID NO 84; and
      v. at least one or more enzymes selected from UGT-L-short (SEQ ID NO 86), UGT-L-long (SEQ ID NO 88) and an enzyme having an amino acid sequence with at least 45% sequence identity to SEQ ID NO 86 or 88,
         into the host.
2. The method of clause 1, wherein F* is FRX, FRXX, FRXA or mixtures thereof, preferably wherein F* is FRXA.
3. A method of making a biosynthetic QA*-F*-C18-A-G, wherein the F* chain is at the C-28 position of QA*, the C-18-A chain is attached to the D-fucose of the F* chain and the G residue is attached at the C-3 position of the rhamnose residue of the F* chain, comprising:
   making QA*-F*-C18-A by the method of clause 2, wherein step b) further comprises introducing a polynucleotide encoding
   (vi.) at least one more more enzymes selected from QS-7-GlcT having the amino acid sequence of SEQ ID NO 90 or an enzyme having an amino acid sequence with at least 70% sequence identity to SEQ ID NO 90, to form QA*-F*-C18-A-G, into the host.
4. The method of clause 1, wherein step a) comprises:
   1) expressing genes required for the biosynthesis of QA* into the host, and
   2) introducing a polynucleotide encoding:
      i. Qs-28-O-FucT (SEQ ID NO 2) or an enzyme with an amino acid sequence with at least 70% sequence identity to SEQ ID NO 2, optionally QsFucSyn (SEQ ID NO 12) or an enzyme with an amino acid sequence with at least 45% sequence identity to SEQ ID NO 12;
      ii. optionally Qs-28-O-RhaT (SEQ ID NO 4) or an enzyme with an amino acid sequence with at least 70% sequence identity to SEQ ID NO 4;
      iii. optionally Qs-28-O-XyIT3 (SEQ ID NO 6) or an enzyme with an amino acid sequence with at least 70% sequence identity to SEQ ID NO 6;
      iv. optionally Qs-28-O-XyIT4 (SEQ ID NO 8) or an enzyme with an amino acid sequence with at least 70% sequence identity to SEQ ID NO 8 and/or Qs-28-O-ApiT4 (SEQ ID NO 10) or an enzyme with an amino acid sequence with at least 70% sequence identity to SEQ ID NO 10 into the host.
5. The method of clause 1, wherein step a) comprises:
   1) expressing genes required for the biosynthesis of QA* into the host, and
   2) introducing a polynucleotide encoding:
      i. Qs-28-O-FucT (SEQ ID NO 2) or an enzyme with an amino acid sequence with at least 70% sequence identity optionally QsFucSyn (SEQ ID NO 12) or an enzyme with an amino acid sequence with at least 45% sequence identity;
      ii. optionally Qs-28-O-RhaT (SEQ ID NO 4) or an enzyme with an amino acid sequence with at least 70% sequence identity;
      iii. optionally Qs-28-O-XyIT3 (SEQ ID NO 6) or an enzyme with an amino acid sequence with at least 70% sequence identity;
      iv. optionally Qs-28-O-ApiT4 (SEQ ID NO 10) or an enzyme with an amino acid sequence with at least 70% sequence identity into the host.
6. The method of clause 1, wherein step a) comprises:
   1) expressing genes required for the biosynthesis of QA* into the host, and
   2) introducing a polynucleotide encoding:
      i. Qs-28-O-FucT (SEQ ID NO 2) or an enzyme with an amino acid sequence with at least 70% sequence identity optionally QsFucSyn (SEQ ID NO 12) or an enzyme with an amino acid sequence with at least 45% sequence identity;
      ii. optionally Qs-28-O-RhaT (SEQ ID NO 4) or an enzyme with an amino acid sequence with at least 70% sequence identity;
      iii. optionally Qs-28-O-XyIT3 (SEQ ID NO 6) or an enzyme with an amino acid sequence with at least 70% sequence identity;
      iv. optionally Qs-28-O-XyIT4 (SEQ ID NO 8) or an enzyme with an amino acid sequence with at least 70% sequence identity and/or Qs-28-O-ApiT4 (SEQ ID NO 10) or an enzyme with an amino acid sequence with at least 70% sequence identity into the host.
7. The method of clause 3, wherein step a) comprises:
   1) expressing genes required for the biosynthesis of QA* into the host, and
   2) introducing a polynucleotide encoding:
      i. Qs-28-O-FucT (SEQ ID NO 2) or an enzyme with an amino acid sequence with at least 70% sequence identity to SEQ ID NO 2, optionally QsFucSyn (SEQ ID NO 12) or an enzyme with an amino acid sequence with at least 45% sequence identity to SEQ ID NO 12;
      ii. Qs-28-O-RhaT (SEQ ID NO 4) or an enzyme with an amino acid sequence with at least 70% sequence identity to SEQ ID NO 4;
      iii. Qs-28-O-XyIT3 (SEQ ID NO 6) or an enzyme with an amino acid sequence with at least 70% sequence identity to SEQ ID NO 6;
      iv. optionally Qs-28-O-XyIT4 (SEQ ID NO 8) or an enzyme with an amino acid sequence with at least 70% sequence identity to SEQ ID NO 8 and/or Qs-28-O-ApiT4 (SEQ ID NO 10) or an enzyme with an amino acid sequence with at least 70% sequence identity to SEQ ID NO 10 into the host.
8. The method of any one of clauses 4 to 7, wherein step 1) comprises introducing a polynucleotide encoding:
   i. QsCSL1 (SEQ ID NO 26) or QsCsIG2 (SEQ ID NO 28), or an enzyme with an amino acid sequence with at least 70% sequence identity to SEQ ID NO 26 or 28;
   ii. optionally Qs-3-O-GaIT (SEQ ID NO 30) or an enzyme with an amino acid sequence with at least 70% sequence identity; and/or
   iii. optionally DN20529_c0_g2_i8 (SEQ ID NO 36), Qs_0283850 (SEQ ID NO 34), or Qs-3-O-RhaT/XyIT (SEQ ID NO 32) or an enzyme with an amino acid sequence with at least 70% sequence identity to SEQ ID No 36, 34 or 32, and/or Qs_0283870 (SEQ ID NO 38) or Qs-3-O-RhaT/XyIT (SEQ ID NO 32) or an enzyme with an amino acid sequence with at least 70% sequence identity to SEQ ID NO 38 or 32; into the host.
9. The method of any one of clauses 4 to 7, wherein step 1) comprises introducing a polynucleotide encoding:
   i. QsCSL1 (SEQ ID NO 26) or QsCsIG2 (SEQ ID NO 28), or an enzyme with an amino acid sequence with at least 70% sequence identity to SEQ ID NO 26 or 28;
   ii. optionally Qs-3-O-GaIT (SEQ ID NO 30) or an enzyme with an amino acid sequence with at least 70% sequence identity; and/or
   iii. optionally DN20529_c0_g2_i8 (SEQ ID NO 36), Qs_0283850 (SEQ ID NO 34), or Qs-3-O-RhaT/XyIT (SEQ ID NO 32) or an enzyme with an amino acid sequence with at least 70% sequence identity to SEQ ID No 36, 34 or 32 into the host.
10. The method of any one of clauses 4 to 7, wherein step 1) further comprises introducing a polynucleotide encoding:
   i. QsCSL1 (SEQ ID NO 26) or QsCsIG2 (SEQ ID NO 28), or an enzyme with an amino acid sequence with at least 70% sequence identity to SEQ ID NO 26 or 28;
   ii. optionally Qs-3-O-GaIT (SEQ ID NO 30) or an enzyme with an amino acid sequence with at least 70% sequence identity; and/or
   iii. optionally Qs_0283870 (SEQ ID NO 38) or Qs-3-O-RhaT/XyIT (SEQ ID NO 32) or an enzyme with an amino acid sequence with at least 70% sequence identity to SEQ ID NO 38 or 32 into the host.
11. The method of any one of clauses 4 to 10, wherein step a)-1) further comprises introducing a polynucleotide encoding:
   i. QsbAS (SEQ ID NO 18) or an enzyme with an amino acid sequence with at least 50% sequence identity to SEQ ID NO 18;
   ii. QsCYP716-C-28 (SEQ ID NO 20), or an enzyme with an amino acid sequence with at least 50% sequence identity to SEQ ID NO 20;
   iii. QsCYP716-C-16a (SEQ ID NO 22), or an enzyme with an amino acid sequence with at least 50% sequence identity to SEQ ID NO 22; and iv. QsCYP714-C-23 (SEQ ID NO 24), or an enzyme with an amino acid sequence with at least 50% sequence identity to SEQ ID NO 24; into the host.
12. The method of any one of clauses 4 to 11, wherein
   amino acid SEQ ID NO 2 is encoded by polynucleotide SEQ ID NO 1;
   amino acid SEQ ID NO 4 is encoded by polynucleotide SEQ ID NO 3;
   amino acid SEQ ID NO 6 is encoded by polynucleotide SEQ ID NO 5;
   amino acid SEQ ID NO 8 is encoded by polynucleotide SEQ ID NO 7;
   amino acid SEQ ID NO 10 is encoded by polynucleotide SEQ ID NO 9.
13. The method of any one of clauses 7 to 12, wherein:
   amino acid SEQ ID NO 26 is encoded by polynucleotide SEQ ID NO 25;
   amino acid SEQ ID NO 28 is encoded by polynucleotide SEQ ID NO 27;
   amino acid SEQ ID NO 30 is encoded by polynucleotide SEQ ID NO 29;
   amino acid SEQ ID NO 32 is encoded by polynucleotide SEQ ID NO 31;
   amino acid SEQ ID NO 34 is encoded by polynucleotide SEQ ID NO 33;
   amino acid SEQ ID NO 36 is encoded by polynucleotide SEQ ID NO 35;
   amino acid SEQ ID NO 38 is encoded by polynucleotide SEQ ID NO 37.
14a. A method of making QA*-F*-C18-A, wherein the F* chain is at the C-28 position of QA*, and the C-18-A chain is attached to the D-fucose of the F* chain, the method comprising treating a molecule comprising QA*-F* with a mixture of enzymes comprising:
   (i) at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64, 62, 60, 58, 56 or 54;
   (ii) at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74) ChSF (SEQ ID NO 76) and an enzyme having an amino acid sequence with at least 50% sequence identity to SEQ ID NO 66, 68, 70, 72, 74 or 76;
   (iii) at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80) and an enzyme having an amino acid sequence with at least 15% sequence identity to SEQ ID NO 80;
   (iv) at least DMOT9 (SEQ ID NO 82) or an enzyme having an amino acid sequence with at least 25% sequence identity to SEQ ID NO 82;
   (v) at least DMOT4 (SEQ ID NO 84) or an enzyme having an amino acid sequence with at least 15% sequence identity to SEQ ID NO 84; and
   (vi) at least one or more enzymes selected from UGT-L-short (SEQ ID NO 86), UGT-L-long (SEQ ID NO 88), and an enzyme having an amino acid sequence with at least 45% sequence identity to SEQ ID NO 86 or 88, in the presence of 2-methylbutyric acid, malonyl-CoA and UDP-β-L-arabinofuranose.
14b. A method of making QA*-F*-C18-Xyl, wherein the F* chain is at the C-28 position of QA*, and the C-18-Xyl chain is attached to the D-fucose of the F* chain, the method comprising treating a molecule comprising QA*-F* with a mixture of enzymes comprising:
   (i) at least one or more enzymes selected from 6CCL (SEQ ID NO 64), 5CCL (SEQ ID NO 62), 4CCL (SEQ ID NO 60), 3CCL (SEQ ID NO 58), 2CCL (SEQ ID NO 56), 1CCL (SEQ ID NO 54) and an enzyme having an amino acid sequence with at least 60% sequence identity to SEQ ID NO 64, 62, 60, 58, 56 or 54;
   (ii) at least one or more enzymes selected from ChSA (SEQ ID NO 66), ChSB (SEQ ID NO 68), ChSC (SEQ ID NO 70), ChSD (SEQ ID NO 72), ChSE (SEQ ID NO 74) ChSF (SEQ ID NO 76) and an enzyme having an amino acid sequence with at least 50% sequence identity to SEQ ID NO 66, 68, 70, 72, 74 or 76;
   (iii) at least one or more enzymes selected from KR11 (SEQ ID NO 78) and an enzyme having an amino acid sequence with at least 20% sequence identity to SEQ ID NO 78, optionally in combination with an enzyme selected from KR23′ (SEQ ID NO 80) and an enzyme having an amino acid sequence with at least 15% sequence identity to SEQ ID NO 80;
   (iv) at least DMOT9 (SEQ ID NO 82) or an enzyme having an amino acid sequence with at least 25% sequence identity to SEQ ID NO 82;
   (v) at least DMOT4 (SEQ ID NO 84) or an enzyme having an amino acid sequence with at least 15% sequence identity to SEQ ID NO 84; and
   (vi) at least one or more enzymes selected from UGT-L-short (SEQ ID NO 86), UGT-L-long (SEQ ID NO 88), and an enzyme having an amino acid sequence with at least 45% sequence identity to SEQ ID NO 86 or 88, in the presence of 2-methylbutyric acid, malonyl-CoA and UDP-Xyl.
15. The method of clause 14, wherein F* is FRX, FRXX, FRXA or mixtures thereof, preferably wherein F* is FRXA.
16. A method of making QA*-F*-C18-A-G, wherein the F* chain is at the C-28 position of QA*, the C-18-A chain is attached to the D-fucose of the F* chain and the G residue is attached at the C-3 position of the rhamnose residue of the F* chain, comprising: making QA*-F*-C18-A by the method of clause 15, and combining QA*-F*-C18-A with an enzyme capable of transferring a D-glucose residue to QA*-F*-C18-A to form QA*-F*-C18-A-G.
17. The method of clause 14, wherein the mixture of enzymes further comprises:
   i. Qs-28-O-FucT (SEQ ID NO 2) or an enzyme with an amino acid sequence with at least 70% sequence identity, optionally QsFucSyn (SEQ ID NO 12) or an enzyme with an amino acid sequence with at least 45% sequence identity to SEQ ID NO 12;
   ii. optionally Qs-28-O-RhaT (SEQ ID NO 4) or an enzyme with an amino acid sequence with at least 70% sequence identity to SEQ ID NO 4;
   iii. optionally Qs-28-O-XyIT3 (SEQ ID NO 6) or an enzyme with an amino acid sequence with at least 70% sequence identity toSEQ ID NO 6;
   iv. optionally Qs-28-O-XyIT4 (SEQ ID NO 8) or an enzyme with an amino acid sequence with at least 70% sequence identity to SEQ ID NO 8 and/or Qs-28-O-ApiT4 (SEQ ID NO 10) or an enzyme with an amino acid sequence with at least 70% sequence identity to SEQ ID NO 10.
18. The method of clause 15 or 16, wherein the mixture of enzymes further comprises:
   i. Qs-28-O-FucT (SEQ ID NO 2) or an enzyme with an amino acid sequence with at least 70% sequence identity, optionally QsFucSyn (SEQ ID NO 12) or an enzyme with an amino acid sequence with at least 45% sequence identity to SEQ ID NO 12;
   ii. Qs-28-O-RhaT (SEQ ID NO 4) or an enzyme with an amino acid sequence with at least 70% sequence identity to SEQ ID NO 4;
   iii. Qs-28-O-XyIT3 (SEQ ID NO 6) or an enzyme with an amino acid sequence with at least 70% sequence identity toSEQ ID NO 6;
   iv. optionally Qs-28-O-XyIT4 (SEQ ID NO 8) or an enzyme with an amino acid sequence with at least 70% sequence identity to SEQ ID NO 8 and/or Qs-28-O-ApiT4 (SEQ ID NO 10) or an enzyme with an amino acid sequence with at least 70% sequence identity to SEQ ID NO 10.
19. The method of clause 14, wherein the mixture of enzymes further comprises:
   i. Qs-28-O-FucT (SEQ ID NO 2) or an enzyme with an amino acid sequence with at least 70% sequence identity to SEQ ID NO 2, optionally QsFucSyn (SEQ ID NO 12) or an enzyme with an amino acid sequence with at least 45% sequence identity to SEQ ID NO 12;
   ii. optionally Qs-28-O-RhaT (SEQ ID NO 4) or an enzyme with an amino acid sequence with at least 70% sequence identity to SEQ ID NO 4;
   iii. optionally Qs-28-O-XyIT3 (SEQ ID NO 6) or an enzyme with an amino acid sequence with at least 70% sequence identity to SEQ ID NO 6;
   iv. optionally Qs-28-O-ApiT4 (SEQ ID NO 10) or an enzyme with an amino acid sequence with at least 70% sequence identity to SEQ ID NO 10.
20. The method of clause 14, wherein the mixture of enzymes further comprises:
   i. Qs-28-O-FucT (SEQ ID NO 2) or an enzyme with an amino acid sequence with at least 70% sequence identity to SEQ ID NO 2, optionally QsFucSyn (SEQ ID NO 12) or an enzyme with an amino acid sequence with at least 45% sequence identity to SEQ ID NO 12;
   ii. optionally Qs-28-O-RhaT (SEQ ID NO 4) or an enzyme with an amino acid sequence with at least 70% sequence identity to SEQ ID NO 4;
   iii. optionally Qs-28-O-XyIT3 (SEQ ID NO 6) or an enzyme with an amino acid sequence with at least 70% sequence identity to SEQ ID NO 6;
   iv. optionally Qs-28-O-XyIT4 (SEQ ID NO 8) or an enzyme with an amino acid sequence with at least 70% sequence identity to SEQ ID NO 8.
21. The method of any one of clauses 14 to 20, wherein the mixture of enzymes further comprises:
   i. QsCSL1 (SEQ ID NO 26) or QsCsIG2 (SEQ ID NO 28), or an enzyme with an amino acid sequence with at least 70% sequence identity to SEQ ID NO 26 or 28;
   ii. optionally Qs-3-O-GaIT (SEQ ID NO 30) or an enzyme with an amino acid sequence with at least 70% sequence identity to SEQ ID NO 30; and/or
   iii. optionally DN20529_c0_g2_i8 (SEQ ID NO 36), Qs_0283850 (SEQ ID NO 34), or Qs-3-O-RhaT/XyIT (SEQ ID NO 32) or an enzyme with an amino acid sequence with at least 70% sequence identity to SEQ ID No 36, 34 or 32, and/or Qs_0283870 (SEQ ID NO 38) or Qs-3-O-RhaT/XyIT (SEQ ID NO 32) or an enzyme with an amino acid sequence with at least 70% sequence identity to SEQ ID NO 38 or 32,
22. The method of any one of clauses 14 to 20, wherein the mixture of enzymes further comprises:
   i. QsCSL1 (SEQ ID NO 26) or QsCsIG2 (SEQ ID NO 28), or an enzyme with an amino acid sequence with at least 70% sequence identity to SEQ ID NO 26 or 28;
   ii. optionally Qs-3-O-GaIT (SEQ ID NO 30) or an enzyme with an amino acid sequence with at least 70% sequence identity to SEQ ID NO 30; and/or
   iii. optionally DN20529_c0_g2_i8 (SEQ ID NO 36), Qs_0283850 (SEQ ID NO 34), or Qs-3-O-RhaT/XyIT (SEQ ID NO 32) or an enzyme with an amino acid sequence with at least 70% sequence identity to SEQ ID No 36, 34 or 32.
23. The method of any one of clauses 14 to 20, wherein the mixture of enzymes further comprises:
   i. QsCSL1 (SEQ ID NO 26) or QsCsIG2 (SEQ ID NO 28), or an enzyme with an amino acid sequence with at least 70% sequence identity to SEQ ID NO 26 or 28;
   ii. optionally Qs-3-O-GaIT (SEQ ID NO 30) or an enzyme with an amino acid sequence with at least 70% sequence identity to SEQ ID NO 30; and/or
   iii. optionally Qs_0283870 (SEQ ID NO 38) or Qs-3-O-RhaT/XylT (SEQ ID NO 32) or an enzyme with an amino acid sequence with at least 70% sequence identity to SEQ ID NO 38 or 32.
24. The method of any one of clauses 14 to 23, wherein the mixture of enzymes further comprises:
   QsbAS (SEQ ID NO 18) an enzyme with an amino acid sequence with at least 50% sequence identity to SEQ ID NO 18;
   QsCYP716-C-28 (SEQ ID NO 20) an enzyme with an amino acid sequence with at least 50% sequence identity to SEQ ID NO 20;
   QsCYP716-C-16a (SEQ ID NO 22) an enzyme with an amino acid sequence with at least 50% sequence identity to SEQ ID N022, and
   QsCYP714-C-23 (SEQ ID NO 24) an enzyme with an amino acid sequence with at least 50% sequence identity to SEQ ID NO 24.
25. The method of any preceding clause, wherein the method further comprises the step of isolating QA*-F*-C18-A.
26. The method of clause 25, wherein QA*-F*-C18-A is QA-F-C18-A, QA-Mono-F-C18-A, QA-Di-F-C18-A, QA-TriX-F-C18-A, QA-TriR-F-C18-A, QA-FR-C18-A, QA-Mono-FR-C18-A, QA-Di-FR-C18-A, QA-TriX-FR-C18-A, QA-TriR-FR-C18-A, QA-FRX-C18-A, QA-Mono-FRX-C18-A, QA-Di-FRX-C18-A, QA-TriX-FRX-C18-A, QA-TriR-FRX-C18-A, QA-FRXX-C18-A, QA-Mono-FRXX-C18-A, QA-Di-FRXX-C18-A, QA-TriX-FRXX-C18-A, QA-TriR-FRXX-C18-A, QA-FRXA-C18-A, QA-Mono-FRXA-C18-A, QA-Di-FRXA-C18-A, QA-TriX-FRXA-C18-A and/or QA-TriR-FRXA-C18-A.
27. The method of clause 26, wherein QA*-F*-C18-A is QA-TriX-FRXX-C18-A, QA-TriX-FRXA-C18-A or mixtures thereof.
28. The method of any one of clauses 3, 7 to 13, 16, 18, and 21 to 24, wherein the method further comprises the step of isolating QA*-F*-C18-A-G.
29. The method of clause 28, wherein QA*-F*-C18-A-G is QA-FRX-C18-A-G, QA-Mono-FRX-C18-A-G, QA-Di-FRX-C18-A-G, QA-TriX-FRX-C18-A-G, QA-TriR-FRX-C18-A-G, QA-FRXX-C18-A-G, QA-Mono-FRXX-C18-A-G, QA-Di-FRXX-C18-A-G, QA-TriX-FRXX-C18-A-G, QA-TriR-FRXX-C18-A-G, QA-FRXA-C18-A-G, QA-Mono-FRXA-C18-A-G, QA-Di-FRXA-C18-A-G, QA-TriX-FRXA-C18-A-G and/or QA-TriR-FRXA-C18-A-G.
30. The method of clause 29, wherein QA*-F*-C18-A-G is QA-TriX-FRXX-C18-A-G, QA-TriX-FRXA-C18-A-G or mixtures thereof.
31. The QA*-F*-C18-A derivative obtainable by the method of clause 25.
32 The QA*-F*-C18-A-G derivative obtainable by the method of clause 28.
33. The QA*-F*-C18-A of clause 31 wherein QA*-F*-C18-A is QA-F-C18-A, QA-Mono-F-C18-A, QA-Di-F-C18-A, QA-TriX-F-C18-A, QA-TriR-F-C18-A, QA-FR-C18-A, QA-Mono-FR-C18-A, QA-Di-FR-C18-A, QA-TriX-FR-C18-A, QA-TriR-FR-C18-A, QA-FRX-C18-A, QA-Mono-FRX-C18-A, QA-Di-FRX-C18-A, QA-TriX-FRX-C18-A, QA-TriR-FRX-C18-A, QA-FRXX-C18-A, QA-Mono-FRXX-C18-A, QA-Di-FRXX-C18-A, QA-TriX-FRXX-C18-A, QA-TriR-FRXX-C18-A, QA-FRXA-C18-A, QA-Mono-FRXA-C18-A, QA-Di-FRXA-C18-A, QA-TriX-FRXA-C18-A and/or QA-TriR-FRXA-C18-A.
34. The QA*-F*-C18-A-G of clause 32 wherein QA*-F*-C18-A-G is QA-FRX-C18-A-G, QA-Mono-FRX-C18-A-G, QA-Di-FRX-C18-A-G, QA-TriX-FRX-C18-A-G, QA-TriR-FRX-C18-A-G, QA-FRXX-C18-A-G, QA-Mono-FRXX-C18-A-G, QA-Di-FRXX-C18-A-G, QA-TriX-FRXX-C18-A-G, QA-TriR-FRXX-C18-A-G, QA-FRXA-C18-A-G, QA-Mono-FRXA-C18-A-G, QA-Di-FRXA-C18-A-G, QA-TriX-FRXA-C18-A-G and/or QA-TriR-FRXA-C18-A-G.
35. The QA*-F*-C18-A of clause 33 wherein QA*-F*-C18-A is QA-TriX-FRXX-C18-A, QA-TriX-FRXA-C18-A or mixtures thereof.
36. The QA*-F*-C18-A-G of clause 34, wherein QA*-F*-C18-A-G is QA-TriX-FRXX-C18-A-G, QA-TriX-FRXA-C18-A-G or mixtures thereof.

### Abbreviations

ArafT—Arabinofuranosyl transferase
AstHMGR—Avena strigosa truncated 3-hydroxy-3-methyl-glutyryl-CoA reductase
C9 acyl unit—(3S,5S,6S)-3,5-dihydroxy-6-methyloctanoyl-CoA or (3S,5S,6S)-3,5-dihydroxy-6-methyloctanoic acid
C18 acyl chain—(3S,5S,6S)-5-((3S,5S,6S)-3,5-dihydroxy-6-methyloctanoyloxy)-3-hydroxy-6-methyloctanoic acid
1CCL—Carboxyl CoA ligase 1
2CCL—Carboxyl CoA ligase 2
3CCL—Carboxyl CoA ligase 3
4CCL—Carboxyl CoA ligase 4
5CCL—Carboxyl CoA ligase 5
6CCL—Carboxyl CoA ligase 6
ChS—Q. saponaria chalcone synthase-like enzyme
ChSA—Chalcone synthase-like A
ChSB—Chalcone synthase-like B
ChSC—Chalcone synthase-like C
ChSD—Chalcone synthase-like D
ChSE—Chalcone synthase-like E
ChSF—Chalcone synthase-like F
CYP—Cytochrome P450
DMOT9—(3S,5S,6S)-3,5-dihydroxy-6-methyloctanoyl-CoA transferase 9
DMOT4—(3S,5S,6S)-3,5-dihydroxy-6-methyloctanoyl-CoA transferase 4
DN20529_c0_g2_i8—Q. saponaria QA-Di α-1,3-L-rhamnosyltransferase
Fucp—D-Fucopyranose
FucSyn—Enzyme boosting the production of fucosylated saponins
FSL—QsFucSyn-Like
Galp—D-Galactopyranose
GIcpA—D-Glucopyranuronic acid
HICCL2—Humulus lupulus CCL2—NCBI accession number JQ740204.1
HICCL4—Humulus lupulus CCL4—NCBI accession number JQ740206.1
HIVPS—Humulus lupulus valerophenone synthase—NCBI accession number AB015430.1
KR11—Keto reductase 11
KR23′—Keto reductase 23′
OSC—Oxidosqualene cyclase
QA—Quillaic acid
QA*:
   QA-Di—3-O-{β-D-galactopyranosyl-(1->2)-β-D-glucopyranosiduronic acid}-quillaic acid
   QA-Mono—3-O-{β-D-glucopyranosiduronic acid}-quillaic acid
   QA-TriR—3-O-{α-L-rhamnopyranosyl-(1->3)-[β-D-galactopyranosyl-(1->2)]-β-D-glucopyranosiduronic acid}-quillaic acid
   QA-TriX—3-O-{β-D-xylopyranosyl-(1->3)-[β-D-galactopyranosyl-(1->2)]-β-D-glucopyranosiduronic acid}-quillaic acid
   QA-Tri(X/R)—QA glycosylated at C-3 position with a branched trisaccharide which is either QA-TriX or QA-TriR
QA*-F* is as listed below, and encompasses mixtures thereof:
   QA-F—28-O-{β-D-fucopyranosyl ester}-quillaic acid
   QA-Mono-F—3-O-{β-D-glucopyranosiduronic acid}-28-O-{β-D-fucopyranosyl ester}-quillaic acid
   QA-Di-F—3-O-{β-D-galactopyranosyl-(1->2)-β-D-glucopyranosiduronic acid}-28-O-{β-D-fucopyranosyl ester}-quillaic acid
   QA-TriX-F—3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid
   QA-TriR-F—3-O-{α-L-rhamnopyranosyl-(1->3)-[β-D-galactopyranosyl-(1->2)]-β-D-glucopyranosiduronic acid}-28-O-{β-D-fucopyranosyl ester}-quillaic acid
   QA-FR—28-O-{α-L-rhamnopyranosyl-(1->2)-β-D-fucopyranosyl ester}-quillaic acid
   QA-Mono-FR—3-O-{β-D-glucopyranosiduronic acid}-28-O-{α-L-rhamnopyranosyl-(1->2)-β-D-fucopyranosyl ester}-quillaic acid
   QA-Di-FR—3-O-{β-D-galactopyranosyl-(1->2)-β-D-glucopyranosiduronic acid}-28-O-{α-L-rhamnopyranosyl-(1->2)-β-D-fucopyranosyl ester}-quillaic acid,
   QA-TriX-FR—3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid,
   QA-TriR-FR—3-O-{α-L-rhamnopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid,
   QA-FRX—28-O-{β-D-xylopyranosyl-(1->4)-α-L-rhamnopyranosyl-(1->2)-β-D-fucopyranosyl ester}-quillaic acid
   QA-Mono-FRX—3-O-{β-D-glucopyranosiduronic acid}-28-O-{β-D-xylopyranosyl-(1->4)-α-L-rhamnopyranosyl-(1->2)-β-D-fucopyranosyl ester}-quillaic acid
   QA-Di-FRX—3-O-{β-D-galactopyranosyl-(1->2)-β-D-glucopyranosiduronic acid}-28-O-{β-D-xylopyranosyl-(1->4)-α-L-rhamnopyranosyl-(1->2)-β-D-fucopyranosyl ester}-quillaic acid
   QA-TriX-FRX—3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid,
   QA-TriR-FRX—3-O-{α-L-rhamnopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid,
   QA-FRXX—28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-Mono-FRXX—3-O-{β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-Di-FRXX—3-O-{β-D-galactopyranosyl-(1->2)-β-D-glucopyranosiduronic acid}-28-0-{β-D-xylopyranosyl-(1->3)-β-D-xylopyranosyl-(1->4)-α-L-rhamnopyranosyl-(1->2)-β-D-fucopyranosyl ester}-quillaic acid
   QA-TriX-FRXX—3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid,
   QA-TriR-FRXX—3-O-{α-L-rhamnopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid,
   QA-FRXA—28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-Mono-FRXA—3-O-{β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid,
   QA-Di-FRXA—3-O-{β-D-galactopyranosyl-(1->2)-β-D-glucopyranosiduronic acid}-28-0-{β-D-apiofuranosyl-(1->3)-β-D-xylopyranosyl-(1->4)-α-L-rhamnopyranosyl-(1->2)-β-D-fucopyranosyl ester}-quillaic acid
   QA-TriX-FRXA—3-O-{β-D-xylopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid,
   QA-TriR-FRXA—3-O-{α-L-rhamnopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
QA*-F*-C18-A:
   -QA-F-C18-A—(2R,3S,4S,5S,6S)-5-(((3S,6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3,4-dihydroxy-6-methyltetrahydro-2H-pyran-2-yl (4aR,5R,6aS,6bR,9S,10S,12aR,14bS)-9-formyl-5,10-dihydroxy-2,2,6a,6b,9,12a-hexamethyl-1,3,4,5,6,6a,6b,7,8,8a,9,10,11,12,12a,12b,13,14b-octadecahydropicene-4a(2H)-carboxylate
   -QA-Mono-F-C18-A—(2S,3S,4S,5R)-6-(((3S,4S,6aR,6bS,8R,8aR,12aS,14bR)-8a-((((2R,3S,4S,5S,6S)-5-(((3S,6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3,4-dihydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-F-C18-A—(2S,3S,4S,5R)-6-(((3S,4S,6aR,6bS,8R,8aR,12aS,14bR)-8a-((((2R,3S,4S,5S,6S)-5-(((3S,6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3,4-dihydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-F-C18-A—(2S,3S,4S,5R)-6-(((3S,4S,6aR,6bS,8R,8aR,12aS,14bR)-8a-((((2R,3S,4S,5S,6S)-5-(((3S,6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3,4-dihydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2R,3R,4S,5R)-3,4,5-trihydroxytetrahydro-2H-pyran-2-yl)oxy)tetrahydro-2H-pyran-2-carboxylic acid
   -QA-TriR-F-C18-A—(2S,3S,4S,5R,6R)-6-(((3S,4S,6aR,6bS,8R,8aR,12aS,14bR)-8a-((((2S,3R,4R,5R,6R)-5-(((3R,6R)-5-(((3R,6R)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3,4-dihydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
   -QA-FR-C18-A—(2R,3S,4R,5S,6S)-5-(((6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyl-3-(((2S,3R,4R,5R,6S)-3,4,5-trihydroxy-6-methyltetrahydro-2H-pyran-2-yl)oxy)tetrahydro-2H-pyran-2-yl (4aR,5R,6aS,6bR,9S,10S,12aR,14bS)-9-formyl-5,10-dihydroxy-2,2,6a,6b,9,12a-hexamethyl-1,3,4,5,6,6a,6b,7,8,8a,9,10,11,12,12a,12b,13,14b-octadecahydropicene-4a(2H)-carboxylate
   -QA-Mono-FR-C18-A—(2S,3S,4S,5R)-6-(((3S,4S,6aR,6bS,8R,8aR,12aS,14bR)-8a-((((2R,3S,4R,5S,6S)-5-(((6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FR-C18-A—(2S,3S,4S,5R)-6-(((3S,4S,6aR,6bS,8R,8aR,12aS,14bR)-8a-((((2R,3S,4R,5S,6S)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FR-C18-A—(2S,3S,4S,5R)-6-(((3S,4S,6aR,6bS,8R,8aR,12aS,14bR)-8a-((((2R,3S,4R,5S,6S)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyl-3-(((2 S,3R,4R,5R,6S)-3,4,5-trihydroxy-6-methyltetrahydro-2H-pyran-2-yl)oxy)tetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2 S,3R,4S,5R,6R)-3,4,5-trihydroxy-6-(hydroxymethyl)tetrahydro-2H-pyran-2-yl)oxy)-4-(((2R,3R,4S,5R)-3,4,5-trihydroxytetrahydro-2H-pyran-2-yl)oxy)tetrahydro-2H-pyran-2-carboxylic acid
   -QA-TriR-FR-C18-A—(2S,3S,4S,5R,6R)-6-(((3S,4S,6aR,6bS,8R,8aR,12aS,14bR)-8a-((((2 S,3R,4S,5R,6R)-5-(((3R,6R)-5-(((3R,6R)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyl-3-(((2 S,3R,5R,6S)-3,4,5-trihydroxy-6-methyltetrahydro-2H-pyran-2-yl)oxy)tetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2 S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
   -QA-FRX-C18-A—(2R,3S,4R,5S,6S)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3-(((2 S,3R,4S,5R,6S)-3,4-dihydroxy-6-methyl-5-(((2 S,3R,4S,5R)-3,4,5-trihydroxytetrahydro-2H-pyran-2-yl)oxy)tetrahydro-2H-pyran-2-yl)oxy)-4-hydroxy-6-methyltetrahydro-2H-pyran-2-yl (4aR,5R,6aS,6bR,9S,10S,12aR,14bS)-9-formyl-5,10-dihydroxy-2,2,6a,6b,9,12a-hexamethyl-1,3,4,5,6,6a,6b,7,8,8a,9,10,11,12,12a,12b,13,14b-octadecahydropicene-4a(2H)-carboxylate
   -QA-Mono-FRX-C18-A—(2S,3S,4S,5R)-6-(((3S,4S,6aR,6bS,8R,8aR,12aS,14bR)-8a-((((2R,3S,4R,5S,6S)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3-(((2S,3R,4S,5R,6S)-3,4-dihydroxy-6-methyl-5-(((2S,3R,4S,5R)-3,4,5-trihydroxytetrahydro-2H-pyran-2-yl)oxy)tetrahydro-2H-pyran-2-yl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FRX-C18-A—(2S,3S,4S,5R)-6-(((3S,4S,6aR,6bS,8R,8aR,12aS,14bR)-8a-((((2R,3S,4R,5S,6S)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3-(((2S,3R,4S,5R,6S)-3,4-dihydroxy-6-methyl-5-(((2S,3R,4S,5R)-3,4,5-trihydroxytetrahydro-2H-pyran-2-yl)oxy)tetrahydro-2H-pyran-2-yl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FRX-C18-A—(2S,3S,4S,5R)-6-(((3S,4S,6aR,6bS,8R,8aR,12aS,14bR)-8a-((((2R,3S,4R,5S,6S)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3-(((2S,3R,4S,5R,6S)-3,4-dihydroxy-6-methyl-5-(((2S,3R,4S,5R)-3,4,5-trihydroxytetrahydro-2H-pyran-2-yl)oxy)tetrahydro-2H-pyran-2-yl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2R,3R,4S,5R)-3,4,5-trihydroxytetrahydro-2H-pyran-2-yl)oxy)tetrahydro-2H-pyran-2-carboxylic acid
   -QA-TriR-FRX-C18-A—(2S,3S,4S,5R,6R)-6-(((3S,4S,6aR,6bS,8R,8aR,12aS,14bR)-8a-((((2S,3R,4S,5R,6R)-5-(((3R,6R)-5-(((3R,6R)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3-(((2S,3R,5R,6S)-3,4-dihydroxy-6-methyl-5-(((2S,4S,5R)-3,4,5-trihydroxytetrahydro-2H-pyran-2-yl)oxy)tetrahydro-2H-pyran-2-yl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
   -QA-FRXX-C18-A—(2S,3R,4S,5R,6R)-3-(((2S,3R,5R,6S)-5-(((2S,4S,5R)-3,5-dihydroxy-4-(((2S,4S,5R)-3,4,5-trihydroxytetrahydro-2H-pyran-2-yl)oxy)tetrahydro-2H-pyran-2-yl)oxy)-3,4-dihydroxy-6-methyltetrahydro-2H-pyran-2-yl)oxy)-5-(((3R,6R)-5-(((3R,6R)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl (4aR,5R,6aS,6bR,9S,10S,12aR,14bS)-9-formyl-5,10-dihydroxy-2,2,6a,6b,9,12a,14b-heptamethyl-1,3,4,5,6,6a,6b,7,8,8a,9,10,11,12,12a,12b,13,14b-octadecahydropicene-4a(2H)-carboxylate
   -QA-Mono-FRXX-C18-A—(2S,3S,4S,5R,6R)-6-(((3S,4S,6aR,6bS,8R,8aR,12aS,14bR)-8a-((((2S,3R,4S,5R,6R)-3-(((2S,3R,5R,6S)-5-(((2S,4S,5R)-3,5-dihydroxy-4-(((2S,4S,5R)-3,4,5-trihydroxytetrahydro-2H-pyran-2-yl)oxy)tetrahydro-2H-pyran-2-yl)oxy)-3,4-dihydroxy-6-methyltetrahydro-2H-pyran-2-yl)oxy)-5-(((3R,6R)-5-(((3R,6R)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FRXX-C18-A—(2S,3S,4S,5R,6R)-6-(((3S,4S,6aR,6bS,8R,8aR,12aS,14bR)-8a-((((2S,3R,4S,5R,6R)-3-(((2S,3R,5R,6S)-5-(((2S,4S,5R)-3,5-dihydroxy-4-(((2S,4S,5R)-3,4,5-trihydroxytetrahydro-2H-pyran-2-yl)oxy)tetrahydro-2H-pyran-2-yl)oxy)-3,4-dihydroxy-6-methyltetrahydro-2H-pyran-2-yl)oxy)-5-(((3R,6R)-5-(((3R,6R)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FRXX-C18-A—(2S,3S,4S,5R,6R)-6-(((3S,4S,6aR,6bS,8R,8aR,12aS,14bR)-8a-((((2S,3R,4S,5R,6R)-3-(((2S,3R,5R,6S)-5-(((2S,4S,5R)-3,5-dihydroxy-4-(((2S,4S,5R)-3,4,5-trihydroxytetrahydro-2H-pyran-2-yl)oxy)tetrahydro-2H-pyran-2-yl)oxy)-3,4-dihydroxy-6-methyltetrahydro-2H-pyran-2-yl)oxy)-5-(((3R,6R)-5-(((3R,6R)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4S,5R)-3,4,5-trihydroxytetrahydro-2H-pyran-2-yl)oxy)tetrahydro-2H-pyran-2-carboxylic acid
   -QA-TriR-FRX-C18-A—(2S,3S,4S,5R,6R)-6-(((3S,4S,6aR,6bS,8R,8aR,12aS,14bR)-8a-((((2S,3R,4S,5R,6R)-5-(((3R,6R)-5-(((3R,6R)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-3-(((2S,3R,5R,6S)-3,4-dihydroxy-6-methyl-5-(((2S,4S,5R)-3,4,5-trihydroxytetrahydro-2H-pyran-2-yl)oxy)tetrahydro-2H-pyran-2-yl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
   -QA-FRXA-C18-A—(2R,3S,4R,5S,6S)-3-(((2S,3R,4S,5R,6S)-5-(((2S,3R,4S,5R)-3,5-dihydroxy-4-(((2S,3R,4R)-4-hydroxy-4-(hydroxymethyl)-3-methyltetrahydrofuran-2-yl)oxy)tetrahydro-2H-pyran-2-yl)oxy)-3,4-dihydroxy-6-methyltetrahydro-2H-pyran-2-yl)oxy)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl (4aR,5R,6aS,6bR,9S,10S,12aR,14bS)-9-formyl-5,10-dihydroxy-2,2,6a,6b,9,12a-hexamethyl-1,3,4,5,6,6a,6b,7,8,8a,9,10,11,12,12a,12b,13,14b-octadecahydropicene-4a(2H)-carboxylate
   -QA-Mono-FRXA-C18-A—(2S,3S,4S,5R)-6-(((3S,4S,6aR,6bS,8R,8aR,12aS,14bR)-8a-((((2R,3S,4R,5S,6S)-3-(((2S,3R,4S,5R,6S)-5-(((2S,3R,4S,5R)-3,5-dihydroxy-4-(((2S,3R,4R)-4-hydroxy-4-(hydroxymethyl)-3-methyltetrahydrofuran-2-yl)oxy)tetrahydro-2H-pyran-2-yl)oxy)-3,4-dihydroxy-6-methyltetrahydro-2H-pyran-2-yl)oxy)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FRXA-C18-A—(2S,3S,4S,5R)-6-(((3S,4S,6aR,6bS,8R,8aR,12aS,14bR)-8a-((((2R,3S,4R,5S,6S)-3-(((2S,3R,4S,5R,6S)-5-(((2S,3R,4S,5R)-3,5-dihydroxy-4-(((2S,3R,4R)-4-hydroxy-4-(hydroxymethyl)-3-methyltetrahydrofuran-2-yl)oxy)tetrahydro-2H-pyran-2-yl)oxy)-3,4-dihydroxy-6-methyltetrahydro-2H-pyran-2-yl)oxy)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FRXA-C18-A—(2S,3S,4S,5R)-6-(((3S,4S,6aR,6bS,8R,8aR,12aS,14bR)-8a-((((2R,3S,4R,5S,6S)-3-(((2S,3R,4S,5R,6S)-5-(((2S,3R,4S,5R)-3,5-dihydroxy-4-(((2S,3R,4R)-4-hydroxy-4-(hydroxymethyl)-3-methyltetrahydrofuran-2-yl)oxy)tetrahydro-2H-pyran-2-yl)oxy)-3,4-dihydroxy-6-methyltetrahydro-2H-pyran-2-yl)oxy)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2R,3R,4S,5R)-3,4,5-trihydroxytetrahydro-2H-pyran-2-yl)oxy)tetrahydro-2H-pyran-2-carboxylic acid
   -QA-TriR-FRXA-C18-A—(2S,3S,4S,5R,6R)-6-(((3S,4S,6aR,6bS,8R,8aR,12aS,14bR)-8a-((((2S,3R,4S,5R,6R)-3-(((2S,3R,4S,5R,6S)-5-(((2S,3R,4S,5R)-4-(((2S,3R,4R)-3,4-dihydroxy-4-(hydroxymethyl)tetrahydrofuran-2-yl)oxy)-3,5-dihydroxytetrahydro-2H-pyran-2-yl)oxy)-3,4-dihydroxy-6-methyltetrahydro-2H-pyran-2-yl)oxy)-5-(((3S,6S)-5-(((3S,6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2 S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
QA*-F*-C18-A-G
   -QA-FRX-C18-A-G—(2S,3R,4S,5R,6R)-5-(((3R,6S)-5-(((3R,6S)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-3-(((2 S,3R,5S,6S)-3-hydroxy-6-methyl-4-(((2R,3S,4R,5R,6S)-3,4,5-trihydroxy-6-(hydroxymethyl)tetrahydro-2H-pyran-2-yl)oxy)-5-(((2 S,4S,5R)-3,4,5-trihydroxytetrahydro-2H-pyran-2-yl)oxy)tetrahydro-2H-pyran-2-yl)oxy)-6-methyltetrahydro-2H-pyran-2-yl (4aR,5R,6aS,6bR,9S,10S,12aR,14bS)-9-formyl-5,10-dihydroxy-2,2,6a,6b,9,12a,14b-heptamethyl-1,3,4,5,6,6a,6b,7,8,8a,9,10,11,12,12a,12b,13,14b-octadecahydropicene-4a(2H)-carboxylate
   -QA-Mono-FRX-C18-A-G—(2S,3S,4S,5R,6R)-6-(((3S,4S,6aR,6bS,8R,8aR,12aS,14bR)-8a-((((2S,3R,4S,5R,6R)-5-(((3R,6S)-5-(((3R,6S)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-3-(((2S,3R,5S,6S)-3-hydroxy-6-methyl-4-(((2R,3S,4R,5R,6S)-3,4,5-trihydroxy-6-(hydroxymethyl)tetrahydro-2H-pyran-2-yl)oxy)-5-(((2 S,4S,5R)-3,4,5-trihydroxytetrahydro-2H-pyran-2-yl)oxy)tetrahydro-2H-pyran-2-yl)oxy)-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FRX-C18-A-G—(2S,3S,4S,5R,6R)-6-(((3S,4S,6aR,6bS,8R,8aR,12aS,14bR)-8a-((((2S,3R,4S,5R,6R)-5-(((3R,6S)-5-(((3R,6S)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-3-(((2S,3R,5S,6S)-3-hydroxy-6-methyl-4-(((2R,3S,4R,5R,6S)-3,4,5-trihydroxy-6-(hydroxymethyl)tetrahydro-2H-pyran-2-yl)oxy)-5-(((2S,4S,5R)-3,4,5-trihydroxytetrahydro-2H-pyran-2-yl)oxy)tetrahydro-2H-pyran-2-yl)oxy)-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FRX-C18-A-G—(2S,3S,4S,5R,6R)-6-(((3S,4S,6aR,6bS,8R,8aR,12aS,14bR)-8a-((((2S,3R,4S,5R,6R)-5-(((3R,6S)-5-(((3R,6S)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-3-(((2S,3R,5S,6S)-3-hydroxy-6-methyl-4-(((2R,3S,4R,5R,6S)-3,4,5-trihydroxy-6-(hydroxymethyl)tetrahydro-2H-pyran-2-yl)oxy)-5-(((2 S,4S,5R)-3,4,5-trihydroxytetrahydro-2H-pyran-2-yl)oxy)tetrahydro-2H-pyran-2-yl)oxy)-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2 S,3R,4S,5R,6R)-3,4,5-trihydroxy-6-(hydroxymethyl)tetrahydro-2H-pyran-2-yl)oxy)-4-(((2S,3R,4S,5R)-3,4,5-trihydroxytetrahydro-2H-pyran-2-yl)oxy)tetrahydro-2H-pyran-2-carboxylic acid
   -QA-FRXX-C18-A-G—(2S,3R,4S,5R,6R)-3-(((2S,3R,5S,6S)-5-(((2S,4S,5R)-3,5-dihydroxy-4-(((2S,4S,5R)-3,4,5-trihydroxytetrahydro-2H-pyran-2-yl)oxy)tetrahydro-2H-pyran-2-yl)oxy)-3-hydroxy-6-methyl-4-(((2R,3S,4R,5R,6S)-3,4,5-trihydroxy-6-(hydroxymethyl)tetrahydro-2H-pyran-2-yl)oxy)tetrahydro-2H-pyran-2-yl)oxy)-5-(((3R,6S)-5-(((3R,6S)-5-(((2S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl (4aR,5R,6aS,6bR,9S,10S,12aR,14bS)-9-formyl-5,10-dihydroxy-2,2,6a,6b,9,12a,14b-heptamethyl-1,3,4,5,6,6a,6b,7,8,8a,9,10,11,12,12a,12b,13,14b-octadecahydropicene-4a(2H)-carboxylate
   -QA-Mono-FRXX-C18-A-G—(2S,3S,4S,5R,6R)-6-(((3S,4S,6aR,6bS,8R,8aR,12aS,14bR)-8a-((((2S,3R,4S,5R,6R)-3-(((2S,3R,5S,6S)-5-(((2S,4S,5R)-3,5-dihydroxy-4-(((2 S,4S,5R)-3,4,5-trihydroxytetrahydro-2H-pyran-2-yl)oxy)tetrahydro-2H-pyran-2-yl)oxy)-3-hydroxy-6-methyl-4-(((2R,3S,4R,5R,6S)-3,4,5-trihydroxy-6-(hydroxymethyl)tetrahydro-2H-pyran-2-yl)oxy)tetrahydro-2H-pyran-2-yl)oxy)-5-(((3R,6S)-5-(((3R,6S)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FRXX-C18-A-G—(2S,3S,4S,5R,6R)-6-(((3S,4S,6aR,6bS,8R,8aR,12aS,14bR)-8a-((((2S,3R,4S,5R,6R)-3-(((2S,3R,5S,6S)-5-(((2S,4S,5R)-3,5-dihydroxy-4-(((2 S,4S,5R)-3,4,5-trihydroxytetrahydro-2H-pyran-2-yl)oxy)tetrahydro-2H-pyran-2-yl)oxy)-3-hydroxy-6-methyl-4-(((2R,3S,4R,5R,6S)-3,4,5-trihydroxy-6-(hydroxymethyl)tetrahydro-2H-pyran-2-yl)oxy)tetrahydro-2H-pyran-2-yl)oxy)-5-(((3R,6S)-5-(((3R,6S)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3,4-dihydroxy-5-(((2 S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FRXX-C18-A-G—(2S,3S,4S,5R,6R)-6-(((3S,4S,6aR,6bS,8R,8aR,12aS,14bR)-8a-((((2S,3R,4S,5R,6R)-3-(((2S,3R,5S,6S)-5-(((2S,4S,5R)-3,5-dihydroxy-4-(((2 S,4S,5R)-3,4,5-trihydroxytetrahydro-2H-pyran-2-yl)oxy)tetrahydro-2H-pyran-2-yl)oxy)-3-hydroxy-6-methyl-4-(((2R,3S,4R,5R,6S)-3,4,5-trihydroxy-6-(hydroxymethyl)tetrahydro-2H-pyran-2-yl)oxy)tetrahydro-2H-pyran-2-yl)oxy)-5-(((3R,6S)-5-(((3R,6S)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2 S,3R,4S,5R,6R)-3,4,5-trihydroxy-6-(hydroxymethyl)tetrahydro-2H-pyran-2-yl)oxy)-4-(((2S,3R,4S,5R)-3,4,5-trihydroxytetrahydro-2H-pyran-2-yl)oxy)tetrahydro-2H-pyran-2-carboxylic acid
   -QA-TriR-FRX-C18-A-G—(2S,3S,4S,5R,6R)-6-(((3S,4S,6aR,6bS,8R,8aR,12aS,14bR)-8a-((((2S,3R,4S,5R,6R)-5-(((3R,6R)-5-(((3R,6S)-5-(((2 S,3S,4S,5R)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-3-(((2S,3R,5S,6S)-3-hydroxy-6-methyl-4-(((2R,3S,4R,5R,6S)-3,4,5-trihydroxy-6-(hydroxymethyl)tetrahydro-2H-pyran-2-yl)oxy)-5-(((2 S,4S,5R)-3,4,5-trihydroxytetrahydro-2H-pyran-2-yl)oxy)tetrahydro-2H-pyran-2-yl)oxy)-6-methyltetrahydro-2H-pyran-2-yl)oxy)carbonyl)-4-formyl-8-hydroxy-4,6a,6b,11,11,12a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2 S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
   -QA-FRXA-C18-A-G—(2S,3R,4S,5S,6R)-3-(((2S,3R,4S,5S,6S)-5-(((2S,3R,4S,5R)-4-(((2S,3R,4R)-3,4-dihydroxy-4-(hydroxymethyl)tetrahydrofuran-2-yl)oxy)-3,5-dihydroxytetrahydro-2H-pyran-2-yl)oxy)-3-hydroxy-6-methyl-4-(((2 S,3R,4S,5S,6R)-3,4,5-trihydroxy-6-(hydroxymethyl)tetrahydro-2H-pyran-2-yl)oxy)tetrahydro-2H-pyran-2-yl)oxy)-4-((5-((5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-5-hydroxy-6-methyltetrahydro-2H-pyran-2-yl (4aR,5R,6aS,6bR,8aR,9S,10S,12aR,12bR,14bS)-9-formyl-5,10-dihydroxy-2,2,6a,6b,9,12a-hexamethyl-1,3,4,5,6,6a,6b,7,8,8a,9,10,11,12,12a,12b,13,14b-octadecahydropicene-4a(2H)-carboxylate
   -QA-Mono-FRXA-C18-A-G—(2S,3S,4S,5R,6R)-6-(((3S,4S,4aR,6aR,6bS,8R,8aR,12aS,14aR,14bR)-8a-((((2S,3R,4S,5S,6R)-3-(((2S,3R,4S,5S,6S)-5-(((2S,3R,4S,5R)-4-(((2 S,3R,4R)-3,4-dihydroxy-4-(hydroxymethyl)tetrahydrofuran-2-yl)oxy)-3,5-dihydroxytetrahydro-2H-pyran-2-yl)oxy)-3-hydroxy-6-methyl-4-(((2S,3R,4S,5S,6R)-3,4,5-trihydroxy-6-(hydroxymethyl)tetrahydro-2H-pyran-2-yl)oxy)tetrahydro-2H-pyran-2-yl)oxy)-4-((5-((5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-5-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4,5-trihydroxytetrahydro-2H-pyran-2-carboxylic acid
   -QA-Di-FRXA-C18-A-G—(2S,3S,4S,5R,6R)-6-(((3S,4S,4aR,6aR,6bS,8R,8aR,12aS,14aR,14bR)-8a-((((2S,3R,4S,5S,6R)-3-(((2S,3R,4S,5S,6S)-5-(((2S,3R,4S,5R)-4-(((2 S,3R,4R)-3,4-dihydroxy-4-(hydroxymethyl)tetrahydrofuran-2-yl)oxy)-3,5-dihydroxytetrahydro-2H-pyran-2-yl)oxy)-3-hydroxy-6-methyl-4-(((2S,3R,4S,5S,6R)-3,4,5-trihydroxy-6-(hydroxymethyl)tetrahydro-2H-pyran-2-yl)oxy)tetrahydro-2H-pyran-2-yl)oxy)-4-((5-((5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-5-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3,4-dihydroxy-5-(((2S,3R,4S,5R,6R)-3,4,5-trihydroxy-6-(hydroxymethyl)tetrahydro-2H-pyran-2-yl)oxy)tetrahydro-2H-pyran-2-carboxylic acid
   -QA-TriX-FRXA-C18-A-G—(2S,3S,4S,5R,6R)-6-(((3S,4S,4aR,6aR,6bS,8R,8aR,12aS,14aR,14bR)-8a-((((2S,3R,4S,5S,6R)-3-(((2S,3R,4S,5S,6S)-5-(((2S,3R,4S,5R)-4-(((2S,3R,4R)-3,4-dihydroxy-4-(hydroxymethyl)tetrahydrofuran-2-yl)oxy)-3,5-dihydroxytetrahydro-2H-pyran-2-yl)oxy)-3-hydroxy-6-methyl-4-(((2S,3R,4S,5S,6R)-3,4,5-trihydroxy-6-(hydroxymethyl)tetrahydro-2H-pyran-2-yl)oxy)tetrahydro-2H-pyran-2-yl)oxy)-4-((5-((5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-5-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4S,5R)-3,4,5-trihydroxytetrahydro-2H-pyran-2-yl)oxy)tetrahydro-2H-pyran-2-carboxylic acid
   -QA-TriR-FRXA-C18-A-G—(2S,3S,4S,5R,6R)-6-(((3S,4S,4aR,6aR,6bR,8R,8aR,12aS,14aR,14bS)-8a-((((2S,3R,4S,5R,6R)-3-(((2S,3R,4S,5S,6S)-5-(((2S,3R,4S,5R)-4-(((2S,3R,4R)-3,4-dihydroxy-4-(hydroxymethyl)tetrahydrofuran-2-yl)oxy)-3,5-dihydroxytetrahydro-2H-pyran-2-yl)oxy)-3-hydroxy-6-methyl-4-(((2S,3R,4S,5S,6R)-3,4,5-trihydroxy-6-(hydroxymethyl)tetrahydro-2H-pyran-2-yl)oxy)tetrahydro-2H-pyran-2-yl)oxy)-5-(((3R,6S)-5-(((3R,6R)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,14a,14b-heptamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4R,5R,6S)-3,4,5-trihydroxy-6-methyltetrahydro-2H-pyran-2-yl)oxy)tetrahydro-2H-pyran-2-carboxylic acid
Qs_0283850—Q. saponaria QA-Di α-1,3-L-rhamnosyltransferase
Qs_0283870—Q. saponaria QA-Di β-1,3-D-xylosyltransferase
QS-7-GlcT-Quillaic acid 28-O-fucoside [1,2]-rhamnoside [1,3]glucosyltransferase (also referred to as QsUGT-BI)
Qs-28-O-ApiT4 —Quillaic acid 28-O-fucoside [1,2]-rhamnoside [1,4] xyloside [1,3] apiosyltransferase
Qs-28-O-FucT—Q. saponaria Quillaic acid 28-O-fucosyltransferase
Qs-28-O-RhaT—Q. saponaria Quillaic acid 28-O-fucoside [1,2]-rhamnosyltransferase
Qs-28-O-XyIT3—Q. saponaria Quillaic acid 28-O-fucoside [1,2]-rhamnoside [1,4]xylosyltransferase
Qs-28-O-XyIT4—Q. saponaria Quillaic acid 28-O-fucoside [1,2]-rhamnoside [1,4]xyloside [1,3]xylosyltransferase
Qs-3-O-GaIT—Q. saponaria QA-Mono β-1,2-D-galactosyltransferase
QsbAS—Q. saponaria β-amyrin synthase
QsCSL1—Q. saponaria cellulose synthase-like enzyme (quillaic acid 3-O-glucuronosyltransferase)
QsCsIG2—Q. saponaria cellulose synthase-like enzyme (quillaic acid 3-O-glucuronosyltransferase)
QsCYP716-C-28—Q. saponaria quillaic acid C-28 oxidase
QsCYP716-C-16a—Q. saponaria quillaic acid C-16a oxidase
QsCYP714-C-23—Q. saponaria quillaic acid C-23 oxidase
QsFSL-1—Enzyme from Q. saponaria boosting the production of fucosylated saponins
QsFSL-2—Enzyme from Q. saponaria boosting the production of fucosylated saponins
QsFucSyn—Enzyme from Q. saponaria boosting the production of fucosylated saponins
QsUGT-BI—Quillaic acid 28-O-fucoside [1,2]-rhamnoside [1,3]glucosyltransferase (also referred to as QS-7-GIcT)Rhap—L-Rhamnopyranose
SoFSL-1—Enzyme from S. officinalis boosting the production of fucosylated saponins
UGT—UDP-dependent glycosyltransferases
UGT-L—Uridine Diphosphate glycosyltransferase-L
Xylp—D-Xylopyranose

### REFERENCES

Arias M A, Van Roey G A, Tregoninh, J S, Moutaftsi M, Coler, R N, Windish, H P, Reed S G, Carter D, Shattock R J, (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144.
Coler R N, Bertholet S, Moutaftsi M, Guderian J A, Windish H P, Baldwin S L, Laighlin, E.M., Duthie, M. S., Fox, C. B., Carter, D., Friede, M., Vedvick, T. S., Reed, S. G. (2011) Development and Characterization of Synthetic Glucopyranosyl Lipid Adjuvant System as a Vaccine Adjuvant. PLoS ONE 6(1): e16333. doi:10.1371/journal.pone.0016333
Fleck J D, Betti A H, da Silva F P, Troian E A, Olivaro C, Ferreira F, Verza S G. (2019). Saponins from Quillaja saponaria and Quillaja brasiliensis: Particular Chemical Characteristics and Biological Activities. Molecules 24(1).
Glässer L, Kuhl M, Jovanovic S, Fritz M, V6geli B et al. (2020). A common approach for absolute quantification of short chain CoA thioesters in prokaryotic and eukaryotic microbes. Microb Cell Fact 19:160 https://doi.org/10.1186/si2934-020-01413-1*
Hou B, Lim E-K, Higgins G S, Bowles D J. (2004). N-glucosylation of cytokinins by glycosyltransferases of Arabidopsis thaliana. J. Biol. Chem. 279, 47822-47832.
Kensil C R, Patel U, Lennick M, Marciani D. Separation and characterization of saponins with adjuvant activity from Quillaja saponaria Molina cortex. (1991). J Immunol. 15; 146(2):431-7. PMID: 1987271.
Louveau T, Osbourn A. (2019). The Sweet Side of Plant-Specialized Metabolism. Cold Spring Harb Perspect Biol. 11(12):a034744. doi: 10.1101/cshperspect.a034744. PMID: 31235546; PMCID: PMC6886449.
Marciani D J. (2018). Elucidating the mechanisms of action of saponin-derived adjuvants. Trends in Pharmacological Sciences. 39(6):573-585.
Ragupathi G, Gardner J, Livingston P, Gin D 2011 Natural and synthetic saponin adjuvant QS-21 for vaccines against cancer. Expert Rev. Vaccines, 10(4) 463-470.
Reed J, Stephenson M J, Miettinen K, Brouwer B, Leveau A, Brett P, Goss R J M, Goossens A, O'Connell M A, Osbourn A. 2017. A translational synthetic biology platform for rapid access to gram-scale quantities of novel drug-like molecules. Metab Eng 42: 185-193.
Sainsbury F, Thuenemann E C, Lomonossoff G P. 2009. pEAQ: versatile expression vectors for easy and quick transient expression of heterologous proteins in plants. Plant Biotechnol J 7(7): 682-693.
Xu H, Zhang F, Liu B, Huhman D V, Sumner L W, et al. (2013). Characterization of the Formation of Branched Short-Chain Fatty Acid:CoAs for Bitter Acid Biosynthesis in Hop Glandular Trichomes. Molecular Plant 6(4):1301-1317

1. A method of making QA*-F*-C18-A, wherein the F* chain is at the C-28 position of QA*, and the C-18-A chain is attached to the D-fucose of the F* chain, the method comprising the step of combining QA*-F*-C18 with an enzyme capable of transferring UDP-β-L-arabinofuranose to QA*-F*-C18 to form QA*-F*-C18-A.

2. The method of claim 1, wherein QA*-F*-C18 is formed by combining QA*-F*-C9 with an enzyme capable of transferring an acyl unit to QA*-F*-C9.

3. The method of claim 2, wherein QA*-F*-C9 is formed by combining (3S,5S,6S)-3,5-dihydroxy-6-methyloctanoyl-CoA and QA*-F* with an enzyme capable of transferring an acyl unit to QA*-F*.

4. The method of claim 3, wherein (3S,5S,6S)-3,5-dihydroxy-6-methyloctanoyl-CoA is formed by combining 2-methylbutyryl-CoA and malonyl-CoA with one or more enzymes capable of adding malonyl-CoA, and one or more enzymes capable of reducing a ketone.

5. The method of claim 4, wherein 2-methylbutyryl-CoA is formed by combining 2-methylbutyric acid with one or more enzymes capable of transferring a coenzyme A to 2-methylbutyric acid.

6. The method of any one of claims 1 to 5, wherein F* is FRX, FRXX, FRXA or mixtures thereof, preferably wherein F* is FRXA.

7. A method of making QA*-F*-C18-A-G, wherein the F* chain is at the C-28 position of QA*, the C-18-A chain is attached to the D-fucose of the F* chain and the G residue is attached at the C-3 position of the rhamnose residue of the F* chain, comprising: making QA*-F*-C18-A by the method of claim 6, and combining QA*-F*-C18-A with an enzyme capable of transferring a D-glucose residue to QA*-F*-C18-A to form QA*-F*-C18-A-G.

8. A method of making QA*-F*-C18-A-G wherein the F* chain is at the C-28 position of QA*, the C-18-A chain is attached to the D-fucose of the F* chain and the G residue is attached at the C-3 position of the rhamnose residue of the F* chain, wherein F* is FRX, FRXX, FRXA or mixtures thereof, preferably wherein F* is FRXA, and wherein the method comprises the step of combining QA*-F* with an enzyme capable of transferring a D-glucose residue to QA*-F* to form QA*-F*-G.

9. The method of claim 8, further comprising the step of combining QA*-F*-G with (3S,5S,6S)-3,5-dihydroxy-6-methyloctanoyl-CoA and an enzyme capable of transferring an acyl unit to QA*-F*-G to form QA*-F*-C9-G.

10. The method of claim 9, further comprising the step of combining QA*-F*-C9-G with an enzyme capable of transferring an acyl unit to QA*-F*-C9-G to form QA*-F*-C18-G.

11. The method of claim 10, further comprising the step of combining QA*-F*-C18-G with an enzyme capable of transferring UDP-β-L-arabinofuranose to QA*-F*-C18-G to form QA*-F*-C18-A-G.

12. The method of any one of claims 9 to 11 wherein (3S,5S,6S)-3,5-dihydroxy-6-methyloctanoyl-CoA is formed by combining 2-methylbutyryl-CoA and malonyl-CoA with one or more enzymes capable of adding malonyl-CoA, and one or more enzymes capable of reducing a ketone.

13. The method of claim 12 wherein 2-methylbutyryl-CoA is formed by combining 2-methylbutyric acid with one or more enzymes capable of transferring a coenzyme A to 2-methylbutyric acid

14. A method of making a biosynthetic QA*-F*-C18-A in a host, wherein the F* chain is at the C-28 position of QA*, and the C-18-A chain is attached to the D-fucose of the F* chain, the method comprising the steps of:
a) expressing genes required for the biosynthesis of QA*-F* into the host, and
b) introducing a polynucleotide encoding:
i. at least one or more enzymes capable of transferring a coenzyme A to 2-methylbutyric acid to form 2-methylbutyryl-CoA;
ii. at least one or more enzymes capable of adding malonyl-CoA, and one or more enzymes capable of reducing a ketone, to form (3S,5S,6S)-3,5-dihydroxy-6-methyloctanoyl-CoA;
iii. at least one or more enzymes capable of transferring an acyl unit to QA*-F* to form QA*-F*-C9;
iv. at least one or more enzymes capable of transferring an acyl unit to QA*-F*-C9 to form QA*-F*-C18; and
v. at least one or more enzymes capable of transferring UDP-β-L-arabinofuranose to QA*-F*-C18 to form QA*-F*-C18-A,
into the host.

15. The method of claim 14, wherein F* is FRX, FRXX, FRXA or mixtures thereof, preferably wherein F* is FRXA.

16. A method of making a biosynthetic QA*-F*-C18-A-G, wherein the F* chain is at the C-28 position of QA*, the C-18-A chain is attached to the D-fucose of the F* chain and the G residue is attached at the C-3 position of the rhamnose residue of the F* chain, comprising:
making QA*-F*-C18-A by the method of claim 15, wherein step b) further comprises introducing a polynucleotide encoding:
vi. at least one or more enzymes capable of transferring a glucose residue to QA*-F*-C18-A to form QA*-F*-C18-A-G,
into the host.

17. The method of any one of claims 5 to 7 or 13 to 16, wherein the one or more enzymes capable of transferring a coenzyme A to 2-methylbutyric acid is selected from carboxyl CoA ligase 6 (6CCL) having the amino acid sequence of SEQ ID NO 64, carboxyl CoA ligase 5 (5CCL) having the amino acid sequence of SEQ ID NO 62, carboxyl CoA ligase 4 (4CCL) having the amino acid sequence of SEQ ID NO 60, carboxyl CoA ligase 3 (3CCL) having the amino acid sequence of SEQ ID NO 58, carboxyl CoA ligase 2 (2CCL) having the amino acid sequence of SEQ ID NO 56, carboxyl CoA ligase 1 (1CCL) having the amino acid sequence of SEQ ID NO 54 or an enzyme having an amino acid sequence with at least 60% sequence identity to SEQ ID NO 64, 62, 60, 58, 56 or 54.

18. The method according to claim 17, wherein the one or more enzymes capable of transferring a coenzyme A to 2-methylbutyric acid is 3CCL having the amino acid sequence of SEQ ID NO 58 or an enzyme having an amino acid sequence with at least 60% sequence identity to SEQ ID NO 58.

19. The method of any one of claims 4 to 7, 12 to 18, wherein the one or more enzymes capable of adding malonyl-CoA is selected from chalcone synthase-like A (ChSA) having the amino acid sequence of SEQ ID NO 66, chalcone synthase-like B (ChSB) having the amino acid sequence of SEQ ID NO 68, chalcone synthase-like C (ChSC) having the amino acid sequence of SEQ ID NO 70, chalcone synthase-like D (ChSD) having the amino acid sequence of SEQ ID NO 72, chalcone synthase-like E (ChSE) having the amino acid sequence of SEQ ID NO 74, chalcone synthase-like F (ChSF) having the amino acid sequence of SEQ ID NO 76 and an enzyme having an amino acid sequence with at least 50% sequence identity to SEQ ID NO 66, 68, 70, 72, 74 or 76.

20. The method of claim 19, wherein the one or more enzymes capable of adding malonyl-CoA is ChSD having the amino acid sequence of SEQ ID NO 72 or an enzyme having an amino acid sequence with at least 50% sequence identity to SEQ ID NO 72.

21. The method of claim 19 or 20, wherein the one or more enzymes capable of adding malonyl-CoA is, or further includes, ChSE having the amino acid sequence of SEQ ID NO 74 or an enzyme having an amino acid sequence with at least 50% sequence identity to SEQ ID NO 74.

22. The method of any one of claims 4 to 7 or 12 to 21, wherein the one or more enzymes capable of reducing a ketone to form (3S,5S,6S)-3,5-dihydroxy-6-methyloctanoyl-CoA is selected from keto reductase 11 (KR11) having the amino acid sequence of SEQ ID NO 78 and an enzyme having an amino acid sequence with at least 20% sequence identity to SEQ ID NO 78, optionally in combination with an enzyme selected from keto reductase 23′ (KR23′) having the amino acid sequence of SEQ ID NO 80 and an enzyme having an amino acid sequence with at least 15% sequence identity to SEQ ID NO 80.

23. The method of any one of claims 3 to 7 or 9 to 22, wherein the enzyme capable of transferring an acyl unit to QA*-F* or QA*-F*-G to form QA*-F*-C9 or QA*-F*-C9-G is (3S,5S,6S)-3,5-dihydroxy-6-methyloctanoyl-CoA transferase 9 (DMOT9) having the amino acid sequence of SEQ ID NO 82 or an enzyme having an amino acid sequence with at least 40% sequence identity to SEQ ID NO 82.

24. The method of any one of claims 2 to 7 or 10 to 23, wherein the enzyme capable of transferring an acyl unit to QA*-F*-C9 or QA*-F*-C9-G to form QA*-F*-C18 or QA*-F*-C18-G is (3S,5S,6S)-3,5-dihydroxy-6-methyloctanoyl-CoA transferase 4 (DMOT4) having the amino acid sequence of SEQ ID NO 84 or an enzyme having an amino acid sequence with at least 50% sequence identity to SEQ ID NO 84.

25. The method of any one of claims 1 to 7 or 11 to 24, wherein the enzyme capable of transferring UDP-β-L-arabinofuranose to QA*-F*-C18 or QA*-F*-C-18-G is selected from uridine diphosphate glycosyltransferase-L-short (UGT-L-short) having the amino acid sequence of SEQ ID NO 86, uridine diphosphate glycosyltransferase-L-long (UGT-L-long) having the amino acid sequence of SEQ ID NO 88 and an enzyme having an amino acid sequence with at least 50% sequence identity to SEQ ID NO 86 or 88.

26. The method of any one of claims 7 to 13 or 16 to 25, wherein the enzyme capable of transferring a glucose residue to QA*-F*-C18-A or QA*-F* to form QA*-F*-C18-A-G of QA*-F*-G is quillaic acid 28-O-fucoside [1,2]-rhamnoside [1,3]glucosyltransferase (QS-7-GlcT) having the amino acid sequence of SEQ ID NO 90 or an enzyme having an amino acid sequence with at least 70% sequence identity to SEQ ID NO 90.

27. The method of any one of claims 14 to 26, wherein the polynucleotide introduced into the host in step b) encodes:
i. at least one or more enzymes selected from 6CCL having the amino acid sequence of SEQ ID NO 64, 5CCL having the amino acid sequence of SEQ ID NO 62, 4CCL having the amino acid sequence of SEQ ID NO 60, 3CCL having the amino acid sequence of SEQ ID NO 58, 2CCL having the amino acid sequence of SEQ ID NO 56, 1CCL having the amino acid sequence of SEQ ID NO 54 and an enzyme having an amino acid sequence with at least 60% sequence identity to SEQ ID NO 64, 62, 60, 58, 56 or 54,
ii. at least one or more enzymes selected from ChSA having the amino acid sequence of SEQ ID NO 66, ChSB having the amino acid sequence of SEQ ID NO 68, ChSC having the amino acid sequence of SEQ ID NO 70, ChSD having the amino acid sequence of SEQ ID NO 72, ChSE having the amino acid sequence of SEQ ID NO 74, ChSF having the amino acid sequence of SEQ ID NO 76 and an enzyme having an amino acid sequence with at least 50% sequence identity to SEQ ID NO 66, 68, 70, 72, 74 or 76,
iii. at least one or more enzymes selected from KR11 having the amino acid sequence of SEQ ID NO 78 and an enzyme having an amino acid sequence with at least 20% sequence identity to SEQ ID NO 78, optionally in combination with an enzyme selected from KR23′ having the amino acid sequence of SEQ ID NO 80, and an enzyme having an amino acid sequence with at least 15% sequence identity to SEQ ID NO 80,
iv. at least one or more enzymes selected from DMOT9 having the amino acid sequence of SEQ ID NO 82, an enzyme having an amino acid sequence with at least 25% sequence identity to SEQ ID NO 82, DMOT4 having the amino acid sequence of SEQ ID NO 84 and an enzyme having an amino acid sequence with at least 15% sequence identity to SEQ ID NO 84; and
v. at least one or more enzymes selected from UGT-L-short having the amino acid sequence of SEQ ID NO 86, UGT-L-long having the amino acid sequence of SEQ ID NO 88 and an enzyme having an amino acid sequence with at least 45% sequence identity to SEQ ID NO 86 or 88.

28. The method of claim 27, wherein step b further comprises introducing a polynucleotide encoding QS-7-GlcT having the amino acid sequence of SEQ ID NO 90 or an enzyme having an amino acid sequence with at least 70% sequence identity to SEQ ID NO 90.

29. The method of claim 27 or claim 28, wherein
amino acid SEQ ID NO 54 is encoded by polynucleotide SEQ ID NO 53;
amino acid SEQ ID NO 56 is encoded by polynucleotide SEQ ID NO 55;
amino acid SEQ ID NO 58 is encoded by polynucleotide SEQ ID NO 57;
amino acid SEQ ID NO 60 is encoded by polynucleotide SEQ ID NO 59;
amino acid SEQ ID NO 62 is encoded by polynucleotide SEQ ID NO 61;
amino acid SEQ ID NO 64 is encoded by polynucleotide SEQ ID NO 63;
amino acid SEQ ID NO 66 is encoded by polynucleotide SEQ ID NO 65;
amino acid SEQ ID NO 68 is encoded by polynucleotide SEQ ID NO 67;
amino acid SEQ ID NO 70 is encoded by polynucleotide SEQ ID NO 69;
amino acid SEQ ID NO 72 is encoded by polynucleotide SEQ ID NO 71;
amino acid SEQ ID NO 74 is encoded by polynucleotide SEQ ID NO 73;
amino acid SEQ ID NO 76 is encoded by polynucleotide SEQ ID NO 75;
amino acid SEQ ID NO 78 is encoded by polynucleotide SEQ ID NO 77;
amino acid SEQ ID NO 80 is encoded by polynucleotide SEQ ID NO 79;
amino acid SEQ ID NO 82 is encoded by polynucleotide SEQ ID NO 81;
amino acid SEQ ID NO 84 is encoded by polynucleotide SEQ ID NO 83;
amino acid SEQ ID NO 86 is encoded by polynucleotide SEQ ID NO 85;
amino acid SEQ ID NO 88 is encoded by polynucleotide SEQ ID NO 87; and/or
amino acid SEQ ID NO 90 is encoded by polynucleotide SEQ ID NO 89.

30. The method of any one of claims 1 to 29, wherein the method further comprises the step of adding 2-methylbutyric acid to an infiltration solution.

31. A carboxyl CoA enzyme having the amino acid sequence of SEQ ID NO 54 (1CCL), SEQ ID NO 56 (2CCL), SEQ ID NO 58 (3CCL), SEQ ID NO 60 (4CCL), SEQ ID NO 62 (5CCL), SEQ ID NO 64 (6CCL) or an enzyme having an amino acid sequence with at least 60% sequence identity to SEQ ID NO 54, 56, 58, 60, 62 or 64.

32. A chalcone synthase-like enzyme having the amino acid sequence of SEQ ID NO 66 (ChSA), SEQ ID NO 68 (ChSB), SEQ ID NO 70 (ChSC), SEQ ID NO 72 (ChSD), SEQ ID NO 74 (ChSE), SEQ ID NO 76 (ChSF) or an enzyme having an amino acid sequence with at least 50% sequence identity to SEQ ID NO 66, 68, 70, 72, 74 or 76.

33. A keto reductase enzyme having the amino acid sequence of SEQ ID NO 78 (KR11) or an enzyme having an amino acid sequence with at least 20% sequence identity to SEQ ID NO 78.

34. A keto reductase enzyme having the amino acid sequence of SEQ ID NO 80 (KR23′) or an enzyme having an amino acid sequence with at least 15% sequence identity to SEQ ID NO 80.

35. An acyl transferase enzyme having the amino acid sequence of SEQ ID NO 82 (DMOT9) or an enzyme having an amino acid sequence with at least 25% sequence identity to SEQ ID NO 82.

36. An acyl transferase enzyme having the amino acid sequence of SEQ ID NO 84 (DMOT4) or an enzyme having an amino acid sequence with at least 15% sequence identity to SEQ ID NO 84.

37. An arabinofuranosyl transferase enzyme having the amino acid sequence of SEQ ID NO 86 (UGT-L-short), SEQ ID NO 88 (UGT-L-long), or an enzyme having an amino acid sequence with at least 45% sequence identity to SEQ ID NO 86 or 88.

38. A polynucleotide which encodes any of the enzymes as claimed in any one of claims 31 to 37.

39. A vector comprising the polynucleotide according to claim 38.

40. A host cell comprising the polynucleotide according to claim 38.

41. A host cell transformed with the vector according to claim 39.

42. A host cell according to claim 40 or claim 41, wherein the host cell is a plant cell or a microbial cell.

43. A biological system of a plant or a microorganism comprising host cells according to any one of claims 40 to 42.

44. A biological system according to claim 43, wherein the biological system is yeast or Nicotiana benthamiana.

45. A method according to any one of claims 1 to 30, wherein the method further includes the step of isolating the QA*-F*-C18-A.

46. A method according to any one of claims 7 to 13 or 16 to 30, wherein the method further includes the step of isolating the QA*-F*-C18-A-G.

47. A QA*-F*-C18-A derivative obtainable by the method of claim 45.

48. The QA*-F*-C18-A derivative of claim 47, wherein the derivative is 2S,3S,4S,5R)-6-(((3S,4S,6aR,6bS,8R,8aR,12aS,14bR)-8a-((((2R,3S,4R,5S,6S)-3-(((2S,3R,4S,5R,6S)-5-(((2S,3R,4S,5R)-3,5-dihydroxy-4-(((2S,3R,4R)-4-hydroxy-4-(hydroxymethyl)-3-methyltetrahydrofuran-2-yl)oxy)tetrahydro-2H-pyran-2-yl)oxy)-3,4-dihydroxy-6-methyltetrahydro-2H-pyran-2-yl)oxy)-5-(((6S)-5-(((6S)-5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-4-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2R,3R,4S,5R)-3,4,5-trihydroxytetrahydro-2H-pyran-2-yl)oxy)tetrahydro-2H-pyran-2-carboxylic acid (QA-TriX-FRXA-C18-A).

49. A QA*-F*-C18-A-G derivative obtainable by the method of claim 46.

50. The QA*-F*-C18-A-G derivative of claim 49, wherein the derivative is (2S,3S,4S,5R,6R)-6-(((3S,4S,4aR,6aR,6bS,8R,8aR,12aS,14aR,14bR)-8a-((((2S,3R,4S,5S,6R)-3-(((2S,3R,4S,5S,6S)-5-(((2S,3R,4S,5R)-4-(((2S,3R,4R)-3,4-dihydroxy-4-(hydroxymethyl)tetrahydrofuran-2-yl)oxy)-3,5-dihydroxytetrahydro-2H-pyran-2-yl)oxy)-3-hydroxy-6-methyl-4-(((2S,3R,4S,5S,6R)-3,4,5-trihydroxy-6-(hydroxymethyl)tetrahydro-2H-pyran-2-yl)oxy)tetrahydro-2H-pyran-2-yl)oxy)-4-((5-((5-(((2R,3R,4R,5S)-3,4-dihydroxy-5-(hydroxymethyl)tetrahydrofuran-2-yl)oxy)-3-hydroxy-6-methyloctanoyl)oxy)-3-hydroxy-6-methyloctanoyl)oxy)-5-hydroxy-6-methyltetrahydro-2H-pyran-2-yl)oxy)carbonyl)-4-formyl-8-hydroxy-4,6a,6b,11,11,14b-hexamethyl-1,2,3,4,4a,5,6,6a,6b,7,8,8a,9,10,11,12,12a,14,14a,14b-icosahydropicen-3-yl)oxy)-3-hydroxy-5-(((2S,3R,4S,5R,6R)-3,4,5-trihydroxy-6-(hydroxymethyl)tetrahydro-2H-pyran-2-yl)oxy)-4-(((2S,3R,4S,5R)-3,4,5-trihydroxytetrahydro-2H-pyran-2-yl)oxy)tetrahydro-2H-pyran-2-carboxylic acid (QA-TriX-FRXA-C18-A-G).

51. The use of the QA*-F*-C18-A derivative according to claim 47 or claim 48, or the QA*-F*-C18-A-G derivative according to claim 49 or 50 as an adjuvant.

52. The use according to claim 51, wherein the adjuvant is a liposomal formulation.

53. The use according to claim 51 or claim 52, wherein the adjuvant further comprises a TLR4 agonist.

54. The use according to claim 53, wherein the TLR4 agonist is 3D-MPL.

55. An adjuvant composition comprising the QA*-F*-C18-A derivative according to claim 47 or claim 48 or the QA*-F*-C18-A-G derivative according to claim 49 or claim 50.


SEQUENCE LISTING (136 sequences)

SEQ ID NO: 1 - DNA, 1374 bp, Quillaja Saponaria
  source 1..1374: mol_type = other DNA
atggagaatgggagagtttacaaatcccatgtcgtggtgctcgcatttcacgggcaaggccatattgttccgttaatccaattatccagacgattggcctggaaaggcatcaaaatcacatttgctacaacacattcttgcaccaaggccattcaaacaggaagtgattcaatttcacttttatcaatttatgatgacataactgatggtgggtttcaaggagaaggaggattcaagggcttccttcagagatttgaagccagtaccacaaggatcttacacgaattcgtcaagaatcatgaaaactcaaagaacccagtaaaatgcttaatatatgatgctaacttaatatgggctctggaaatggcaaagcaattgggtattgctactgctgcatttgtgtttccttcttgggctgccattgccacctactatcccttttatttagaggtgtatgcggatcagcagataaagaaggtagatcctttcacaatgcctgacttacctccacaacttggacttccaaatatggcatctctcggttcagattcgggtcaacactcccccatactcaaactcatgttgcaacagttagaaaattttgggaaagctgactggatcctgtctcacgcatttgaacagtttgaacaagaggtacttgactggatgagaaatatcagcccagtaacaacaattggtccaactctgccatctgtttatcttgatggtaggctaaaagatgacacagattacggttacaatttgtacaagccagatagtgatacctgcatgaagtggctagacactaaggaaactgaatcagtggtttatatatcatttggcagtgttgcagatttgatcccagaacagatgacagaaataacaaactccctgaagaaaatgagcagcaactttctgtgggtggtgaaggaaactgaaaaaaacaacctccctagcagctttgttgaggagacaaaagaaaagggattggtagtgacttggtgcccccagttgaaggtgttgtctcatcctgcagtgggttgtttcattacacactgtggaacaaattccatatttgagtcagtatgctttgcagtgccaatggtgggaatgccacagttttgtgatcaaatgcctaatgcatatttcatggagaaggtctggaaagtaggtgttaggccaagtttggatgacaatggtgtcgtcactggagaagaaattgagcgatgtataaaagtagttaccgaaggagagagtgggcaagagattaagaagaaacttgtgcagtggaaagagcttgcaaaagaggcagtggacgagggtggaagttcagataagcacattgatgaattcattgctggaatcacaacttga

SEQ ID NO: 2 - protein, 457 aa, Quillaja Saponaria
MENGRVYKSHVVVLAFHGQGHIVPLIQLSRRLAWKGIKITFATTHSCTKAIQTGSDSISLLSIYDDITDGGFQGEGGFKGFLQRFEASTTRILHEFVKNHENSKNPVKCLIYDANLIWALEMAKQLGIATAAFVFPSWAAIATYYPFYLEVYADQQIKKVDPFTMPDLPPQLGLPNMASLGSDSGQHSPILKLMLQQLENFGKADWILSHAFEQFEQEVLDWMRNISPVTTIGPTLPSVYLDGRLKDDTDYGYNLYKPDSDTCMKWLDTKETESVVYISFGSVADLIPEQMTEITNSLKKMSSNFLWVVKETEKNNLPSSFVEETKEKGLVVTWCPQLKVLSHPAVGCFITHCGTNSIFESVCFAVPMVGMPQFCDQMPNAYFMEKVWKVGVRPSLDDNGVVTGEEIERCIKVVTEGESGQEIKKKLVQWKELAKEAVDEGGSSDKHIDEFIAGITT

SEQ ID NO: 3 - DNA, 1422 bp, Quillaja Saponaria
  source 1..1422: mol_type = other DNA
atggcaaaaactgataagcagcttcacatcgccatgttcccatggctagctatgggtcatatattcccaaactttgagctcgctaagctctttgctcaaaagggtcactcaattactttaatctccaccccacgaaacatcagtcgtctccctcaaatccctacacatttagagcaattgattaaattagtcagcttgcctatattacccaaacacaaagcaaatctcccagagaatgcagagtccaccatggacgttacccccaataaagtcccataccttaagatggcctatgatggtcttcaggagtcgttgactcaattactgaaatcttcagctcccgattggattctatatgactttgctgctgactggttaccaccacttgttcacagccttcaaatccgctgtgttttcttcgtggtatcccctgcgtggaatctttgcttctttgacactcccaaaccacagttgggcagtgctgctgtttttcgaacaaagcctgaagactatcttcgccctcccagttgggttcctttccattcaaatattgggctaaagcttcacgaggtgaagaaaatgtttgaaggggtttcagataaagaaacaggggtcactgtaagttttaacttcaacaaagcagtttcgagctgtgacttgttttctttccgcagctgctatgaactcgaatcagaatggctgaacctggtggaggatatttacaagaggcctgtagttccagtgggcgtaattccaccctcttttcaagtcagaattgtgaatgaagaagacaacaaaccagagtggttaaagatccaatcttggttagataaacaagagcaaggatcggtggtatacatagcatttggcagtgagcttaagctgggccaacaagatctcaccgaattagctcttggacttgagctttctgggttgccattcttttgggcacttagaaagcagcaagacagctcatcagtagatttaccagatgggtttgaggaccgagtcagtgatcgtggagttgtttgcagagactgggtgccccaacttaagatcctagctcacgggtcaattgggggttatttgactcactgtggttcaggttcagtgatagagggacttcattttgggcgtgttcttgttatgctgccctatttactagaccaagcattatatgctagagtattggaggagaaaaagctgggggttgagataccaaggaacgaacaagatgggtcttttactaggagctcagtggccaagtctgtgaagttggccatagtggatgaggggggaagtatttacagggacaaagccaaagagatgggcttggtattcagtgacaaagatcgtcatgaacaatacattgagaatttccttcaacaccttcaacacaaaagggaacctttccaaatttaa

SEQ ID NO: 4 - protein, 473 aa, Quillaja Saponaria
MAKTDKQLHIAMFPWLAMGHIFPNFELAKLFAQKGHSITLISTPRNISRLPQIPTHLEQLIKLVSLPILPKHKANLPENAESTMDVTPNKVPYLKMAYDGLQESLTQLLKSSAPDWILYDFAADWLPPLVHSLQIRCVFFVVSPAWNLCFFDTPKPQLGSAAVFRTKPEDYLRPPSWVPFHSNIGLKLHEVKKMFEGVSDKETGVTVSFNFNKAVSSCDLFSFRSCYELESEWLNLVEDIYKRPVVPVGVIPPSFQVRIVNEEDNKPEWLKIQSWLDKQEQGSVVYIAFGSELKLGQQDLTELALGLELSGLPFFWALRKQQDSSSVDLPDGFEDRVSDRGVVCRDWVPQLKILAHGSIGGYLTHCGSGSVIEGLHFGRVLVMLPYLLDQALYARVLEEKKLGVEIPRNEQDGSFTRSSVAKSVKLAIVDEGGSIYRDKAKEMGLVFSDKDRHEQYIENFLQHLQHKREPFQI

SEQ ID NO: 5 - DNA, 1395 bp, Quillaja Saponaria
  source 1..1395: mol_type = other DNA
atggctgctgcagctcccaatcacaggctccacatagcattcttcccatggttagcatttggtcacataaaccctttctttgagcttgcgaagctcattgctcaaaagggtcatcatatttctttcatttccaccccaagaaacatccaacgcctttcacaagttcctccacaattagcagattccatagatctagtgagcttaccagtaatccataattcaaacctcccagaaaacgcagagtccaccatggacattccacctgataaaaccccttaccttgggatgcttcacgacagtctcaaagaaccccttactcaattccttcaaactcattcccctgattggattctgtatgacttttcagctggttggcttgctgccatagtagaagaccttggtatctcccacggctacttttctatcattccctgttggaacataggcttcaatggacgccaaatgaatggtttccaaaagccagatatttcattgccctccgccgtgtcgcttaagaaatatgaggtgaagaaaatcatggatttggtcaaatcttttcccaaaattttggatgagtcggccactaaatccatagcttcgcattcgacctgtgaagtaatttttatacgaaattgccccgagattgaagcagattggttcgactatacttcgaaaattttcgataaaccagtggttccggtgggcgtagtgcccccatctgtgcacataactaacaaagagaaggacgagcatttcaacaaatggttggagatcaaagaatggttggatcaacaagacagaggttctgtaatttatatagcttttggaactgaatcactgccaaatcaggatgaaatcaccatgcttgctcaagggcttgagctatgtgggcttcctttcttttgggcattaaggaagtctaatgtggcttctgatcagccaaattcagactcagttgagctaccggagggatttgaagaacaaaccaaaggtcgtggaattgtgtggacgagttgggcacctcaacagagaattctgggtcacaattcaattgggggttttgtgactcactgtggttggagttcagtaatagaaggaattcactatggacggccactgattatgttccctctaacagtcgaacagtctctgaatgctaggattttgggggagaagaagttgggtatggaagtacccagagaagatgatgggtcttttacaggtgaagttgtggcagagacattgaagctggtattgctggaccaagatgggaaagtttacagggacaaggtaacagagatgagtaaggtatttggggacaaagacaaacatgagaaatacatgggtgatctacttgaattcttcaaaaattacaggtctcttaagaggaattag

SEQ ID NO: 6 - protein, 464 aa, Quillaja Saponaria
MAAAAPNHRLHIAFFPWLAFGHINPFFELAKLIAQKGHHISFISTPRNIQRLSQVPPQLADSIDLVSLPVIHNSNLPENAESTMDIPPDKTPYLGMLHDSLKEPLTQFLQTHSPDWILYDFSAGWLAAIVEDLGISHGYFSIIPCWNIGFNGRQMNGFQKPDISLPSAVSLKKYEVKKIMDLVKSFPKILDESATKSIASHSTCEVIFIRNCPEIEADWFDYTSKIFDKPVVPVGVVPPSVHITNKEKDEHFNKWLEIKEWLDQQDRGSVIYIAFGTESLPNQDEITMLAQGLELCGLPFFWALRKSNVASDQPNSDSVELPEGFEEQTKGRGIVWTSWAPQQRILGHNSIGGFVTHCGWSSVIEGIHYGRPLIMFPLTVEQSLNARILGEKKLGMEVPREDDGSFTGEVVAETLKLVLLDQDGKVYRDKVTEMSKVFGDKDKHEKYMGDLLEFFKNYRSLKRN

SEQ ID NO: 7 - DNA, 1530 bp, Quillaja Saponaria
  source 1..1530: mol_type = other DNA
atggactccacccacttgcagccggccactactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 8 - protein, 509 aa, Quillaja Saponaria
MDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 9 - DNA, 1530 bp, Quillaja Saponaria
  source 1..1530: mol_type = other DNA
atggactccacccacttgcagccggccactactcctctgaaaattcacgtcatacctttcatagctccgggtcacattatcccactctctgaattggctcgtatctttgcttcacgcggtgagcatgtgactatcataaccactcctttcaacgctgacctacttcagaaatccattgacgaagacagagattccggcgagcacattggcatccacaccgttgaatggtcagccacagagctaggccttcctcatggggttgaaaatctcagcaacgtcactgacttggagaccggcattaagctacacagggccctcgtgctaatgcagaaacagatggaagatttcatgacccgaaatccacccgattgtataattgccgacacgttttacccgtgggcctccgaatttgctaatcggatgggtatcccgagactcattttcttcccggggtgtactttcgcgctctgtttgatggaatctattcgatctcccgactccccgcaccgaagattgagttcggattcggacccatttgtggttccgggtcttcctcacccgatcatcttgacccgttctcaacttccggaacacgatcgagaggacatagcggacccggctgcccaattcatggatcagtgtaaagaagctgcgatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagaggggcttttcaaaagtgacgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttatttgggtggttcgcggcaaagataaacaaatcgatgttaatgacgatgaggagaagacatggttgcctaaagggtttgaggaaagaatgaaaacagaaaataaaggtttgattgtaaggggatgggccccacaggtgctggttttggatcatccatcgttgggatgtttcttgacgcattgcggctggaattctacgattgagggaatcacagcaggtgttcctttgatcacgtggccagtatactccgagcaattctataatgagaagctaatcacgcaggtgcatggtaatggggtgggggttggttcagaggagtggatcatgctgttcagcgtcgctaaaagtttggtaagtagagacaaaattgagaatgctgtgaggaagataatggacggtggtgatgaggctttagaaatcagaaggcgggcccgggaacttggagaaaaagctaggaaagctgcttcaattggggggtcctccgacaataatctaaccgcagcaattgaagaccttaagcggttgagagaggaccgttcgaagctgaaaacaaaaacaatttga

SEQ ID NO: 10 - protein, 509 aa, Quillaja Saponaria
MDSTHLQPATTPLKIHVIPFIAPGHIIPLSELARIFASRGEHVTIITTPFNADLLQKSIDEDRDSGEHIGIHTVEWSATELGLPHGVENLSNVTDLETGIKLHRALVLMQKQMEDFMTRNPPDCIIADTFYPWASEFANRMGIPRLIFFPGCTFALCLMESIRSPDSPHRRLSSDSDPFVVPGLPHPIILTRSQLPEHDREDIADPAAQFMDQCKEAAMKSYGIILNNFAEIETEYTEHYKKITGHKVWHIGPAAAIVHRNAKEKAEGLFKSDEHDNNLVINWLNSKEPNSVVYVCFGSGCQFPDKQLYEIACGLELSGHQFIWVVRGKDKQIDVNDDEEKTWLPKGFEERMKTENKGLIVRGWAPQVLVLDHPSLGCFLTHCGWNSTIEGITAGVPLITWPVYSEQFYNEKLITQVHGNGVGVGSEEWIMLFSVAKSLVSRDKIENAVRKIMDGGDEALEIRRRARELGEKARKAASIGGSSDNNLTAAIEDLKRLREDRSKLKTKTI

SEQ ID NO: 11 - DNA, 897 bp, Quillaja Saponaria
  source 1..897: mol_type = other DNA
atggcagaagcaacgcagaggtatgctgttgtgacaggatctaataagggaattggatttgggatatgcaagcagttggcttctaagggaatcaaagtagtgttaacagctagagatgagaagagaggtcttgaagcagttgagaaattgaaagaaattagtctggctggtcatgtggtttttcatcaactcgatgtgtctgatcctgctagtgttactagccttgaagatttcatcaaaacccagtttgggaagctagatattctggtaaacaatgctgggataacaggaacaactgtagatgctgatgctttagcagcttcaggcttcggtacagggggtgaacgtaagcctattgattggagtaagttagtgatacagacttatgaatcagttgaaaaagctttcaacacaaactattacggtggcaaaagaatgacagaagcacttatacccctcctccagctatcagactcacccaggattgttaatgtttcctctgctatgggacagttagagaatatacctagtggatgggcaaaggaagtgctcacagatgttgataacctaacagaaggaaaattggatgaggtttcaacccagtttttgaaagatttcaaagagggttcattggaaaccaaaggctggcctagtcttatgtcttcttatatagtctcaaaagctgttttaaatgcctacacaaggattcttgctaagaaatacccagctttctgcatcaattgtgtagatcctggctatgtgaagacagacataaaccatcatactggccaattaagtgttgatgaaggtgctgaaagtcctgtaagactggccttgctgcctaatggtggtccttctggcgtgttcttctccaggacagaagaagcaccattttga

SEQ ID NO: 12 - protein, 298 aa, Quillaja Saponaria
MAEATQRYAVVTGSNKGIGFGICKQLASKGIKVVLTARDEKRGLEAVEKLKEISLAGHVVFHQLDVSDPASVTSLEDFIKTQFGKLDILVNNAGITGTTVDADALAASGFGTGGERKPIDWSKLVIQTYESVEKAFNTNYYGGKRMTEALIPLLQLSDSPRIVNVSSAMGQLENIPSGWAKEVLTDVDNLTEGKLDEVSTQFLKDFKEGSLETKGWPSLMSSYIVSKAVLNAYTRILAKKYPAFCINCVDPGYVKTDINHHTGQLSVDEGAESPVRLALLPNGGPSGVFFSRTEEAPF

SEQ ID NO: 13 - DNA, 1197 bp, Quillaja Saponaria
  source 1..1197: mol_type = other DNA
atgccaactttgtacaaaaaagcaggcttaatggcgtcggcgtcaagggtagatctggatggtaatcagataaagccgatgacaatttgcatgatcggtgccggtgggttcattgggtcccacctctgcgagaagattatggcggagacaccgcacaaggttctggcattagatgtctacaatgacaagatcaagcacttactggagccggattctcttcaatggaaagatcgcatccaattccaccgcatcaacattaagcacgattcgaggctcgaaggtctcatcaagatggcagatctgactataaatctggctgctatttgtactcccgcggattacaacacccgtcctctggacacaatttatagcaatttcattgacgctcttcctgtggtaaagtactgttcggagaataacaagcgtctcattcatttctctacgtgtgaagtgtatgggaaaacgattgggagctttctcccaaaagacagccctcttctaaaggatcctgaatattttgttcttaaagaagatgcctccccatgcatatttggtcctattgaaaagcagagatggtcctacgcatgtgcaaagcaattgattgagaggctggtttatgctgagggtgctgagaatggccttgagttcactattgtgcgacctttcaattggattggccccagaatggatttcatacctggcattgatggtccaagtgaaggtgttccacgggttctggcatgctttagtaacaatctccttcgcggtgagccactcaaactcgttgatggtggccaatcccagagaacttttgtttatatcaaggatgcaattgaagctgttttgttgatgattgaaaaccctgccagggctaatggtcatatttttaatgtgggtaaccctcacaatgaagttacagttcggcaacttgctgaaatgatgaccgaggtctattctaaggtaagtggagaaccgtctcttgaggtgcctaccattgatgtaagctccaaagaattttatggtgaaggatacgatgatagtgacaagagaattcctgacatgaccataatcaacaggcaacttggctggaaccctaagacatcgctctgggatcttcttgagtcgaccctcacctatcaacataggacatatgcagaagctattaagaaatcaattgcgaaaccagttgccagctag

SEQ ID NO: 14 - protein, 398 aa, Quillaja Saponaria
MPTLYKKAGLMASASRVDLDGNQIKPMTICMIGAGGFIGSHLCEKIMAETPHKVLALDVYNDKIKHLLEPDSLQWKDRIQFHRINIKHDSRLEGLIKMADLTINLAAICTPADYNTRPLDTIYSNFIDALPVVKYCSENNKRLIHFSTCEVYGKTIGSFLPKDSPLLKDPEYFVLKEDASPCIFGPIEKQRWSYACAKQLIERLVYAEGAENGLEFTIVRPFNWIGPRMDFIPGIDGPSEGVPRVLACFSNNLLRGEPLKLVDGGQSQRTFVYIKDAIEAVLLMIENPARANGHIFNVGNPHNEVTVRQLAEMMTEVYSKVSGEPSLEVPTIDVSSKEFYGEGYDDSDKRIPDMTIINRQLGWNPKTSLWDLLESTLTYQHRTYAEAIKKSIAKPVAS

SEQ ID NO: 15 - DNA, 1275 bp, Avena strigosa
  source 1..1275: mol_type = other DNA
atggcgcccgagaaaatgcccgaggaggacgaggaaatcgtcgccggggtcgtcgcagggaagatcccctcctacgtgctcgagaccaggctaggcgactgccgcagggcagccgggatccgccgcgaggcgctgcgccggatcaccggcagggagatcgacggccttcccctcgacggcttcgactacgactcgattctcggacagtgctgcgagatgcccgtcgggtacgtgcagctgccggtcggcgtcgcggggccgctcgtcctcgacggccgccgcatatacgtcccgatggccaccacggagggctgcctaatcgccagcaccaaccgcggatgcaaggccattgccgagtccggaggcgcatccagcgtcgtgtaccgcgacgggatgacccgcgcccccgtagcccgcttcccctccgcacgacgcgccgcagagctcaagggcttcctggagaatccggccaactacgacaccctgtccgtggtctttaacagatcaagcagatttgcaaggctgcagggggtcaagtgcgccatggctgggaggaacttgtacatgaggttcacctgcagcaccggggatgccatggggatgaacatggtctccaagggcgtccaaaatgtgctcgactatctgcaggaggacttccctgacatggacgttgtcagcatctcaggcaacttttgttccgacaagaaatcagctgctgtaaactggattgaaggccgtggaaagtccgtggtttgtgaggcagtaatcagagaggaagttgtccacaaggttctcaagaccaacgttcagtcactcgtggagttgaatgtgatcaagaaccttgctggctcagcagttgctggtgctcttgggggtttcaacgcccacgcaagcaacatcgtaacggctatcttcattgccactggtcaggatcctgcacagaatgtggagagctcacagtgtatcactatgttggaagctgtaaatgatggcagagaccttcacatctccgttacaatgccatctatcgaggtgggcacagttggtggaggcacgcagctggcctcacagtcggcctgcttggacctactgggcgtcaaaggcgccaacagggaatctccggggtcgaacgctaggctgctggccacggtggtggctggtgccgtcctagctggggagctgtccctcatctccgcccaagctgccggccatctggtccagagccacatgaaatacaacagatccagcaaggacatgtccaagatcgcctgctga

SEQ ID NO: 16 - protein, 424 aa, Avena strigosa
MAPEKMPEEDEEIVAGVVAGKIPSYVLETRLGDCRRAAGIRREALRRITGREIDGLPLDGFDYDSILGQCCEMPVGYVQLPVGVAGPLVLDGRRIYVPMATTEGCLIASTNRGCKAIAESGGASSVVYRDGMTRAPVARFPSARRAAELKGFLENPANYDTLSVVFNRSSRFARLQGVKCAMAGRNLYMRFTCSTGDAMGMNMVSKGVQNVLDYLQEDFPDMDVVSISGNFCSDKKSAAVNWIEGRGKSVVCEAVIREEVVHKVLKTNVQSLVELNVIKNLAGSAVAGALGGFNAHASNIVTAIFIATGQDPAQNVESSQCITMLEAVNDGRDLHISVTMPSIEVGTVGGGTQLASQSACLDLLGVKGANRESPGSNARLLATVVAGAVLAGELSLISAQAAGHLVQSHMKYNRSSKDMSKIAC

SEQ ID NO: 17 - DNA, 2277 bp, Quillaja Saponaria
  source 1..2277: mol_type = other DNA
atgtggaggctgaagatagcagaaggtggttccgatccatatctgttcagcacaaacaacttcgtgggtcgccagacatgggagttcgaaccggaggccggcacacctgaggagcgagcagaggtcgaagctgcccgccaaaacttttacaacaaccgttaccaggtcaagccctgtgacgacctcctttggagatatcagttcctgagagagaagaatttcaaacaaacaataccgcctgtcaaggttgaagatggccaagaaattacttatgagatggccacaacctcaatgcagagggcggcccgtcacctatcagccttgcaggccagcgatggccattggccagctcaaattgctggccccttgttcttcatgccacccttggtcttttgtgtgtacattactgggcatcttaatacagtattcccatctgaacatcgcaaagaaatccttcgttacatgtactatcaccagaacgaagatggtgggtggggactgcacatagagggtcacagcaccatgttttgcacagcactcaactacatttgtatgcgtatccttggggaaggaccagaggggggtcaagacaatgcttgtgccagagcacgaatgtggattcttgatcatggtggtgtaacacatattccatcttggggaaagacctggctttcgatacttggtctatttgagtggtctggaagcaatccaatgcctccagagttttggatccttccttcatttcttcctatgcatccagcaaaaatgtggtgctattgccggatggtttacatgcccatgtcttatttatatgggaaaaggtttgttggcccaatcacgcctctcattgttcagttaagagaggaaatacacactcaaaattaccatgaaatcaactggaagtcagtccgccatctatgtgcaaaggaggatatctactatccccatccactcatccaagatttgatttgggacagtttgtacatactaacggagcctcttctcactcgctggcccttgaacaagttggtgcgggagagggctctccaagtaacaatgaagcatatccactatgaagatgaaaatagtcgatacataaccattggatgtgtggaaaaggtgttatgtatgcttgcttgttgggttgatgatccaaatggagatgctttcaagaagcaccttgctcgagtcccagattacgtatgggtctctgaagatggaattactatgcagagttttggtagtcaagaatgggatgctggctttgccgtccaggctctgcttgcttctaatcttaccgaggaacttggccctgctcttgccaaaggacatgacttcataaagcaatctcaggttaaggacaatccttcaggtgacttcaaaagcatgtatcgtcacatttctagaggatcatggaccttctctgaccaagatcatggatggcaagtttctgattgcactgcagaaggtctgaagtgttgcctgcttttgtcgatgttgccaccagaaattgttggtgaaaaaatggaaccacaaaggctatttgattctgtcaatgtgctgctctctctacagagcaaaaaaggtggtttagctgcctgggagccagcaggggcgcaagattggttggaattactcaatcccacagaattttttgcggacattgtcgttgagcatgaatatgttgaatgtactggatcagcaattcaggcattagttttgttcaagaagctgtatccggggcacaggaaaaaagagattgacagtttcattacaaatgctgtccggttccttgagaatacacaaacggcagatggctcttggtatggaaactggggagtttgcttcacctatggttgttggttcgcactgggagggctagcagcagctggcaagacttacaacaactgtcctgcaatacgcaaagctgttaatttcctacttacaacacaaagagaagacggtggttggggagaaagctatctttcaagcccaaaaaagatatatgtacccctggaaggaagccgatcaaatgtggtacatactgcatgggctatgatgggtctaattcatgctgggcaggctgaaagagactcaactcctcttcatcgtgcagcaaagttgatcatcaattatcaactagaaaatggcgattggccgcaacaggaaatcactggagtattcatgaaaaactgcatgttacattaccctatgtacagaaacatctacccaatgtgggctcttgcagaataccggaggcgggttccattgccttaa

SEQ ID NO: 18 - protein, 758 aa, Quillaja Saponaria
MWRLKIAEGGSDPYLFSTNNFVGRQTWEFEPEAGTPEERAEVEAARQNFYNNRYQVKPCDDLLWRYQFLREKNFKQTIPPVKVEDGQEITYEMATTSMQRAARHLSALQASDGHWPAQIAGPLFFMPPLVFCVYITGHLNTVFPSEHRKEILRYMYYHQNEDGGWGLHIEGHSTMFCTALNYICMRILGEGPEGGQDNACARARMWILDHGGVTHIPSWGKTWLSILGLFEWSGSNPMPPEFWILPSFLPMHPAKMWCYCRMVYMPMSYLYGKRFVGPITPLIVQLREEIHTQNYHEINWKSVRHLCAKEDIYYPHPLIQDLIWDSLYILTEPLLTRWPLNKLVRERALQVTMKHIHYEDENSRYITIGCVEKVLCMLACWVDDPNGDAFKKHLARVPDYVWVSEDGITMQSFGSQEWDAGFAVQALLASNLTEELGPALAKGHDFIKQSQVKDNPSGDFKSMYRHISRGSWTFSDQDHGWQVSDCTAEGLKCCLLLSMLPPEIVGEKMEPQRLFDSVNVLLSLQSKKGGLAAWEPAGAQDWLELLNPTEFFADIVVEHEYVECTGSAIQALVLFKKLYPGHRKKEIDSFITNAVRFLENTQTADGSWYGNWGVCFTYGCWFALGGLAAAGKTYNNCPAIRKAVNFLLTTQREDGGWGESYLSSPKKIYVPLEGSRSNVVHTAWAMMGLIHAGQAERDSTPLHRAAKLIINYQLENGDWPQQEITGVFMKNCMLHYPMYRNIYPMWALAEYRRRVPLP

SEQ ID NO: 19 - DNA, 1443 bp, Quillaja Saponaria
  source 1..1443: mol_type = other DNA
atggagcacttgtatctctcccttgtgctcctgtttgtttcctcaatctccctctccctcttcttcctgttctacaaacacaaatctatgttcaccggggccaacctaccacctggtaaaatcggttacccattgatcggagagagcttggagttcttgtccacgggatggaagggccacccggagaaattcatcttcgatcgcatgagcaagtactcatcccaaatcttcaagacctcgattttaggggaaccaacggcggtgttcccgggagccgtatgcaacaagttcctcttctccaacgagaacaagctggtgaatgcatggtggcctgcctccgtggacaagatctttccttcctcactccagacatcctccaaagaagaggccaagaagatgaggaagttgcttcctcagtttctcaagcccgaagctctgcaccgctacattggtattatggattctattgcccagagacactttgccgatagctgggaaaacaaaaaccaagtcattgtctttcctctagcaaagaggtatactttctggctggcttgccgtttgttcattagcgtcgaggatccgacccacgtatccagatttgctgacccgttccaacttttggccgccggaatcatatcaatcccaatcgacttgccagggacaccgttccgcaaggcaatcaatgcgtcccagttcatcaggaaggaattgttggccatcatcaggcagagaaagatcgatttgggtgaagggaaggcatctccgacgcaggacatactgtctcacatgttgctcacatgcgacgagaacggacaatacatgaatgaattggacattgccgacaagattcttggcttgttggtcggcggacatgacactgccagtgccgcttgcactttcattgtcaagttcctcgctgagcttccccacatttatgaacaagtctacaaggagcaaatggagattgcaaaatcaaaagtgccaggagagttgttgaattgggaggacatccaaaagatgaaatattcgtggaacgtagcttgtgaagtgatgagacttgcccctccactccaaggagctttcagggaagccattactgacttcgtcttcaacggtttctccattccaaaaggctggaagttgtactggagcgcaaattccacccacaaaagtccggattatttccctgagcccgacaagttcgacccaactagattcgaaggaaatggacctgcgccttacacctttgttccatttgggggaggacccaggatgtgcccgggcaaagagtatgcccgattggaaatacttgtgttcatgcataacttggtgaagaggttcaagtgggagaaattggttcctgatgaaaagattgtggttgatccaatgcccattccagcaaagggtcttcctgttcgcctttatcctcacaaagcttga

SEQ ID NO: 20 - protein, 480 aa, Quillaja Saponaria
MEHLYLSLVLLFVSSISLSLFFLFYKHKSMFTGANLPPGKIGYPLIGESLEFLSTGWKGHPEKFIFDRMSKYSSQIFKTSILGEPTAVFPGAVCNKFLFSNENKLVNAWWPASVDKIFPSSLQTSSKEEAKKMRKLLPQFLKPEALHRYIGIMDSIAQRHFADSWENKNQVIVFPLAKRYTFWLACRLFISVEDPTHVSRFADPFQLLAAGIISIPIDLPGTPFRKAINASQFIRKELLAIIRQRKIDLGEGKASPTQDILSHMLLTCDENGQYMNELDIADKILGLLVGGHDTASAACTFIVKFLAELPHIYEQVYKEQMEIAKSKVPGELLNWEDIQKMKYSWNVACEVMRLAPPLQGAFREAITDFVFNGFSIPKGWKLYWSANSTHKSPDYFPEPDKFDPTRFEGNGPAPYTFVPFGGGPRMCPGKEYARLEILVFMHNLVKRFKWEKLVPDEKIVVDPMPIPAKGLPVRLYPHKA

SEQ ID NO: 21 - DNA, 1506 bp, Quillaja Saponaria
  source 1..1506: mol_type = other DNA
atgatatataataatgatagtaatgataatgaattagtaatcagctcagttcagcaaccatccatggatcctttcttcatttttggcttacttctcttggctctctttctctctgtttcttttcttctctacctttcccgtagagcctatgcttctctccccaaccctccgccggggaagctcggcttccccgtcgtcggcgagagtctcgaatttctctccacccgacgcaaaggtgttcctgagaaattcgtcttcgacagaatggccaaatactgtcgggatgtctttaagacatcaatattgggagcaaccaccgccgtcatgtgcggcaccgccggtaacaaattcttgttctccaacgagaaaaaacacgtcactggttggtggccgaaatctgtagagctgattttcccaacctcacttgagaaatcatccaacgaagaatccatcatgatgaaacaattccttcccaacttcttgaaaccagaacctttgcagaagtacatacccgttatggacataattacccaaagacacttcaatacaagctgggaaggacgcaacgtggtcaaagtgtttcctacggctgccgaattcaccacgttgctggcttgtcgggtattcctcagtgttgaggatcccattgaagtagccaagatttcagagccatttgaaatcttagctgctgggtttctttcaatacccataaatcttccgggtaccaaattaaataaagcggttaaggcagcggatcagattagagacgcaattgtacagattttgaaacggagaagggttgaaattgcggagaataaagcaaatggaatgcaagatatagcgtccatgttgttgacgacaccaactaatgctgggttttatatgaccgaggctcacatttctgagaaaattttgggtatgattgttggtggccgtgatactgctagtactgttatcaccttcatcatcaagtatttggcagagaatcctgaaatttataataaggtctatgaggagcaaatggaagtggtaaagtcaaagaaaccaggtgagttgctgaactgggaagatgtgcagaaaatgaagtactcttggtgcgtagcatgtgaagctatgcgacttgctcctcctgttcaaggtggtttcaaggtggccattaatgactttgtgtattctgggttcaacattcgcaagggttggaagttatattggagtgccattgcaacacacatgaatccagaatatttcccagaacctgagaaattcaacccctcaaggtttgaagggaagggaccagtaccttacagcttcgtacccttcggaggcggacctcggatgtgtcccgggaaagagtattcccggctggaaacacttgttttcatgcatcatttggtgacgaggtacaattgggagaaagtgtatcccacagagaagataacagtggatccaatgccattccctgtcaacggcctccccattcgccttattcctcacaagcaccaatga

SEQ ID NO: 22 - protein, 501 aa, Quillaja Saponaria
MIYNNDSNDNELVISSVQQPSMDPFFIFGLLLLALFLSVSFLLYLSRRAYASLPNPPPGKLGFPVVGESLEFLSTRRKGVPEKFVFDRMAKYCRDVFKTSILGATTAVMCGTAGNKFLFSNEKKHVTGWWPKSVELIFPTSLEKSSNEESIMMKQFLPNFLKPEPLQKYIPVMDIITQRHFNTSWEGRNVVKVFPTAAEFTTLLACRVFLSVEDPIEVAKISEPFEILAAGFLSIPINLPGTKLNKAVKAADQIRDAIVQILKRRRVEIAENKANGMQDIASMLLTTPTNAGFYMTEAHISEKILGMIVGGRDTASTVITFIIKYLAENPEIYNKVYEEQMEVVKSKKPGELLNWEDVQKMKYSWCVACEAMRLAPPVQGGFKVAINDFVYSGFNIRKGWKLYWSAIATHMNPEYFPEPEKFNPSRFEGKGPVPYSFVPFGGGPRMCPGKEYSRLETLVFMHHLVTRYNWEKVYPTEKITVDPMPFPVNGLPIRLIPHKHQ

SEQ ID NO: 23 - DNA, 1524 bp, Quillaja Saponaria
  source 1..1524: mol_type = other DNA
atgtggttcacagtaggattggtcttggttttcgccctattcatacgtctctacagcagtctgtggttgaagcctcgtgcaactcggattaagcttagcaatcaaggaattaaaggtccaaaaccagcatttcttctgggtaatgttgcagagatgagaagatttcaatctaagcttccaaaatctgaactcaaacaaggccaagtttctcatgattgggcttctaaatctctgtttccatttttcagtctttggtcccagaaatacggaaatacgttcgtgttctcattggggaacatacaggtgctctatgtttctgatcatgagttggtgaaagaaattaatcagaatacctctttagatttgggcaaacccaagtacctgcagaaggagcgtggccctttgctgggacaaggtattttgacctccaatggacagctttgggcgtaccagagaaaaatcatgactcctgaactctacaaggagaaaatcaagggcatgtgcgagttgatggtggaatctgtagcttggttggttgaggaatggggaacgaagatccaagctgagggtggggcagcagacattagaatagacgaggatcttagaagcttctctggtgatgtaatttcaaaagcttgttttgggagctgctatgccggagggagggaaatctttcttaggctcagagctcttcaacaccaaattgcttccaaagccttactcatgggcttccctggattaaagtacctgcccattaagagcaacagagagatatggagattggagaaggagatcttccagctgattatgaagctggctgaagatagaaaaaaagaacaacatgagagagacctattacagattataattgagggagctaaaagtagtgatctgagttcggaagcaatggcaaaattcattgtggacaactgcaagaatgtctacttggctggccatgaaactactgcaatgtctgctggttggactttgcttctcttggctaatcatcctgagtggcaagcccgtgtccgtgatgagattttacaagtcaccgagggccgcaatcctgattttgacatgctgcacaagatgaaactgttaacaatggtaattcaggaggcactgcgactctacccaacagtcatattcatgtcaagagaagcattggaagatattaatgttggaaacatccaagttccaaaaggtgttaacatatggatacctgtggtaaatcttcaaagggacacaacggtatggggtgcagacgcaaacgagtttaatcctgaaaggtttgccaatggagttaacaattcatgcaaggttccacaactttacctaccatttggagctggacctcgcatttgtcctggaattaatctggccatgactgagatcaagatacttctgtgtatcctgctcaccaagttttcgttttcagtttcacccaactatcgccactcaccggtgtttaaattggtgcttgagcctgaaaatggaatcaatgtcatcatgaagaagctctaa

SEQ ID NO: 24 - protein, 507 aa, Quillaja Saponaria
MWFTVGLVLVFALFIRLYSSLWLKPRATRIKLSNQGIKGPKPAFLLGNVAEMRRFQSKLPKSELKQGQVSHDWASKSLFPFFSLWSQKYGNTFVFSLGNIQVLYVSDHELVKEINQNTSLDLGKPKYLQKERGPLLGQGILTSNGQLWAYQRKIMTPELYKEKIKGMCELMVESVAWLVEEWGTKIQAEGGAADIRIDEDLRSFSGDVISKACFGSCYAGGREIFLRLRALQHQIASKALLMGFPGLKYLPIKSNREIWRLEKEIFQLIMKLAEDRKKEQHERDLLQIIIEGAKSSDLSSEAMAKFIVDNCKNVYLAGHETTAMSAGWTLLLLANHPEWQARVRDEILQVTEGRNPDFDMLHKMKLLTMVIQEALRLYPTVIFMSREALEDINVGNIQVPKGVNIWIPVVNLQRDTTVWGADANEFNPERFANGVNNSCKVPQLYLPFGAGPRICPGINLAMTEIKILLCILLTKFSFSVSPNYRHSPVFKLVLEPENGINVIMKKL

SEQ ID NO: 25 - DNA, 2142 bp, Quillaja Saponaria
  source 1..2142: mol_type = other DNA
atgaaatccccctctaacccaaatcagaaacccatcctccacacttgtacaattcagcagcctcgtgctacccttaacaaaattcatagtcttattcatttctcagccatacttgtcctattttattaccggataacccgtctattcttcaccgacgatttcaaggtacccaagttactatggactctaatgacaatctccgagttcattcttgccttcatttgggttctcatccaacctttccggtggcgaccggtgtcccgttccgtcataccagagaatatgccgaaggacatcagtttgccggcggtggacgtgtttgtatgcacagctgaccctcaaaaagaacccacagtggaggtgatgaacacaattttatcagccatggctttagactacccggcggagaagctcgccgtgtatctttccgatgatgggggttctgctgtcaccttatatgctataaaagaagcttgttgttttgctaagatgtggcttccgttttgtaacaagtatgggatcaaatcaaggtgtcccgaggcttatttttcaaagcttgccgctgacgagtggcttcaccggagtgtggaattcgtggcagaagaaaaggaggtcaaggctaattatgaagagttcaagagaaatgtgcagaaatttggtgagcaacaagaaaacagtcgtgttgtgcatgatcgtcaccctcatgttgagattatacacaataattggaataacgaagaccaagctcatgagatgccactccttgtttatgtctctcgtgaaagaagaccatctcaccatcctcgattcaaagctggagctcttaacacccttcttcgagtttctggcatcatcagcaacagcccctacatactggttctagactgtgacatgtactgcaatgacccaacctcagctagacaagcaatgtgcttccatcttgatccccaactgtctaaaaatcttgcttttgtacaattccctcaaatattctataacgctagtaagaatgacgtctatgatgcccaagtcagggcggcataccagacaaagtggcagggtatggatggacttcaaggaccaattttttctggcactggcttttacttaaagaggaaggcaatgtatggaaaccctgatcaagatgataattgtctactcaagccatataagaaatttggcatgtctggagaatttgtagaatcacttaaggtccttaacgaacaagatggtacccagaagaaattattggatggatttttacaagaggccaaactattggcctcgtgtgcctatgaaacaaagacaagttggggtaaagagattggattctcatatgactgtttaatagagagcactttcactggttatcttttgcactgcagagggtggatatctgtttatctttatcccaagagaccatgttttttaggatgctgtcctactgatatgaaggatgccatggttcaatataccaagtggatgtctgagctattttcaattgctatctcaagattcaatcctctgctctatggggtggcaagaatgtccattcttcaaagcctgtgttatggatcctttacactggcgcctattttgtcatttcctttgttcttatatggaacggttcctcaattatgcctcttgaaaggcatatctttgtttccaaaggtttcggacccatggtttgctgtgtttgcagctatctttgtatcctccctgtgtcaacactggttcgaggtcctctcttgtgatggtacatttacgacttggtgtaatgaacagcggagttggcttataaagtcggtttccggtagtttgtttggagttgtgggcgcaatcttgcagcggctaggcttgaagacaaagtttagtttatcaaacaaagccatggacaaagaaaagctggagaaatatgaaaagggtaaatttaatttccaaggggctgccatgttcatggttcctgtgtctattttagtcatactgaacacattttgcttcctcggtgggttttggaaagtgatcataatgaagaatatcctggacatgtttggacaactttctctctctgcctacgttctggttctcagttgtccagttcttgaagggatgttaactagaatcagcaagaaaatggtctga

SEQ ID NO: 26 - protein, 713 aa, Quillaja Saponaria
MKSPSNPNQKPILHTCTIQQPRATLNKIHSLIHFSAILVLFYYRITRLFFTDDFKVPKLLWTLMTISEFILAFIWVLIQPFRWRPVSRSVIPENMPKDISLPAVDVFVCTADPQKEPTVEVMNTILSAMALDYPAEKLAVYLSDDGGSAVTLYAIKEACCFAKMWLPFCNKYGIKSRCPEAYFSKLAADEWLHRSVEFVAEEKEVKANYEEFKRNVQKFGEQQENSRVVHDRHPHVEIIHNNWNNEDQAHEMPLLVYVSRERRPSHHPRFKAGALNTLLRVSGIISNSPYILVLDCDMYCNDPTSARQAMCFHLDPQLSKNLAFVQFPQIFYNASKNDVYDAQVRAAYQTKWQGMDGLQGPIFSGTGFYLKRKAMYGNPDQDDNCLLKPYKKFGMSGEFVESLKVLNEQDGTQKKLLDGFLQEAKLLASCAYETKTSWGKEIGFSYDCLIESTFTGYLLHCRGWISVYLYPKRPCFLGCCPTDMKDAMVQYTKWMSELFSIAISRFNPLLYGVARMSILQSLCYGSFTLAPILSFPLFLYGTVPQLCLLKGISLFPKVSDPWFAVFAAIFVSSLCQHWFEVLSCDGTFTTWCNEQRSWLIKSVSGSLFGVVGAILQRLGLKTKFSLSNKAMDKEKLEKYEKGKFNFQGAAMFMVPVSILVILNTFCFLGGFWKVIIMKNILDMFGQLSLSAYVLVLSCPVLEGMLTRISKKMV

SEQ ID NO: 27 - DNA, 2124 bp, Quillaja Saponaria
  source 1..2124: mol_type = other DNA
atggcgaccgtctcctccctccacacttgcactgtacagcaaccccgtgcagccattaatcgaattcacattttcttacactttattgccatacttttcctcttttactaccgggtcaccggtcttttctatgacaatgcagtacccactttagcttggtctctaatgaccttagctgagttgattttcgccttcgtttgggtgctcagccaagccttccggtggcgcccggtgttgcgttcagttattcctgagaggattcccaaagatgtacgattgcccgcggtggatatcttaatttgtacggctgacccattaaaggaaccgacggtggaggtgatgaacactgtcttgtccgccatggcattggactatcctgcggagaatctggctgtatatctttctgatgacgggggttctccggtcaccttatttgctatgaagcaagtgggtccgtttgctaagctgtggcttccgttttgcaacaagtacggaatcaaaacaaggcatcctgagtcttttttctcggcatttgcggatgacgaaaggcttcaccggagtgatgaattcagggcagaggaggaggcgatcaaggacaaatatgaagaatttaagagaactatagagaaatatggtggagaaggaaaaaatagtcatgttgtacaagaccggcctcctcatgtggagattatacatgacactaggaagattagagagaacagtgaagaccaagctgtgcctcttcttgtctacgtctctcgtgagaaaagaccatcctacaattctcggttcaaagcaggagctctgaacacccttcttcgagtttctggggtaatcagcaatagcccatatgtattggtgttagactgtgacatgtactgcaatgatccaacatcagctagacaagcaatgtgcttccatcttgatccacaaatgtctcgcactctctcttttgtacaattcccccaggttttctacaatgttagtaaaaatgatatctatgatggccaagctagggcagcctttaagacaaagtggcaaggtatggatggactacgtgggccactgctttctggtactggcttttatttgaagaggaagtccttgtatggaagtccaaaccaagaagatgattgtttacttgagccccataagaattttggaaagtgtgacaagctcatagaatcagtaaaggtcatttatgaacgtgatgtttcaataaaggcagattcatcagatgccattttgcaagatgccaaacaattagcatcttgtccctatgaaacaaacacaagctggggcaaagaggttgggttctcgtatgactgcttattagagagtacattcacaggttatctgttgcactgcagagggtggacatcagtttatctttatccaaagaagccatgtttcttagggtgtactccagttgatatgaaggaagccatggttcagtatacgaagtggatttctgaattatttttacttgctatctcaagattcaaccctctgacatttgggatatccagaatgtccattctccagagcatgtgttacggataccttacaatcatgcccattttatctgttgctatgatcttctatgccacagttcctcaattgtgcctcttgagaggcgtacctctgtttcccaaggtttcagacccatggtttgcagtgttcctagcaatatttgtgtcctccctctgtcagcacttaattgaagtcctcacgagtgatggcacgctcaagacttggtggaatgaacaaagaaattgggtgataaagtctggttccggtagcgtatttggagctctgagtggaatattgaagtggtttggcatgaagattaaatttggtttatcaaacaaagccgtggacaaagaaaagcttgagaaatatgaaaagggtaagtttgatttccaaggggctgccatgtttatggttcccttaactatatcagtcatcttgaacacattatgccttatcggtggtttatggagagtaatcacacttaaaaacttcgaagagatgtcagggcagttcatcatctccttgtactttctagctctcagctatccaattcttgaagggttactaagaaaaggcaagggaaaggcctaa

SEQ ID NO: 28 - protein, 707 aa, Quillaja Saponaria
MATVSSLHTCTVQQPRAAINRIHIFLHFIAILFLFYYRVTGLFYDNAVPTLAWSLMTLAELIFAFVWVLSQAFRWRPVLRSVIPERIPKDVRLPAVDILICTADPLKEPTVEVMNTVLSAMALDYPAENLAVYLSDDGGSPVTLFAMKQVGPFAKLWLPFCNKYGIKTRHPESFFSAFADDERLHRSDEFRAEEEAIKDKYEEFKRTIEKYGGEGKNSHVVQDRPPHVEIIHDTRKIRENSEDQAVPLLVYVSREKRPSYNSRFKAGALNTLLRVSGVISNSPYVLVLDCDMYCNDPTSARQAMCFHLDPQMSRTLSFVQFPQVFYNVSKNDIYDGQARAAFKTKWQGMDGLRGPLLSGTGFYLKRKSLYGSPNQEDDCLLEPHKNFGKCDKLIESVKVIYERDVSIKADSSDAILQDAKQLASCPYETNTSWGKEVGFSYDCLLESTFTGYLLHCRGWTSVYLYPKKPCFLGCTPVDMKEAMVQYTKWISELFLLAISRFNPLTFGISRMSILQSMCYGYLTIMPILSVAMIFYATVPQLCLLRGVPLFPKVSDPWFAVFLAIFVSSLCQHLIEVLTSDGTLKTWWNEQRNWVIKSGSGSVFGALSGILKWFGMKIKFGLSNKAVDKEKLEKYEKGKFDFQGAAMFMVPLTISVILNTLCLIGGLWRVITLKNFEEMSGQFIISLYFLALSYPILEGLLRKGKGKA

SEQ ID NO: 29 - DNA, 1479 bp, Quillaja Saponaria
  source 1..1479: mol_type = other DNA
atggtggagtctccagcagatcatgatgtgctcaaaatcattgtccttccatgggtaacctcaggtcacatgattcccatggtagatgcagccagactatttgctatgcatggtgcagatgttaccatcatcaccaccccagctaatgcccttacattccagaaatccgtcgaccgtgatttcaattccggtcgtttaatcagaactcacacccttaaattccctgcagcagaagttggtgtacctgaaggagttgaaaacttcaacaatacttcccctgaaatgacctccaaagtctaccttggagtctcaatgctccgagaaccaacccaacaattgattgaggatctgcgtccagattgtcttatcactgatatgttctatccttgggctgtggatgttgctgacaaattaggcattccaaggctaatttttcaaggtcctggaagttttggtttgtcagctatgcattctatcaaacagtatgagccctttaagtcagtaacttcagatactgagacattcccactacctggattgccgcataaggtagagatgacaaggttgcagataccaaaatgggttcgtgagccaaatgggtacactcaattgatgggcagggtaaaagattcggagagaagaagctatgggtcattggtgaatagcttttatgacttcgaaggcccttatgaagagcactataggaaggcaacaggacagagggtttggagcattggaccagtttcagtttgggtgaaccaagatgctgcagataaggttggaagaggacaggatcttgttgctgaagaccaaaacagctggttgaattggctcaattccaaagagaaaaactctgttctgtatgtaagttttgggagcatggccaagttcccatctgctcagcttcttgaaatagctcatgggcttgaagcttcaggtcatagtttcatctgggttgtcagaaaagttgacggggatgatgatgtagacgtgtggcttccagattttgagaagaaaatgaaagagaacaacaagggtttcatcataaggaattgggcaccacaattgctcatattggaccatccagcaattggaggtttgctgaatcacagtggatggaattcagtactggaaggtgctacagcaggcttgccaatgatcacttggcctctgtatgccgagcatttttacaatgaaaggttggttctagatgtgttgaaaattggagtaccagttggggtgaaggagtggaagaacttgcatgaggtgggtgagttggtgagaagggatgcaattgccaaggcaattaaattgttaatgggtagtggagaagaagctgaggtaatgaggaaaaaagccaaagagcttggtgttggagcaaagaaaggtattcaggttggaggttcttctcataccaatttgatagcagtgattgatgagttaaagtcactaaagaaatcaagaattcagggtgtctga

SEQ ID NO: 30 - protein, 492 aa, Quillaja Saponaria
MVESPADHDVLKIIVLPWVTSGHMIPMVDAARLFAMHGADVTIITTPANALTFQKSVDRDFNSGRLIRTHTLKFPAAEVGVPEGVENFNNTSPEMTSKVYLGVSMLREPTQQLIEDLRPDCLITDMFYPWAVDVADKLGIPRLIFQGPGSFGLSAMHSIKQYEPFKSVTSDTETFPLPGLPHKVEMTRLQIPKWVREPNGYTQLMGRVKDSERRSYGSLVNSFYDFEGPYEEHYRKATGQRVWSIGPVSVWVNQDAADKVGRGQDLVAEDQNSWLNWLNSKEKNSVLYVSFGSMAKFPSAQLLEIAHGLEASGHSFIWVVRKVDGDDDVDVWLPDFEKKMKENNKGFIIRNWAPQLLILDHPAIGGLLNHSGWNSVLEGATAGLPMITWPLYAEHFYNERLVLDVLKIGVPVGVKEWKNLHEVGELVRRDAIAKAIKLLMGSGEEAEVMRKKAKELGVGAKKGIQVGGSSHTNLIAVIDELKSLKKSRIQGV

SEQ ID NO: 31 - DNA, 1515 bp, Quillaja Saponaria
  source 1..1515: mol_type = other DNA
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttaaaccacccggcgatcggtggtttcatgacgcattgtggttggaatgcggtggtggaagcactttcattcggtgttccgactattacgcttccagttttctcggagcagttttatactgagagactgatatcacaagtgctcaagactggtgtggaggttggtgcagagaagtggacctatgcatttgatgcggggaaatatccggtgagtagggaaaagatagcgacggcggtgaagaagatattagacgatggagaagaggcagaaggaatgagaaagcgggccagggagatgaaagaaaaagcccaaaaaagtgttgaagaaggtggatcctcttataataatttaacggctatgattgaagatcttaaagaatttagggctaacaatggcaaggctgcacaagatcatgaatcgtga

SEQ ID NO: 32 - protein, 504 aa, Quillaja Saponaria
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAVVEALSFGVPTITLPVFSEQFYTERLISQVLKTGVEVGAEKWTYAFDAGKYPVSREKIATAVKKILDDGEEAEGMRKRAREMKEKAQKSVEEGGSSYNNLTAMIEDLKEFRANNGKAAQDHES

SEQ ID NO: 33 - DNA, 1485 bp, Quillaja Saponaria
  source 1..1485: mol_type = other DNA
atggtctccggcgacgacgacgtttctcgtcggccactgaaagtttactttattgcacacccctcacctggccatattgcccctctaaccaaaatagcccaactctttgctgcacgtggtgagcacgtgactattcttactactcccgccaatgtccacttccatgagaaatccatcgacaaaggaaagacttccggctatcatgttaacatccacgccgttaaatttccttctaaagaggtcggtctccccgacggcatcgaaaacttctctcacgcctccgataatgaaacagcagccaaaatttgggccggattctccatgcttcaaactgaaatggagcaatatatggaacaaaacccacccgattgcattgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattccgggtctacccgaccccataacaattacccgagctcaactgcccgacggtgccttttctgtcgtcaaagaacaagttagtgaagctgagttgaaaagcttcggaatggtgatcaacgggttttccgaactcgaaaccgaatacatcgagtattacaagaatatcatgggtcgaaaacggatttggcatgtcggaccccttcagctcatttaccaaaacgatgaccccaaagttcagaggagccagaagacagcggtcgtgagtgacaacgagttagtgagttggcttgactcgaagaaacccgactcagtgatttacatttccttcggtagtgcaattcgtttctctaataagcagctctatgaaatagcatgtggattagaagcttccggctacccatttttgtgggccttactttgggtgccagaagatgacgacgacgtgggcaacaaatggttgcctgatttcgaagaaagaataaaaagagaaaataagggaataattttcagggggtgggccccacagatgttaatcttaaaccacccggcgatcggtggtttcatgacgcattgtggttggaatgcggtggtggaagcgctttctttcggtgttccgactattacgcttccggttttctcggagcagttttatactgagagactgatatcacaagtgctcaagactggtgtcgaggtcggtgcagagaagtggacctatgcatttgatgcggggaaatatccggtgagtcgggaaaagatagcgacggcggtgaagaagatattagactgtggagaagaggcagaaggaatgagaaagcgggccagggagatgaaagaaaaagcccaaaaaagtgttgaagaaggtgggtcctcttataataatttaacggctatgattgaagatcttaaagaatttagggctaacaatggcaaggttgcatga

SEQ ID NO: 34 - protein, 494 aa, Quillaja Saponaria
MVSGDDDVSRRPLKVYFIAHPSPGHIAPLTKIAQLFAARGEHVTILTTPANVHFHEKSIDKGKTSGYHVNIHAVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVVSDNELVSWLDSKKPDSVIYISFGSAIRFSNKQLYEIACGLEASGYPFLWALLWVPEDDDDVGNKWLPDFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDCGEEAEGMRKRAREMKEKAQKSVEEGGSSYNNLTAMIEDLKEFRANNGKVA

SEQ ID NO: 35 - DNA, 1491 bp, Quillaja Saponaria
  source 1..1491: mol_type = other DNA
atggtctccggcgacgataccgtttcacggccactgatagtttactttattgcacacccctcacctggccatattgcccctctaaccaaaatagcccaactcttcgctgcacgtggtgagcacgtcactattcttactactcccgccaatgtccacttccatgagaaatccatcgacaaaagaaagaattccggctatcatgttaacatccacaccgttaaatttccttctaaagaggtcggtctccctgacggcatcgaaaacttctctcacgcctccgataatgaaacagcagccaaaatttgggccggattctccatgcttcaaactgaaatggagcaatatatggaacaaaacccacccgattgcatcgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattcccggtctacccgaccccataacaattacccgagctcaactccccgacggtgccttttctgtcgtcaaagaacaagttagtgaagctgagttgaaaagcttcggaatggtgatcaacgggttttccgaactcgaaactgaatacatcgagtattacaagaatatcatgggtcgcaaacggatttggcatgtcggaccccttcagctaatttaccaaaacgacgaccccaaagttcagaggagccagaagacagcggtcttgagtgacaacgagttagtgagttggcttgactcgaagaaacccgactcagtgatttacatttccttcggtagtgcaattcgtttctctaataagcagctctatgaaatcgcatgtggattagaagcttccggctacccatttttgtgggccttactttgggtgccagaagatgatgacgacgtgggcaacaaatggttgccgggtttcgaagaaagaataaaaagagaaaataagggaataattttcagggggtgggccccacagatgttaatcttaaaccacccggcgatcggtggtttcatgacgcattgtggttggaatgcggtggtggaagcactttcattcggtgttccgactattacgcttccagttttctcggagcagttttatactgagagactgatatcacaagtgctcaagactggtgtggaggttggtgcagagaagtggacctatgcatttgatgcggggaaatatccggtgagtagggaaaagatagcgacggcggtgaagaagatattagacgatggagaagaggcagaaggaatgagaaagcgggccagggagatgaaagaaaaagcccaaaaaagtgttgaagaaggtggatcctcttataataatttaacggctatgattgaagatcttaaagaatttagggctaacaatggcaaggctgcaatgaaatcatga

SEQ ID NO: 36 - protein, 496 aa, Quillaja Saponaria
MVSGDDTVSRPLIVYFIAHPSPGHIAPLTKIAQLFAARGEHVTILTTPANVHFHEKSIDKRKNSGYHVNIHTVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVLSDNELVSWLDSKKPDSVIYISFGSAIRFSNKQLYEIACGLEASGYPFLWALLWVPEDDDDVGNKWLPGFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDDGEEAEGMRKRAREMKEKAQKSVEEGGSSYNNLTAMIEDLKEFRANNGKAAMKS

SEQ ID NO: 37 - DNA, 1515 bp, Quillaja Saponaria
  source 1..1515: mol_type = other DNA
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttgaatcacccggcgatcggtggtttcatgacgcattgtggttggaatgcggcggtggaggcgctttcttccggtgttccgattattacatttccggttttctcggatcagttttataatgaaaggctgatatcacaagtgcataagtgtggggtgggggttggtacggaggcgtggagctatgcattcgatgccgggaagaatccggtgggtcgggaaaagataatgacggcggtgaagaagatattagacggtggagaagaggcggaaggaatgagaaagagggcccgggagctgaaagaaatagctaaaagaagtgtggaagaaggtgggtcctcttataataatttaacggctatgattcaagatctgaaagaatttagagctaacaatggcaaggctgcacaagatcatgaatcgtga

SEQ ID NO: 38 - protein, 504 aa, Quillaja Saponaria
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAAVEALSSGVPIITFPVFSDQFYNERLISQVHKCGVGVGTEAWSYAFDAGKNPVGREKIMTAVKKILDGGEEAEGMRKRARELKEIAKRSVEEGGSSYNNLTAMIQDLKEFRANNGKAAQDHES

SEQ ID NO: 39 - DNA, 1053 bp, Acanthocystis turfacea chlorella
  source 1..1053: mol_type = other DNA
atgaatagtcaggagtatactcctaaatcagtccttgtaactggtggggcaggtttcattggcagccatgtcgtgatgaaattggtccagaggtatccggaatgcaaggtcgttgttcttgacaaaatggattactgcgctaccttaaataatcttgctacggtacgagatgcccccaattttaaatttgtaaaaggtgatatacaaagcactgatcttttagctcacgtgctcaaacaggaaaagatagataccattatgcactttgccgcccagacccatgtagataatagctttggcaatagtttagcatttacgatgaacaacgtatacggcactcacgtccttctcgaatgtgcccgattgtatggtggtgttcagagattcattaacgtgtcaactgatgaggtctacggtgagagttccttgggaaagaaggaggggttggacgaacactcctccctcgaaccgacaaatccttatgccgccgcaaaggctggagctgaaatgatggctagagcatatcatacgtcatacaaactcccggtaatagtcacgcgtggcaataatgtatatggtccgcaccagttccccgaaaaaatgatccccaaatttattctcagggcaaccagaggcctcgatttgccaatacatggtgatggcggcgccctgcgatcatacctttacgtcgatgatgttgccgaagcttatattacaattcttttaaagggcaatgttggagagacctataacatcggcacgcaaaaggagcgatccgttgtggacgtggcccatgacatctgcaagatttttaaccgagactcagatacagctatatggcacgttaaagaccgagccttcaatgatcgacgttattttatttctgataaaaaattacttgacctcggctggcaagaaaaaaccacctgggaggacggtcttaagcaaactgtagggtggtatttgcaacatgcaactaggtcctactgggatcacggcaacatggaattagctttagacgcccatccgacacttcaagttcctaaattctaa

SEQ ID NO: 40 - protein, 350 aa, Acanthocystis turfacea chlorella [sequence omitted]

SEQ ID NO: 41 - DNA, 693 bp, Aggregatibacter actinomycetemcomitans
  source 1..693: mol_type = other DNA
atgataattggcaacggtatgctggcaaaagcctttgaatcattccataagcgaacttacaattacataatatttgcatccggagtgagcaactcaaacgaaacttccttcgagaatttcaacagagagaaggaattgcttcttgaagtcctggagcaatataaagacaaaactatcgtttactttagttcctgctccatatacgattctagtttgacgaattctttgtatgtctaccacaaaatgtgtatggagagactggtgcgtgaaaactccaagaattatctcatagcccgtctcccccaagttattggtaaaacgtattcaccaaccattgtcaacttcctttttaacaaaatcaaaaatagggagtgtttcagcatattcggaaaagctcaccgaaattttatcgacgtggatgatgtcgttaaggtcaccaattacttattgaaggagggtctgttcattaacagtattgtgaacttggcaagcacgcaccatacctccatgtacgaattaatcttatatttggaaaaaataagtaatcaacgtgccttctataatgttgagaacaaagggtctaggtactttattgatgtttcaatactgcaggatgtttatcagaagctggggatcaaatttgacaaagattacgtagaaaaggttatcaacaagtactacgctattaagtaa

SEQ ID NO: 42 - protein, 230 aa, Aggregatibacter actinomycetemcomitans
MIIGNGMLAKAFESFHKRTYNYIIFASGVSNSNETSFENFNREKELLLEVLEQYKDKTIVYFSSCSIYDSSLTNSLYVYHKMCMERLVRENSKNYLIARLPQVIGKTYSPTIVNFLFNKIKNRECFSIFGKAHRNFIDVDDVVKVTNYLLKEGLFINSIVNLASTHHTSMYELILYLEKISNQRAFYNVENKGSRYFIDVSILQDVYQKLGIKFDKDYVEKVINKYYAIK

SEQ ID NO: 43 - DNA, 927 bp, Anoxybacillus tepidamans
  source 1..927: mol_type = other DNA
atgaagaggatactgatactcggatgcggttacctgggtttaaatctcgcaaactatttttgtaaaaaaaattatgatgtctcagtgatagggagaaagtctgtctatagcaattttttggaagaggagatagagttcatagaagatgatatcaaaaatataaatagttataagcacatgtttaatgaggagacaaccgtcatttacgccataggaagtattaacgcaaataactattttatggacctgaggaatgatatagaaaactcatacatccccttcattaacctccttaactttctttccgaaaagtatattcaaaagttcgtctttctctcttcagccggaacagtctatgggaacgtgaataagaattatataagcgagaatgagattcttaacccaatttcaatctatggtttgcagaaagccttctttgaacaactgataaggattaaaaacaatgaggctagccatttcaggtatttgatcttcagaatatctaacccctatgggggaatcaacattccgaacaagaatcagggaattattccgacgttagtgtacaaagccgtgaacaatgagcctttcgaactttgggcatcaatcaataccatccgtgattatatttacatcgatgaccttagcgaattgatctacaaaacaatctatctggacatttataacgagaccctcaatctcgggtccggtaaaggaacatcaatcaagcaactcattagcctcgtggaggagattttgggaaagaagatcactattcttgaaaagccccccataaagactaacgttttgaaaaatatacttgatatttctaagctcgtcaacaccgtaggctacgaaccaaagatcagcattgaagagggtattagccgttacatcaacactattttaacgaagaacattttttaa

SEQ ID NO: 44 - protein, 308 aa, Anoxybacillus tepidamans
MKRILILGCGYLGLNLANYFCKKNYDVSVIGRKSVYSNFLEEEIEFIEDDIKNINSYKHMFNEETTVIYAIGSINANNYFMDLRNDIENSYIPFINLLNFLSEKYIQKFVFLSSAGTVYGNVNKNYISENEILNPISIYGLQKAFFEQLIRIKNNEASHFRYLIFRISNPYGGINIPNKNQGIIPTLVYKAVNNEPFELWASINTIRDYIYIDDLSELIYKTIYLDIYNETLNLGSGKGTSIKQLISLVEEILGKKITILEKPPIKTNVLKNILDISKLVNTVGYEPKISIEEGISRYINTILTKNIF

SEQ ID NO: 45 - DNA, 951 bp, Echerichia coli
  source 1..951: mol_type = other DNA
atggatgctcgtaaaaatggggtattaataaccggtggagctgggttcataggtaaagccttaataactgaaatggtcgaacgtcaaattcccctggtgtcatttgacatcagcgataagcccgacagtttgccagagctttccgaatatttcaactggtataaattctcataccttgagagttcacagaggattaaagagcttcacgaaatagtttccaggcataacatcaaaacggtcatccatttagctacaactatgtttccccacgaatccaaaaagaacatcgataaggattgcttagaaaacgtttatgccaacgtgtgtttctttaagaatttatatgaaaacggctgtgaaaaaattatcttcgcctcatcaggtggcaccgtatatgggaagtctgatacacccttctccgaagacgatgccctgcttcccgaaatttcctacggactgtccaaggttatgactgaaacttatctccgattcatagccaaggaattgaatgggaagtccatctctctcagaatatctaacccctatggtgaggggcaaaggattgacgggaaacaaggagtcattccaattttcctcaataaaatcagcaacgacatccccatcgacatcattggctctatcgaatcaaagcgagactacatttatatttcagatctcgtacaagctttcatgtgctctctggaatatgaaggtcacgaagacatatttaatataggttctggggaaagcataactctgaagaaattgatcgagacgattgagttcaagctgaacaagaaggctgtgattggatttcaagatccgatccacaccaatgccaatggtataattctcgacatcaaacgagccatggcagaactcggctggaggcccaccgtggtcctggatgatggcatcgataaattaatcaagagcattcgatgcaagtaa

SEQ ID NO: 46 - protein, 316 aa, Echerichia coli
MDARKNGVLITGGAGFIGKALITEMVERQIPLVSFDISDKPDSLPELSEYFNWYKFSYLESSQRIKELHEIVSRHNIKTVIHLATTMFPHESKKNIDKDCLENVYANVCFFKNLYENGCEKIIFASSGGTVYGKSDTPFSEDDALLPEISYGLSKVMTETYLRFIAKELNGKSISLRISNPYGEGQRIDGKQGVIPIFLNKISNDIPIDIIGSIESKRDYIYISDLVQAFMCSLEYEGHEDIFNIGSGESITLKKLIETIEFKLNKKAVIGFQDPIHTNANGIILDIKRAMAELGWRPTVVLDDGIDKLIKSIRCK

SEQ ID NO: 47 - DNA, 897 bp, Quillaja Saponaria
  source 1..897: mol_type = other DNA
atggcagaagcaacagagaggtatgctgttgtgacaggatctaataaaggaattggatttgggatatgcaagcagctggcttctaaggggattacagtagtgctaacagctagagatgataagagaggtcttgaagcagttgagaaattgaaagaatttgatctgcatggtcatgtgctttttcatcaacttgatgtgtctgatacagctagtgttactagccttgcagattttatcaaaacccagtttgggaaactagatatcttggtaaacaatgcaggtataactggaaccactgtagatgctgatgctttagcatcttcaggctatggtactgggggtgaacgtaaacctattgattggaataaaatagtgatagagacttatgaatcagttgaaaaagctatcaacaccaactattatggagccaaaagaatggctgaagcacttataccccttcttcaagtatcagactcaccaaggattgttaatgcttcctctcctatggcaaagctagagaatattccaagtggatggggtaaggaagtgctaagtgatgttgatagcctaacagaagagaaacttgatgagatgttgacccaattattgaaagatttcaaagagggttcattagaaaccaaaggctggcctactcttatgtcttcgtatataatctcaaaagctgctttaaatgcctacacaaggattcttgctaagaagtacccatctttctgcatcaattgtgtagaccctggtcatgtgaagactgacataaatcgtcacaccggccacttaagtattgatgaaggtgctgaaagccatgtgagattggccctgctgcctgatggtggcccttctggacatttcttctccaggactgaagagacaccattttga

SEQ ID NO: 48 - protein, 298 aa, Quillaja Saponaria
MAEATERYAVVTGSNKGIGFGICKQLASKGITVVLTARDDKRGLEAVEKLKEFDLHGHVLFHQLDVSDTASVTSLADFIKTQFGKLDILVNNAGITGTTVDADALASSGYGTGGERKPIDWNKIVIETYESVEKAINTNYYGAKRMAEALIPLLQVSDSPRIVNASSPMAKLENIPSGWGKEVLSDVDSLTEEKLDEMLTQLLKDFKEGSLETKGWPTLMSSYIISKAALNAYTRILAKKYPSFCINCVDPGHVKTDINRHTGHLSIDEGAESHVRLALLPDGGPSGHFFSRTEETPF

SEQ ID NO: 49 - DNA, 915 bp, Quillaja Saponaria
  source 1..915: mol_type = other DNA
atgggttcagatggaagggatgtagcagagaggtatgcagtggttacaggtgcaaacaaaggcataggcctagaaaccgtgcggcaactagcgtctcacggcattacagttgtgttgacagctcgagatgagaagagagggactgaagccacaagaaagctccaccagctgggtttgtcaaatttgattttccatcagctggatgttttagaccctgttagcattcagtcactggccaagttcatccaagacaaatttggcaggcttgatatcctggttaataatgctggagcatctggacttgcagctgatgagaaagctctgaaggcattaaacatagataatgcagcttggctctcaggcaaggccgccaatttagttcaaggaattgtcacacatacctatgagcaaggcgaagaatgcataaatacaaactattatggtgtcaaaagggtgacggaagctctcctaccgctgttacaactttcccctataggagcaaggataataaatgtttcctcttgcaggggtgagctaaagaggattccaatgaacgtaagaaatgaactgggcgacatcaaagttctgactgaaggcagaatagatgcaattttgatgaaatttctacacgattttaaggataatgcacttgagtccaacggatggacattgatggggcctgcttatagcatttcgaaggccagtctcaatgcctacactagacttcttgccaaaaagtaccccgagatgctcattaactgtgttcatcctggttatgtcaacacagatatgacttggcatagagggatactgacggtagaagagggtgctaaaggcccagccatgctagctcttttgcaagatggaggacctacaggttgctattttgatagtactcaacaggcagaattttaa

SEQ ID NO: 50 - protein, 304 aa, Quillaja Saponaria
MGSDGRDVAERYAVVTGANKGIGLETVRQLASHGITVVLTARDEKRGTEATRKLHQLGLSNLIFHQLDVLDPVSIQSLAKFIQDKFGRLDILVNNAGASGLAADEKALKALNIDNAAWLSGKAANLVQGIVTHTYEQGEECINTNYYGVKRVTEALLPLLQLSPIGARIINVSSCRGELKRIPMNVRNELGDIKVLTEGRIDAILMKFLHDFKDNALESNGWTLMGPAYSISKASLNAYTRLLAKKYPEMLINCVHPGYVNTDMTWHRGILTVEEGAKGPAMLALLQDGGPTGCYFDSTQQAEF

SEQ ID NO: 51 - DNA, 909 bp, S. officianali
  source 1..909: mol_type = other DNA
atggctgaagcatcctcatttcttgcacagaaaaggtatgcggtcgtgacaggagcaaacaaaggactaggactagaaatatgcggacagcttgcttcacagggggtgacggtactgctgacatccagagatgaaaaacgaggcttagaagccattgaggagcttaagaaatcggggattaattcggaaaatcttgaatatcatcagctggatgttactaagccagctagtttcgcttctctggccgatttcatcaaggccaaatttggcaagcttgatatcctggtgaacaatgcagggatcagcggtgttattgtagattatgcagctttaatggaagccattcgccgtcgaggggcagagatcaattacgatggagtgatgaaacagacctacgagctagcagaggaatgcttgcaaacaaattactatggtgtgaaaagaaccattaatgctctccttccgctacttcagttttccgattcaccaaggatcgtcaatgtttcctccgatgttggcctccttaagaaaatacccggcgagagaatcagagaagccttaggcgacgtggaaaaacttacggaagaaagcgtggacgggattttagacgagtttctaagagatttcaaggaaggcaagatcgcagagaaaggttggcctacgtttaagagcgcctattcaatctcaaaggcggcgctcaattcgtacacgagggttttagcacggaaatacccgtcgatcatcatcaactgtgtctgcccgggtgtcgtcaaaaccgatatcaatcttaaaatgggccacttgacggttgaagaaggcgcggccagtcccgtgaggttagcactcatgccccttggttcgccttccggcctgttctatactcgaaacgaagtaactccatttgaatga

SEQ ID NO: 52 - protein, 302 aa, S. officianali
MAEASSFLAQKRYAVVTGANKGLGLEICGQLASQGVTVLLTSRDEKRGLEAIEELKKSGINSENLEYHQLDVTKPASFASLADFIKAKFGKLDILVNNAGISGVIVDYAALMEAIRRRGAEINYDGVMKQTYELAEECLQTNYYGVKRTINALLPLLQFSDSPRIVNVSSDVGLLKKIPGERIREALGDVEKLTEESVDGILDEFLRDFKEGKIAEKGWPTFKSAYSISKAALNSYTRVLARKYPSIIINCVCPGVVKTDINLKMGHLTVEEGAASPVRLALMPLGSPSGLFYTRNEVTPFE

SEQ ID NO: 53 - DNA, 1680 bp, Quillaja Saponaria
  source 1..1680: mol_type = other DNA
atggatcaattaaatccaaagcctgtaaactcatctcctctctcaccattaggcttcttggatagagctgccactgtgtacggtgactgcacctctatcatctacaataacatctcatacacgtggtcggatactcaccgtcgatgtcttcagctcgcttcctccatctcatccataggcatccagagaggccacgtcgtctccgttgtggcccccaatattccggccatgtacgagcttcacttcgctgttcctatgtccggtgctgtactcaacaatatcaatactcgacttgacgctcgtaccatctccatactcctgcttcacagcgaatcaaagctcgtcttcgttgaccttctctctcattctcttatcctcgagtcattatctctgtttccaccgcaaatcacacggccccgtctcatccttatcaccgatgatggggtcgacgctacacctaatccatcgcgtgattttcacgacacatatgaagggttattgagcaaaggagatcccaggtttatatgggtccggcctaactctgactgggacccaatagttctgaactatacgtccgggacgacgtcgtctccaaaaggagtcgtccactgccaccgaggagttttcatcatcactcttaattcgctcatagattggtccgtatcgaaacaaccggtttacttgtggaccctacccatgtttcactccaatggctggagctacccgtggggcatggcagctgttggtgggaccaacatttgcttccgtaagatcgacgcgcctacgatatacggtttgattgacaaacacagcgtgactcacatgtgtgccgcgcctgtggtactcaatatgttatctaatttcaacagaaccgaaccgatgaaaaacacggttcatattatgacaggcggagctccaccgcctgctgccatactctttcgggccgagtcgctgggttttgtagtggatcacgggtacgggttaaccgaaacatgtggggttatgatttcttgcgcttggaaatcgcagtggaaccggttgccggcgacggagagagcaaggctgaaatcaaggcaaggagtgagaacaattgggatgacggaggtggatgtggtggatcccgagtcaggactgagtgtaaaacgagacgggtcatcactcggtgagattgttctaagaggtgggtgcgtgatgttgggttatttcaaagacccaattggtacctccaaaagcttgaaagaaaatggttggttttatacaggggatgtgggtgtgatgcacccagatgggtatttagaaatcaaagatagatccaaggatgtaataataagcggcggagaaaatataagtagtgtggaggtggagggggcgttatatacacatccgtcggttgacgaggcggccgtggtggctcgaccagatgagttttggggggagacaccgtgtgcatttctgaaattgaaagatgggttggataagaaaccaaaggagagagagataatggagcactgtagagctacattgccacattacatggtgcctaaaacggtggcgtttatggaagagcttccaaaaacttcaactgggaaaattcagaaacacgtgcttagggagaaggcaaaagaaatgggatctttaacaggtggagtgacggacccgttgacagctagtcggttgtaa

SEQ ID NO: 54 - protein, 559 aa, Quillaja Saponaria
MDQLNPKPVNSSPLSPLGFLDRAATVYGDCTSIIYNNISYTWSDTHRRCLQLASSISSIGIQRGHVVSVVAPNIPAMYELHFAVPMSGAVLNNINTRLDARTISILLLHSESKLVFVDLLSHSLILESLSLFPPQITRPRLILITDDGVDATPNPSRDFHDTYEGLLSKGDPRFIWVRPNSDWDPIVLNYTSGTTSSPKGVVHCHRGVFIITLNSLIDWSVSKQPVYLWTLPMFHSNGWSYPWGMAAVGGTNICFRKIDAPTIYGLIDKHSVTHMCAAPVVLNMLSNFNRTEPMKNTVHIMTGGAPPPAAILFRAESLGFVVDHGYGLTETCGVMISCAWKSQWNRLPATERARLKSRQGVRTIGMTEVDVVDPESGLSVKRDGSSLGEIVLRGGCVMLGYFKDPIGTSKSLKENGWFYTGDVGVMHPDGYLEIKDRSKDVIISGGENISSVEVEGALYTHPSVDEAAVVARPDEFWGETPCAFLKLKDGLDKKPKEREIMEHCRATLPHYMVPKTVAFMEELPKTSTGKIQKHVLREKAKEMGSLTGGVTDPLTASRL

SEQ ID NO: 55 - DNA, 1653 bp, Quillaja Saponaria
  source 1..1653: mol_type = other DNA
atggaagatcttaagccaagcccagcaaattcatccccattaacgacattgggtttcttagaaagagcagccaccgtttacggtgattgcacttctatcatctacaacagcacctcatacacgtggacaaatactcaccgtcgatgtcttcaggttgcctcatcacttacatcactcggtatcaaacgaggccacgtggtctccgttgtggcccccaacatcccagccatgtacgagcttcacttcgctgttcccatgacaggtgctattctcaacgcaatcaaccttcgcctagacgcccgcgccatctctataatcctccgtcacgccgaatcaaatctcatctttgtcgattatctttcacgcgctcttgtccttgaagctttgtctttatttccatcacatatccaacgtccaaagctcatccttatcacagacgatagggtcgaaacactatcacccaccgttgatttccttgatacatatgaaggactggtgatgaaaggagatcctgggtttaactgggtccgacccatcagtgagtgggacccaatggtgttaaactacacgtcaggtacaacatcatctccaaagggtgtggtccattgtcatagaggaatattcgtcaccactgttgactctctgattgagtggaccgtccctaaacagccagtttacttgtggactctagccatgtttcatgctaatgggtggagcttcccgtgggggattgctgccttaggtgggaccaacatctgtgtccgtaaatttgatgcggatattgtgcaccgtctgatcgagcaacatggtgtaacccacatgtgcggtgcacctgtggtgctcaacatgttgtcaaagttaccaaatctaaaaccgctcaagaacccggttcatgttttcgctgcaggagctccaccacccgctgctattttatctcggtccgaatcgttgggttttgtggtgactcatgtctatgggttgactgaaacgggtgggttagtcgtgtcatgtgcttggaaacgtcagtggaaccggtttccggcgacagagcgagctcggttaaaggcaaggcaaggagtgaggacgattgggatgacggagatagacgtggtggatccggaatcaggaataagtgtgaaacgggacgggtcgtcactcggtgaggtcgtcttgcggggaggagcagtgatgttgggttatctgaaagatccgacgggtacctccaaatgtttgaaagaaaatggttggttctacaccggggacgttggtgtaatgcacccagatgggtatttagaaatcaaagaccgatcgaaggacgtaattatcagcggcggagagaatttgagcagcgtggaggttgagtccgtattgtacacccacccagcaattaacgaagcagctgttgtagcccgacccgacgaattctggggtgagactccatgtgcgtttgtgagctttaaaagccaactggataagaaacctacagagaaagatatcattgaatattgtagagctaagatgccgcattacatggtgcccaaaacggtggtgtttaaggaagagcttccaaaaacatcaaccgggaaaattcagaaatttttgctcagagagattgccaaaagtatgggcccactaagatcgagtcggatgtga

SEQ ID NO: 56 - protein, 550 aa, Quillaja Saponaria
MEDLKPSPANSSPLTTLGFLERAATVYGDCTSIIYNSTSYTWTNTHRRCLQVASSLTSLGIKRGHVVSVVAPNIPAMYELHFAVPMTGAILNAINLRLDARAISIILRHAESNLIFVDYLSRALVLEALSLFPSHIQRPKLILITDDRVETLSPTVDFLDTYEGLVMKGDPGFNWVRPISEWDPMVLNYTSGTTSSPKGVVHCHRGIFVTTVDSLIEWTVPKQPVYLWTLAMFHANGWSFPWGIAALGGTNICVRKFDADIVHRLIEQHGVTHMCGAPVVLNMLSKLPNLKPLKNPVHVFAAGAPPPAAILSRSESLGFVVTHVYGLTETGGLVVSCAWKRQWNRFPATERARLKARQGVRTIGMTEIDVVDPESGISVKRDGSSLGEVVLRGGAVMLGYLKDPTGTSKCLKENGWFYTGDVGVMHPDGYLEIKDRSKDVIISGGENLSSVEVESVLYTHPAINEAAVVARPDEFWGETPCAFVSFKSQLDKKPTEKDIIEYCRAKMPHYMVPKTVVFKEELPKTSTGKIQKFLLREIAKSMGPLRSSRM

SEQ ID NO: 57 - DNA, 1632 bp, Quillaja Saponaria
  source 1..1632: mol_type = other DNA
atggaagatcttaaaccaagctctgcaaattcatgccctttaaccacgcttggatttttggatagagcagccaccgtttacggtgattgcacctctatcatttacaatagtacctcatacacgtggtcaaccacccaccgtcgatgtctccaggttgcttcatctctcgcatcactcggcatcaagccaggccatgtcatctccgttttagcccccaatattcctgctatgtacgagcttcactttgctgttccaatgactggtgctatacttaacaccatcaatctccgtcttgacgcccgtaacatctccgtcatactccgtcacgctgagtcagatctcatctttgtcgattgtctttcacgggctactgttcttgaagctttatctttatttccaccgcatgttccccgtccccggctcatccttattacggatgatgcggtcgattcttcatcacctaccgttgattttattgatacttatgatggactgattgagaaaggggatcctgaattcaagtgggtccgacctagtagtgagtgggacccaatgactttgaattacacttctggtactacatcatcgccaaagggtgtggttcattgccatagaggggttttcatgatgacaattgattctttgattgactgggccgtacctaagcagccggtatatttgtgggccctacctatgttccatgctaatggatggaccttcccctgggggattgctgccatgggagggaccaatatctgtgtacgaaaattcaacgcggaattgatttaccgtttgatcgagcaatatgatgtgagccacatgtgtggtgcacctgtggtactcaacatgctatcaaattcgcctgaacagaaaccgctaaagaacccggttcagattctcactgctggagctccaccacctgcaacggttctcttccgaactgaatcactgggtttcgttgtgagtcatgggtacgggttgactgaaacagctgggttagtcgtttcctgtgcttggaagcaacaatggaatcgtttaccggcgaccgagagagctcgattgaaagcaaggcaaggagtgaggacaattgggatgacggcgatagacgtggtggaacccgaatcgggtatcagtgtgaaacgggacgggtcgtcactcggtgaaattgtgctacgaggcgggtcgattatgttgggttatctgaaagatctaaagggtacctccaaatccatgaaaaatggttggttccatactggagacgtgggtgtgatgcacccagatgggtacatagaaatcaaagatcgattgaaggatgtaattatcagcggtggagagaatctgagcagcgtggaggtggaatcaatgctgtacacccacccggatgttatcgaagcggcagtagtggcccgacccgatgagttctggggtgagacgccatgtgcatttgtgagcttaaaaggtggattggatgtgaaaccaacagagaaagagataattgaatattgtagatcaaagttgccccactttatggcgcccaaaactgtggtttttcaggaggagcttccaaaaacatccaccgggaaaattcagaagtttctgctcagagagattgccagaggtaagggcccataa

SEQ ID NO: 58 - protein, 543 aa, Quillaja Saponaria
MEDLKPSSANSCPLTTLGFLDRAATVYGDCTSIIYNSTSYTWSTTHRRCLQVASSLASLGIKPGHVISVLAPNIPAMYELHFAVPMTGAILNTINLRLDARNISVILRHAESDLIFVDCLSRATVLEALSLFPPHVPRPRLILITDDAVDSSSPTVDFIDTYDGLIEKGDPEFKWVRPSSEWDPMTLNYTSGTTSSPKGVVHCHRGVFMMTIDSLIDWAVPKQPVYLWALPMFHANGWTFPWGIAAMGGTNICVRKFNAELIYRLIEQYDVSHMCGAPVVLNMLSNSPEQKPLKNPVQILTAGAPPPATVLFRTESLGFVVSHGYGLTETAGLVVSCAWKQQWNRLPATERARLKARQGVRTIGMTAIDVVEPESGISVKRDGSSLGEIVLRGGSIMLGYLKDLKGTSKSMKNGWFHTGDVGVMHPDGYIEIKDRLKDVIISGGENLSSVEVESMLYTHPDVIEAAVVARPDEFWGETPCAFVSLKGGLDVKPTEKEIIEYCRSKLPHFMAPKTVVFQEELPKTSTGKIQKFLLREIARGKGP

SEQ ID NO: 59 - DNA, 1653 bp, Quillaja Saponaria
  source 1..1653: mol_type = other DNA
atggaaaaccttaagccaaccccagcaaattcctcgcctttaacgacactgggtttcttagaaagagcagccactgtttacggtgattgcacgtctatcatctacaataccacctcatacacgtggtcagatactcaccgtcgatgtctccaggttgcttcatctctagcatcactcggcatcaaaagaggccacgtggtttccgttgtggcccccaatatccctgccatgtacgaacttcactttgctgttcccatgacaggtgctgttctcaacacaatcaatcttcgtcttgacgcccgcaccatctctgtgatcctgcgtcacgccgaatcaaaactcataattgtcgattgtctttcacgcgctgttgtccttgaagctttatctctatttccacccaatattcaacgcccaatgctcatccttatcacggatgatacggttgacacttcatcacctaccgttgatttccttgatacgtatgaaggattggtgaagaaaggagatccagggtttaaatgggtccgacccatcaatgagtgggacccaatggtattgaactacacgtctggtacaacatctgctccaaagggtgtcgtcaattgtcatagagggatattcatcgttaccgttgattctttgattgagtgggcggttcccaaacagccggtttacttgtggaccctagcgatgttccatgctaatgggtggagctacccgtgggggattgctgctgtgggtgggaccaacatctgcatccgtaaattcgattcggatattgtataccgtctgattgaacaacgcggtgtcacccacatgtgtggtgcacccgtggtgcttaacatgttgtcaaattcacccaatcaaaaaccattgaagaacccggttcagattctaacggccggagctccaccaccagatgcaattctgtttcggaccgagtcgttgggtttcgtggtgagtcatggatatgggttgactgaaacggctggattagtcgtgtactgtgcttggaaacaacagtggaatcggttgccggcgacggagagggcacggcttaaagcaagacaaggagtgagaacaattgggatgactgaaatggatgtggtggatccggaaacaggaattagtgtgaaacgagacgggtcgtcactcggtgaggttgtcttacgaggcgggtcggtaatgttgggttatctcaaagatccaacgggtacatccaattgtatgaaagaaaatggttggttctacaccggagacgtgggtgtgatgcacccagatgggtatttacaaatcaaagaccgatctaaggacgtaatcatcagcggcggagagaatctgagcagcgtggaggttgagtctatattgtatacccacccggcaattaacgaagcagctgtggtagcccgacccgacgaattctggggcgagacgccatgtgcatttgtgagctttaaaagtcatttggataagaaacctgtggagaaagagataatggaatactgtagagctaagttgccacattacatggtgcccaaaacggtgatcttcaaggaagagcttccaaaaacttcaacagggaaaattcagaagtttttactcagagaggttgctaaaaatatgggcccaccaaggtcgagtcggatgtga

SEQ ID NO: 60 - protein, 550 aa, Quillaja Saponaria
MENLKPTPANSSPLTTLGFLERAATVYGDCTSIIYNTTSYTWSDTHRRCLQVASSLASLGIKRGHVVSVVAPNIPAMYELHFAVPMTGAVLNTINLRLDARTISVILRHAESKLIIVDCLSRAVVLEALSLFPPNIQRPMLILITDDTVDTSSPTVDFLDTYEGLVKKGDPGFKWVRPINEWDPMVLNYTSGTTSAPKGVVNCHRGIFIVTVDSLIEWAVPKQPVYLWTLAMFHANGWSYPWGIAAVGGTNICIRKFDSDIVYRLIEQRGVTHMCGAPVVLNMLSNSPNQKPLKNPVQILTAGAPPPDAILFRTESLGFVVSHGYGLTETAGLVVYCAWKQQWNRLPATERARLKARQGVRTIGMTEMDVVDPETGISVKRDGSSLGEVVLRGGSVMLGYLKDPTGTSNCMKENGWFYTGDVGVMHPDGYLQIKDRSKDVIISGGENLSSVEVESILYTHPAINEAAVVARPDEFWGETPCAFVSFKSHLDKKPVEKEIMEYCRAKLPHYMVPKTVIFKEELPKTSTGKIQKFLLREVAKNMGPPRSSRM

SEQ ID NO: 61 - DNA, 1680 bp, Quillaja Saponaria
  source 1..1680: mol_type = other DNA
atggagcaactcaacccaaaacctgttcactcatcgcctctcactccattaggtttcctggatagagctgcaaccgtatacggtgactgcacctctataatctacaacaacatgtcctacacgtggtctcttactcaccgtcgatgtctccagctagcttcatctctttcatctctcggcattaaaagaggccacgtcgtctccgttttgtctcccaatattcctgccatgtacgagcttcacttcgctgttcccatgtccggtgctgtactcaacaacatcagcaccagactcaacgcccgtaccgtctctatactcctgcgtcatagcgaatcaaagcttgtcttcgttgactctctctcttcgtctcttatccttgaatcgctttctcttttcccaccgcatgttcctccgccacgtctcgtccttatcaccgatgatgcgttcgaggctacaccgaatcaaccacgtgatttccatgacacttatgaagagtttgtcagtaagggagatcccggtttcaactggatccagcctaaatccgattgggacccaatggttctcaactacacttccggcacgacgtcgtctcccaaaggagttgtccactgccaccgaagtatcttcatcactaccctcgattctcttgtagattggtctgtaccgaaagaaccggtttacttatggactctacccatgtttcactcaaatggatggacctacccgtgggccatggcagcagttggtggtaccaacatttgcttccgtaaaatcgacgcgccaacaatctacggtctgattgacaaacacggcgtgactcacatgtgcgctgcacctgtggtgctcaacatgttatcaaactttaacagaaccgacccgttgaagaacccggttcatattttaacgggaggagctccaccacccgcttccatactctcccggacggagtcattgggtttcttggtgagtcacgggtacgggttaaccgaaaccggtggggttacgatttcttgcgcttggaaatcgaagtggaatttgttgccggcaacggagagagctaggttgaagtcaaggcagggagtgagaacagcaggggtgacggaggtggacgtggtagatcccgagtcaggactcagtgtgaaacgggacgggtcgtcacttggtgagatcgttttacgaggtgggtgtgtaatgttgggttatttcaaagacccgattggaaccaacaaatgcttgaaagaaaatggttggttttacacaggagatgtgggtgtgatgcacccagatgggtatatagaaataaaagatagatccaaggatataataattagcggcggagtgaatataagcagtgtagaggtagagacggtgttgtatacaaatccggcggtggatgaggctgcggtggtggctcgacctgatgagtattggggggaaacgccgtgtgggtttgtgaaattgaaagatgggttagatagaaaaccaaaggaaagagagataatggaatattgcagagctaaattgccacattacatggtgcccaaaacggtggtatttatggaagagcttccaaaatcttcaactggaaaaattcagaagcatgtgcttagggagaaggcaaaagccatggggccattaacaggtaaagccatggggtcgttgacagatagtcggttgtag

SEQ ID NO: 62 - protein, 559 aa, Quillaja Saponaria
MEQLNPKPVHSSPLTPLGFLDRAATVYGDCTSIIYNNMSYTWSLTHRRCLQLASSLSSLGIKRGHVVSVLSPNIPAMYELHFAVPMSGAVLNNISTRLNARTVSILLRHSESKLVFVDSLSSSLILESLSLFPPHVPPPRLVLITDDAFEATPNQPRDFHDTYEEFVSKGDPGFNWIQPKSDWDPMVLNYTSGTTSSPKGVVHCHRSIFITTLDSLVDWSVPKEPVYLWTLPMFHSNGWTYPWAMAAVGGTNICFRKIDAPTIYGLIDKHGVTHMCAAPVVLNMLSNFNRTDPLKNPVHILTGGAPPPASILSRTESLGFLVSHGYGLTETGGVTISCAWKSKWNLLPATERARLKSRQGVRTAGVTEVDVVDPESGLSVKRDGSSLGEIVLRGGCVMLGYFKDPIGTNKCLKENGWFYTGDVGVMHPDGYIEIKDRSKDIIISGGVNISSVEVETVLYTNPAVDEAAVVARPDEYWGETPCGFVKLKDGLDRKPKEREIMEYCRAKLPHYMVPKTVVFMEELPKSSTGKIQKHVLREKAKAMGPLTGKAMGSLTDSRL

SEQ ID NO: 63 - DNA, 1680 bp, Quillaja Saponaria
  source 1..1680: mol_type = other DNA
atggagcaactcaacccaaaacctgttcactcatcgcctctcactccattaggtttcctggatagagctgcaaccgtatacggtgactgcacctctataatctacaacaacatgtcctacacgtggtctcttactcaccgtcgatgtctccagctagcttcatctctttcatctctcggcattaaaagaggccacgtcgtctccgttttgtctcccaatattcctgccatgtacgagcttcacttcgctgttcccatgtccggtgctgtactcaacaacatcagcaccagactcaacgcccgtaccgtctctatactcctgcgtcatagcgaatcaaagcttgtcttcgttgactctctctcttcgtctcttatccttgaatcgctttctcttttcccaccgcatgttcctccgccacgtctcgtccttatcaccgatgatgcgttcgaggctacaccgaatcaaccacgtgatttccatgacacttatgaagagtttgtcagtaagggagatcccggtttcaactggatccggcctaaatccgattgggacccaatggttctcaactacacttccggcacgacgtcgtctcccaaaggagttgtccactgccaccgaagtatcttcatcactaccctcgattctcttgtagattggtctgtaccgaaagaaccggtttacttatggactctacccatgtttcactcaaatggatggacctacccgtgggccatggcagcagttggtggtaccaacatttgcttccgtaaaatcgacgcgccaacaatctacggtctgattgacaaacacggcgtgactcacatgtgcgctgcacctgtggtgctcaacatgttatcaaactttaacagaaccgacccgttgaagaacccggttcatattttaacgggaggagctccaccacccgcttccatactctcccggacggagtcattgggtttcttggtgagtcacgggtacgggttaaccgaaaccggtggggttacgatttcttgcgcttggaaatcgaagtggaatttgttgccggcaacggagagagctaggttgaagtcaaggcagggagtgagaacagcaggggtgacggaggtggacgtggtagatcccgagtcaggactcagtgtgaaacgggacgggtcgtcacttggtgagatcgttttacgaggtgggtgtgtaatgttgggttatttcaaagacccgattggaaccaacaaatgcctgaaagaaaatggttggttttacacaggagatgtgggtgtgatgcacccagatggttatatagaaataaaagatagatccaaggatataataattagcggcggagtgaatataagcagtgtagaggtagagacggtgttgtatacaaatccggcagtggatgaggctgcggtggtggctcgacctgatgagtattggggggaaacgccgtgtgggtttgtgaaattgaaagatgggttagatagaaaaccaaaggaaagagagataatggaatattgcagagctaaattgccacattacatggtgcccaaaacggtggtatttatggaagagcttccaaaatcttcaactggaaaaattcagaagcatgtgcttagggagaaggcaaaagccatggggccattaacaggtaaagccatggggtcgttgacagatagtcggttgtag

SEQ ID NO: 64 - protein, 559 aa, Quillaja Saponaria
MEQLNPKPVHSSPLTPLGFLDRAATVYGDCTSIIYNNMSYTWSLTHRRCLQLASSLSSLGIKRGHVVSVLSPNIPAMYELHFAVPMSGAVLNNISTRLNARTVSILLRHSESKLVFVDSLSSSLILESLSLFPPHVPPPRLVLITDDAFEATPNQPRDFHDTYEEFVSKGDPGFNWIRPKSDWDPMVLNYTSGTTSSPKGVVHCHRSIFITTLDSLVDWSVPKEPVYLWTLPMFHSNGWTYPWAMAAVGGTNICFRKIDAPTIYGLIDKHGVTHMCAAPVVLNMLSNFNRTDPLKNPVHILTGGAPPPASILSRTESLGFLVSHGYGLTETGGVTISCAWKSKWNLLPATERARLKSRQGVRTAGVTEVDVVDPESGLSVKRDGSSLGEIVLRGGCVMLGYFKDPIGTNKCLKENGWFYTGDVGVMHPDGYIEIKDRSKDIIISGGVNISSVEVETVLYTNPAVDEAAVVARPDEYWGETPCGFVKLKDGLDRKPKEREIMEYCRAKLPHYMVPKTVVFMEELPKSSTGKIQKHVLREKAKAMGPLTGKAMGSLTDSRL

SEQ ID NO: 65 - DNA, 1170 bp, Quillaja Saponaria
  source 1..1170: mol_type = other DNA
atggtgactgtggaggaagttcgcaaggctcaacgggcggaaggccctgccacgatcatggcaattggcacagctacacctctacattgtgtggagcaaagttcatatccggattattattttcgtatcacaaatagtgagcataaaacggaattaaaagaaaagttcaagcgaatgtgtgacaaatccatgattaagaagcgatacatgcacttgacagaggagattttgaaagagaatccaagtgtttgtgcctatatggcaccttcactggatgctaggcaggatatggtagtggttgaggtaccaaggctaggaaaagaggcagcaactaaggccatcaaggaatggggccagcccaaatccaagattacccacttagtcttttgcaccactagtggtgttgacatgcctggggctgactatcagctcacaaaactcttgggccttcgcccacatgtgaagcggctaatgatgtaccaacaagggtgctttgccggaggtacagttctccggctagccaaagacctagctgaaaacaacaaaggtgctcgtgtacttgtggtgtgttcggagatcacagccgtcacattccggggacctagtgatactcaccttgacagtcttgtgggccaagcattgtttggtgatggagctgccgcaatcattattggctcggacccaattcctgaagttgagaagcccttgtttgaactagtatcagcggcccaaacaattcttccggatagtgacggggctattgatggacaccttcgtgaagttgggcttacatttcatctcctcaaggatgttcctggactcatttcaaacaacattgagaaaagcttaaatgaagctttccaaccattaggtatctcagattggaactcactcttttggattgcacatccaggtgggccagcaattttggatcaagttgagcttaaattgggcctcaagcccgaaaaaatgcgggcaacccgacacgtgcttagcgagtatggtaacatgtcaagcgcgtgtgtgttgtttatcatggatgagatgaggaagaagtctgttgaagaaggacttggaaccagtggagaagggcttgaatggggtgtgctattcggctttggacctgggcttactgttgaaactgttgtgcttcatagcgtgtctgcttaa

SEQ ID NO: 66 - protein, 389 aa, Quillaja Saponaria
MVTVEEVRKAQRAEGPATIMAIGTATPLHCVEQSSYPDYYFRITNSEHKTELKEKFKRMCDKSMIKKRYMHLTEEILKENPSVCAYMAPSLDARQDMVVVEVPRLGKEAATKAIKEWGQPKSKITHLVFCTTSGVDMPGADYQLTKLLGLRPHVKRLMMYQQGCFAGGTVLRLAKDLAENNKGARVLVVCSEITAVTFRGPSDTHLDSLVGQALFGDGAAAIIIGSDPIPEVEKPLFELVSAAQTILPDSDGAIDGHLREVGLTFHLLKDVPGLISNNIEKSLNEAFQPLGISDWNSLFWIAHPGGPAILDQVELKLGLKPEKMRATRHVLSEYGNMSSACVLFIMDEMRKKSVEEGLGTSGEGLEWGVLFGFGPGLTVETVVLHSVSA

SEQ ID NO: 67 - DNA, 1179 bp, Quillaja Saponaria
  source 1..1179: mol_type = other DNA
atggcatccatcgaagcaatccgaaaggcccagcgtgccgaaggccctgccaccattttagccattggcaccgccacgccctccaattatgttcttcaagctgaatatcccgactactacttccgcatcaccaacagcgaacacatgacggatctcaaagagaaattcaagcgcatgtgtgacaaatccatgataaagaagcgatacttgtacttggatgaagaaattctgaaagagaacccaaatttatgtgcctacatggcaccttcccttgatgctcgtcaggatatagcagttgttgaagtaccaaagctgggaaaagaagcagctgtaaaggcaataaaacaatggggtcagcccaaatcaaagattacccatcttgtgttctgcaccacatctggtgtggacatgcccggatgtgattaccagctcactaaacttcttggccttagaccctctgtcaaacgactcatgatgtaccaacaaggttgtttcgccggtggaaccgtcctccgtcttgccaaggatttagctgaaaataataaaggtgcacgcgttcttgtcgtttgttctgagatcacagccgttactttccgtgggccctccgatactcacttggactctatggtgggtcaggcattgttcggtgacggcgcgggtgctcttattatcggttctgaacctgacaccaccgttgaacgcccaatttttcagctcgtgtcggcggcacaaacgatcttaccggactcggacggcgccatcgacggtcatttacgagaggcggggctcacgtttcacttgctaaaggatgtgcctgggattatatccaagaacatagagaagagcttaaacgaagcgtttaatccaattggtataagcgactggaactcgttattctggattgttcatccgggtggacccgcaattctggatcaagtggaggtcaagcttggacttaaggaagagaagctgagagcaacacgtaatgttttgagtgagtacggtaacatgtccagtgcttgtgtgctgtttatattggatgaaatgaggaagaaatcggtggaggaagggaaagcaaccaccggagacggtttggaatggggtgttctatttggtttcggaccagggctgacggtagagacggtggtgttgcacagtgttccggttgaaaccaattaa

SEQ ID NO: 68 - protein, 392 aa, Quillaja Saponaria
MASIEAIRKAQRAEGPATILAIGTATPSNYVLQAEYPDYYFRITNSEHMTDLKEKFKRMCDKSMIKKRYLYLDEEILKENPNLCAYMAPSLDARQDIAVVEVPKLGKEAAVKAIKQWGQPKSKITHLVFCTTSGVDMPGCDYQLTKLLGLRPSVKRLMMYQQGCFAGGTVLRLAKDLAENNKGARVLVVCSEITAVTFRGPSDTHLDSMVGQALFGDGAGALIIGSEPDTTVERPIFQLVSAAQTILPDSDGAIDGHLREAGLTFHLLKDVPGIISKNIEKSLNEAFNPIGISDWNSLFWIVHPGGPAILDQVEVKLGLKEEKLRATRNVLSEYGNMSSACVLFILDEMRKKSVEEGKATTGDGLEWGVLFGFGPGLTVETVVLHSVPVETN

SEQ ID NO: 69 - DNA, 1170 bp, Quillaja Saponaria
  source 1..1170: mol_type = other DNA
atggtgactgtggaggaagttcgcaaggctcaacgagcggaaggccctgccacaatcatggcaattggcacagctactcctccaaattgtgtagagcagagttcatatccggattactattttcgtatcacaaacagcgagcacaagacagaattgaaagagaaattcaagcgcatgtgtgacaaatcaaatattaagaagaggtacatgtacttgacagaggagattttgaaggaaaacccaaatgtttgtgcctatatggcaccttcacttgatgctaggcaggacatggttgtggttgaagtaccaaggctagggaaagaggcagcaactaaggccatcaaggaatggggccagcccaagtccaaaattacccacttggtcttttgcaccactagtggtgtagacatgcctggggctgactaccagcttactaaattgttgggcctccgcccacacgtcaagcggttgatgatgtaccaacaaggttgttttgccggtggcacggtgctccggctagccaaagacttggctgaaaacaacaaaggtgctcgtgtgcttgtggtgtgttctgaaattactgctgtcacattccgtgggcctagtgatactcacctggacagtcttgtgggccaagcattgtttggtgatggagctgctgctatcatcattggttcggatccaattccagaggttgagaagcccttatttgaactagtatcagcagcccaaactatccttccggatagtgatggggctatagatgggcatctccgtgaagttgggcttacatttcatcttcttaaggatgttcctgggctgatttcaaagaatgttgagaaaagcctaaatgaagcattccaaccattaggtatctcagactggaactcaattttttggattgcacatccaggtggtccagcaattttggaccaagttgagcttaaattgggccttaaaccagaaaaaatgcaggcaacccgacatgttcttagtgaatatggtaacatgtccagtgcctgtgtgttgtttataatggatgagatgagaaagaagtctgttgaaaatggacttggtaccactggagaaggacttgaatggggtgtgctgtttggatttggacctgggctcactgttgaaaccgttgtgcttcatagtctggctgcctaa

SEQ ID NO: 70 - protein, 389 aa, Quillaja Saponaria
MVTVEEVRKAQRAEGPATIMAIGTATPPNCVEQSSYPDYYFRITNSEHKTELKEKFKRMCDKSNIKKRYMYLTEEILKENPNVCAYMAPSLDARQDMVVVEVPRLGKEAATKAIKEWGQPKSKITHLVFCTTSGVDMPGADYQLTKLLGLRPHVKRLMMYQQGCFAGGTVLRLAKDLAENNKGARVLVVCSEITAVTFRGPSDTHLDSLVGQALFGDGAAAIIIGSDPIPEVEKPLFELVSAAQTILPDSDGAIDGHLREVGLTFHLLKDVPGLISKNVEKSLNEAFQPLGISDWNSIFWIAHPGGPAILDQVELKLGLKPEKMQATRHVLSEYGNMSSACVLFIMDEMRKKSVENGLGTTGEGLEWGVLFGFGPGLTVETVVLHSLAA

SEQ ID NO: 71 - DNA, 1203 bp, Quillaja Saponaria
  source 1..1203: mol_type = other DNA
atggctccggtgaccaacactaacggagttcagtcccagttggcaactaataggggtgtagcagcaatactagccatgggtacagcaactccatccaatcgtatcattcagtctgattaccctgatttttatttcacagtcaccggaagcgaccacatgaccgatttgaaaaacaagttcaggagaatttgtgagagatcaacgattgaaaaacgttacctacatctcactgaggaaatccacaaggcgcaccctaacatctctgaccccaaagctccttcctttgatgtccgtcaagaaatactttctgaggaagtaccaaaacttggcaaggaggcagctttgaaggccatcaaagaatggggccaacccgaatcaaaaatcactcaccttatattctatacaacttcttgcttaaacttgcccggcggtgacctttatctttgtgaacttcttggtctcgacccctcagtcaacaggctcatgttatattaccaaggctgccatgccggagggacagccctccgtgttgccaaggacatagccgagaacaatgccggttcacgtgtccttgttgtttgcgcagaattgagtacagtggtaacgtttcaaggtccgtgtgctacccagattgatagtctcgtaatgcaagccctgtttggtgatggtgcagttgccctgatcattggtgcaaatcctaatacctcaatcgaacgtccattgtttcaactagtgttggcttcacaaacaactatccctaaatctgagtatgcaattcaaggatatttgcgcgaaaagggtgccctttattatatgaataaagacatccctgttttgatatctaacaatgtcaagaaggttgtaacagatgcaatgatgaattcagttggcttgacaatgggtgagaatgattggaactccttgttttacctagttcatccgggtggaccgagtattctcgacgcggttgagaagcaacttggattggagaaggagaaactaagggcaagtaggcatgtgttgagggagtttggaaacatgtggagtaccactgtgttatttgcaatggatgaaatgaggaggaaatcggtggtggaagggaagagcaccaccggtgaagggttggagttaggtgttttattaggatttgggccaggtttaacggtggagacagttgtattgcgcagtgtcccagttgtgtaa

SEQ ID NO: 72 - protein, 400 aa, Quillaja Saponaria
MAPVTNTNGVQSQLATNRGVAAILAMGTATPSNRIIQSDYPDFYFTVTGSDHMTDLKNKFRRICERSTIEKRYLHLTEEIHKAHPNISDPKAPSFDVRQEILSEEVPKLGKEAALKAIKEWGQPESKITHLIFYTTSCLNLPGGDLYLCELLGLDPSVNRLMLYYQGCHAGGTALRVAKDIAENNAGSRVLVVCAELSTVVTFQGPCATQIDSLVMQALFGDGAVALIIGANPNTSIERPLFQLVLASQTTIPKSEYAIQGYLREKGALYYMNKDIPVLISNNVKKVVTDAMMNSVGLTMGENDWNSLFYLVHPGGPSILDAVEKQLGLEKEKLRASRHVLREFGNMWSTTVLFAMDEMRRKSVVEGKSTTGEGLELGVLLGFGPGLTVETVVLRSVPVV

SEQ ID NO: 73 - DNA, 1197 bp, Quillaja Saponaria
  source 1..1197: mol_type = other DNA
atgggatccttggatcaagattttcaaccaaaccaggtaccaacgattcgtgcggatgcagccacagtactagccattggaacggcaaacccacccaattgtattccccaagctgagttcgctgatttctatttccgagtaaccaacagcaaccacatgactgacctcaagaagaaattcaagcgcatctgtgaaaaatcatccataaagaaacgttacgtgcatctgacagagaaaattctgaatgaaaatcccaacatatgtaccttcaaagagccatctcttacagcacgtcaaaacatagcaaccgaggaaataccaaagcttggtaaagaagcagcactcaaagccattgaagaatggggaagacctaaatcggaaatcacccaccttgtattttgcactattggtggtgctgaaatgcctagtgcagattatcaacttgttcatcttttaggtcttaacccatctgtgaagaggttcatgctttactaccaaggttgctttgcaggtggctctgtactccgtttagcaaaagatcttgctgagaacaatgttggggctcgtgttcttgtagtgtgtgctgaggtttcaatcccttattttcatgggccttctgaagctcattttgattccaatttgatatggcaaatgttgtgtggggatggtgcaggagctgctatcgttggatcaaatccacgtaaattgatcgaacggccgttgtttcaacttgtgtctgcttcccaaactatagtacctaattcagagggtgcaattaagggaactttgagtgaaatgggtattatttttaatttggttgataaggtaccagtgttgataggaaacaacatcgagaattgtttggaggaagcatttgggtcactgggtattagtaattggaattcattgttttgggtagcccatccaggtggcccagccattttgaaccaaatcgaagagaaactgaaactcaaagaagagaaattaaaagcaagtagacatgtattaagcgaatacgggaatatgtcgagtgcttgtgtgttttttatattggacgagatgagaaggaggtctaaggaggaaggaaaaatcactactggtgatggattggagtggggggttttatttggatttgggcctgggttaactgtggaaaccattgtgctgcgtagcatttctacaaatgaggcccattaa

SEQ ID NO: 74 - protein, 398 aa, Quillaja Saponaria
MGSLDQDFQPNQVPTIRADAATVLAIGTANPPNCIPQAEFADFYFRVTNSNHMTDLKKKFKRICEKSSIKKRYVHLTEKILNENPNICTFKEPSLTARQNIATEEIPKLGKEAALKAIEEWGRPKSEITHLVFCTIGGAEMPSADYQLVHLLGLNPSVKRFMLYYQGCFAGGSVLRLAKDLAENNVGARVLVVCAEVSIPYFHGPSEAHFDSNLIWQMLCGDGAGAAIVGSNPRKLIERPLFQLVSASQTIVPNSEGAIKGTLSEMGIIFNLVDKVPVLIGNNIENCLEEAFGSLGISNWNSLFWVAHPGGPAILNQIEEKLKLKEEKLKASRHVLSEYGNMSSACVFFILDEMRRRSKEEGKITTGDGLEWGVLFGFGPGLTVETIVLRSISTNEAH

SEQ ID NO: 75 - DNA, 1176 bp, Quillaja Saponaria
  source 1..1176: mol_type = other DNA
atggcatccgtcgaagcaatccggaaagcccagcgtgccgatggtcctgccaccattttagcaatcggcaccgccacgccttccaactttattcttcaagctgactatcccgactactacttccgcatcacaaatagcgagcacatgactgatctcaaggaaaaattcaagcgcatgtgtgacaagtccatgataaagaagcgttacatgcacttgacagaagaaattcttaaagagaatcaaaacctatgtgcctacatggcaccttcccttgatgctcgtcaggatattgttgtcgtggaagtaccaaagctaggaaaagaggcagcagtgaaggccataaaacagtggggccagcccaaatcaaagattacccatcttgtgttctgcaccacatctggcgtggacatgcccggatgtgattaccagctcaccaagcttcttggccttagaccctccgtgaaacgacttatgatgtaccaacaaggttgttttgctggtgggaccgtcctccgtcttgccaaggacttagctgaaaacaataaaggtgctcgtgtccttgtcgtttgttccgagatcaccgccgttactttccgtggcccttccgatacacacttggactctatggtgggtcaggctttgttcggcgacggtgccggtgctttaatcatcggtgctgatcccgacacgagggttgaacgcccattgtttgagcttgtgtctgcgtcacagacgatcctacccgactctgacggagccattgacggtcatttaagagaggtggggcttacgtttcacttgttaaaggacgtgcctgggattatatctaaaaacatagagaagagcttaaacgaggcgtttagcccgattggcataagtgattggaattcgttattctggattgcacatccgggtgggcccgcaattctggatcaagtggaagccaaacttggccttaaggaagagaaattgaaagcgacacgtaatgtgttgagtgagtacgggaacatgtcgagtgcttgtgtgttgtttatattggatgaaatgaggaagaaatcggtggaggaagggaagggaaccaccggtgatggtttggaatggggtgttctattcgggttcgggccgggtctcacggtggagacggtggtgttgcacagtgttccgataagttattga

SEQ ID NO: 76 - protein, 391 aa, Quillaja Saponaria
MASVEAIRKAQRADGPATILAIGTATPSNFILQADYPDYYFRITNSEHMTDLKEKFKRMCDKSMIKKRYMHLTEEILKENQNLCAYMAPSLDARQDIVVVEVPKLGKEAAVKAIKQWGQPKSKITHLVFCTTSGVDMPGCDYQLTKLLGLRPSVKRLMMYQQGCFAGGTVLRLAKDLAENNKGARVLVVCSEITAVTFRGPSDTHLDSMVGQALFGDGAGALIIGADPDTRVERPLFELVSASQTILPDSDGAIDGHLREVGLTFHLLKDVPGIISKNIEKSLNEAFSPIGISDWNSLFWIAHPGGPAILDQVEAKLGLKEEKLKATRNVLSEYGNMSSACVLFILDEMRKKSVEEGKGTTGDGLEWGVLFGFGPGLTVETVVLHSVPISY

SEQ ID NO: 77 - DNA, 972 bp, Quillaja Saponaria
  source 1..972: mol_type = other DNA
atggcggcggccgaaagtgaaagggtatgtgtaacgggggctggaggatttgtggcctcttggcttgttaagcatctcctctccaagggatatttagtccatggaaccatcagagaacccggtaatcacaagtatgctcatttggagaaactggagaaagcttccgagaagcttaaactcttcaaggcagatgtcttaaactacgattctctttactcagcaatagaaggatgctctggagttttccatgttgctagtcccgctcctgcagggcctgtatcaaatcctgaggtagagctgttcgagcctgctgtgaaaggaacacttaatgtacttaaagcttgtgttgaagcaaaagtaaagcgagttgtggttgtatcatctatatctgctgttgtgctgaaccccaagtggccaaaggaaaaagtcatggatgagtcctgctggtctgatacagaatactgcagaaacactcagaactggtattatgtttccaaaacagaagcagagtgggaggctctggaatttgcaaaaacaaatgggattgatgttgtaacagtctgtccggcccttgtctttgggccactgcttcaatccactgtgaatgctagtagttttggtctcgccatgcttatgaaagggttccagtcgacaatggagaacaagaatttggatatagtagacgtgcgggatttggctgatgcattgcttttagtgtatgagaagcgcgaggctgaaggaagatacatatgtctagcacactctgtaaagacgcaggaaatggtcgataaacttaggactctgtatcccaactatggctaccccagcaagattgttgatgtagaagaagacacgaggttgagctccgacaaactgcacaagttgggctggaccacattcaggccattggaggaaacttttactgattctgttgaaagctatcgaaaggcaggagttctcgaaactaactag

SEQ ID NO: 78 - protein, 323 aa, Quillaja Saponaria
MAAAESERVCVTGAGGFVASWLVKHLLSKGYLVHGTIREPGNHKYAHLEKLEKASEKLKLFKADVLNYDSLYSAIEGCSGVFHVASPAPAGPVSNPEVELFEPAVKGTLNVLKACVEAKVKRVVVVSSISAVVLNPKWPKEKVMDESCWSDTEYCRNTQNWYYVSKTEAEWEALEFAKTNGIDVVTVCPALVFGPLLQSTVNASSFGLAMLMKGFQSTMENKNLDIVDVRDLADALLLVYEKREAEGRYICLAHSVKTQEMVDKLRTLYPNYGYPSKIVDVEEDTRLSSDKLHKLGWTTFRPLEETFTDSVESYRKAGVLETN

SEQ ID NO: 79 - DNA, 963 bp, Quillaja Saponaria
  source 1..963: mol_type = other DNA
atggaatgctgtatagtagctaggctaaaaacccaacctttatggtttgctttgctttttactttgggtgttctctcacttttgaaattatcttttgcttttctcaaatgggtctatgttaattttcttaggcctcccaaaaacctcaagaaatacgggtcgtgggcagttgtttctggaccaactgatggtattggaaagggttttgcttttcaattggctcgtaaagggcttaatctggtcttagttggtcgaaatccagataagctgaaagatgtttctgattccattttagctaaatttgagaaaacccagattaagactgttgttgttgattttactggtgatttatccgagggtattgttaggattcgtgaagccattgaaggattggatgttgggattctaattaacaatgtcggagtttcctatccttatgctaggttcttccacgaggtagatgagcgactcttgaagaatttgattaaagttaatgttgaaggcactacgaaggttacacaagctgttctgtctgggatgctcaagaagaagaaaggagctattgtgaatattggctctggggacgcaaatatacttccttgtgatcctctttatgctgtttatgcagccacaaaatcgtatatcgaccaattctcgaggtgtctctatgttgagtacaagggcagtgggattgacgtccagtgccaggttcctatgtatgtggctacaaagatggcatcaattaggaattcttcctttcttgttccttcaatagatggctatgccaaagcaggtttgcggtggataggctatgagcctctatgcacaccttactggcctcattctcttatttgggctttggcagactcattgccagagtctgtcgtccatgcatggcgcttgaagttcaaccttgcaattcgaaagagggggcagcttaaagatgcaaggaagaaagaataa

SEQ ID NO: 80 - protein, 320 aa, Quillaja Saponaria
MECCIVARLKTQPLWFALLFTLGVLSLLKLSFAFLKWVYVNFLRPPKNLKKYGSWAVVSGPTDGIGKGFAFQLARKGLNLVLVGRNPDKLKDVSDSILAKFEKTQIKTVVVDFTGDLSEGIVRIREAIEGLDVGILINNVGVSYPYARFFHEVDERLLKNLIKVNVEGTTKVTQAVLSGMLKKKKGAIVNIGSGDANILPCDPLYAVYAATKSYIDQFSRCLYVEYKGSGIDVQCQVPMYVATKMASIRNSSFLVPSIDGYAKAGLRWIGYEPLCTPYWPHSLIWALADSLPESVVHAWRLKFNLAIRKRGQLKDARKKE

SEQ ID NO: 81 - DNA, 1401 bp, Quillaja Saponaria
  source 1..1401: mol_type = other DNA
atgatggaggtacataccacatcggaaaattgcattaagccctcacaacccactccttcccaccttcaaaatttgaaactctctaatcatcatagccaagcacccgatatccgtaccaacctcaccttcttcttctcctctaattttaacaatccagtccagcctggtgaccatgacgctactaccaattttacactccaatccaaacttgttcagaattcattggctacaactctcacaattctctacccttttgctggtcgattccgaaacgacgacaccatcatttgcaaagacgatggcgccttctttattgaagcaaaaaccgacaccaaactttctgactttcttgcccagccggacttgcctctagctataatggacaaattagtccccgtagctaccgacgccaagtataatggttctctgctaatcctgaaatttactttgtttggctgcggtggctcagccgtaaccatctcaataactcacaagatttccgatctagcaaccattttgacactgcttaattgctggactgctttatctcgtggtggtgatggtggtggttccagtccgtttattcagcctgatttgaatttcattggccgccctgttccaagtacgagtgaggttcctcctccatcttctggaaagaattttattcctccaaatagcaagtacgttaccaaaaggttcatattcagtgcagcaaagattaaagaactgaaagccagagtcatcaacaagattcgtaaagaagaagacaatgttttcccttcccgtgtggatgtagtgctggcactcatttggaaatgcgctttggcttctgttaattctggttccagatctggaaatgcacaaacatttaggccgtcggtaatgatgcaagccgtgaacctcagaaaccgtacagatccaccattaccggaatcttcgattgggaacttggcaatactattaccggtgtgggtggagaaagaggaagacacggaattacatgaacttgttagcagattgttaacagtgaaagtgcgtgctaacagattgaagaagaaataccaaggttatgaagatccagagcaagttattatttcaatggaatctgattcagtgaaggaaataatagaggttcggaagaaattgaaggatttcagtacttatgttgcggcgagtgtggtgaatgctccattgtatgacgttgattttgggtggggaaaaccagcatgggtgaccagcacgcccaacacagtaatggcgaattctatatatttgttggataccaaagacgccggtgggattgaagttttgatgaatatgttgaaagaggaagacatgattgtctttgaaagcaatcaggagttgcttcagtctgccatggttaatcccactatcatttga

SEQ ID NO: 82 - protein, 466 aa, Quillaja Saponaria
MMEVHTTSENCIKPSQPTPSHLQNLKLSNHHSQAPDIRTNLTFFFSSNFNNPVQPGDHDATTNFTLQSKLVQNSLATTLTILYPFAGRFRNDDTIICKDDGAFFIEAKTDTKLSDFLAQPDLPLAIMDKLVPVATDAKYNGSLLILKFTLFGCGGSAVTISITHKISDLATILTLLNCWTALSRGGDGGGSSPFIQPDLNFIGRPVPSTSEVPPPSSGKNFIPPNSKYVTKRFIFSAAKIKELKARVINKIRKEEDNVFPSRVDVVLALIWKCALASVNSGSRSGNAQTFRPSVMMQAVNLRNRTDPPLPESSIGNLAILLPVWVEKEEDTELHELVSRLLTVKVRANRLKKKYQGYEDPEQVIISMESDSVKEIIEVRKKLKDFSTYVAASVVNAPLYDVDFGWGKPAWVTSTPNTVMANSIYLLDTKDAGGIEVLMNMLKEEDMIVFESNQELLQSAMVNPTII

SEQ ID NO: 83 - DNA, 1458 bp, Quillaja Saponaria
  source 1..1458: mol_type = other DNA
atggctgaagctttgaaattgaaagtagttgatgaattccaagtttctccaccacaagattcagttcccacaaccactctccctctctctttttttgacattccatttctttttatctctcaagggaaacgtattttcttctatgaattccctcacccaactcatcatttcatggaagctgttcttcctcatctcaaacaatctctttccctcactcttaaacactttttcccagttgctgccaatattgtatttcctccacaacccaacaaaccccatgtcctctttgactccggttcctcctccgtctccttcaccgtcgctgaatccaccgccgatttcaactacctgattactgattccccacgcgccgttagttctgttgacccatttgtccccaaaatccctcccgctcgtgtcttggacgacggcacacggttgtttcctctcttggccattcaagccactgtcataccaaactctggattcagcatttgtatcagatttcaccatggaattggtgatggaatggcatttcagcattttgtaagatcctgggcatttcaatgtaagaaattaattagcacaaaaccagaagaggaagaagaaggaggaaaacattcttccctcccaattttcaataatgggtctaccattccagacccaaatggggtcgagctttgtttcttgaactttttatggaagtggacacctgcacttctaaaaatggcagatgggtccggatccgagttcaactcgacccataatagtgtcaatgttgatgccacattcaaggttacacaaacccatattgataagctaaagcaattgatctctctgaaattttacaatgggaatgagtcagagtcagagccagttgaaaaagcaccattacgtttatcaacctttgtagtatcatgcgccttaatttgggtttgtttagcaaaatcagaggagaacacttacattgatgatgaagaaccttacaattttacttttgtagctgattgtagaggtcgttttgagttttcttctgtacctgcaacatattttgggaattgtgtggctcctgtttatgcaacactaaagaggaaggagattgtgggagagaatgggattgttgaagcagcaaaagctataagaaataaaatcaaggaattggagaaaaatggagttttgaaagttgcagaaagatggatgttagatgctatagagaaaacaacaacaaagcataatgttatggttgctggttcaccaaaattgggtgtttatgaaatggattttggttggggaagtcctaagaagagtgatgtgcttggtaaccctggttctattttgatttctttgtccgaatcaaaggacaagaatctaggtgggattgaagtgggtgtgggacttgaaaagacccaaatgaataatttcattcagacatttgatcaaacattgaagatactctaa

SEQ ID NO: 84 - protein, 485 aa, Quillaja Saponaria
MAEALKLKVVDEFQVSPPQDSVPTTTLPLSFFDIPFLFISQGKRIFFYEFPHPTHHFMEAVLPHLKQSLSLTLKHFFPVAANIVFPPQPNKPHVLFDSGSSSVSFTVAESTADFNYLITDSPRAVSSVDPFVPKIPPARVLDDGTRLFPLLAIQATVIPNSGFSICIRFHHGIGDGMAFQHFVRSWAFQCKKLISTKPEEEEEGGKHSSLPIFNNGSTIPDPNGVELCFLNFLWKWTPALLKMADGSGSEFNSTHNSVNVDATFKVTQTHIDKLKQLISLKFYNGNESESEPVEKAPLRLSTFVVSCALIWVCLAKSEENTYIDDEEPYNFTFVADCRGRFEFSSVPATYFGNCVAPVYATLKRKEIVGENGIVEAAKAIRNKIKELEKNGVLKVAERWMLDAIEKTTTKHNVMVAGSPKLGVYEMDFGWGSPKKSDVLGNPGSILISLSESKDKNLGGIEVGVGLEKTQMNNFIQTFDQTLKIL

SEQ ID NO: 85 - DNA, 1473 bp, Quillaja Saponaria
  source 1..1473: mol_type = other DNA
atgggttccagtgcaaagcctcttcattttgtattgtttcctcatttggcaccaggccatcttctccccttatttgacactgcaagactgttggcacagcgtggtgctactgttacaatattcacctcaccaggaaacgcatcccgttttgaatcacttgtttctcgtgccgtttcgtcaggctactcaatccaattcgttcaagtccaaattccattcaaagaagcaggactacctgaagggtgtgagaattttgacatgctacagtcttatgacttggcattcaaactttacactgcaattagattcatgcacaagcctgcagaaatattgtttgaagcactaccactcaaaccaagttgcataatctctgacgtctgtgttccatggacacttcaaattgctcgaaaacataatattccaaggattactttccatatcttctcaactttctttctcttgagctcctacaagtttggaatgtcccaggttactcagaacacattcacagaaacagagcatgttgccatgtcagaaattcctgaccaaaatgatccgattacagaaatccagacacaagcacaactgagtcaaaaactgatgcattattctatccaaatgatggctgcagatataaaatcatatggcgcaatcgtaaatagctctgaagcagtggagccagagtctattgaggagtacaaaaagctaaggaatgggaaggcctggtgtgttggccccgtttggctgtgcaataagaatgagttagataaggctgagagaggtaacaaatcctccattgatgagcattattgcttggagtggcttaatttgcaggaagaaaactctgtagtctatgcctgccttggaagtgtagccaacttgagtgcttcacaactaatagaacttggattggggttagaagcatctaaaagaccatttgtttgggttataaggggaggggataatttaaaagaattggaaaagtggattaaggaagatgggtttgaggaaaggattaaagggaaaggtcttttaattagagggtgggcaccacaagtactaattttgtcccactctgccattgggggattcataacacattgtgggtggggttcaatacttgaagcaataacttgtggtgttccgataattacatggcccttatttgctgatcaattttgcaatcagattaatgttgcacaagtgttaaaaattggtgtgaaagttggggcagaggatcttgtaaacaatggagagggagagaagattgaagcattggtgaagaaggaagacattaagagggcaatagaagaactgatggatgaaggtgaagaaagtgaagagagaagaagaagagtgagagaaatgggagagatggcaaagaaagctgcagaagaagggggttcttctcatcttgatctcacttcgctcattgaagatatcaaacaacaatcaactagcacgcaggaatga

SEQ ID NO: 86 - protein, 490 aa, Quillaja Saponaria
MGSSAKPLHFVLFPHLAPGHLLPLFDTARLLAQRGATVTIFTSPGNASRFESLVSRAVSSGYSIQFVQVQIPFKEAGLPEGCENFDMLQSYDLAFKLYTAIRFMHKPAEILFEALPLKPSCIISDVCVPWTLQIARKHNIPRITFHIFSTFFLLSSYKFGMSQVTQNTFTETEHVAMSEIPDQNDPITEIQTQAQLSQKLMHYSIQMMAADIKSYGAIVNSSEAVEPESIEEYKKLRNGKAWCVGPVWLCNKNELDKAERGNKSSIDEHYCLEWLNLQEENSVVYACLGSVANLSASQLIELGLGLEASKRPFVWVIRGGDNLKELEKWIKEDGFEERIKGKGLLIRGWAPQVLILSHSAIGGFITHCGWGSILEAITCGVPIITWPLFADQFCNQINVAQVLKIGVKVGAEDLVNNGEGEKIEALVKKEDIKRAIEELMDEGEESEERRRRVREMGEMAKKAAEEGGSSHLDLTSLIEDIKQQSTSTQE

SEQ ID NO: 87 - DNA, 1515 bp, Quillaja Saponaria
  source 1..1515: mol_type = other DNA
atgccattcattcgtaatactccaaaagcaaagaaggcagccatgggttccagtgcaaagcctcttcattttgtattgtttcctcatttggcaccaggccatcttctccccttatttgacactgcaagactgttggcacagcgtggtgctactgttacaatattcacctcaccaggaaacgcatcccgttttgaatcacttgtttctcgtgccgtttcgtcaggctactcaatccaattcgttcaagtccaaattccattcaaagaagcaggactacctgaagggtgtgagaattttgacatgctacagtcttatgacttggcattcaaactttacactgcaattagattcatgcacaagcctgcagaaatattgtttgaagcactaccactcaaaccaagttgcataatctctgacgtctgtgttccatggacacttcaaattgctcgaaaacataatattccaaggattactttccatatcttctcaactttctttctcttgagctcctacaagtttggaatgtcccaggttactcagaacacattcacagaaacagagcatgttgccatgtcagaaattcctgaccaaaatgatccgattacagaaatccagacacaagcacaactgagtcaaaaactgatgcattattctatccaaatgatggctgcagatataaaatcatatggcgcaatcgtaaatagctctgaagcagtggagccagagtctattgaggagtacaaaaagctaaggaatgggaaggcctggtgtgttggccccgtttggctgtgcaataagaatgagttagataaggctgagagaggtaacaaatcctccattgatgagcattattgcttggagtggcttaatttgcaggaagaaaactctgtagtctatgcctgccttggaagtgtagccaacttgagtgcttcacaactaatagaacttggattggggttagaagcatctaaaagaccatttgtttgggttataaggggaggggataatttaaaagaattggaaaagtggattaaggaagatgggtttgaggaaaggattaaagggaaaggtcttttaattagaggttgggcaccacaagtactaattttgtcccactctgccattgggggattcataacacattgtgggtggggttcaatacttgaagcaataacttgtggtgttccgataattacatggcccttatttgctgatcaattttgcaatcagattaatgttgcacaagtgttaaaaattggtgtgaaagttggggcagaggatcttgtaaacaatggagagggagagaagattgaagcattggtgaagaaggaagacattaagagggcaatagaagaactgatggatgaaggtgaagaaagtgaagagagaagaagaagagtgagagaaatgggagagatggcaaagaaagctgcagaagaagggggttcttctcatcttgatctcacttcgcttattgaagatatcaaacaacaatcaactagcacgcaggaatga

SEQ ID NO: 88 - protein, 504 aa, Quillaja Saponaria
MPFIRNTPKAKKAAMGSSAKPLHFVLFPHLAPGHLLPLFDTARLLAQRGATVTIFTSPGNASRFESLVSRAVSSGYSIQFVQVQIPFKEAGLPEGCENFDMLQSYDLAFKLYTAIRFMHKPAEILFEALPLKPSCIISDVCVPWTLQIARKHNIPRITFHIFSTFFLLSSYKFGMSQVTQNTFTETEHVAMSEIPDQNDPITEIQTQAQLSQKLMHYSIQMMAADIKSYGAIVNSSEAVEPESIEEYKKLRNGKAWCVGPVWLCNKNELDKAERGNKSSIDEHYCLEWLNLQEENSVVYACLGSVANLSASQLIELGLGLEASKRPFVWVIRGGDNLKELEKWIKEDGFEERIKGKGLLIRGWAPQVLILSHSAIGGFITHCGWGSILEAITCGVPIITWPLFADQFCNQINVAQVLKIGVKVGAEDLVNNGEGEKIEALVKKEDIKRAIEELMDEGEESEERRRRVREMGEMAKKAAEEGGSSHLDLTSLIEDIKQQSTSTQE

SEQ ID NO: 89 - DNA, 1320 bp, Quillaja saponaria
  source 1..1320: mol_type = other DNA
atggcggatcgagtcataaacagctacaaaaagcttcacgtagtgctgtttccatggctggcctttggtcacatgataccatttttagagcttgcaaagctgatagcccaaaaaggtcacaaaatttcctatatctcaacacccagaaacatccgacgcctacctaaaattccatcccatttatccaacaaccttaatttcatagaattcccactgcctcatatacccaatcttcatgaaaatgtggaatcaacaaacgacgtcacacatgacaatcctattgcctattatttacttattaaagcccttgagggtttgcaacaacctatcaccaccttccttgaaacctcagatccagattggataattcatgacgtttttcctcaatggataaccgcaacagccagtaggctccgcatttcacatgctttttacacaacttcatctgctttgagaacggcttccaattattctccgttaatgtcctctgaattaccacaggatattgctaccgatttcactactaaatccacaaatcttcttgctgccaaggttttggttatccgtagctgtctagagcttgaacccaaggaatttgaacagtgcaaaaatctatgcgtgtccaaaacggtaattccattgggcgttgtcccaccgtcaattcaggtgaacgataacatttctaatattaatgatgacgacaacgactgggttaagattgttgagtggttaaaccaagggaaagagaaaggttctgtcatttatgtagcacttggttctgaggtttcactgagtgaacaagatttgaaggagtttgcacttggtttggaactatcagggttgtcattcttttgggtgttcaggaatactggatcgtatgggttaccggctgggtttgaagaccgagttaagggtcggggaattgtctggactagctgggctccgcaggtgagtatattaggacacgagtcaattggtggattcttgagtcatgggggttggagttctgtaatagaaagtctaagttttgggattccacttgttgtttttccatttggagctgatcaagggattaatgcaaagcaattggagggtaaaaatgcaggggtggaaatacccagatcagaaggtactgggtcatttacaaggaagtctgtggctgatttgttgaggctggtggtggtggaggaggagggaaaggtttacagggatggtgccaaggaattgaggaaactatttggggacaaggatttgaaccacaaatacattgacaactttgttaagtacatggaagaacatatcacgaatgcagctaattga

SEQ ID NO: 90 - protein, 439 aa, Quillaja saponaria
MADRVINSYKKLHVVLFPWLAFGHMIPFLELAKLIAQKGHKISYISTPRNIRRLPKIPSHLSNNLNFIEFPLPHIPNLHENVESTNDVTHDNPIAYYLLIKALEGLQQPITTFLETSDPDWIIHDVFPQWITATASRLRISHAFYTTSSALRTASNYSPLMSSELPQDIATDFTTKSTNLLAAKVLVIRSCLELEPKEFEQCKNLCVSKTVIPLGVVPPSIQVNDNISNINDDDNDWVKIVEWLNQGKEKGSVIYVALGSEVSLSEQDLKEFALGLELSGLSFFWVFRNTGSYGLPAGFEDRVKGRGIVWTSWAPQVSILGHESIGGFLSHGGWSSVIESLSFGIPLVVFPFGADQGINAKQLEGKNAGVEIPRSEGTGSFTRKSVADLLRLVVVEEEGKVYRDGAKELRKLFGDKDLNHKYIDNFVKYMEEHITNAAN

SEQ ID NO: 91 - DNA, 54 bp, synthetic construct
  misc_feature 1..54: note = PCR Primer - UGT-BI_attB1F
  source 1..54: mol_type = other DNA
ggggacaagtttgtacaaaaaagcaggcttaatggcggatcgagtcataaacag

SEQ ID NO: 92 - DNA, 55 bp, synthetic construct
  misc_feature 1..55: note = PCR Primer - UGT-BI_attB2R
  source 1..55: mol_type = other DNA
ggggaccactttgtacaagaaagctgggtatcaattagctgcattcgtgatatgc

SEQ ID NO: 93 - DNA, 61 bp, synthetic construct
  misc_feature 1..61: note = PCR primer - Qs1CCL_attB1F
  source 1..61: mol_type = other DNA
ggggacaagtttgtacaaaaaagcaggcttaatggatcaattaaatccaaagcctgtaaac

SEQ ID NO: 94 - DNA, 53 bp, synthetic construct
  misc_feature 1..53: note = PCR primer - Qs1CCL_attB2R
  source 1..53: mol_type = other DNA
ggggaccactttgtacaagaaagctgggtattacaaccgactagctgtcaacg

SEQ ID NO: 95 - DNA, 56 bp, synthetic construct
  misc_feature 1..56: note = PCR primer - Qs2CCL_attB1F
  source 1..56: mol_type = other DNA
ggggacaagtttgtacaaaaaagcaggcttaatggaagatcttaagccaagcccag

SEQ ID NO: 96 - DNA, 55 bp, synthetic construct
  misc_feature 1..55: note = PCR primer - Qs2CCL_attB2R
  source 1..55: mol_type = other DNA
ggggaccactttgtacaagaaagctgggtatcacatccgactcgatcttagtggg

SEQ ID NO: 97 - DNA, 57 bp, synthetic construct
  misc_feature 1..57: note = PCR primer - Qs3CCL_attB1F
  source 1..57: mol_type = other DNA
ggggacaagtttgtacaaaaaagcaggcttaatggaagatcttaaaccaagctctgc

SEQ ID NO: 98 - DNA, 52 bp, synthetic construct
  misc_feature 1..52: note = PCR primer - Qs3CCL_attB2R
  source 1..52: mol_type = other DNA
ggggaccactttgtacaagaaagctgggtactatgtgtggttggagagaggc

SEQ ID NO: 99 - DNA, 54 bp, synthetic construct
  misc_feature 1..54: note = PCR primer - Qs4CCL_attB1F
  source 1..54: mol_type = other DNA
ggggacaagtttgtacaaaaaagcaggcttaatggaaaaccttaagccaacccc

SEQ ID NO: 100 - DNA, 51 bp, synthetic construct
  misc_feature 1..51: note = PCR primer - Qs4CCL_attB2R
  source 1..51: mol_type = other DNA
ggggaccactttgtacaagaaagctgggtatcacatccgactcgaccttgg

SEQ ID NO: 101 - DNA, 54 bp, synthetic construct
  misc_feature 1..54: note = PCR primer - Qs5CCL_attB1F
  source 1..54: mol_type = other DNA
ggggacaagtttgtacaaaaaagcaggcttaatggagcaactcaacccaaaacc

SEQ ID NO: 102 - DNA, 56 bp, synthetic construct
  misc_feature 1..56: note = PCR primer - Qs5CCL_attB2R
  source 1..56: mol_type = other DNA
ggggaccactttgtacaagaaagctgggtactacaaccgactatctgtcaacgacc

SEQ ID NO: 103 - DNA, 54 bp, synthetic construct; identical to SEQ ID NO: 101
  misc_feature 1..54: note = PCR primer - Qs6CCL_attB1F
  source 1..54: mol_type = other DNA

SEQ ID NO: 104 - DNA, 56 bp, synthetic construct; identical to SEQ ID NO: 102
  misc_feature 1..56: note = PCR primer - Qs6CCL_attB2R
  source 1..56: mol_type = other DNA

SEQ ID NO: 105 - DNA, 54 bp, synthetic construct
  misc_feature 1..54: note = PCR primer - QsChSA_attB1F
  source 1..54: mol_type = other DNA
ggggacaagtttgtacaaaaaagcaggcttaatggtgactgtggaggaagttcg

SEQ ID NO: 106 - DNA, 54 bp, synthetic construct
  misc_feature 1..54: note = PCR primer - QsChSA_attB2R
  source 1..54: mol_type = other DNA
ggggaccactttgtacaagaaagctgggtattaagcagacacgctatgaagcac

SEQ ID NO: 107 - DNA, 53 bp, synthetic construct
  misc_feature 1..53: note = PCR primer - QsChSB_attB1F
  source 1..53: mol_type = other DNA
ggggacaagtttgtacaaaaaagcaggcttaatggcatccatcgaagcaatcc

SEQ ID NO: 108 - DNA, 55 bp, synthetic construct
  misc_feature 1..55: note = PCR primer - QsChSB_attB2R
  source 1..55: mol_type = other DNA
ggggaccactttgtacaagaaagctgggtattaattggtttcaaccggaacactg

SEQ ID NO: 109 - DNA, 54 bp, synthetic construct; identical to SEQ ID NO: 105
  misc_feature 1..54: note = PCR primer - QsChSC_attB1F
  source 1..54: mol_type = other DNA

SEQ ID NO: 110 - DNA, 54 bp, synthetic construct
  misc_feature 1..54: note = PCR primer - QsChSC_attB2R
  source 1..54: mol_type = other DNA
ggggaccactttgtacaagaaagctgggtattaggcagccagactatgaagcac

SEQ ID NO: 111 - DNA, 51 bp, synthetic construct
  misc_feature 1..51: note = PCR primer - QsChSD_attB1F
  source 1..51: mol_type = other DNA
ggggacaagtttgtacaaaaaagcaggcttaatggctccggtgaccaacac

SEQ ID NO: 112 - DNA, 52 bp, synthetic construct
  misc_feature 1..52: note = PCR primer - QsChSD_attB2R
  source 1..52: mol_type = other DNA
ggggaccactttgtacaagaaagctgggtattacacaactgggacactgcgc

SEQ ID NO: 113 - DNA, 56 bp, synthetic construct
  misc_feature 1..56: note = PCR primer - QsChSE_attB1F
  source 1..56: mol_type = other DNA
ggggacaagtttgtacaaaaaagcaggcttaatgggatccttggatcaagattttc

SEQ ID NO: 114 - DNA, 56 bp, synthetic construct
  misc_feature 1..56: note = PCR primer - QsChSE_attB2R
  source 1..56: mol_type = other DNA
ggggaccactttgtacaagaaagctgggtattaatgggcctcatttgtagaaatgc

SEQ ID NO: 115 - DNA, 52 bp, synthetic construct
  misc_feature 1..52: note = PCR primer - QsChSF_attB1F
  source 1..52: mol_type = other DNA
ggggacaagtttgtacaaaaaagcaggcttaatggcatccgtcgaagcaatc

SEQ ID NO: 116 - DNA, 58 bp, synthetic construct
  misc_feature 1..58: note = PCR primer - QsChSF_attB2R
  source 1..58: mol_type = other DNA
ggggaccactttgtacaagaaagctgggtatcaataacttatcggaacactgtgcaac

SEQ ID NO: 117 - DNA, 54 bp, synthetic construct
  misc_feature 1..54: note = PCR primer - QsKR11_attB1F
  source 1..54: mol_type = other DNA
ggggacaagtttgtacaaaaaagcaggcttaatggcggcggccgaaagtgaaag

SEQ ID NO: 118 - DNA, 65 bp, synthetic construct
  misc_feature 1..65: note = PCR primer - QsKR11_attB2R
  source 1..65: mol_type = other DNA
ggggaccactttgtacaagaaagctgggtactagttagtttcgagaactcctgcctttcgatagc

SEQ ID NO: 119 - DNA, 64 bp, synthetic construct
  misc_feature 1..64: note = PCR primer - QsKR23'_attB1F
  source 1..64: mol_type = other DNA
ggggacaagtttgtacaaaaaagcaggcttaatggaatgctgtatagtagctaggctaaaaacc

SEQ ID NO: 120 - DNA, 62 bp, synthetic construct
  misc_feature 1..62: note = PCR primer - QsKR23'_attB2R
  source 1..62: mol_type = other DNA
ggggaccactttgtacaagaaagctgggtattattctttcttccttgcatctttaagctgcc

SEQ ID NO: 121 - DNA, 62 bp, synthetic construct
  misc_feature 1..62: note = PCR primer - QsDMOT4_attB1F
  source 1..62: mol_type = other DNA
ggggacaagtttgtacaaaaaagcaggcttaatggctgaagctttgaaattgaaagtagttg

SEQ ID NO: 122 - DNA, 67 bp, synthetic construct
  misc_feature 1..67: note = PCR primer - QsDMOT4_attB2R
  source 1..67: mol_type = other DNA
ggggaccactttgtacaagaaagctgggtattagagtatcttcaatgtttgatcaaatgtctgaatg

SEQ ID NO: 123 - DNA, 56 bp, synthetic construct
  misc_feature 1..56: note = PCR primer - QsDMOT9_attB1F
  source 1..56: mol_type = other DNA
ggggacaagtttgtacaaaaaagcaggcttaatgatggaggtacataccacatcgg

SEQ ID NO: 124 - DNA, 59 bp, synthetic construct
  misc_feature 1..59: note = PCR primer - QsDMOT9_attB2R
  source 1..59: mol_type = other DNA
ggggaccactttgtacaagaaagctgggtatcaaatgatagtgggattaaccatggcag

SEQ ID NO: 125 - DNA, 53 bp, synthetic construct
  misc_feature 1..53: note = PCR primer - QsUGT-L-short_attB1F
  source 1..53: mol_type = other DNA
ggggacaagtttgtacaaaaaagcaggcttaatgggttccagtgcaaagcctc

SEQ ID NO: 126 - DNA, 57 bp, synthetic construct
  misc_feature 1..57: note = PCR primer - QsUGT-L-short_attB2R
  source 1..57: mol_type = other DNA
ggggaccactttgtacaagaaagctgggtatcattcctgcgtgctagttgattgttg

SEQ ID NO: 127 - DNA, 54 bp, synthetic construct
  misc_feature 1..54: note = PCR primer - QsUGT-L-long_attB1F
  source 1..54: mol_type = other DNA
ggggacaagtttgtacaaaaaagcaggcttaatgccattcattcgtaatactcc

SEQ ID NO: 128 - DNA, 50 bp, synthetic construct
  misc_feature 1..50: note = PCR primer - QsUGT-L-long_attB2R
  source 1..50: mol_type = other DNA
ggggaccactttgtacaagaaagctgggtatcattcctgcgtgctagttg

SEQ ID NO: 129 - DNA, 31 bp, synthetic construct
  misc_feature 1..31: note = Oligonucleotide for His-tag addition to UGT-L-long by PCR amplification
  source 1..31: mol_type = other DNA
attctgcccaaattcgatgccattcattcgt

SEQ ID NO: 130 - DNA, 52 bp, synthetic construct
  misc_feature 1..52: note = Oligonucleotide for His-tag addition to UGT-L-long by PCR amplification
  source 1..52: mol_type = other DNA
tgatgcataccggtcgtcagtggtggtggtggtggtgttcctgcgtgctagt

SEQ ID NO: 131 - DNA, 57 bp, synthetic construct
  source 1..57: mol_type = other DNA
  misc_feature 1..57: note = QsTD_0222940_attB1F
ggggacaagtttgtacaaaaaagcaggcttaatggagacactgtgttactcgccatc

SEQ ID NO: 132 - DNA, 60 bp, synthetic construct
  source 1..60: mol_type = other DNA
  misc_feature 1..60: note = QsTD_0222940_attB2R
ggggaccactttgtacaagaaagctgggtattaatgcatcaaaagctggaaatcttcatc

SEQ ID NO: 133 - DNA, 1842 bp, Quillaja Saponaria
  source 1..1842: mol_type = other DNA
  misc_feature 1..1842: note = Q. saponaria Threonine Deaminase wild-type (Qs0222940.1)
atggagacactgtgttactcgccatctcaggcacctctctttcgccagaagcgtttgggagaattggcgcgtacttctgtccccaaaaccaataattgcagcagagaactacccatcaagcctttcatcactgccacgctatccaaacaggcagaagagatttctcagaagccctctttagctgctacgacggttacttcccgcgaagaccaattaacggtgtctcctttgccttctcgtaaaagggtctcgccggattctctatgttatccatccggttacgttggggccgtcccggaccgtacggtttcctctggaggtgatgatataatcaatgcgatgagttatttgacgaatatactttcttctaaggtttacgatgtggccattgaatcgccgttacagctcgcgacaaaactctctgaacgcttgcgggtgaacatttggctcaagagggaggatttacaacctgttttctcattcaagctccgtggtgcatataatatgatggcaaaacttccgaaggaactgttggaaaaaggggtcatctgctcttcagctggaaatcatgcccaaggggttgcattagcttccaagagactgggctgcagtgctgtgattgttatgcctgttactactccagaaatcaagtggagatctgttgagaagctgggtgcaacagttgttcttgtgggggattcttatgatgaagcacaagcatatgctaagaatcgtggaaatgaagagggccgttctttcatacctccttttgatcatcccgatgtaataatggggcagggaacagttgggatggaaattgttcgccaaatgcaacgtcctgtgcatgcaatctttgttccagtaggaggtggtggactaatcgctggtattgcagcttatgtaaagcgggtctctccagaggtgaaggttattggagttgagccaactgatgcaaatgcaatggcattgtcattacatcatggccagagagtgattttggaccaggtcggaggctttgcagatggtgtagctgttaaggaggttggggaagaaacttttcgcttatgcaaggagttgattgatggtgtggttctcgtaagccgtgattctatttgtgcctcaataaaggacatgtttgaggagaaaaggagcattttagaaccagcaggtgctcttgcccttgctggagctgaagcatactgcaagtattatggtctcaagggagaaaatgtcgtagctataaccagtggggcaaacatgaactttgataaactgagagtggtgactgaactagctaatgttggtcgtaaacaagaagccgtccttgcaactgttttgccagaggagaatggcagttttaaacatttttgtcaattggtgggccacatgaacatcacggaattcaaatacagatgtaattctgataagggtgctgttgttctgtacagtgttggagttcatacagtttctgaactaagagcaatgaagaagcggatggaatcttctaagcttaaaacttacaatcttacagaaagtgacttggttaaagatcacttgcgctacctgatgggcggccggtcaaatattgagaatgaggttctttgccgtttcatctttccggagaggcctggtgctctgatgaagttcttggactcctttagtccacgttggaatataagtttgttccattaccgtggacagggtgaaactggtgcaaatgtattggttggtatgcaggtaccatgtagtgacatggatgacttccatgattgtgctaacagacttggatatgagtacacggtggtaaccaatgatgaagatttccagcttttgatgcattaa

SEQ ID NO: 134 - DNA, 1842 bp, Quillaja Saponaria
  source 1..1842: mol_type = other DNA
  misc_feature 1..1842: note = Q. saponaria Threonine Deaminase mutant P540L (Qs0222940.1)
atggagacactgtgttactcgccatctcaggcacctctctttcgccagaagcgtttgggagaattggcgcgtacttctgtccccaaaaccaataattgcagcagagaactacccatcaagcctttcatcactgccacgctatccaaacaggcagaagagatttctcagaagccctctttagctgctacgacggttacttcccgcgaagaccaattaacggtgtctcctttgccttctcgtaaaagggtctcgccggattctctatgttatccatccggttacgttggggccgtcccggaccgtacggtttcctctggaggtgatgatataatcaatgcgatgagttatttgacgaatatactttcttctaaggtttacgatgtggccattgaatcgccgttacagctcgcgacaaaactctctgaacgcttgcgggtgaacatttggctcaagagggaggatttacaacctgttttctcattcaagctccgtggtgcatataatatgatggcaaaacttccgaaggaactgttggaaaaaggggtcatctgctcttcagctggaaatcatgcccaaggggttgcattagcttccaagagactgggctgcagtgctgtgattgttatgcctgttactactccagaaatcaagtggagatctgttgagaagctgggtgcaacagttgttcttgtgggggattcttatgatgaagcacaagcatatgctaagaatcgtggaaatgaagagggccgttctttcatacctccttttgatcatcccgatgtaataatggggcagggaacagttgggatggaaattgttcgccaaatgcaacgtcctgtgcatgcaatctttgttccagtaggaggtggtggactaatcgctggtattgcagcttatgtaaagcgggtctctccagaggtgaaggttattggagttgagccaactgatgcaaatgcaatggcattgtcattacatcatggccagagagtgattttggaccaggtcggaggctttgcagatggtgtagctgttaaggaggttggggaagaaacttttcgcttatgcaaggagttgattgatggtgtggttctcgtaagccgtgattctatttgtgcctcaataaaggacatgtttgaggagaaaaggagcattttagaaccagcaggtgctcttgcccttgctggagctgaagcatactgcaagtattatggtctcaagggagaaaatgtcgtagctataaccagtggggcaaacatgaactttgataaactgagagtggtgactgaactagctaatgttggtcgtaaacaagaagccgtccttgcaactgttttgccagaggagaatggcagttttaaacatttttgtcaattggtgggccacatgaacatcacggaattcaaatacagatgtaattctgataagggtgctgttgttctgtacagtgttggagttcatacagtttctgaactaagagcaatgaagaagcggatggaatcttctaagcttaaaacttacaatcttacagaaagtgacttggttaaagatcacttgcgctacctgatgggcggccggtcaaatattgagaatgaggttctttgccgtttcatctttctggagaggcctggtgctctgatgaagttcttggactcctttagtccacgttggaatataagtttgttccattaccgtggacagggtgaaactggtgcaaatgtattggttggtatgcaggtaccatgtagtgacatggatgacttccatgattgtgctaacagacttggatatgagtacacggtggtaaccaatgatgaagatttccagcttttgatgcattaa

SEQ ID NO: 135 - DNA, 22 bp, synthetic construct
  source 1..22: mol_type = other DNA
  misc_feature 1..22: note = QsTD_P540L_Q5F
ttcatctttctggagaggcctg

SEQ ID NO: 136 - DNA, 19 bp, synthetic construct
  source 1..19: mol_type = other DNA
  misc_feature 1..19: note = QsTD_P540L_Q5R
acggcaaagaacctcattc